(19) United States
(12) Patent Application Publication
(10) Pub. No.: US 2025/0346593 A1
(43) Pub. Date: Nov. 13, 2025

(54) IMIDAZO(1,2-1)PYRIDINE DERIVATIVES AS RIPK2 INHIBITORS

(71) Applicant: Interline Therapeutics, Inc., South San Francisco, CA (US)

(72) Inventors: Mario G. Cardozo, San Francisco, CA (US); Zuojun Guo, Pleasanton, CA (US); Robert Joseph Moreau, Walnut Creek, CA (US); Naomi S. Rajapaksa, Belmont, CA (US); Meera Rao, San Francisco, CA (US); David C. Tully, Danville, CA (US); Steffen Greßies, Berlin (DE); Kai Thede, Berlin (DE); Zachary K. Sweeney, San Francisco, CA (US)

(21) Appl. No.: 18/871,455

(22) PCT Filed: Jun. 9, 2023
(86) PCT No.: PCT/US2023/024988

§ 371 Date: Dec. 3, 2024

Related U.S. Application Data
(60) Provisional application No. 63/351,115, filed on Jun. 10, 2022.

(51) Int. Cl. C07D 471/04; A61K 31/437; A61K 31/444; A61K 31/4545; A61K 31/496; A61K 31/497; A61K 31/506; A61K 31/5377

(52) U.S. Cl. CPC C07D 471/04; A61K 31/437; A61K 31/444; A61K 31/4545; A61K 31/496; A61K 31/497; A61K 31/506; A61K 31/5377

(57) ABSTRACT

The present application relates to compounds of Formula (I), as defined herein, and pharmaceutically acceptable salts thereof, and compositions comprising same. Also described are methods of treating the diseases and disorders disclosed herein, with the compounds of Formula (I), and pharmaceutically acceptable salts thereof, and the compositions comprising same.

### TECHNICAL FIELD

The present application relates to the fields of chemistry and biology, in particular to compounds of Formula (I), as defined herein, and pharmaceutically acceptable salts thereof, and compositions comprising same. Also described are methods of treating the diseases and disorders disclosed herein, with the compounds of Formula (I), and pharmaceutically acceptable salts thereof, and the compositions comprising same.

### BACKGROUND

Receptor interacting protein kinase 2 (RIPK2) is a serine-threonine protein kinase, and is a signaling molecule downstream of nucleotide-binding oligomerization domain 1 (NOD1), NOD2, and Toll-like receptors (TLRs). The RIPK2 protein includes a kinase domain (KD), an intermediate domain (INTD), and a caspase activation and recruitment domain (CARD). The CARD domain of RTPK2 mediates interaction with NOD1 and NOD2. RIPK2 is expressed in the cytoplasm of antigen-presenting cells including dendritic cells and macrophages, and is also expressed in T cells and epithelial cells.

NOD receptors function in the innate immune system, detecting bacterial pathogens by binding to diaminopimelic acid or muramyl dipeptide residues present in bacterial peptidoglycans. Interactions between RIPK2 and NOD1, NOD2 and TLRs trigger the release of pro-inflammatory cytokines including TNF-α, IL-6, and IL-12/23p40, and RIPK2-mediated induction of NF-kappa-B-dependent inflammatory responses. Activation of RIPK2 and dysregulation of the RIPK2-NOD signaling pathways may also have a role in the pathogenesis of various inflammatory diseases. RIPK2 has been reported to be a prognostic indicator and candidate therapeutic target for various cancers.

### SUMMARY

Some embodiments provide a compound of Formula (I):

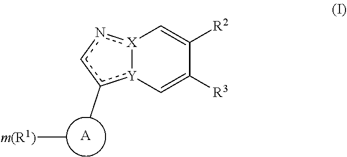

or a pharmaceutically acceptable salt thereof, wherein:
Ring A is phenyl, 5-10 membered heteroaryl, or 5-10 membered heterocyclyl;
one of X and Y is N and the other of X and Y is C;
each [symbol] is a single bond or a double bond, such that the bicyclic ring system of Formula (I) is imidazo[1,2-a]pyridine or pyrazolo[1,5-a]pyridine;
m is 0, 1, 2, 3, or 4;
each R¹ is independently selected from the group consisting of:
   (i) halogen,
   (ii) cyano,
   (iii) C1-C6 alkyl optionally substituted with hydroxyl, C1-C6 alkoxy, or —S(O₂)C1-C6 alkyl,
   (iv) C1-C6 haloalkyl,
   (v) C1-C6 haloalkoxy,
   (vi) C3-C6 cycloalkyl,
   (vii) —NR^{A}R^{B},
   (viii) C1-C6 alkoxy optionally substituted with hydroxyl or phenyl,
   (ix) 4-8 membered heterocyclyl optionally substituted with 1-2 substituents independently selected from the group consisting of hydroxyl, C1-C6 alkyl, and C1-C6 haloalkyl,
   (x) —S(O₂)C1-C6 alkyl,
   (xi) hydroxyl,
   (xii) nitro,
   (xiii) —S(O₂)C3-C6 cycloalkyl, and
   (xiv) when Ring A is phenyl, R¹ is further selected from —S(O₂)NR^{A}R^{A};
R^{A} is hydrogen or C1-C6 alkyl;
R^{B} is
   (i) hydrogen,
   (ii) —S(O₂)C1-C6 alkyl,
   (iii) C3-C6 cycloalkyl optionally substituted with hydroxyl or C1-C6 alkoxy,
   (iv) 4-8 membered heterocyclyl optionally substituted with 1-2 substituents independently selected from hydroxyl and C1-C6 haloalkyl, or
   (v) C1-C6 alkyl optionally substituted with 1-4 substituents independently selected from:
      (a) halogen,
      (b) hydroxyl,
      (c) —NR^{C}R^{D},
      (d) C1-C6 alkoxy,
      (e) C1-C6 haloalkoxy,
      (f) C3-C6 cycloalkyl optionally substituted with hydroxyl,
      (g) phenyl optionally substituted with C1-C6 alkoxy,
      (h) 5-6 membered heteroaryl optionally substituted with C1-C6 alkyl,
      (j) 4-8 membered heterocyclyl optionally substituted with hydroxyl, —C(═O)C1-C6 alkyl, or C1-C6 alkyl,
      (k) C1-C6 thioalkyl, and
      (l) —S(═NR)(═O)C1-C6 alkyl;
R² is hydrogen, halogen, C1-C6 alkoxy, or C1-C6 haloalkoxy;
R³ is
   (i) C1-C6 thioalkyl,

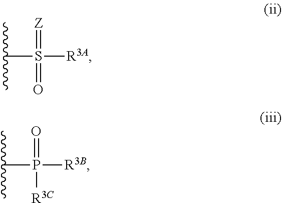

(iv) 4-8 membered heterocyclyl optionally substituted with 1-2 substituents independently selected from halogen and C1-C6 alkyl,
(v) C1-C6 alkyl optionally substituted with NR^{E}R^{F} or hydroxyl,
(vi) —CO₂H,
(vii) —C(═O)NR^{E}R^{F},
(viii) C1-C6 alkoxy optionally substituted with 4-10 membered heterocyclyl optionally substituted with C1-C6 alkoxy,
(ix) hydrogen;
Z is O or NR⁴;
R^{3A} is C1-C6 haloalkyl, C3-C6 cycloalkyl, or C1-C6 alkyl optionally substituted with C3-C6 cycloalkyl;
R^{3B} and R^{3C} are each independently C3-C6 cycloalkyl or C1-C6 alkyl optionally substituted with C3-C6 cycloalkyl;
R⁴ is hydrogen or C1-C6 alkyl; and
each R^{C} and R^{D} are each independently hydrogen, —C(═O)OC1-C6 alkyl, or C1-C6 alkyl optionally substituted with oxo;
each R^{E} and R^{F} are each independently hydrogen or C1-C6 alkyl; and
wherein
(i) when X is C, Y is N, Ring A is 4-pyridyl, R² is C1-C6 alkoxy, R³ is —S(O₂)—C1-C6 alkyl, and m is 1, then R¹ is not —NH₂ or —NH(C═O)CH₃;
(ii) when X is C, Y is N, Ring A is 4-pyridyl, R² is C1-C6 alkoxy, R³ is —S(O₂)—C1-C6 alkyl, m is 2, and one R¹ is fluoro, NH-(p-methoxybenzyl), or —NH₂, then the other R¹ is not halogen;
(iii) when X is C, Y is N, Ring A is phenyl, R² is C1-C6 alkoxy, R³ is —S(O₂)—C1-C6 alkyl, and m is 1, R¹ is not fluoro, cyano, or unsubstituted C1-C6 alkoxy;
(iv) when X is C, Y is N, Ring A is phenyl, R² is C1-C6 alkoxy, R³ is —S(O₂)—C1-C6 alkyl, m is 2, and one R¹ is halogen, then the other R¹ is not unsubstituted C1-C6 alkoxy;
(v) when X is C, Y is N, Ring A is 4-pyridyl, R² is C1-C6 haloalkoxy, R³ is —S(O₂)—C1-C6 alkyl, m is 2, and one R¹ is —NH₂, then the other R¹ is not halogen;
(vi) when X is C, Y is N, R² is C1-C3 alkoxy, R³ is —S(O₂)—C1-C4 alkyl, Ring A is phenyl, and m is 1, R¹ is not halogen, —NH₂, cyano, or unsubstituted C1-C6 alkoxy;
(vii) when X is C, Y is N, R² is ethoxy, R³ is —S(O₂)—C1-C4 alkyl, and m is 2, and one R¹ is halogen, then the other R¹ is not —NH₂ or unsubstituted alkoxy; and
(viii) when X is C, Y is N, R² is C1-C2 haloalkoxy, R³ is —S(O₂)—C1-C4 alkyl, and m is 2, and one R¹ is halogen, then the other R¹ is not —NH₂; and
(ix) when X is C, Y is N, R² is C1-C3 alkoxy, R³ is —S(O₂)—C1-C4 alkyl, and m is 0, then Ring A is not 1H-indazole or 1H-benzo[d]imidazole.

In some embodiments, Formula (I) is:

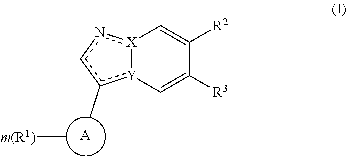

or a pharmaceutically acceptable salt thereof, wherein:
Ring A is phenyl, 5-10 membered heteroaryl, or 5-10 membered heterocyclyl;
one of X and Y is N and the other of X and Y is C;
each [symbol] is a single bond or a double bond, such that the bicyclic ring system of Formula (I) is imidazo[1,2-a]pyridine or pyrazolo[1,5-a]pyridine;
m is 0, 1, 2, or 3;
each R¹ is independently selected from the group consisting of:
   (i) halogen,
   (ii) cyano,
   (iii) C1-C6 alkyl optionally substituted with hydroxyl,
   (iv) C1-C6 haloalkyl,
   (v) C1-C6 haloalkoxy,
   (vi) C3-C6 cycloalkyl,
   (vii) —NR^{A}R^{B}
   (viii) C1-C6 alkoxy optionally substituted with hydroxyl or phenyl, and
   (ix) 4-8 membered heterocyclyl optionally substituted with 1-2 substituents independently selected from the group consisting of hydroxyl, C1-C6 alkyl, and C1-C6 haloalkyl;
R^{A} is hydrogen or C1-C6 alkyl;
R^{B} is
   (i) hydrogen,
   (ii) —S(O₂)C1-C6 alkyl,
   (iii) C3-C6 cycloalkyl optionally substituted with hydroxyl or C1-C6 alkoxy,
   (iv) 4-8 membered heterocyclyl optionally substituted with hydroxyl, or
   (v) C1-C6 alkyl optionally substituted with 1-4 substituents independently selected from: halogen, hydroxyl, —NR^{C}R^{D}, C1-C6 alkoxy, C1-C6 haloalkoxy, C3-C6 cycloalkyl, phenyl optionally substituted with C1-C6 alkoxy, 5-6 membered heteroaryl optionally substituted with C1-C6 alkyl, and 4-8 membered heterocyclyl optionally substituted with —C(═O)C1-C6 alkyl or C1-C6 alkyl;
R² is hydrogen, halogen, C1-C6 alkoxy, or C1-C6 haloalkoxy;
R³ is
   (i) C1-C6 thioalkyl,

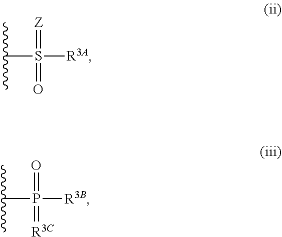

(iv) 4-8 membered heterocyclyl optionally substituted with 1-2 substituents independently selected from halogen and C1-C6 alkyl,
(v) C1-C6 alkyl optionally substituted with NR^{E}R^{F} or hydroxyl,
(vi) —CO₂H,
(vii) —C(═O)NR^{E}R^{F},
(viii) C1-C6 alkoxy optionally substituted with 4-10 membered heterocyclyl optionally substituted with C1-C6 alkoxy, or
(ix) hydrogen;
Z is O or NR⁴;
R^{3A} is C1-C6 haloalkyl, C3-C6 cycloalkyl, or C1-C6 alkyl optionally substituted with C3-C6 cycloalkyl;
R^{3B} and R^{3C} are each independently C3-C6 cycloalkyl or C1-C6 alkyl optionally substituted with C3-C6 cycloalkyl;
R⁴ is hydrogen or C1-C6 alkyl; and
each R^{C} and R^{D} are each independently hydrogen or C1-C6 alkyl optionally substituted with oxo;
each R^{E} and R^{F} are each independently hydrogen or C1-C6 alkyl; and
wherein
(x) when X is C, Y is N, Ring A is 4-pyridyl, R² is C1-C6 alkoxy, R³ is —S(O₂)—C1-C6 alkyl, and m is 1, then R¹ is not —NH₂ or —NH(C═O)CH₃;
(xi) when X is C, Y is N, Ring A is 4-pyridyl, R² is C1-C6 alkoxy, R³ is —S(O₂)—C1-C6 alkyl, m is 2, and one R¹ is fluoro, NH-(p-methoxybenzyl), or —NH₂, then the other R¹ is not halogen;
(xii) when X is C, Y is N, Ring A is phenyl, R² is C1-C6 alkoxy, R³ is —S(O₂)—C1-C6 alkyl, and m is 1, R¹ is not fluoro, cyano, or unsubstituted C1-C6 alkoxy;
(xiii) when X is C, Y is N, Ring A is phenyl, R² is C1-C6 alkoxy, R³ is —S(O₂)—C1-C6 alkyl, m is 2, and one R¹ is halogen, then the other R¹ is not unsubstituted C1-C6 alkoxy;
(xiv) when X is C, Y is N, Ring A is 4-pyridyl, R² is C1-C6 haloalkoxy, R³ is —S(O₂)—C1-C6 alkyl, m is 2, and one R¹ is —NH₂, then the other R¹ is not halogen;
(xv) when X is C, Y is N, R² is C1-C3 alkoxy, R³ is —S(O₂)—C1-C4 alkyl, Ring A is phenyl, and m is 1, R¹ is not halogen, —NH₂, cyano, or unsubstituted C1-C6 alkoxy;
(xvi) when X is C, Y is N, R² is ethoxy, R³ is —S(O₂)—C1-C4 alkyl, and m is 2, and one R¹ is halogen, then the other R¹ is not —NH₂ or unsubstituted alkoxy; and
(xvii) when X is C, Y is N, R² is C1-C2 haloalkoxy, R³ is —S(O₂)—C1-C4 alkyl, and m is 2, and one R¹ is halogen, then the other R¹ is not —NH₂; and
(xviii) when X is C, Y is N, R² is C1-C3 alkoxy, R³ is —S(O₂)—C1-C4 alkyl, and m is 0, then Ring A is not 1H-indazole or 1H-benzo[d]imidazole.

Also provided herein is a pharmaceutical composition comprising a compound of Formula (I), or a pharmaceutically acceptable salt thereof, and at least one pharmaceutically acceptable excipient.

Also provided herein is a method of treating a RIPK2-associated disease or disorder in a subject in need thereof, comprising administering to the subject an effective amount of a compound of Formula (I), or a pharmaceutically acceptable salt thereof, or a pharmaceutical composition comprising subject an effective amount of a compound of Formula (I), or a pharmaceutically acceptable salt thereof.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety, unless expressly indicated otherwise. In case of conflict, the present specification, including definitions, will control.

Other features and advantages of the disclosure will be apparent from the following detailed description and from the claims.

### DETAILED DESCRIPTION

Biologics and small molecules targeting pro-inflammatory signaling pathways have been used to successfully treat inflammatory and other diseases in patients, however, a significant fraction of patients are refractory to existing therapies. Therefore, there exists a need for identification of novel therapeutic molecules that modulate or inhibit these pathways, such as the compounds of Formula (I), and pharmaceutically acceptable salts thereof described herein.

### Definitions

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties. In case of conflict, the present specification, including definitions, will control.

The term “about” when referring to a number or a numerical range means that the number or numerical range referred to is an approximation, for example, within experimental variability and/or statistical experimental error, and thus the number or numerical range may vary up to ±10% of the stated number or numerical range.

The phrase “therapeutically effective amount” means an amount of compound that, when administered to a subject in need of such treatment, is sufficient to (i) treat a disease or disorder as described herein (e.g., a RIPK2-associated disease or disorder), (ii) attenuate, ameliorate, or eliminate one or more symptoms of the particular disease or disorder, or (iii) delay the onset of one or more symptoms of the particular disease or disorder described herein.

As used herein, terms “treat” or “treatment” refer to therapeutic or palliative measures. Beneficial or desired clinical results include, but are not limited to, alleviation, in whole or in part, of symptoms associated with a disease or disorder, diminishment of the extent of a neurological disorder, stabilized (i.e., not worsening) state of a disease or disorder, delay or slowing of disease progression, amelioration or palliation of the disease state (e.g., one or more symptoms of the disease or disorder), and remission (whether partial or total), whether detectable or undetectable and can be determined by various clinical assessments including clinical evaluation and self-reporting. “Treatment” can also mean prolonging survival as compared to expected survival if not receiving treatment.

The term “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described hereinform with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid, and organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “pharmaceutically acceptable excipients”), such as stabilizers, diluents, dispersing agents, suspending agents, thickening agents, and/or other excipients. The pharmaceutical composition facilitates administration of the compound to an organism.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The term “halo” or “halogen” refers to one of the halogens, group 17 of the periodic table. In particular the term refers to fluorine, chlorine, bromine and iodine. Preferably, the term refers to fluorine or chlorine.

The term “oxo” refers to a divalent doubly bonded oxygen atom (i.e., “═O”). As used herein, oxo groups are attached to carbon atoms to form carbonyls.

The term “alkyl” refers to a saturated acyclic hydrocarbon radical that may be a straight chain or branched, containing the indicated number of carbon atoms. For example, C₁₋₁₀ indicates that the group may have from 1 to 10 (inclusive) carbon atoms in it. Non-limiting examples include methyl, ethyl, iso-propyl, tert-butyl, n-hexyl.

The term “alkenyl” refers to an acyclic hydrocarbon radical that may be a straight chain or branched, containing the indicated number of carbon atoms and one or more carbon-carbon double bonds. Non-limiting examples include ethylenyl and allyl.

The term “haloalkyl” refers to an alkyl, in which one or more hydrogen atoms is/are replaced with an independently selected halogen.

The term “hydroxyalkyl” refers to an alkyl group as described herein, in which one or more hydrogen atoms is/are replaced with one or more hydroxyl groups, as described herein.

The term “alkoxy” refers to an —O-alkyl radical (e.g., —OCH₃).

The term “thioalkyl” refers to an alkyl group as described herein, which is attached to a molecule via a sulfur atom (e.g., —SCH₃).

The term “haloalkoxy” refers to a haloalkyl group which is attached to a molecule via an oxygen atom (e.g., —OCF₃).

The term “alkoxyalkyl” refers to an alkyl group as described herein, in which one or more hydrogen atoms is/are replaced with one or more alkoxy groups as described herein.

As used herein, the term “cyano” refers to a —CN radical.

As used herein, the term “nitro” refers to a —NO₂ radical.

As used herein, the term “hydroxyl” refers to an —OH radical.

As used herein, the term “amino” refers to a —NH₂ radical.

As used herein, the term “phosphate” refers to a —P(═O)₂(OH)₂ radical.

As used herein, the term “heteroaryl” refers to a 5-14 membered mono-, bi-, or tricyclic group wherein at least one ring in the system is aromatic; and wherein one or more carbon atoms in at least one ring in the system is/are replaced with an heteroatom independently selected from the group consisting of N, O, S, B, Si, and P. For example, there may be 1, 2 or 3 heteroatoms, optionally 1 or 2. A heteroaryl may further contain one or more oxo, N-oxide, S-oxide, and/or S,S-dioxide groups, valence permitting. Non-limiting examples of heteroaryl groups include furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, 2-pyridone, pyridazine, pyrimidine, pyrazine, purine, pteridine, quinoline, isoquinoline, quinazoline, quinoxaline, cinnoline, triazine, 2,3-dihydro-1H-pyrrolo[2,3-b]pyridine, 3,4-dihydro-2H-pyrido[3,2-b][1,4]oxazine, 1′,2′-dihydrospiro[cyclopropane-1,3′-pyrrolo[2,3-b]pyridine], and 3′,4′-dihydrospiro [cyclopropane-1,2′-pyrido[3,2-b][1,4]oxazine].

For purposes of clarification, heteroaryl also includes aromatic lactams, aromatic cyclic ureas, or vinylogous analogs thereof, in which each ring nitrogen adjacent to a carbonyl is tertiary (i.e., all three valences are occupied by non-hydrogen substituents), such as one or more of pyridone

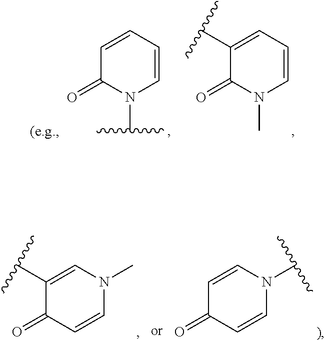

pyrimidone

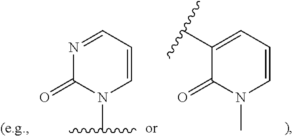

pyridazinone

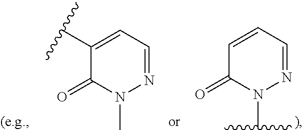

pyrazinone

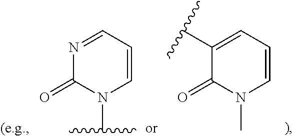

and imidazolone

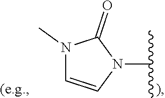 [SMILES: *n1ccn(C)c1=O]

wherein each ring nitrogen adjacent to a carbonyl is tertiary (i.e., the oxo group (i.e., “═O”) herein is a constituent part of the heteroaryl ring).

As used herein, the term “cycloalkyl” refers to a saturated or partially unsaturated mono-, bi-, or tricyclic carbon group having 3 to 20 carbon atoms. Bicyclic and tricyclic cycloalkyl groups include fused, spiro, and bridged ring systems. Non-limiting examples of cycloalkyl groups include cyclopropyl, cyclohexyl, spiro[2.3]hexyl, and bicyclo[1.1.1]pentyl.

The term “aryl” refers to a 6-20 carbon mono-, bi-, tri- or polycyclic group wherein at least one ring in the system is aromatic (e.g., 6-carbon monocyclic, 10-carbon bicyclic, or 14-carbon tricyclic aromatic ring system. Examples of aryl groups include phenyl, naphthyl, tetrahydronaphthyl, and the like.

The term “heterocyclyl” refers to a saturated or partially unsaturated hydrocarbon monocyclic, bicyclic, or tricyclic ring system having from 3 to 20 ring atoms, that is not aromatic, and having at least one heteroatom within the ring system selected from the group consisting of N, O, S, B, Si, and P. Bicyclic and tricyclic heterocyclyl groups include fused, spiro, and bridged ring systems. A heterocyclyl group may be denoted as a “5 to 10 membered heterocyclyl group,” which is a ring system containing 5, 6, 7, 8, 9 or 10 atoms at least one being a heteroatom. A heterocycle may further contain one or more oxo, thiocarbonyl, N-oxide, S-oxide, and/or S,S-dioxide groups, valence permitting, so as to make the definition include oxo-systems and thio-systems such as lactams, lactones, cyclic imides, cyclic thioimides and cyclic carbamates. A heterocyclyl group may be bonded to the rest of the molecule through any carbon atom or through a heteroatom such as nitrogen. Exemplary heterocyclyl groups include, but are not limited to 1,3-dioxolane, 1,4-dioxolane, maleimide, succinimide, dioxopiperazine, hydantoin, imidazoline, imidazolidine, isoxazoline, isoxazolidine, oxazoline, oxazolidine, oxazolidinone, thiazoline, thiazolidine, morpholine, oxirane, piperidine N-oxide, piperidine, piperazine, pyrrolidine, pyrrolidone, pyrrolidione, 4-piperidone, pyrazoline, pyrazolidine, 2-oxopyrrolidine, pyrrolidinyl, tetrahydrofuryl, thiolanyl, pyrazolinyl, oxathiolanyl, isoxazolidinyl, isothiazolidinyl, pyrrolinyl, pyrrolidinonyl, pyrazolidinyl, imidazolinyl, dioxolanyl, sulfolanyl, thiazolidedionyl, succinimidyl, dihydrofuranonyl, pyrazolidinonyl, oxazolidinyl, isoxazolidinonyl, hydantionyl, thiohydantionyl, imidazolidinonyl, oxazolidinonyl, thiazolidinonyl, oxathiolanonyl, dioxolanonyl, dioxazolidinonyl, oxadiazolidinonyl, triazolidinonyl, triazolidinethionyl, oxadiazolidinethionyl, dioxazolidinethionyl, dioxolanethionyl, oxazolidinethionyl, imidazolidinethionyl, isothiazolidinonyl, piperidinyl, tetrahydropyranyl, thianyl, morpholinyl, thiomorpholinyl, dioxanyl, piperazinyl, dithianyl, oxazinyl, tetrahydropyranonyl, piperidinonyl, dioxanonyl, oxazinanonyl, morpholinonyl, thiomorpholinonyl, piperazinonyl, tetrahydropyrimidinonyl, piperidinedionyl, oxazinanedionyl, dihydropyrimidindione, tetrahydropyridazinonyl, triazinanonyl, oxadiazinanonyl, dioxazinanonyl, morpholinedionyl, piperazinedionyl, piperazinetrionyl, triazinanedionyl and 2-azaspiro[3.3]heptanyl.

The term “saturated” as used in this context means only single bonds present between constituent atoms.

As used herein, when a ring is described as being “partially unsaturated”, it means said ring has one or more additional degrees of unsaturation (in addition to the degree of unsaturation attributed to the ring itself, e.g., one or more double or triple bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

As used herein, a squiggly line depicts the point of attachment of an atom or moiety to the indicated atom or group in the remainder of the molecule.

Whenever a group is described as being “optionally substituted” that group may be unsubstituted or substituted with one or more of the indicated substituents. When a group is substituted, that substitution can include the sharing of a carbon atom between the parent group and the substitution to form a spiro ring. For example, an n-butyl group substituted with cyclopropyl includes both

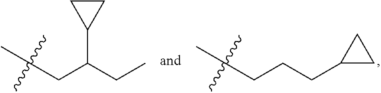

amongst others.

For the avoidance of doubt, and unless otherwise specified, for rings and cyclic groups (e.g., carbocycle, aryl, cycloalkyl, heterocyclyl, heteroaryl, and the like described herein) containing a sufficient number of ring atoms to form bicyclic or higher order ring systems (e.g., tricyclic, polycyclic ring systems), it is understood that such rings and cyclic groups encompass those having fused rings, including those in which the points of fusion are located (i) on adjacent ring atoms (e.g., [x.x.0] ring systems, in which 0 represents a zero atom bridge

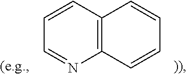

(ii) a single ring atom (spiro-fused ring systems)

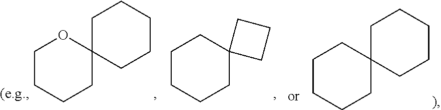

or (iii) a contiguous array of ring atoms (bridged ring systems having all bridge lengths >0)

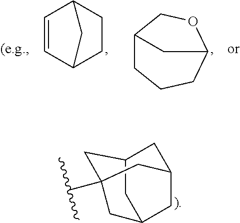

In addition, any compound or structure given herein, is also intended to represent unlabeled forms as well as isotopically labeled forms of the compounds. These forms of compounds are referred to as “isotopically enriched.” Isotopically enriched compounds have structures depicted herein, except that one or more atoms are replaced by an atom having a selected atomic mass or mass number.

Examples of isotopes that can be incorporated into the disclosed compounds include isotopes of hydrogen, carbon, nitrogen, oxygen, phosphorous, fluorine, chlorine and iodine, such as ²H, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Various isotopically enriched compounds of the present disclosure, for example those into which radioactive isotopes such as ¹³C and ¹⁴C are incorporated. Such isotopically enriched compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The term“isotopically enriched” compounds includes“deuterated” compounds described herein in which one or more hydrogens is/are replaced by deuterium, such as a hydrogen on a carbon atom. Such compounds exhibit increased resistance to metabolism and are thus useful for increasing the half-life of any compound when administered to a mammal, particularly a human. Such compounds are synthesized by means known in the art, for example by employing starting materials in which one or more hydrogens have been replaced by deuterium. Indeed, isotopically enriched compounds of this disclosure can generally be prepared by carrying out the procedures disclosed in the schemes or in the examples and preparations described below by substituting a readily available isotopically enriched reagent for a non-isotopically enriched reagent.

Deuterium enriched compounds of the present disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index relative to the corresponding non-enriched compound.

The concentration of a heavier isotope, such as deuterium, may be defined by an isotopic enrichment factor. In some embodiments, the positions noted as “H” or “hydrogen” in the compounds described herein have hydrogen at its natural abundance isotopic composition. In some embodiments, the positions noted as “H” or “hydrogen” in the compounds described herein have hydrogen enriched in deuterium above its natural abundance isotopic composition, i.e., the compound is a deuterium enriched compound. Examples of deaerated groups in the compounds described herein include, but are not limited to deuteromethine

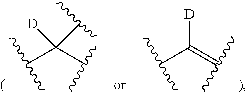

monodeuteromethylene

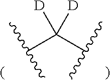 [SMILES: *C(*)([2H])[2H]]

and dideuteromethylene

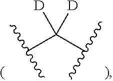 [SMILES: *C(*)([2H])[2H]]

trideuteromethyl

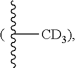

trideuteromethoxy

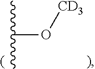

and the like. Compounds of the present disclosure also include deuterium enriched compounds at the alpha position of an oxo group, such as

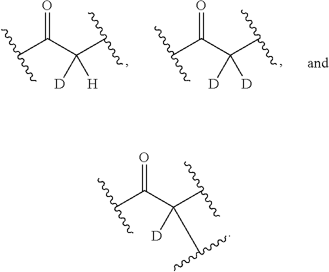 [SMILES: *C(=O)C(*)[2H] ; *C(=O)C(*)([2H])[2H] ; *C(=O)C(*)(*)[2H]]

In addition, the compounds generically or specifically disclosed herein are intended to include all tautomeric forms. Thus, by way of example, a compound containing the moiety:

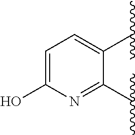 [SMILES: *c1ccc(O)nc1*]

encompasses the tautomeric form containing the moiety:

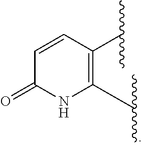 [SMILES: *c1ccc(=O)[nH]c1*]

Similarly, a pyridinyl or pyrimidinyl moiety that is described to be optionally substituted with hydroxyl encompasses pyridone or pyrimidone tautomeric forms.

The compounds provided herein may encompass various stereochemical forms. The compounds also encompass enantiomers (e.g., R and S isomers), diastereomers, as well as mixtures of enantiomers (e.g., R and S isomers) including racemic mixtures and mixtures of diastereomers, as well as individual enantiomers and diastereomers, which arise as a consequence of structural asymmetry in certain compounds. Unless otherwise indicated, when a disclosed compound is named or depicted by a structure without specifying the stereochemistry (e.g., a “flat” structure) and has one or more chiral centers, it is understood to represent all possible stereoisomers of the compound. Likewise, unless otherwise indicated, when a disclosed compound is named or depicted by a structure that specifies the stereochemistry (e.g., a structure with “wedge” and/or “dashed” bonds) and has one or more chiral centers, it is understood to represent the indicated stereoisomer of the compound.

The details of one or more embodiments of this disclosure are set forth in the accompanying drawings and the description below. Other features and advantages of the present disclosure will be apparent from the description and from the claims.

A “RIPK2 inhibitor” as defined herein includes any compound exhibiting RIPK2 inhibition activity. In some embodiments, a RIPK2 inhibitor is selective for RIPK2. Exemplary RIPK2 inhibitors can exhibit inhibition activity (IC₅₀) against a RIPK2 of less than about 1000 nM, less than about 500 nM, less than about 200 nM, less than about 100 nM, less than about 50 nM, less than about 25 nM, less than about 10 nM, less than about 5 nM, or less than about 1 nM as measured in an assay as described herein. In some embodiments, a RIPK2 inhibitor can exhibit inhibition activity (IC₅₀) against RIPK2 of less than about 25 nM, less than about 10 nM, less than about 5 nM, or less than about 1 nM as measured in an assay as provided herein.

### Compounds of Formula (I)

Some embodiments provide a compound of Formula (I):

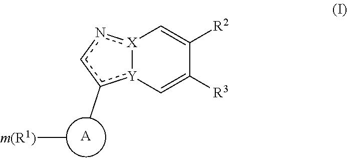

or a pharmaceutically acceptable salt thereof, wherein:
Ring A is phenyl, 5-10 membered heteroaryl, or 5-10 membered heterocyclyl;
one of X and Y is N and the other of X and Y is C;
each [symbol] is a single bond or a double bond, such that the bicyclic ring system of Formula (I) is imidazo[1,2-a]pyridine or pyrazolo[1,5-a]pyridine;
m is 0, 1, 2, 3, or 4;
each R¹ is independently selected from the group consisting of:
   (i) halogen,
   (ii) cyano,
   (iii) C1-C6 alkyl optionally substituted with hydroxyl, C1-C6 alkoxy, or —S(O₂)C1-C6 alkyl,
   (iv) C1-C6 haloalkyl,
   (v) C1-C6 haloalkoxy,
   (vi) C3-C6 cycloalkyl,
   (vii) —NR^{A}R^{B}
   (viii) C1-C6 alkoxy optionally substituted with hydroxyl or phenyl,
   (ix) 4-8 membered heterocyclyl optionally substituted with 1-2 substituents independently selected from the group consisting of hydroxyl, C1-C6 alkyl, and C1-C6 haloalkyl,
   (x) —S(O₂)C1-C6 alkyl,
   (xi) hydroxyl,
   (xii) nitro,
   (xiii) —S(O₂)C3-C6 cycloalkyl, and
   (xiv) when Ring A is phenyl, R¹ is further selected from —S(O₂)NR^{A}R^{A};
R^{A} is hydrogen or C1-C6 alkyl;
R^{B} is
   (i) hydrogen,
   (ii) —S(O₂)C1-C6 alkyl,
   (iii) C3-C6 cycloalkyl optionally substituted with hydroxyl or C1-C6 alkoxy,
   (iv) 4-8 membered heterocyclyl optionally substituted with 1-2 substituents independently selected from hydroxyl and C1-C6 haloalkyl, or
   (v) C1-C6 alkyl optionally substituted with 1-4 substituents independently selected from:
      (a) halogen,
      (b) hydroxyl,
      (c) —NR^{C}R^{D},
      (d) C1-C6 alkoxy,
      (e) C1-C6 haloalkoxy,
      (f) C3-C6 cycloalkyl optionally substituted with hydroxyl,
      (g) phenyl optionally substituted with C1-C6 alkoxy,
      (h) 5-6 membered heteroaryl optionally substituted with C1-C6 alkyl,
      (j) 4-8 membered heterocyclyl optionally substituted with hydroxyl, —C(═O)C1-C6 alkyl, or C1-C6 alkyl,
      (k) C1-C6 thioalkyl, and
      (l) —S(═NR)(═O)C1-C6 alkyl;
R² is hydrogen, halogen, C1-C6 alkoxy, or C1-C6 haloalkoxy;
R³ is
   (i) C1-C6 thioalkyl,

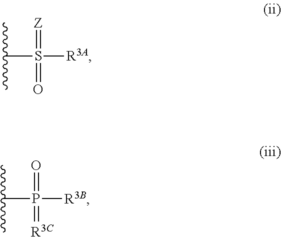

(iv) 4-8 membered heterocyclyl optionally substituted with 1-2 substituents independently selected from halogen and C1-C6 alkyl,
(v) C1-C6 alkyl optionally substituted with NR^{E}R^{F} or hydroxyl,
(vi) —CO₂H,
(vii) —C(═O)NR^{E}R^{F},
(viii) C1-C6 alkoxy optionally substituted with 4-10 membered heterocyclyl optionally substituted with C1-C6 alkoxy,
(ix) hydrogen;
Z is O or NR⁴;
R^{3A} is C1-C6 haloalkyl, C3-C6 cycloalkyl, or C1-C6 alkyl optionally substituted with C3-C6 cycloalkyl;
R^{3B} and R^{3C} are each independently C3-C6 cycloalkyl or C1-C6 alkyl optionally substituted with C3-C6 cycloalkyl;
R⁴ is hydrogen or C1-C6 alkyl; and
each R^{C} and R^{D} are each independently hydrogen, —C(═O)OC1-C6 alkyl, or C1-C6 alkyl optionally substituted with oxo;
each R^{E} and R^{F} are each independently hydrogen or C1-C6 alkyl; and
wherein
when X is C, Y is N, Ring A is 4-pyridyl, R² is C1-C6 alkoxy, R³ is —S(O₂)—C1-C6 alkyl, and m is 1, then R¹ is not —NH₂ or —NH(C═O)CH₃;
when X is C, Y is N, Ring A is 4-pyridyl, R² is C1-C6 alkoxy, R³ is —S(O₂)—C1-C6 alkyl, m is 2, and one R¹ is fluoro, NH-(p-methoxybenzyl), or —NH₂, then the other R¹ is not halogen;
when X is C, Y is N, Ring A is phenyl, R² is C1-C6 alkoxy, R³ is —S(O₂)—C1-C6 alkyl, and m is 1, R¹ is not fluoro, cyano, or unsubstituted C1-C6 alkoxy;
when X is C, Y is N, Ring A is phenyl, R² is C1-C6 alkoxy, R³ is —S(O₂)—C1-C6 alkyl, m is 2, and one R¹ is halogen, then the other R¹ is not unsubstituted C1-C6 alkoxy;
when X is C, Y is N, Ring A is 4-pyridyl, R² is C1-C6 haloalkoxy, R³ is —S(O₂)—C1-C6 alkyl, m is 2, and one R¹ is —NH₂, then the other R¹ is not halogen;
when X is C, Y is N, R² is C1-C3 alkoxy, R³ is —S(O₂)—C1-C4 alkyl, Ring A is phenyl, and m is 1, R¹ is not halogen, —NH₂, cyano, or unsubstituted C1-C6 alkoxy;
when X is C, Y is N, R² is ethoxy, R³ is —S(O₂)—C1-C4 alkyl, and m is 2, and one R¹ is halogen, then the other R¹ is not —NH₂ or unsubstituted alkoxy; and
when X is C, Y is N, R² is C1-C2 haloalkoxy, R³ is —S(O₂)—C1-C4 alkyl, and m is 2, and one R¹ is halogen, then the other R¹ is not —NH₂; and
when X is C, Y is N, R² is C1-C3 alkoxy, R³ is —S(O₂)—C1-C4 alkyl, and m is 0, then Ring A is not 1H-indazole or 1H-benzo[d]imidazole.

In some embodiments, X is C, Y is N, and the bicyclic ring system of Formula (I) is imidazo[1,2-a]pyridine.

In some embodiments, X is N, Y is C, and the bicyclic ring system of Formula (I) is pyrazolo[1,5-a]pyridine.

In some embodiments, Ring A is a 5-10 membered heteroaryl. In some embodiments, Ring A is selected from the group consisting of indazolyl, indolyl, benzimidazolyl, azaindolyl, benzofuranyl, benzothiophenyl, benzoxazolyl, benzothiazolyl, quinolinyl, or isoquinolinyl. In some embodiments, Ring A is indazolyl.

In some embodiments, Ring A is a 5-6 membered heteroaryl. In some embodiments, Ring A is selected from the group consisting of pyrrolyl, pyrazolyl, imidazolyl, triazolyl, tetrazolyl, furanyl, thiophenyl, oxazolyl, isoxazolyl, isothiazolyl, thiazolyl, pyridinyl, pyrimidinyl, pyrazinyl, pyridazinyl, and pyridonyl.

In some embodiments, Ring A is pyridinyl. In some embodiments, Ring A is 2-pyridinyl or 3-pyridinyl. In some embodiments, Ring A is 4-pyridinyl.

In some embodiments, Ring A is

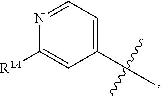

wherein R^{1A} is independently selected from R¹. In some embodiments, Ring A is

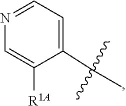

wherein R^{1A} is independently selected from R. In some embodiments, Ring A is

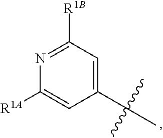

wherein R^{1A} and R^{1B} are each independently selected from R¹. In some embodiments, Ring A is

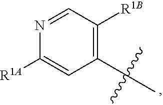

wherein R^{1A} and R^{1B} are each independently selected from R¹. In some embodiments, Ring A is

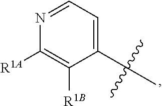

wherein R^{1A} and R^{1B} are each independently selected from R¹. In some embodiments, Ring A

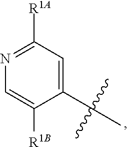

wherein R^{1A} and R^{1B} are each independently selected from R¹.

In some embodiments, Ring A is

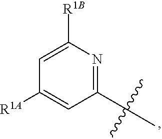

wherein R^{1A} and R^{1B} are each independently selected from R¹. In some embodiments, Ring A is

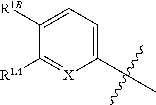

wherein R^{1A} and R^{1B} are each independently selected from R¹. In some embodiments, Ring A is

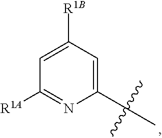

wherein R^{1A} and R^{1B} are each independently selected from R¹. In some embodiments, Ring A is

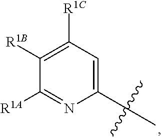

wherein R^{1A}, R^{1B}, and R^{1C} are each independently selected from R¹.
In some embodiments, Ring A is

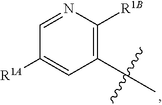

wherein R^{1A} and R^{1B} are each independently selected from R¹. In some embodiments, Ring A is

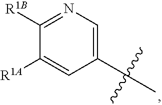

wherein R^{1A} and R^{1B} are each independently selected from R¹. In some embodiments, Ring A is

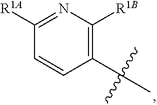

wherein R^{1A} and R^{1B} are each independently selected from R¹.

In some embodiments, Ring A is pyridonyl. In some embodiments, Ring A is selected from the group consisting of 4-pyridonyl, 5-pyridonyl, and 6-pyridonyl.

In some embodiments, Ring A is pyrazolyl. In some embodiments, Ring A is 1-pyrazolyl (i.e., the pyrazolyl is linked to the remaining portion of Formula (I) at the 1-position nitrogen). In some embodiments, Ring A is 3-pyrazolyl. In some embodiments, Ring A is 4-pyrazolyl. In some embodiments, Ring A is 5-pyrazolyl. In some embodiments, Ring A is oxazolonyl.

In some embodiments, Ring A is a 5-10 membered heterocyclyl. In some embodiments, Ring A is 5-6 membered heterocyclyl. In some embodiments, Ring A is selected from the group consisting of pyrrolidinyl, tetrahydrofuryl, pyrazolinyl, imidazolinyl, piperidinyl, tetrahydropyranyl, and morpholinyl.

In some embodiments, Ring A is pyrrolidinyl. In some embodiments, Ring A is 1-pyrrolidinyl (i.e., the pyrrolidinyl is linked to the remaining portion of Formula (I) at the 1-position nitrogen). In some embodiments, Ring A is morpholinyl. In some embodiments, Ring A is 1-morpholinyl (i.e., the morpholinyl is linked to the remaining portion of Formula (I) at the 1-position nitrogen).

In some embodiments, Ring A is phenyl. In some embodiments, Ring A is

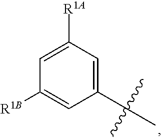

wherein R^{1A} and R^{1B} are each independently selected from R¹. In some embodiments, Ring A is

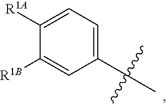

wherein R^{1A} and R^{1B} are each independently selected from R¹. In some embodiments, Ring A is

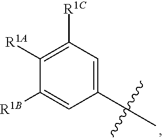

wherein R^{1A}, R^{1B} and R^{1C} are each independently selected from R¹.

In some embodiments, 1-3 R¹ are independently halogen. In some embodiments, 1 or 2 R¹ are independently halogen. In some embodiments, one R¹ is halogen. In some embodiments, one R¹ is fluoro. In some embodiments, one R¹ is chloro.

In some embodiments, one R¹ is cyano.

In some embodiments, one or two R¹ is independently C1-C6 alkyl optionally substituted with hydroxyl, C1-C6 alkoxy, or —S(O₂)C1-C6 alkyl. In some embodiments, one R¹ is C1-C6 alkyl optionally substituted with hydroxyl, C1-C6 alkoxy, or —S(O₂)C1-C6 alkyl. In some embodiments, one R¹ is C1-C6 alkyl substituted with hydroxyl. In some embodiments, one R¹ is C1-C3 alkyl substituted with hydroxyl. In some embodiments, one R¹ is hydroxymethyl or hydroxyethyl. In some embodiments, one R¹ is hydroxypropyl. In some embodiments, one R¹ is C1-C6 alkyl substituted with C1-C6 alkoxy. In some embodiments, one R¹ is C1-C3 alkyl substituted with C1-C6 alkoxy. In some embodiments, one R¹ is C1-C6 alkyl substituted with methoxy. In some embodiments, one R¹ is C1-C6 alkyl substituted with —S(O₂)C1-C6 alkyl. In some embodiments, one R¹ is C1-C6 alkyl substituted with —S(O₂)CH₃ alkyl. In some embodiments, one R¹ is unsubstituted C1-C6 alkyl. In some embodiments, one R¹ is unsubstituted C1-C3 alkyl. In some embodiments, one R¹ is methyl or ethyl.

In some embodiments, one or two R¹ is independently C1-C6 haloalkyl. In some embodiments, one R¹ is C1-C6 haloalkyl. In some embodiments, one R¹ is C1-C3 haloalkyl. In some embodiments, one R¹ is trifluoromethyl.

In some embodiments, one or two R¹ is independently C1-C6 haloalkoxy. In some embodiments, one R¹ is C1-C6 haloalkoxy. In some embodiments, one R¹ is C1-C3 haloalkoxy. In some embodiments, one R¹ is trifluoromethoxy.

In some embodiments, one or two R¹ is independently C3-C6 cycloalkyl. In some embodiments, one R¹ is C3-C6 cycloalkyl. In some embodiments, one R¹ is cyclopropyl.

In some embodiments, one or two R¹ is independently C1-C6 alkoxy optionally substituted with hydroxyl or phenyl. In some embodiments, one R¹ is C1-C6 alkoxy optionally substituted with hydroxyl or phenyl. In some embodiments, one or two R¹ is C1-C6 alkoxy substituted with hydroxyl. In some embodiments, one R¹ is C1-C6 alkoxy substituted with hydroxyl. In some embodiments, one R¹ is C1-C3 alkoxy substituted with hydroxyl. In some embodiments, one R¹ is —O(CH₂)₃OH. In some embodiments, one R¹ is C1-C6 alkoxy substituted with phenyl. In some embodiments, one R¹ is C1-C3 alkoxy substituted with phenyl. In some embodiments, one R¹ is —OCH₂— phenyl.

In some embodiments, one or two R¹ is independently unsubstituted C1-C6 alkoxy. In some embodiments, one R¹ is unsubstituted C1-C6 alkoxy. In some embodiments, two R¹ are independently unsubstituted C1-C6 alkoxy. In some embodiments, one R¹ is unsubstituted C1-C3 alkoxy. In some embodiments, one R¹ is methoxy.

In some embodiments, one R¹ is 4-8 membered heterocyclyl optionally substituted with 1-2 substituents independently selected from the group consisting of hydroxyl, C1-C6 alkyl, and C1-C6 haloalkyl. In some embodiments, one R¹ is 4-8 membered heterocyclyl substituted with 1 substituent selected from the group consisting of hydroxyl, C1-C6 alkyl, and C1-C6 haloalkyl. In some embodiments, one R¹ is 4-8 membered heterocyclyl substituted with 2 substituents independently selected from the group consisting of hydroxyl, C1-C6 alkyl, and C1-C6 haloalkyl.

In some embodiments, one R¹ is 4-8 membered heterocyclyl substituted with hydroxyl.

In some embodiments, one R¹ is 4-8 membered heterocyclyl substituted with C1-C6 alkyl. In some embodiments, one R¹ is 4-8 membered heterocyclyl substituted with C1-C3 alkyl. In some embodiments, one R¹ is 4-8 membered heterocyclyl substituted with methyl.

In some embodiments, one R¹ is 4-8 membered heterocyclyl substituted with C1-C6 haloalkyl. In some embodiments, one R¹ is 4-8 membered heterocyclyl substituted with C1-C3 haloalkyl. In some embodiments, one R¹ is 4-8 membered heterocyclyl substituted with trifluoromethyl.

In some embodiments, one R¹ is 4-8 membered heterocyclyl substituted with hydroxyl and C1-C6 alkyl. In some embodiments, one R¹ is 4-8 membered heterocyclyl substituted with hydroxyl and C1-C3 alkyl. In some embodiments, one R¹ is 4-8 membered heterocyclyl substituted with hydroxyl and methyl.

In some embodiments, one R¹ is 4-8 membered heterocyclyl substituted with hydroxyl and C1-C6 haloalkyl. In some embodiments, one R¹ is 4-8 membered heterocyclyl substituted with hydroxyl and C1-C3 haloalkyl. In some embodiments, one R¹ is 4-8 membered heterocyclyl substituted with hydroxyl and trifluoromethyl.

In some embodiments, one R¹ is 4-8 membered heterocyclyl substituted with C1-C6 alkyl and C1-C6 haloalkyl. In some embodiments, one R¹ is 4-8 membered heterocyclyl substituted with C1-C3 alkyl and C1-C3 haloalkyl. In some embodiments, one R¹ is 4-8 membered heterocyclyl substituted with methyl and trifluoromethyl.

In some embodiments, one R¹ is 4-8 membered heterocyclyl substituted with two hydroxyl. In some embodiments, one R¹ is 4-8 membered heterocyclyl substituted with two independently selected C1-C6 alkyl. In some embodiments, one R¹ is 4-8 membered heterocyclyl substituted with two independently selected C1-C3 alkyl. In some embodiments, one R¹ is 4-8 membered heterocyclyl substituted with two independently selected C1-C6 haloalkyl. In some embodiments, one R¹ is 4-8 membered heterocyclyl substituted with two independently selected C1-C3 haloalkyl.

In some embodiments, one R¹ is piperidinyl substituted with hydroxyl. In some embodiments, one R¹ is 2-azaspiro[3.3]heptan-2-yl substituted with hydroxyl. In some embodiments, one R¹ is 2-azaspiro[3.3]heptan-2-yl substituted with hydroxyl and trifluoromethyl. In some embodiments, one R¹ is piperazinyl substituted with methyl. In some embodiments, one R¹ is 4-methylpiperazin-1-yl.

In some embodiments, one R¹ is unsubstituted 4-8 membered heterocyclyl. In some embodiments, one R¹ is morpholinyl or 2,6-diazaspiro[3.3]heptan-2-yl. In some embodiments, one R¹ is morpholinyl. In some embodiments, one R¹ is 2,6-diazaspiro[3.3]heptan-2-yl.

In some embodiments, one R¹ is —S(O₂)C1-C6 alkyl. In some embodiments, one R¹ is —S(O₂)C1-C3 alkyl. In some embodiments, one R¹ is —S(O₂)CH₃.

In some embodiments, one R¹ is hydroxyl.

In some embodiments, one R¹ is —NR^{A}R^{B}.

In some embodiments, one R¹ is nitro.

In some embodiments, one R¹ is —S(O₂)C3-C6 cycloalkyl.

In some embodiments, when Ring A is phenyl, R¹ is further selected from —S(O₂)NR^{A}R^{A}. In some embodiments, when Ring A is phenyl, R is —S(O₂)NR^{A}R^{A}.

In some embodiments, R^{A} is hydrogen.

In some embodiments, R^{A} is C1-C6 alkyl. In some embodiments, R^{A} is methyl.

In some embodiments, R^{A} is hydrogen and R^{B} is
(i) hydrogen,
(ii) —S(O₂)C1-C6 alkyl,
(iii) C3-C6 cycloalkyl optionally substituted with hydroxyl or C1-C6 alkoxy,
(iv) 4-8 membered heterocyclyl optionally substituted with 1-2 substituents independently selected from hydroxyl and C1-C6 haloalkyl, or
(v) C1-C6 alkyl optionally substituted with 1-4 substituents independently selected from:
   (a) halogen,
   (b) hydroxyl,
   (c) —NR^{C}R^{D},
   (d) C1-C6 alkoxy,
   (e) C1-C6 haloalkoxy,
   (f) C3-C6 cycloalkyl optionally substituted with hydroxyl,
   (g) phenyl optionally substituted with C1-C6 alkoxy,
   (h) 5-6 membered heteroaryl optionally substituted with C1-C6 alkyl,
   (j) 4-8 membered heterocyclyl optionally substituted with hydroxyl, —C(═O)C1-C6 alkyl, or C1-C6 alkyl,
   (k) C1-C6 thioalkyl, and
   (l) —S(═NR^{E})(═O)C1-C6 alkyl.

In some embodiments, R^{A} is hydrogen and R^{B} is selected from the group consisting of: (ii) —S(O₂)C1-C6 alkyl, (iii) C3-C6 cycloalkyl optionally substituted with hydroxyl or C1-C6 alkoxy, (iv) 4-8 membered heterocyclyl optionally substituted with 1-2 substituents independently selected from hydroxyl and C1-C6 haloalkyl, and (v) C1-C6 alkyl optionally substituted with 1-4 substituents independently selected from: halogen, hydroxyl, —NR^{C}R^{D}, C1-C6 alkoxy, C1-C6 haloalkoxy, C3-C6 cycloalkyl, phenyl optionally substituted with C1-C6 alkoxy, 5-6 membered heteroaryl optionally substituted with C1-C6 alkyl, and 4-8 membered heterocyclyl optionally substituted with —C(═O)C1-C6 alkyl or C1-C6 alkyl.

In some embodiments, R^{A} is C1-C6 alkyl and R^{B} is selected from the group consisting of (ii) —S(O₂)C1-C6 alkyl, (iii) C3-C6 cycloalkyl optionally substituted with hydroxyl or C1-C6 alkoxy, (iv) 4-8 membered heterocyclyl optionally substituted with 1-2 substituents independently selected from hydroxyl and C1-C6 haloalkyl, and (v) C1-C6 alkyl optionally substituted with 1-4 substituents independently selected from: halogen, hydroxyl, —NR^{C}R^{D}, C1-C6 alkoxy, C1-C6 haloalkoxy, C3-C6 cycloalkyl, phenyl optionally substituted with C1-C6 alkoxy, 5-6 membered heteroaryl optionally substituted with C1-C6 alkyl, and 4-8 membered heterocyclyl optionally substituted with —C(═O)C1-C6 alkyl or C1-C6 alkyl.

In some embodiments, R^{B} is —S(O₂)C1-C6 alkyl. In some embodiments, R^{B} is —S(O₂)C1-C3 alkyl. In some embodiments, R^{B} is —S(O₂)(CH₂)₂CH₃.

In some embodiments, R^{B} is C3-C6 cycloalkyl optionally substituted with hydroxyl or C1-C6 alkoxy. In some embodiments, R^{B} is C3-C6 cycloalkyl substituted with hydroxyl. In some embodiments, R^{B} is cyclobutyl, cyclopentyl, or cyclohexyl, each substituted with hydroxyl. In some embodiments, R^{B} is C3-C6 cycloalkyl substituted with C1-C6 alkoxy. In some embodiments, R^{B} is C3-C6 cycloalkyl substituted with C1-C3 alkoxy. In some embodiments, R^{B} is cyclobutyl substituted with C1-C3 alkoxy. In some embodiments, R^{B} is cyclobutyl substituted with methoxy. In some embodiments, R^{B} is unsubstituted C3-C6 cycloalkyl.

In some embodiments, R^{B} is 4-8 membered heterocyclyl optionally substituted with 1-2 substituents independently selected from hydroxyl and C1-C6 haloalkyl. In some embodiments, R^{B} is 4-8 membered heterocyclyl substituted with 1-2 substituents independently selected from hydroxyl and C1-C6 haloalkyl. In some embodiments, R^{B} is 4-8 membered heterocyclyl substituted with hydroxyl and C1-C6 haloalkyl. In some embodiments, R^{B} is 4-8 membered heterocyclyl substituted with C1-C6 haloalkyl. In some embodiments, R^{B} is 4-8 membered heterocyclyl substituted with trifluoromethyl. In some embodiments, R^{B} is 4-8 membered heterocyclyl substituted with hydroxyl. In some embodiments, R^{B} is unsubstituted 4-8 membered heterocyclyl. In some embodiments, R^{B} is morpholinyl.

In some embodiments, R^{B} is C1-C6 alkyl optionally substituted with 1-4 substituents independently selected from:
(a) halogen,
(b) hydroxyl,
(c) —NR^{C}R^{D},
(d) C1-C6 alkoxy,
(e) C1-C6 haloalkoxy,
(f) C3-C6 cycloalkyl optionally substituted with hydroxyl,
(g) phenyl optionally substituted with C1-C6 alkoxy,
(h) 5-6 membered heteroaryl optionally substituted with C1-C6 alkyl,
(j) 4-8 membered heterocyclyl optionally substituted with hydroxyl, —C(═O)C1-C6 alkyl, or C1-C6 alkyl,
(k) C1-C6 thioalkyl, and
(l) —S(═NR^{E})(═O)C1-C6 alkyl.

In some embodiments, R^{B} is C1-C6 alkyl optionally substituted with 1-3 substituents independently selected from:
(a) halogen,
(b) hydroxyl,
(c) —NR^{C}R^{D},
(d) C1-C6 alkoxy,
(e) C1-C6 haloalkoxy,
(f) C3-C6 cycloalkyl optionally substituted with hydroxyl,
(g) phenyl optionally substituted with C1-C6 alkoxy,
(h) 5-6 membered heteroaryl optionally substituted with C1-C6 alkyl,
(j) 4-8 membered heterocyclyl optionally substituted with hydroxyl, —C(═O)C1-C6 alkyl, or C1-C6 alkyl,
(k) C1-C6 thioalkyl, and
(l) —S(═NR^{E})(═O)C1-C6 alkyl.

In some embodiments, R^{B} is C1-C6 alkyl optionally substituted with two substituents independently selected from:
(a) halogen,
(b) hydroxyl,
(c) —NR^{C}R^{D},
(d) C1-C6 alkoxy,
(e) C1-C6 haloalkoxy,
(f) C3-C6 cycloalkyl optionally substituted with hydroxyl,
(g) phenyl optionally substituted with C1-C6 alkoxy,
(h) 5-6 membered heteroaryl optionally substituted with C1-C6 alkyl,
(j) 4-8 membered heterocyclyl optionally substituted with hydroxyl, —C(═O)C1-C6 alkyl, or C1-C6 alkyl,
(k) C1-C6 thioalkyl, and
(l) —S(═NR^{E})(═O)C1-C6 alkyl.

In some embodiments, R^{B} is C1-C6 alkyl optionally substituted with one of:
(a) halogen,
(b) hydroxyl,
(c) —NR^{C}R^{D},
(d) C1-C6 alkoxy,
(e) C1-C6 haloalkoxy,
(f) C3-C6 cycloalkyl optionally substituted with hydroxyl,
(g) phenyl optionally substituted with C1-C6 alkoxy,
(h) 5-6 membered heteroaryl optionally substituted with C1-C6 alkyl,
(j) 4-8 membered heterocyclyl optionally substituted with hydroxyl, —C(═O)C1-C6 alkyl, or C1-C6 alkyl,
(k) C1-C6 thioalkyl, and
(l) —S(═NR^{E})(═O)C1-C6 alkyl.

In some embodiments, R^{B} is C1-C6 alkyl optionally substituted with 1-4 substituents independently selected from: halogen, hydroxyl, —NR^{C}R^{D}, C1-C6 alkoxy, C1-C6 haloalkoxy, C3-C6 cycloalkyl, phenyl optionally substituted with C1-C6 alkoxy, 5-6 membered heteroaryl optionally substituted with C1-C6 alkyl, and 4-8 membered heterocyclyl optionally substituted with —C(═O)C1-C6 alkyl or C1-C6 alkyl.

In some embodiments, R^{B} is C1-C6 alkyl optionally substituted with 1-3 substituents independently selected from: halogen, hydroxyl, —NR^{C}R^{D}, C1-C6 alkoxy, C1-C6 haloalkoxy, C3-C6 cycloalkyl, phenyl optionally substituted with C1-C6 alkoxy, 5-6 membered heteroaryl optionally substituted with C1-C6 alkyl, and 4-8 membered heterocyclyl optionally substituted with —C(═O)C1-C6 alkyl or C1-C6 alkyl.

In some embodiments, R^{B} is C1-C6 alkyl optionally substituted with 1-2 substituents independently selected from: halogen, hydroxyl, —NR^{C}R^{D}, C1-C6 alkoxy, C1-C6 haloalkoxy, C3-C6 cycloalkyl, phenyl optionally substituted with C1-C6 alkoxy, 5-6 membered heteroaryl optionally substituted with C1-C6 alkyl, and 4-8 membered heterocyclyl optionally substituted with —C(═O)C1-C6 alkyl or C1-C6 alkyl.

In some embodiments, R^{B} is C1-C6 alkyl optionally substituted with halogen, hydroxyl, —NR^{C}R^{D}, C1-C6 alkoxy, C1-C6 haloalkoxy, C3-C6 cycloalkyl, phenyl optionally substituted with C1-C6 alkoxy, 5-6 membered heteroaryl optionally substituted with C1-C6 alkyl, or 4-8 membered heterocyclyl optionally substituted with —C(═O)C1-C6 alkyl or C1-C6 alkyl.

In some embodiments, R^{B} is C1-C6 alkyl substituted with 1-4 halogen. In some embodiments, R^{B} is C1-C3 alkyl substituted with 1-4 halogen. In some embodiments, R^{B} is C1-C3 alkyl substituted with 1-3 halogen. In some embodiments, R^{B} is C1-C6 alkyl substituted with 1-3 halogen and hydroxyl. In some embodiments, R^{B} is C1-C3 alkyl substituted with 1-3 halogen and hydroxyl.

In some embodiments, R^{B} is propyl substituted with 1-2 halogen and hydroxyl. In some embodiments, R^{B} is ethyl, propyl, or butyl each substituted with 1-3 halogen. In some embodiments, R^{B} is propyl or butyl, each substituted with 1-3 fluoro and one hydroxyl.

In some embodiments, R^{B} is C1-C6 alkyl substituted with 1-3 hydroxyl. In some embodiments, R^{B} is C2-C5 alkyl substituted with 1-2 hydroxyl. In some embodiments, R^{B} is ethyl, propyl, butyl, pentyl, each substituted with 1-2 hydroxyl. In some embodiments, R^{B} is hydroxyethyl. In some embodiments, R^{B} is hydroxypropyl. In some embodiments, R^{B} is propyl substituted with two hydroxyl. In some embodiments, R^{B} is sec-butyl or isoamyl, each substituted with hydroxyl.

In some embodiments, R^{B} is C1-C6 alkyl substituted with 1-4 independently selected C1-C6 alkoxy. In some embodiments, R^{B} is C2-C5 alkyl substituted with 1-2 independently selected C1-C3 alkoxy. In some embodiments, R^{B} is C2-C6 alkyl substituted with methoxy.

In some embodiments, R^{B} is C1-C6 alkyl substituted with 1-4 independently selected C1-C6 haloalkoxy. In some embodiments, R^{B} is C1-C6 alkyl substituted with 1-2 independently selected C1-C3 alkoxy. In some embodiments, R^{B} is C1-C6 alkyl substituted with —OCHF₂.

In some embodiments, R^{B} is C1-C6 alkyl substituted with C3-C6 cycloalkyl. In some embodiments, R^{B} is C1-C6 alkyl substituted with cyclopropyl. In some embodiments, R^{B} is C1-C6 alkyl substituted with cyclobutyl.

In some embodiments, R^{B} is C1-C6 alkyl substituted with phenyl optionally substituted with C1-C6 alkoxy. In some embodiments, R^{B} is C1-C3 alkyl substituted with phenyl optionally substituted with C1-C6 alkoxy. In some embodiments, R^{B} is C1-C6 alkyl substituted with phenyl. In some embodiments, R^{B} is —CH₂-phenyl. In some embodiments, R^{B} is C1-C6 alkyl substituted with phenyl substituted with C1-C6 alkoxy. In some embodiments, R^{B} is C1-C3 alkyl substituted with phenyl substituted with C1-C6 alkoxy. In some embodiments, R^{B} is methyl substituted with phenyl substituted with C1-C3 alkoxy. In some embodiments, R^{B} is methyl substituted with 4-methoxyphenyl.

In some embodiments, R^{B} is C1-C6 alkyl substituted with 5-6 membered heteroaryl optionally substituted with C1-C6 alkyl. In some embodiments, R^{B} is C1-C3 alkyl substituted with 5-6 membered heteroaryl optionally substituted with C1-C6 alkyl. In some embodiments, R^{B} is C1-C6 alkyl substituted with 5-6 membered heteroaryl substituted with C1-C6 alkyl. In some embodiments, R^{B} is C1-C3 alkyl substituted with 5-6 membered heteroaryl substituted with C1-C6 alkyl. In some embodiments, R^{B} is C1-C6 alkyl substituted with a 5-6 membered heteroaryl. In some embodiments, R^{B} is C1-C3 alkyl substituted with a 5-6 membered heteroaryl. In some embodiments, R^{B} is C1-C3 alkyl substituted with pyridonyl optionally substituted with C1-C3 alkyl. In some embodiments, R^{B} is methyl substituted with pyridonyl substituted with methyl. In some embodiments, R^{B} is methyl substituted with unsubstituted pyridonyl.

In some embodiments, R^{B} is C1-C6 alkyl substituted with a 4-8 membered heterocyclyl optionally substituted with —C(═O)C1-C6 alkyl or C1-C6 alkyl. In some embodiments, R^{B} is C1-C2 alkyl substituted with 4-6 membered heterocyclyl optionally substituted with —C(═O)C1-C6 alkyl or C1-C6 alkyl. In some embodiments, R^{B} is C1-C2 alkyl substituted with 4-6 membered heterocyclyl, wherein the 4-6 membered heterocyclyl is selected from the group consisting of 2-oxopiperidin-4-yl, 6-oxopiperidin-3-yl, piperidin-4-yl, and 2-oxopyrrolidin-1-yl, each optionally substituted with methyl. In some embodiments, R^{B} is C1-C2 alkyl substituted with a 4-6 membered heterocyclyl, wherein the 4-6 membered heterocyclyl is selected from the group consisting of unsubstituted 2-oxopiperidin-4-yl, 6-oxopiperidin-3-yl, piperidin-4-yl, and 2-oxopyrrolidin-1-yl. In some embodiments, R^{B} is C1-C2 alkyl substituted with 1-acetylpiperidinyl.

In some embodiments, R^{B} is C1-C6 alkyl optionally substituted with —NR^{C}R^{D}. In some embodiments, R^{B} is C1-C6 alkyl substituted with —NR^{C}R^{D}. In some embodiments, R^{B} is C2-C6 alkyl substituted with —NR^{C}R^{D}. In some embodiments, R^{B} is C2-C3 alkyl substituted with —NR^{C}R^{D}.

In some embodiments, R^{A} and R^{B} are the same. In some embodiments, R^{A} and R^{B} are different. In some embodiments, R^{A} and R^{B} are each hydrogen. In some embodiments, one of R^{A} and R^{B} is hydrogen and the other of R^{A} and R^{B} is not hydrogen.

In some embodiments, R^{A} and R^{B} are each independently selected C1-C6 alkyl. In some embodiments, R^{A} and R^{B} are each independently selected C1-C3 alkyl. In some embodiments, R^{A} and R^{B} are each methyl.

In some embodiments, R^{C} and R^{D} are the same. In some embodiments, R^{C} and R^{D} are different. In some embodiments, R^{C} and R^{D} are each hydrogen. In some embodiments, one of R^{C} and R^{D} is hydrogen and the other of R^{C} and R^{D} is C1-C6 alkyl optionally substituted with oxo. In some embodiments, one of R^{C} and R^{D} is hydrogen and the other of R^{C} and R^{D} is C1-C2 alkyl optionally substituted with oxo. In some embodiments, one of R^{C} and R^{D} is hydrogen and the other of R^{C} and R^{D} is acyl. In some embodiments, R^{C} and R^{D} are each an independently selected C1-C6 alkyl each optionally substituted with oxo. In some embodiments, R^{C} and R^{D} are each an independently selected C1-C6 alkyl. In some embodiments, R^{C} and R^{D} are each methyl. In some embodiments, one of R^{C} and R^{D} is hydrogen and the other of R^{C} and R^{D} is —C(═O)OC1-C6 alkyl.

In some embodiments, R^{B} is C1-C6 alkyl optionally substituted with 1-2 substituents independently selected from: halogen, hydroxyl, —NR^{C}R^{D}, C1-C6 alkoxy, C1-C6 haloalkoxy, C3-C6 cycloalkyl, phenyl optionally substituted with C1-C6 alkoxy, 5-6 membered heteroaryl optionally substituted with C1-C6 alkyl, and 4-8 membered heterocyclyl optionally substituted with —C(═O)C1-C6 alkyl or C1-C6 alkyl.

In some embodiments, R^{B} is C1-C6 alkyl optionally substituted with 1-2 substituents independently selected from: halogen, hydroxyl, —NR^{C}R^{D}, C1-C6 alkoxy, C1-C6 haloalkoxy, C3-C6 cycloalkyl, phenyl substituted with C1-C6 alkoxy, 5-6 membered heteroaryl substituted with C1-C6 alkyl, and 4-8 membered heterocyclyl substituted with —C(═O)C1-C6 alkyl or C1-C6 alkyl.

In some embodiments, R^{B} is C1-C6 alkyl optionally substituted with 1-2 substituents independently selected from: halogen, hydroxyl, —NR^{C}R^{D}, C1-C6 alkoxy, C1-C6 haloalkoxy, C3-C6 cycloalkyl, phenyl, 5-6 membered heteroaryl, and 4-8 membered heterocyclyl.

In some embodiments, R^{B} is C1-C6 alkyl optionally substituted with 1-2 substituents independently selected from: fluoro, hydroxyl, —NR^{C}R^{D}, methoxy, trifluoromethoxy, C3-C6 cycloalkyl, phenyl optionally substituted with methoxy, 5-6 membered heteroaryl optionally substituted with methyl, and 4-8 membered heterocyclyl optionally substituted with —C(═O)CH₃ or methyl.

In some embodiments, R^{B} is C1-C6 alkyl optionally substituted with 1-2 substituents independently selected from: fluoro, hydroxyl, —NR^{C}R^{D}, methoxy, trifluoromethoxy, C3-C6 cycloalkyl, phenyl, 5-6 membered heteroaryl, and 4-8 membered heterocyclyl.

In some embodiments, when R^{B} is a substituted C1-C6 alkyl, the C1-C6 alky can form a spirocycle with a cycloalkyl or heterocyclyl group. In some embodiments, R^{B} is selected from the group consisting of

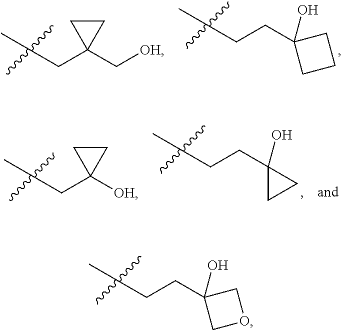 [SMILES: *CC1(CO)CC1 ; *CCC1(O)CCC1 ; *CC1(O)CC1 ; *CCC1(O)CC1 ; *CCC1(O)COC1]

wherein the wavy line represents the point of connect to the —NR^{A}R^{B} nitrogen atom.

In some embodiments, R^{B} is selected from the group consisting of:

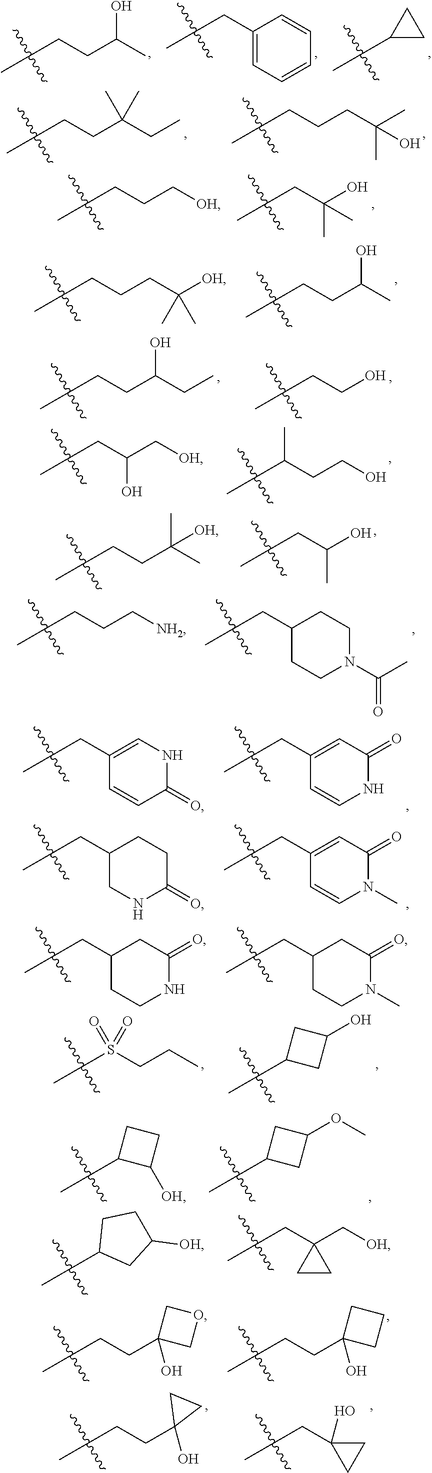

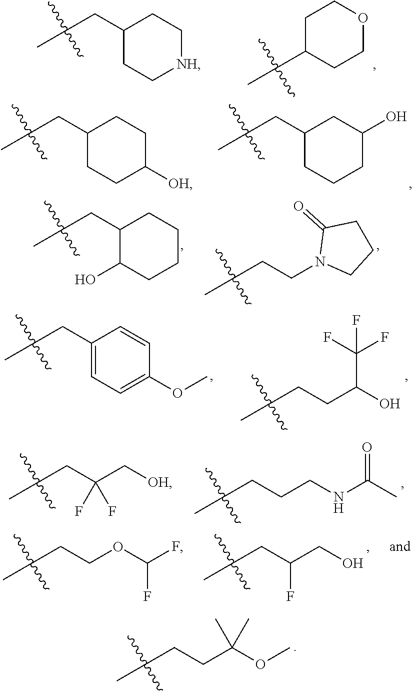

In some embodiments, m is 0.

In some embodiments, m is 1.

In some embodiments, m is 1 and R¹ is selected from the group consisting of fluoro, amino, and —NHC1-C6 alkyl optionally substituted with hydroxyl. In some embodiments, m is 1 and R¹ is fluoro. In some embodiments, m is 1 and R¹ is amino. In some embodiments, m is 1 and R¹ is —NHC1-C6 alkyl optionally substituted with hydroxyl. In some embodiments, m is 1 and R¹ is —NHC1-C6 alkyl substituted with hydroxyl.

In some embodiments, m is 2.

In some embodiments, m is 2 and each R¹ is independently selected from the group consisting of fluoro, amino, and —NHC1-C6 alkyl optionally substituted with 1-2 substituents independently selected from hydroxyl and C3-C6 cycloalkyl. In some embodiments, m is 2 and each R¹ is independently selected from the group consisting of fluoro, amino, and —NHC1-C6 alkyl substituted with 1-2 substituents independently selected from hydroxyl and C3-C6 cycloalkyl. In some embodiments, m is 2 and each R¹ is independently selected from the group consisting of fluoro, amino, and —NHC1-C6 alkyl substituted with hydroxyl or C3-C6 cycloalkyl. In some embodiments, m is 2 and each R¹ is independently selected from the group consisting of fluoro, amino, and —NHC1-C6 alkyl optionally substituted with 2 substituents independently selected from hydroxyl and C3-C6 cycloalkyl.

In some embodiments, m is 3.

In some embodiments, m is 3 and each R¹ is independently selected from the group consisting of fluoro, cyano, C1-C6 alkoxy, —NHS(O₂)C1-C6 alkyl, and —NHC1-C6 alkyl optionally substituted with hydroxyl. In some embodiments, m is 3, two R¹ are independently selected from fluoro and C1-C6 alkoxy, and the third R¹ is cyano, —NHS(O₂)C1-C6 alkyl, or —NHC1-C6 alkyl optionally substituted with hydroxyl.

In some embodiments, m is 4.

In some embodiments, m is 4 and each R¹ is independently selected from the group consisting of fluoro, cyano, C1-C6 alkoxy, —NHS(O₂)C1-C6 alkyl, and —NHC1-C6 alkyl optionally substituted with hydroxyl. In some embodiments, m is 4, two R¹ are independently selected from fluoro and C1-C6 alkoxy, and the remaining two R¹ are independently selected from cyano, —NHS(O₂)C1-C6 alkyl, or —NHC1-C6 alkyl optionally substituted with hydroxyl.

In some embodiments, R² is hydrogen.

In some embodiments, R² is halogen. In some embodiments, R² is chloro. In some embodiments, R² is fluoro.

In some embodiments, R² is C1-C6 alkoxy. In some embodiments, R² is C1-C3 alkoxy. In some embodiments, R² is methoxy. In some embodiments, R² is ethoxy.

In some embodiments, R² is C1-C6 haloalkoxy. In some embodiments, R² is trifluoromethoxy. In some embodiments, R² is 2,2,2-trifluorethoxy.

In some embodiments, R³ is hydrogen.

In some embodiments, R³ is C1-C6 thioalkyl. In some embodiments, R³ is C3-C4 thioalkyl. In some embodiments, R³ is

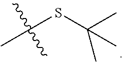 [SMILES: *SC(C)(C)C]

In some embodiments, R³ is —CO₂H.

In some embodiments, R³ is —C(═O)NR^{E}R^{F}.

In some embodiments, R³ is C1-C6 alkyl optionally substituted with NR^{E}R^{F} or hydroxyl. In some embodiments, R³ is C1-C6 alkyl substituted with NR^{E}R^{F} or hydroxyl. In some embodiments, R³ is C1-C6 alkyl substituted with NR^{E}R^{F}. In some embodiments, R³ is C1-C6 alkyl optionally substituted with hydroxyl. In some embodiments, R³ is an unsubstituted C1-C6 alkyl.

In some embodiments, R^{E} and R^{F} are the same. In some embodiments, R^{E} and R^{F} are different. In some embodiments, each of R^{E} and R^{F} is hydrogen. In some embodiments, each of R^{E} and R^{F} is an independently selected C1-C6 alkyl. In some embodiments, each of R^{E} and R^{F} is methyl. In some embodiments, one of R^{E} and R^{F} is hydrogen and the other of R^{E} and R^{F} is C1-C6 alkyl. In some embodiments, R^{E} is hydrogen. In some embodiments, R^{E} is C1-C6 alkyl. In some embodiments, R^{F} is hydrogen. In some embodiments, R^{F} is C1-C6 alkyl.

In some embodiments, R³ is C1-C6 alkoxy optionally substituted with 4-10 membered heterocyclyl optionally substituted with C1-C6 alkoxy. In some embodiments, R³ is C1-C3 alkoxy optionally substituted with 4-10 membered heterocyclyl optionally substituted with C1-C6 alkoxy. In some embodiments, R³ is methoxy or ethoxy optionally substituted with 4-10 membered heterocyclyl optionally substituted with C1-C6 alkoxy. In some embodiments, R³ is C1-C6 alkoxy substituted with 4-10 membered heterocyclyl optionally substituted with C1-C6 alkoxy. In some embodiments, R³ is C1-C3 alkoxy substituted with 4-6 membered heterocyclyl optionally substituted with C1-C6 alkoxy. In some embodiments, R³ is methoxy or ethoxy substituted with 4-6 membered heterocyclyl optionally substituted with C1-C3 alkoxy. In some embodiments, R³ is C1-C3 alkoxy substituted with an unsubstituted 4-10 membered heterocyclyl. In some embodiments, R³ is C1-C3 alkoxy substituted with an unsubstituted 4-6 membered heterocyclyl. In some embodiments, R³ is C1-C6 alkoxy substituted with 4-10 membered heterocyclyl substituted with C1-C6 alkoxy. In some embodiments, R³ is C1-C3 alkoxy substituted with 4-6 membered heterocyclyl substituted with C1-C6 alkoxy. In some embodiments, R³ is methoxy or ethoxy substituted with 4-6 membered heterocyclyl optionally substituted with C1-C3 alkoxy.

In some embodiments, R³ is unsubstituted C1-C6 alkoxy. In some embodiments, R³ is unsubstituted C1-C3 alkoxy. In some embodiments, R³ methoxy. In some embodiments, R³ ethoxy.

In some embodiments, R³ is 4-8 membered heterocyclyl optionally substituted with 1-2 substituents independently selected from halogen and C1-C6 alkyl. In some embodiments, R³ is 4-8 membered heterocyclyl substituted with 1-2 substituents independently selected from halogen and C1-C6 alkyl. In some embodiments, R³ is 4-8 membered heterocyclyl substituted with 1 substituent selected from halogen and C1-C6 alkyl. In some embodiments, R³ is 4-8 membered heterocyclyl substituted with 2 substituents independently selected from halogen and C1-C6 alkyl. In some embodiments, R³ is 4-8 membered heterocyclyl substituted with 2 independently halogen. In some embodiments, R³ is 4-8 membered heterocyclyl substituted with 2 fluoro. In some embodiments, R³ is 4-8 membered heterocyclyl substituted with 2 independently selected C1-C6 alkyl. In some embodiments, R³ is 4-8 membered heterocyclyl substituted with 2 methyl groups. In some embodiments, R³ is an unsubstituted 4-8 membered heterocyclyl.

In some embodiments, R³ is

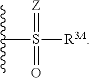

In some embodiments, Z is O.

In some embodiments, Z is NR⁴. In some embodiments, R⁴ is hydrogen.

In some embodiments, R⁴ is C1-C6 alkyl. In some embodiments, R⁴ is C1-C3 alkyl. In some embodiments, R⁴ is methyl.

In some embodiments, R^{3A} is C1-C6 haloalkyl. In some embodiments, R^{3A} is C3-C4 haloalkyl.

In some embodiments, R^{3A} is C3-C6 cycloalkyl. In some embodiments, R^{3A} is cyclopropyl.

In some embodiments, R^{3A} is C1-C6 alkyl optionally substituted with C3-C6 cycloalkyl. In some embodiments, R^{3A} is C1-C6 alkyl substituted with C3-C6 cycloalkyl. In some embodiments, R^{3A} is C1-C3 alkyl substituted with C3-C6 cycloalkyl. In some embodiments, R^{3A} is C1-C2 alkyl substituted with C3-C6 cycloalkyl. In some embodiments, R^{3A} is C1-C2 alkyl substituted with C3-C6 cycloalkyl, wherein the C1-C2 alkyl of R^{3A} forms a spirocycloalkyl with C3-C6 cycloalkyl. In some embodiments, R^{3A} is

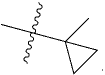 [SMILES: *C1(C)CC1]

In some embodiments, R^{3A} is an unsubstituted C1-C6 alkyl. In some embodiments, R^{3A} is an unsubstituted C3-C4 alkyl. In some embodiments, R^{3A} is isopropyl. In some embodiments, R^{3A} is tert-butyl.

In some embodiments, R³ is

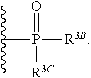

In some embodiments, R^{3B} and R^{3C} are the same. In some embodiments, R^{3B} and R^{3C} are different.

In some embodiments, R^{3B} is C3-C6 cycloalkyl. In some embodiments, R^{3B} is C3-C4 cycloalkyl.

In some embodiments, R^{3B} is C1-C6 alkyl substituted with C3-C6 cycloalkyl. In some embodiments, R^{3B} is C1-C3 alkyl substituted with C3-C6 cycloalkyl. In some embodiments, R^{3B} is C1-C2 alkyl substituted with C3-C6 cycloalkyl. In some embodiments, R^{3B} is C1-C2 alkyl substituted with C3-C6 cycloalkyl, wherein the C1-C2 alkyl of R^{3B} forms a spirocycloalkyl with C3-C6 cycloalkyl. In some embodiments, R^{3B} is

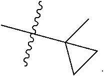 [SMILES: *C1(C)CC1]

In some embodiments, R^{3B} is an unsubstituted C1-C6 alkyl. In some embodiments, R^{3B} is methyl or ethyl.

In some embodiments, R^{3C} is C3-C6 cycloalkyl. In some embodiments, R^{3C} is C3-C4 cycloalkyl.

In some embodiments, R^{3C} is C1-C6 alkyl substituted with C3-C6 cycloalkyl. In some embodiments, R^{3C} is C1-C3 alkyl substituted with C3-C6 cycloalkyl. In some embodiments, R^{3C} is C1-C2 alkyl substituted with C3-C6 cycloalkyl. In some embodiments, R^{3C} is C1-C2 alkyl substituted with C3-C6 cycloalkyl, wherein the C1-C2 alkyl of R^{3C} forms a spirocycloalkyl with C3-C6 cycloalkyl. In some embodiments, R^{3C} is

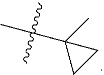 [SMILES: *C1(C)CC1]

In some embodiments, R^{3C} is an unsubstituted C1-C6 alkyl. In some embodiments, R^{3C} is methyl or ethyl.

In some embodiments, the compound of Formula (I) is Formula (I-A):

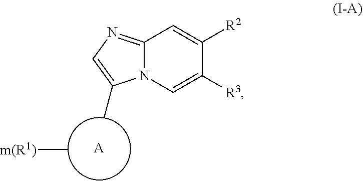

or a pharmaceutically acceptable salt thereof.

In some embodiments, the compound of Formula (I) is Formula (I-B):

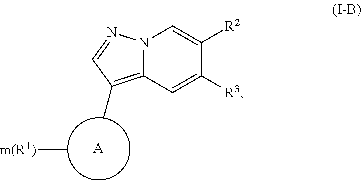

or a pharmaceutically acceptable salt thereof.

In some embodiments, the compound of Formula (I) is Formula (I-C):

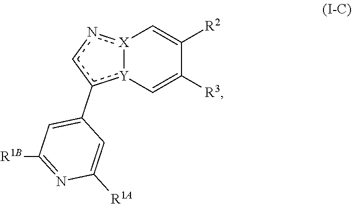

or a pharmaceutically acceptable salt thereof, wherein R^{1A} and R^{1B} are each independently selected from R¹.

In some embodiments, the compound of Formula (I) is Formula (I-D):

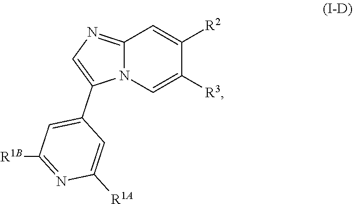

or a pharmaceutically acceptable salt thereof, wherein R^{1A} and R^{1B} are each independently selected from R¹.

In some embodiments, the compound of Formula (I) is Formula (I-E):

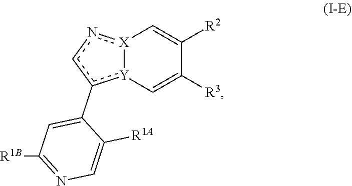

or a pharmaceutically acceptable salt thereof, wherein R^{1A} and R^{1B} are each independently selected from R¹.

In some embodiments, the compound of Formula (I) is Formula (I-F):

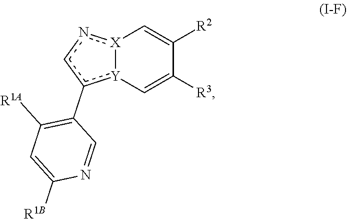

or a pharmaceutically acceptable salt thereof, wherein R^{1A} and R^{1B} are each independently selected from R¹.

In some embodiments, the compound of Formula (I) is Formula (I-G):

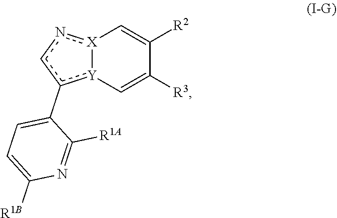

or a pharmaceutically acceptable salt thereof, wherein R^{1A} and R^{B} are each independently selected from R¹. In some embodiments, the compound of Formula (I) is Formula (I-H):

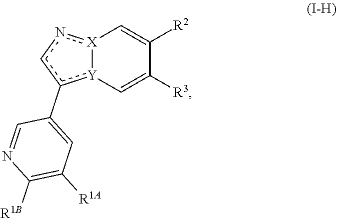

or a pharmaceutically acceptable salt thereof, wherein R^{1A} and R^{1B} are each independently selected from R¹.

In some embodiments, the compound of Formula (I) is Formula (I-I):

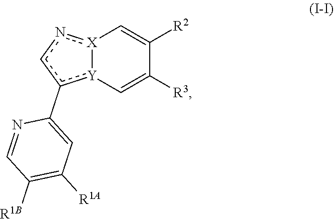

or a pharmaceutically acceptable salt thereof, wherein R^{1A} and R^{1B} are each independently selected from R¹.

In some embodiments, the compound of Formula (I) is Formula (I-J):

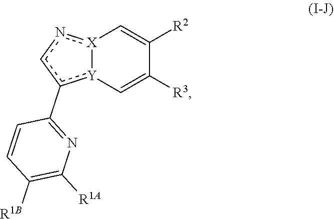

or a pharmaceutically acceptable salt thereof, wherein R^{1A} and R^{1B} are each independently selected from R¹.

In some embodiments, the compound of Formula (I) is Formula (I-K):

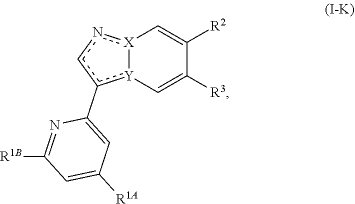

or a pharmaceutically acceptable salt thereof, wherein R^{1A} and R^{1B} are each independently selected from R¹.

In some embodiments, the compound of Formula (I) is Formula (I-L):

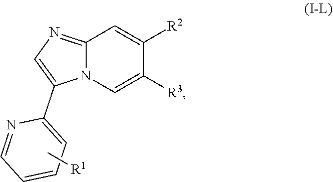

or a pharmaceutically acceptable salt thereof.

In some embodiments, the compound of Formula (I) is Formula (I-M):

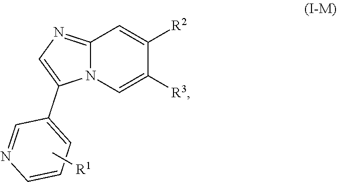

or a pharmaceutically acceptable salt thereof.

In some embodiments, the compound of Formula (I) is Formula (I-N):

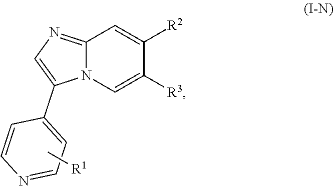

or a pharmaceutically acceptable salt thereof.

In some embodiments, the compound of Formula (I) is Formula (I-O):

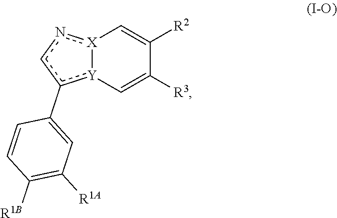

or a pharmaceutically acceptable salt thereof, wherein R^{1A} and R^{1B} are each independently selected from R¹.

In some embodiments, the compound of Formula (I) is Formula (I-P):

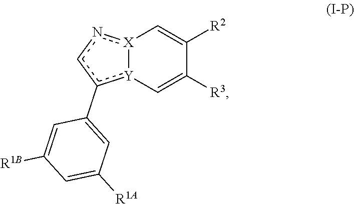

or a pharmaceutically acceptable salt thereof, wherein R^{1A} and R^{1B} are each independently selected from R¹.

In some embodiments, the compound of Formula (I) is Formula (I-Q):

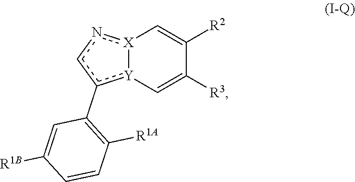

In some embodiments, the compound of Formula (I) is Formula (I-R):

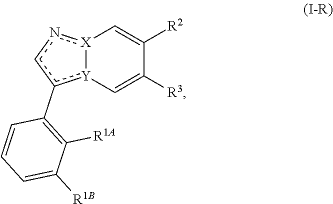

or a pharmaceutically acceptable salt thereof, wherein R^{1A} and R^{1B} are each independently selected from R¹.

In some embodiments, the compound of Formula (I) is Formula (I-S):

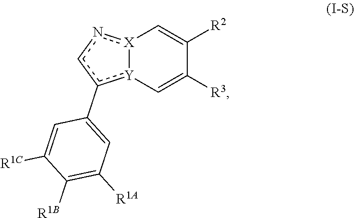

or a pharmaceutically acceptable salt thereof, wherein R^{1A}, R^{1B} and R^{1C} are each independently selected from R¹.

In some embodiments, the compound of Formula (I) is Formula (I-T):

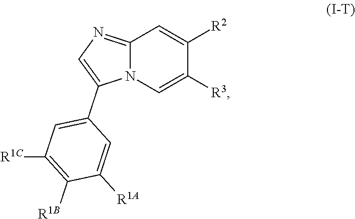

or a pharmaceutically acceptable salt thereof, wherein R^{1A}, R^{1B} and R^{1C} are each independently selected from R¹.

In some embodiments, the compound of Formula (I) is Formula (I-U):

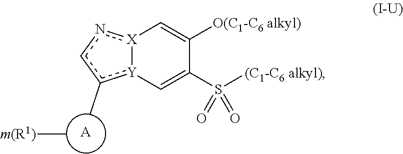

or a pharmaceutically acceptable salt thereof.

In some embodiments, the compound of Formula (I) is Formula (I-V):

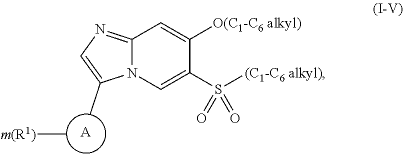

or a pharmaceutically acceptable salt thereof.

In some embodiments, the compound of Formula (I) is Formula (I-W):

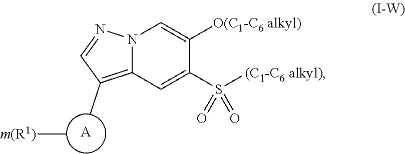

or a pharmaceutically acceptable salt thereof.

In some embodiments, the compound of Formula (I) is Formula (I-X):

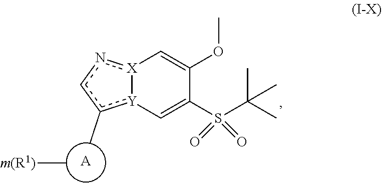

or a pharmaceutically acceptable salt thereof.

In some embodiments, R² is C1-C6 alkoxy and R³ is

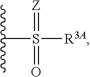

wherein Z is O.

In some embodiments of Formulae (I-A), (I-B), (I-C), (I-D), and (I-E), R² is C1-C6 alkoxy and R³ is

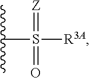

wherein Z is O and R^{3A} is C1-C6 alkyl or C3-C6 cycloalkyl.

In some embodiments, m is 2, wherein one R¹ is halogen and the other R¹ is —NR^{A}R^{B}.

In some embodiments of Formulae (I-A) and (I-B), m is 2, wherein one R¹ is halogen and the other R¹ is —NR^{A}R^{B}.

In some embodiments of Formulae (I-C), (I-D), and (I-E), one of R^{1A} and R^{B} is halogen and the other one R^{1A} and R^{1B} is —NR^{A}R^{B}.

In some embodiments, m is 2, wherein one R¹ is C1-C6 alkoxy optionally substituted with hydroxyl or phenyl and the other R¹ is —NR^{A}R^{B}.

In some embodiments of Formulae (I-A) and (I-B), m is 2, wherein one R¹ is C1-C6 alkoxy optionally substituted with hydroxyl or phenyl and the other R¹ is —NR^{A}R^{B}.

In some embodiments of Formulae (I-C), (I-D), and (I-E), one of R^{1A} and R^{1B} is C1-C6 alkoxy optionally substituted with hydroxyl or phenyl and the other one R^{1A} and R^{1B} is —NR^{A}R^{B}.

In some embodiments, m is 2, wherein one R¹ is C1-C6 alkyl optionally substituted with hydroxyl and the other R¹ is —NR^{A}R^{B}.

In some embodiments of Formulae (I-A) and (I-B), m is 2, wherein one R¹ is C1-C6 alkyl optionally substituted with hydroxyl and the other R¹ is —NR^{A}R^{B}.

In some embodiments of Formulae (I-C), (I-D), and (I-E), one of R^{1A} and R^{1B} is C1-C6 alkyl optionally substituted with hydroxyl and the other one R^{1A} and R^{1B} is —NR^{A}R^{B}.

In some embodiments, m is 2, wherein one R¹ is C1-C6 haloalkyl and the other R¹ is —NR^{A}R^{B}.

In some embodiments of Formulae (I-A) and (I-B), m is 2, wherein one R¹ is C1-C6 haloalkyl and the other R¹ is —NR^{A}R^{B}.

In some embodiments of Formulae (I-C), (I-D), and (I-E), one of R^{1A} and R^{1B} is C1-C6 haloalkyl and the other one R^{1A} and R^{1B} is —NR^{A}R^{B}.

In some embodiments, m is 2, wherein one R¹ is halogen and the other R¹ is 4-8 membered heterocyclyl optionally substituted with 1-2 substituents independently selected from the group consisting of hydroxyl, C1-C6 alkyl, and C1-C6 haloalkyl.

In some embodiments of Formulae (I-A) and (I-B), m is 2, wherein one R¹ is halogen and the other R¹ is 4-8 membered heterocyclyl optionally substituted with 1-2 substituents independently selected from the group consisting of hydroxyl, C1-C6 alkyl, and C1-C6 haloalkyl.

In some embodiments of Formulae (I-C), (I-D), and (I-E), one of RIA and R^{1B} is halogen and the other one R^{1A} and R^{1B} is a 4-8 membered heterocyclyl optionally substituted with 1-2 substituents independently selected from the group consisting of hydroxyl, C1-C6 alkyl, and C1-C6 haloalkyl.

In some embodiments of Formulae (I-A), (I-B), (I-C), (I-D), and (I-E), R^{A} and R^{B} are both hydrogen.

In some embodiments of Formulae (I-A), (I-B), (I-C), (I-D), and (I-E), R^{A} is hydrogen and R^{B} is —S(O₂)C1-C6 alkyl.

In some embodiments of Formulae (I-A), (I-B), (I-C), (I-D), and (I-E), R^{A} is hydrogen and R^{B} is C3-C6 cycloalkyl optionally substituted with hydroxyl or C1-C6 alkoxy.

In some embodiments of Formulae (I-A), (I-B), (I-C), (I-D), and (I-E), R^{A} is hydrogen and R^{B} is 4-8 membered heterocyclyl optionally substituted with hydroxyl.

In some embodiments of Formulae (I-A), (I-B), (I-C), (I-D), and (I-E), R^{A} is hydrogen and R^{B} is C1-C6 alkyl optionally substituted with 1-4 substituents independently selected from: halogen, hydroxyl, —NR^{C}R^{D}, C1-C6 alkoxy, C1-C6 haloalkoxy, C3-C6 cycloalkyl, phenyl optionally substituted with C1-C6 alkoxy, 5-6 membered heteroaryl optionally substituted with C1-C6 alkyl, and 4-8 membered heterocyclyl optionally substituted with —C(═O)C1-C6 alkyl or C1-C6 alkyl.

In some embodiments of Formulae (I-A) to (I-X), R³ is —S(O₂)-tert-Butyl, R² is —OMe, and Ring A is pyridyl.

In some embodiments of Formulae (I-A) to (I-X), R³ is —S(O₂)-tert-Butyl, R² is —OMe, and Ring A is phenyl.

In some embodiments of Formulae (I-A) to (I-X), R³ is —S(O₂)-tert-Butyl, R² is —OMe, Ring A is phenyl or pyridyl and at least one of R¹ is F.

In some embodiments of Formulae (I-A) to (I-X), R³ is —S(O₂)-tert-Butyl, R² is —OMe, Ring A is phenyl or pyridyl and at least one of R¹ is C1-C4 alkoxy.

In some embodiments of Formulae (I-A) to (I-X), R³ is —S(O₂)-tert-Butyl, R² is —OMe, Ring A is phenyl or pyridyl and at least one of R¹ is —NH₂.

In some embodiments, R³ is —S(O₂)-tert-Butyl, R² is —OMe, and Ring A is phenyl.

In some embodiments, R³ is —S(O₂)-tert-Butyl, R² is —OMe, and Ring A is 6-membered heteroaryl.

In some embodiments, R³ is —S(O₂)-tert-Butyl, R² is —OMe, and Ring A is pyridyl.

In some embodiments, R³ is —S(O₂)-tert-Butyl, R² is —OMe, and Ring A is pyrimidinyl.

In some embodiments, R³ is —S(O₂)-tert-Butyl, R² is —OMe, and Ring A is 5-membered heteroaryl.

In some embodiments, when X is C, Y is N, Ring A is 4-pyridyl, R² is C1-C6 alkoxy, R³ is —S(O₂)—C1-C6 alkyl, and m is 1, then R¹ is not —NH₂ or —NH(C═O)CH₃.

In some embodiments, when X is C, Y is N, Ring A is 4-pyridyl, R² is C1-C6 alkoxy, R³ is —S(O₂)—C1-C6 alkyl, m is 2, and one R¹ is fluoro, NH-(p-methoxybenzyl), or —NH₂, then the other R¹ is not halogen.

In some embodiments, when X is C, Y is N, Ring A is phenyl, R² is C1-C6 alkoxy, R³ is —S(O₂)—C1-C6 alkyl, and m is 1, R¹ is not fluoro, cyano, or unsubstituted C1-C6 alkoxy.

In some embodiments, when X is C, Y is N, Ring A is phenyl, R² is C1-C6 alkoxy, R³ is —S(O₂)—C1-C6 alkyl, m is 2, and one R¹ is halogen, then the other R¹ is not unsubstituted C1-C6 alkoxy.

In some embodiments, when X is C, Y is N, Ring A is 4-pyridyl, R² is C1-C6 haloalkoxy, R³ is —S(O₂)—C1-C6 alkyl, m is 2, and one R¹ is —NH₂, then the other R¹ is not halogen.

In some embodiments, when X is C, Y is N, R² is C1-C3 alkoxy, R³ is —S(O₂)—C1-C4 alkyl, Ring A is phenyl, and m is 1, R¹ is not halogen, —NH₂, cyano, or unsubstituted C1-C6 alkoxy.

In some embodiments, when X is C, Y is N, R² is ethoxy, R³ is —S(O₂)—C1-C4 alkyl, and m is 2, and one R¹ is halogen, then the other R¹ is not —NH₂ or unsubstituted alkoxy.

In some embodiments, when X is C, Y is N, R² is C1-C2 haloalkoxy, R³ is —S(O₂)—C1-C4 alkyl, and m is 2, and one R¹ is halogen, then the other R¹ is not —NH₂.

In some embodiments, when X is C, Y is N, R² is C1-C3 alkoxy, R³ is —S(O₂)—C1-C4 alkyl, and m is 0, then Ring A is not 1H-indazole or 1H-benzo[d]imidazole.

In some embodiments, the compounds described herein are not any compound disclosed in He, et al., ACS Med. Chem. Lett., Vol. 8, pp. 1048-1053 (2017), which is herein incorporated by references solely for purposes of excluding the compounds disclosed therein.

In some embodiments, the compounds described herein are not selected from 4-[6-[(1,1-dimethylethyl)sulfonyl]-7-methoxyimidazo[1,2-a]pyridin-3-yl]-2-pyridinamine; 3-(2-amino-6-chloro-4-pyridinyl)-6-[(1,1-dimethylethyl)sulfonyl]imidazo[1,2-a]pyridin-7-ol; 3-(2,6-difluoro-4-pyridinyl)-6-[(1,1-dimethylethyl)sulfonyl]-7-ethoxyimidazo[1,2-a]pyridine; 6-chloro-4-[6-[(1,1-dimethylethyl)sulfonyl]-7-methoxyimidazo[1,2-a]pyridin-3-yl]-2-pyridinamine; N-[4-[6-[(1,1-dimethylethyl)sulfonyl]-7-methoxyimidazo[1,2-a]pyridin-3-yl]-2-pyridinyl]acetamide; 4-[6-[(1,1-dimethylethyl)sulfonyl]-7-ethoxyimidazo[1,2-a]pyridin-3-yl]-6-fluoro-2-pyridinamine; 6-chloro-4-[6-[(1,1-dimethylethyl)sulfonyl]-7-ethoxyimidazo[1,2-a]pyridin-3-yl]-2-pyridinamine; 6-chloro-4-[6-[(1,1-dimethylethyl)sulfonyl]-7-(1-methylethoxy)imidazo[1,2-a]pyridin-3-yl]-2-pyridinamine; 6-chloro-4-[6-[(1,1-dimethylethyl)sulfonyl]-7-(2-methoxyethoxy)imidazo[1,2-a]pyridin-3-yl]-2-pyridinamine; 6-chloro-4-[7-(cyclopropyloxy)-6-[(1,1-dimethylethyl)sulfonyl]imidazo[1,2-a]pyridin-3-yl]-2-pyridinamine; 6-chloro-4-[6-[(1,1-dimethylethyl)sulfonyl]-7-(2,2,2-trifluoroethoxy)imidazo[1,2-a]pyridin-3-yl]-2-pyridinamine; 6-chloro-4-[6-[(1,1-dimethylethyl)sulfonyl]-7-[2-(4-methyl-1-piperazinyl)ethoxy]imidazo[1,2-a]pyridin-3-yl]-2-pyridinamine; and 4-[6-[(1,1-Dimethylethyl)sulfonyl]-7-ethoxyimidazo[1,2-a]pyridin-3-yl]-6-fluoro-N-[(4-methoxyphenyl)methyl]-2-pyridinamine, or a pharmaceutically acceptable salt of any of the foregoing.

In some embodiments, the compounds of Formula (I), include the compounds of Examples 1-340 and pharmaceutically acceptable salts and solvates thereof. In some embodiments, the compounds of Examples 1-340 are in the free base form. In some embodiments, the compounds of Examples 1-340 are in salt form, e.g., pharmaceutically acceptable salt form.

The ability of test compounds to act as RIPK2 inhibitors may be demonstrated by the biological assays described herein. IC₅₀ values are shown in Table A.

### Methods of Treatment

The compounds and compositions disclosed herein are effective for modulating the activity of RIPK2. In some embodiments, the compounds and compositions disclosed herein are RIPK2 inhibitors.

The term “RIPK2-associated disease or disorder” as used herein refers to diseases or disorders associated with or having a dysregulation of a RIPK2 gene, a RIPK2 protein, or the expression or activity or level of any (e.g., one or more) of the same (e.g., any of the types of dysregulation of a RIPK2 gene, a RIPK2 protein, a RIPK2 protein domain, or the expression or activity or level of any of the same described herein).

An exemplary sequence of human RIPK2 is shown below (UniParc Accession No. UPI00001338F2):

MNGEAICSALPTIPYHKLADLRYLSRGASGTVSSARHADWRVQVAVKHL
HIHTPLLDSERKDVLREAEILHKARFSYILPILGICNEPEFLGIVTEYM
PNGSLNELLHRKTEYPDVAWPLRFRILHEIALGVNYLHNMTPPLLHHDL
KTQNILLDNEFHVKIADFGLSKWRMMSLSQSRSSKSAPEGGTIIYMPPE
NYEPGQKSRASIKHDIYSYAVITWEVLSRKQPFEDVTNPLQIMYSVSQG
HRPVINEESLPYDIPHRARMISLIESGWAQNPDERPSFLKCLIELEPVL
RTFEEITFLEAVIQLKKTKLQSVSSAIHLCDKKKMELSLNIPVNHGPQE
ESCGSSQLHENSGSPETSRSLPAPQDNDFLSRKAQDCYFMKLHHCPGNH
SWDSTISGSQRAAFCDHKTTPCSSAIINPLSTAGNSERLQPGIAQQWIQ
SKREDIVNQMTEACLNQSLDALLSRDLIMKEDYELVSTKPTRTSKVRQL
LDTTDIQGEEFAKVIVQKLKDNKQMGLQPYPEILVVSRSPSLNLLQNKS
M

Some embodiments provide a method of treating a RIPK2-associated disease or disorder in a subject in need thereof, comprising administering to the subject an effective amount of a compound of Formula (I), or a pharmaceutically acceptable salt thereof, or a pharmaceutical composition comprising an effective amount of a compound of Formula (I), or a pharmaceutically acceptable salt thereof.

Some embodiments provide a method of treating a RIPK2-associated disease or disorder in a subject in need thereof, comprising (a) determining that the subject is suffering from a RIPK2-associated disease or disorder; and (b) administering to the subject an effective amount of a compound of Formula (I), or a pharmaceutically acceptable salt thereof, or a pharmaceutical composition comprising an effective amount of a compound of Formula (I), or a pharmaceutically acceptable salt thereof.

Some embodiments provide a method of treating a RIPK2-associated disease or disorder in a subject previously identified or diagnosed as having a RIPK2-associated disease or disorder, the method comprising administering to the subject an effective amount of a compound of Formula (I), or a pharmaceutically acceptable salt thereof, or a pharmaceutical composition comprising an effective amount of a compound of Formula (I), or a pharmaceutically acceptable salt thereof.

In some embodiments, the RIPK2-associated disease or disorder is a cardiovascular disease, an allergic disorder, an autoimmune disease, an inflammatory disease, a cardiovascular disease, a fibrotic disease, or a disease associated with abnormal cell growth.

In some embodiments, the RIPK2-associated disease or disorder is a Type I hypersensitivity (allergic) reaction. In some embodiments, the Type I hypersensitivity (allergic) reaction is allergic inflammation. In some embodiments, the allergic inflammation is allergic rhinitis, allergic asthma, allergic conjunctivitis, atopic- and vernal keratoconjunctivitis, or atopic dermatitis.

In some embodiments, the RIPK2-associated disease or disorder is an autoimmune disease. In some embodiments, the autoimmune disease is Crohn's disease, ulcerative colitis, rheumatoid arthritis, multiple sclerosis, encephalomyelitis, systemic lupus erythematosus, psoriasis, lupus nephritis, immune thrombocytopenic purpura, Sjogren's syndrome, ankylosing spondylitis, psoriatic arthritis, juvenile dermatomyositis, juvenile rheumatoid arthritis, juvenile spondyloarthopathy, non-radiographic spondyloarthopathy, Behcet's disease, dermatomyositis, diabetes mellitus type 1, Goodpasture's syndrome, Graves' disease, Guillain-Barre syndrome, Hashimoto's disease, mixed connective tissue damage, myasthenia gravis, narcolepsy, pemphigus vulgaris, pernicious anemia, polymyositis, primary biliary cirrhosis, temporal arteritis, or vasculitis. In some embodiments, the autoimmune disease is Crohn's disease, ulcerative colitis, inflammatory bowel disease, or multiple sclerosis. In some embodiments, the autoimmune disease is Crohn's disease. In some embodiments, the autoimmune disease is ulcerative colitis. In some embodiments, the autoimmune disease is inflammatory bowel disease. In some embodiments, the autoimmune disease is multiple sclerosis.

In some embodiments, the RIPK2-associated disease or disorder is a metabolic disease. In some embodiments, the metabolic disease is dysglycemia, type 2 diabetes, non-alcoholic fatty liver disease (including non-alcoholic steatohepatitis), or obesity.

In some embodiments, the RIPK2-associated disease or disorder is an inflammatory disease. In some embodiments, the inflammatory disease is chronic lung inflammatory disease, osteoarthritis, inflammatory arthritis, asthma, early onset sarcoidosis, sarcoidosis, eczema, allergic eczema, uveitis, reactive arthritis, chronic inflammation, chronic prostatitis, inflammatory bowel disease, glomerulonephritis, bursitis, carpal tunnel syndrome, tendinitis, inflammation of the lung (e.g., chronic obstructive pulmonary disease), pelvic inflammatory disease, transplant rejection, vasculitis, regional enteritis, distal ileitis, regional ileitis, and terminal ileitis, central areolar choroidal dystrophy, macular degeneration, retinosis pigmentosa, adult vitelliform disease, pattern dystrophy, diabetic retinopathy, BEST disease, myopic degeneration, central serous retinopathy, Stargardt's disease, Cone-Rod dystrophy, North Carolina dystrophy, infectious retinitis, inflammatory retinitis, uveitis, toxic retinitis, or systemic inflammatory response syndrome. In some embodiments, the inflammatory disease is inflammatory bowel disease.

In some embodiments, the RIPK2-associated disease or disorder isgranulomatous inflammatory disease. In some embodiments, the granulomatous inflammatory disease is Wegener's granulomatosis, Churg-Strauss syndrome, relapsing polychondritis, polyarteritis nodosa, giant cell arteritis, primary biliary cirrhosis, hepatic granulomatous disease, Langerhan's granulomatosis, granulomatous enteritis, orofacial granulomatosis, or Peyronie's disease.

In some embodiments, the RIPK2-associated disease or disorder is a cardiovascular disease. In some embodiments, the cardiovascular disease is atherosclerosis, thrombosis, myocardial infarction, stroke, aortic aneurysm, arterial hypertension, sickle cell crisis, or ischemia-reperfusion injury.

In some embodiments, the RIPK2-associated disease is lethal systemic inflammatory response syndrome, chronic gut and skin inflammation, or acute pancreatitis.

In some embodiments, the RIPK2-associated disease or disorder is a fibrotic disease. In some embodiments, the fibrotic disease is scleroderma, asbestosis, or idiopathic pulmonary fibrosis.

In some embodiments, the RIPK2-associated disease or disorder comprises neuroinflammation. In some embodiments, the RIPK2-associated disease or disorder is Alzheimer's disease, amyotrophic lateral sclerosis (ALS), Parkinson's disease, Huntington's disease, Lewy body disease, Niemann-Pick disease, type C1 (NPC1), Friedreich's ataxia, spinal muscular atrophy, corticobasal degeneration, progress supranuclear palsy (PSP), or multiple system atrophy (MSA).

In some embodiments, the RIPK2-associated disease or disorder is a disease related to abnormal cell growth. In some embodiments, the disease related to abnormal cell growth is cancer, including hematological malignancies and solid tumors.

Hematological malignancies include, but are not limited to leukemias, such as acute myeloid leukemia, chronic myelogenous leukemia, chronic lymphocytic leukemia, B-cell chronic lymphocytic leukemia, and lymphomas and myelomas, such as B-cell lymphoma (e.g., mantle cell lymphoma), T-cell lymphoma (e.g., peripheral T-cell lymphoma), non-Hodgkin's lymphoma, and multiple myeloma.

Solid tumors include lung cancer (small cell lung cancer and non-small cell lung cancer), pancreatic cancer, colon cancer, breast cancer, genitourinary cancer, skin cancer, bone cancer, prostate cancer, liver cancer, brain cancer, laryngeal cancer, gall bladder cancer, rectal cancer, parathyroid cancer, thyroid cancer, adrenal cancer, neural tissue cancer, bladder cancer, head and neck cancer, stomach cancer, gastric cancer, bronchial cancer, and kidney cancer (e.g., renal clear cell carcinoma), colorectal cancer, clear cell carcinoma, basal cell carcinoma, squamous cell carcinoma, esophageal cancer, metastatic skin carcinoma, osteosarcoma, Ewing's sarcoma, reticulum cell sarcoma, Kaposi's sarcoma, giant cell tumor, islet cell tumor, acute and chronic lymphocytic and granulocytic tumors, hairy-cell tumor, adenoma, medullary carcinoma, pheochromocytoma, mucosal neuromas, intestinal ganglioneuromas, hyperplastic corneal nerve tumor, marfanoid habitus tumor, Wilms' tumor, seminoma, ovarian tumor, leiomyomata tumor, cervical dysplasia, neuroblastoma, retinoblastoma, myelodysplastic syndrome, rhabdomyosarcoma, astrocytoma, malignant hypercalcemia, polycythemia vera, adenocarcinoma, glioblastoma multiforma, glioma, and malignant melanoma.

In some embodiments, the RIPK2-associated disease or disorder is a disease related to abnormal cell growth that is a non-malignant proliferative disease. In some embodiments, the non-malignant proliferative disease is benign prostatic hypertrophy, restenosis, hyperplasia, synovial proliferation disorder, idiopathic plasmacytic lymphadenopathy, or retinopathy.

In some embodiments, the RIPK2-associated disease or disorder is selected from the group consisting of: avascular necrosis, calcium pyrophosphate dihydrate crystal deposition disease (pseudo gout), Blau syndrome, Ehlers-Danlos syndrome, fibromyalgia, Fifth disease, giant cell arteritis, gout, Lyme disease, Marfan syndrome, myositis, osteoarthritis, osteogenesis imperfecta, osteoporosis, Paget's disease, Raynaud's phenomenon, reactive arthritis, reflex sympathetic dystrophy syndrome, spinal stenosis, and Still's disease.

In some embodiments, the RIPK2-associated disease or disorder is a cancer associated with chronic inflammation. In some embodiments, the cancer associated with chronic inflammation that can be treated (including reduction in the likelihood of recurrence) include colitis-associated colorectal cancer, gastric cancer, gastric mucosal lymphoma, lung cancer, hepatocellular carcinoma, thyroid cancer, breast cancer, oral cancer, head and neck cancer, nasopharyngeal carcinoma, endometrial cancer, uterine cancer, ovarian cancer, prostate cancer, bladder cancer, pancreatic cancer, esophageal cancer, skin cancer, and non-Hodgkin lymphoma.

Some embodiments provide a method of treating inflammatory bowel disease in a subject in need thereof, comprising administering to the subject an effective amount of a compound of Formula (I), or a pharmaceutically acceptable salt thereof, or a pharmaceutical composition comprising an effective amount of a compound of Formula (I), or a pharmaceutically acceptable salt thereof.

Some embodiments provide a method of treating inflammatory bowel disease in a subject in need thereof, comprising (a) determining that the subject is suffering from inflammatory bowel disease; and (b) administering to the subject an effective amount of a compound of Formula (I), or a pharmaceutically acceptable salt thereof, or a pharmaceutical composition comprising an effective amount of a compound of Formula (I), or a pharmaceutically acceptable salt thereof.

Some embodiments provide a method of treating inflammatory bowel disease in a subject previously identified or diagnosed as having inflammatory bowel disease, the method comprising administering to the subject an effective amount of a compound of Formula (I), or a pharmaceutically acceptable salt thereof, or a pharmaceutical composition comprising an effective amount of a compound of Formula (I), or a pharmaceutically acceptable salt thereof.

Some embodiments provide a method of treating Crohn's disease in a subject in need thereof, comprising administering to the subject an effective amount of a compound of Formula (I), or a pharmaceutically acceptable salt thereof, or a pharmaceutical composition comprising an effective amount of a compound of Formula (I), or a pharmaceutically acceptable salt thereof.

Some embodiments provide a method of treating Crohn's disease in a subject in need thereof, comprising (a) determining that the subject is suffering from Crohn's disease; and (b) administering to the subject an effective amount of a compound of Formula (I), or a pharmaceutically acceptable salt thereof, or a pharmaceutical composition comprising an effective amount of a compound of Formula (I), or a pharmaceutically acceptable salt thereof.

Some embodiments provide a method of treating Crohn's disease in a subject previously identified or diagnosed as having Crohn's disease, the method comprising administering to the subject an effective amount of a compound of Formula (I), or a pharmaceutically acceptable salt thereof, or a pharmaceutical composition comprising an effective amount of a compound of Formula (I), or a pharmaceutically acceptable salt thereof.

In some embodiments, the subject is a human.

### Inhibiting RIPK2 Activity

Some embodiments provide a method for inhibiting RIPK2 activity in a mammalian cell, comprising contacting the mammalian cell with a compound of Formula (I), or a pharmaceutically acceptable salt thereof. In some embodiments, the mammalian cell comprises a RIPK2 protein.

Also provided is a method for inhibiting RIPK2 activity in a mammalian cell comprising a RIPK2 protein, the method comprising contacting the mammalian cell with a compound of Formula (I), or a pharmaceutically acceptable salt thereof.

In some embodiments, the contacting is in vitro. In some embodiments, the contacting is in vivo. In some embodiments, the amount of a compound of Formula (I), or a pharmaceutically acceptable salt thereof, is sufficient to inhibit RIPK2 activity in the cell. In some embodiments, the contacting is in vivo, wherein the method comprises administering a therapeutically effective amount of a compound of Formula (I), or a pharmaceutically acceptable salt thereof, to a subject having a mammalian cell having RIPK2 activity. In some embodiments, the mammalian cell is a mammalian immune cell. In some embodiments, the mammalian cell is an cancer cell.

In some embodiments, the RIPK2 activity is inhibited by about 10% to about 99%, for example, about 10% to about 50%, about 25% to about 75%, about 50% to about 99%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or any value in between.

As used herein, the term “contacting” refers to the bringing together of indicated moieties in an in vitro system or an in vivo system. For example, “contacting” RIPK2 (e.g., a RIPK2 protein) with a compound provided herein includes the administration of a compound provided herein to a subject, such as a human, having a RIPK2 protein, as well as, for example, introducing a compound provided herein into a sample containing a mammalian cellular or purified preparation containing a RIPK2 protein.

### Pharmaceutical Compositions

Some embodiments provide a pharmaceutical composition comprising a compound of Formula (I), or a pharmaceutically acceptable salt thereof, and at least one pharmaceutically acceptable excipient.

### EXAMPLES

### Materials and Methods

The compounds provided herein, including salts thereof, can be prepared using known organic synthesis techniques and can be synthesized according to any of numerous possible synthetic routes.

The reactions for preparing the compounds provided herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of the compounds provided herein can involve the protection and deprotection of various chemical groups. The need for protection and deprotection, and the selection of appropriate protecting groups, can be readily determined by one skilled in the art. The chemistry of protecting groups can be found, for example, in Protecting Group Chemistry, 1^{st} Ed., Oxford University Press, 2000; March's Advanced Organic Chemistry: Reactions, Mechanisms, and Structure, 5^{th} Ed., Wiley-Interscience Publication, 2001; and Peturssion, S. et al., “Protecting Groups in Carbohydrate Chemistry,” J. Chem. Educ., 74(11), 1297 (1997).

### Intermediates

### Intermediate 1

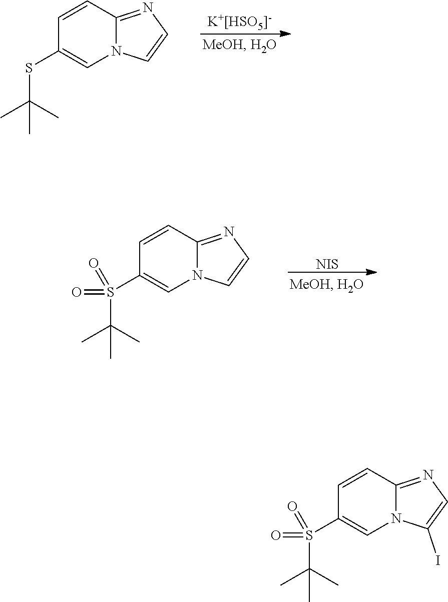

6-(tert-butylsulfonyl)imidazo[1,2-a]pyridine: 6-(tert-butylthio)imidazo[1,2-a]pyridine (3.38 g, 16.4 mmol, prepared as described in U.S. Published Application No. US 2018/0072717, which is incorporated by reference herein in its entirety) was provided in a mixture of methanol (69 mL) and water (23 mL). Potassium 3-sulfotrioxidan-1-ide (15.1 g, 24.6 mmol) was added, and the mixture was stirred at rt for 18 h. The suspension was filtered, and the solids removed. The solvent was evaporated. The crude was purified using a preparative HPLC in three runs. The batches were combined to give the title compound. [M+H]⁺=239.3. ¹H NMR (400 MHz, DMSO-d₆) δ ppm 9.34 (dd, J=1.9, 0.9 Hz, 1H), 8.24 (s, 1H), 7.87 (d, J=1.3 Hz, 1H), 7.81 (d, J=9.4 Hz, 1H), 7.57 (dd, J=9.5, 1.9 Hz, 1H), 1.31 (s, 9H).

6-(tert-butylsulfonyl)-3-iodoimidazo[1,2-a]pyridine: 6-(tert-butylsulfonyl)imidazo[1,2-a]pyridine (2.75 g, 11.5 mmol) and N-iodo-succinimide (3.12 g, 13.8 mmol) were combined in a mixture of methanol (79 mL) and water (31 mL). The mixture was stirred for 20 h at 24° C. Water and a saturated aqueous solution of sodium bicarbonate were added, and the organic solvent was evaporated. The residue was extracted with dichloromethane and the combined organic phases were filtered and concentrated. The crude product was purified by flash chromatography (silica gel, dichloromethane/ethanol gradient) to give the title compound. [M+H]⁺=365.2. ¹H NMR (400 MHz, DMSO-d₆) δ ppm 8.55 (dd, J=1.9, 0.9 Hz, 1H), 7.95 (s, 1H), 7.82 (dd, J=9.5, 0.9 Hz, 1H), 7.54 (dd, J=9.4, 1.8 Hz, 1H), 1.33 (s, 9H).

### Intermediate 2

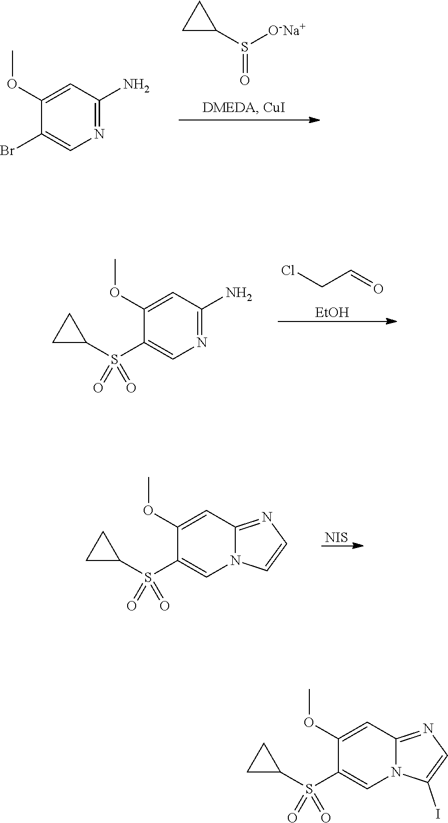

5-(Cyclopropylsulfonyl)-4-methoxypyridin-2-amine: To a stirred solution of 5-bromo-4-methoxypyridin-2-amine (0.6 g, 2.95 mmol, 1.0 eq), sodium cyclopropanesulfinate (0.95 g, 7.38 mmol, 2.5 eq) in DMF (6 mL) was added DMEDA (0.160 mL, 1.47 mmol, 0.5 eq) at rt, and the reaction mixture was degassed with N₂ gas for 10 min. To it was then added CuI (0.113 g, 0.59 mmol, 0.2 eq) and again degassed for 10 min with nitrogen. The resulting mixture was heated to 120° C. for 24 h. After the completion of reaction (followed by TLC and LCMS), the reaction mixture was concentrated under reduced pressure. The resulting residue was diluted with DCM (50 mL) and washed twice with a saturated aqueous NaHCO₃ solution (2×20 mL). The organic layer was separated, dried over anhydrous Na₂SO₄, filtered, and concentrated under vacuum. The crude compound was purified by flash column chromatography over silica gel (230-400 mesh) eluting with 1-5% MeOH/DCM to obtain the title compound. [M+H]⁺=229.24.

6-(Cyclopropylsulfonyl)-7-methoxyimidazo [1,2-a]pyridine: To a stirred solution of 5-(cyclopropylsulfonyl)-4-methoxypyridin-2-amine (0.13 g, 0.56 mmol, 1.0 eq) in EtOH (1.5 mL) was added 2-chloroacetaldehyde (0.223 g, 2.83 mmol, 5.0 eq) at rt and then heated to 80° C. for 4 h. After the completion of reaction (followed by TLC and LCMS), the mixture was allowed to cool to rt and was concentrated under vacuum. The crude residue was purified by flash column chromatography over silica gel (230-400 mesh) eluting with 1-5% MeOH/DCM to obtain the title compound [M+H]⁻=253.22.

6-(Cyclopropylsulfonyl)-3-iodo-7-methoxyimidazo [1,2-a]pyridine: To a stirred solution of 6-(cyclopropylsulfonyl)-7-methoxyimidazo [1, 2-a]pyridine (80 mg, 0.31 mmol, 1.0 eq) in DCM (0.8 mL), MeOH (0.1 mL) was added NIS (85.7 mg, 0.38 mmol, 1.2 eq) at 0° C. The resulting mixture was warmed to rt and stirred for 2 h. After completion of reaction, the reaction mass was concentrated under reduced pressure. The residue so obtained was diluted with DCM (100 mL) and washed with a saturated aqueous NaHCO3 solution. The organic layer was dried over anhydrous Na₂SO₄, filtered, and concentrated under vacuum. The crude compound was purified by flash column chromatography over silica gel (230-400 mesh) eluting with 1-5% MeOH/DCM to obtain the title compound. [M+H]⁺=379.0; H NMR (400 MHz, DMSO-d₆) δ 8.49 (s, 1H), 7.56 (s, 1H), 7.71 (s, 1H), 7.31 (s, 1H), 4.00 (s, 3H), 3.12-3.08 (m, 1H), 1.18-1.15 (m, 2H), 1.10-1.08 (m, 2H).

### Intermediate 3

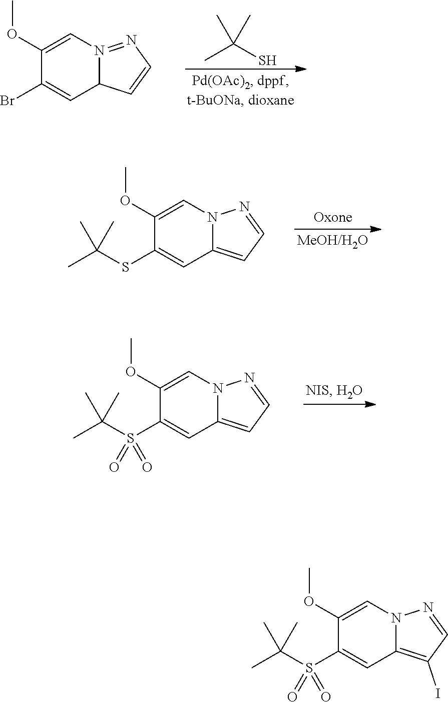

5-(tert-butylthio)-6-methoxypyrazolo[1,5-a]pyridine: A mixture of 5-bromo-6-methoxypyrazolo[1,5-a]pyridine (227 mg, 899.77 μmol, 1 eq), Pd(OAc)₂ (4.04 mg, 18.00 mol, 0.02 eq), dppf (19.95 mg, 35.99 μmol, 0.04 eq) and t-BuONa (129.71 mg, 1.35 mmol, 1.5 eq) in dioxane (5 mL) was degassed and purged with N₂ 3 times, and then 2-methylpropane-2-thiol (162.29 mg, 1.80 mmol, 202.62 μL, 2 eq) was added. The mixture was stirred at 90° C. for 12 h under a N₂ atmosphere before the reaction mixture was filtered and concentrated under reduced pressure to give a residue. The residue was purified by column chromatography (SiO₂, DCM/MeOH=20/1 to 10/1) to give the title compound. [M+H]⁺=237.1. ¹H NMR (400 MHz, DMSO-d₆) δ ppm 8.41 (s, 1H), 7.90 (d, J=2.4 Hz, 1H), 7.85 (s, 1H), 6.60 (d, J=2.4 Hz, 1H), 3.83 (s, 3H), 1.28 (s, 9H).

5-(tert-butylsulfonyl)-6-methoxypyrazolo[1,5-a]pyridine: A mixture of 5-(tert-butylthio)-6-methoxypyrazolo[1,5-a]pyridine (188 mg, 715.94 μmol, 1 eq) and Oxone® (660.21 mg, 1.07 mmol, 1.5 eq) in MeOH (1.5 mL) and H₂O (0.5 mL) was stirred at 25° C. for 12 h under a N₂ atmosphere. The title compound was obtained. [M+H]=269.0.

5-(tert-butylsulfonyl)-3-iodo-6-methoxypyrazolo[1,5-a]pyridine: To a solution of 5-(tert-butylsulfonyl)-6-methoxypyrazolo[1,5-a]pyridine (200 mg, 745.35 μmol, 1 eq) in H₂O (1 mL) was added NIS (251.53 mg, 1.12 mmol, 1.5 eq). The resulting mixture was stirred at 20° C. for 12 h before the mixture was quenched with saturated Na₂SO₃ (5 mL) and extracted with EtOAc (30 mL). The combined organic phase was dried over anhydrous sodium sulfate, filtered, and concentrated to give a residue. The residue was purified by column chromatography (SiO₂, petroleum ether/EtOAc=10/1 to 3/1) to give the title compound. [M+H]⁺=394.9. ¹H NMR (400 MHz, DMSO-d₆) δ ppm 8.79 (s, 1H), 8.22 (s, 1H), 7.87 (s, 1H), 3.90 (s, 3H), 1.31 (s, 9H).

### Intermediate 4

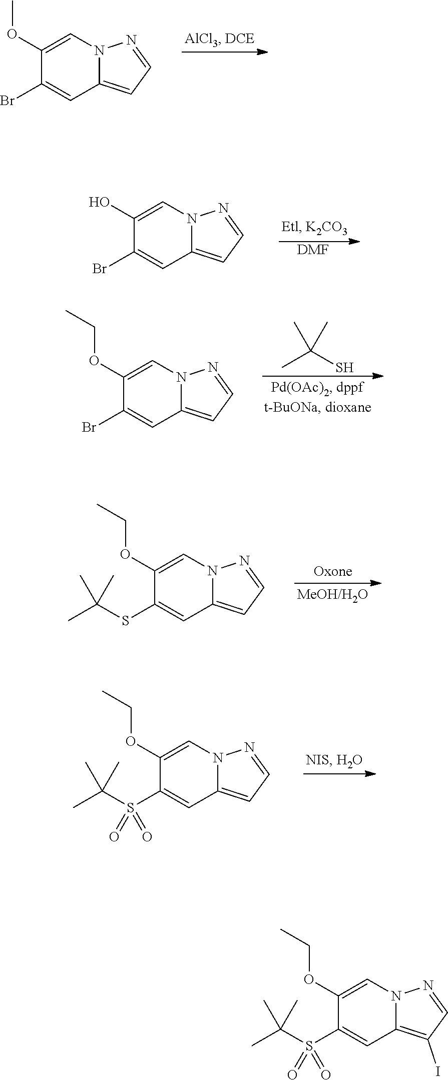

5-bromopyrazolo[1,5-a]pyridin-6-ol: To a solution of 5-bromo-6-methoxypyrazolo[1,5-a]pyridine (500 mg, 1.98 mmol, 1 eq) in DCE (8 mL) was added AlCl₃ (1.32 g, 9.91 mmol, 541.53 μL, 5 eq). The mixture was stirred at 80° C. for 1 h before it was quenched with saturated Na₂SO₄ (5 mL), neutralized with saturated NaHCO₃, and extracted with DCM (3×40 mL). The organic phase was dried over Na₂SO₄, filtered, and concentrated under reduced pressure to give the title compound. [M+H]⁺=214.8. ¹H NMR (400 MHz, DMSO-d₆) δ ppm 10.33 (s, 1H), 8.21 (s, 1H), 8.02 (s, 1H), 7.83 (d, J=1.6 Hz, 1H), 6.47 (d, J=2.0 Hz, 1H).

5-bromo-6-ethoxypyrazolo[1,5-a]pyridine: A mixture of 5-bromopyrazolo[1,5-a]pyridin-6-ol (180.00 mg, 844.95 μmol, 1 eq), K₂CO₃ (350.33 mg, 2.53 mmol, 3 eq) and iodoethane (171.32 mg, 1.10 mmol, 87.85 μL, 1.3 eq) in DMF (25 mL) was degassed and purged with N₂ 3 times, and then the mixture was stirred at 80° C. for 2 h under a N₂ atmosphere. The reaction mixture was diluted with brine (20 mL) and extracted with EtOAc (3×10 mL), dried over Na₂SO₄, filtered, and concentrated under reduced pressure to give a residue, which was purified by column chromatography (SiO₂, petroleum ether/EtOAc=1/0 to 4/1) to give the title compound. ¹H NMR (400 MHz, DMSO-d₆) δ ppm 8.51 (s, 1H), 8.08 (s, 1H), 7.91 (d, J=2.4 Hz, 1H), 6.52 (d, J=2.4 Hz, 1H), 4.12 (q, J=6.8 Hz, 2H), 1.39 (t, J=7.2 Hz, 3H).

5-(tert-butylthio)-6-ethoxypyrazolo[1,5-a]pyridine: A mixture of 5-bromo-6-ethoxypyrazolo[1,5-a]pyridine (190 mg, 709.30 μmol, 1 eq), 2-methylpropane-2-thiol (108.75 mg, 1.21 mmol, 135.76 μL, 1.7 eq), dppf (15.73 mg, 28.37 mol, 0.04 eq), Pd(OAc)₂ (3.18 mg, 14.19 mol, 0.02 eq) and t-BuONa (204.50 mg, 2.13 mmol, 3 eq) in dioxane (5 mL) was degassed and purged with N₂ 3 times, and then the mixture was stirred at 90° C. for 12 h under a N₂ atmosphere. After the reaction was cooled to room temperature, the mixture was diluted with saturated NH₄Cl (20 mL) and extracted with EtOAc (4×10 mL). The combined organic extracts were dried over Na₂SO₄, filtered, and concentrated under reduced pressure to give a residue, which was purified by column chromatography (SiO₂, petroleum ether/EtOAc=100/0 to 80/20) to give the title compound. [M+H]⁺=251.0. ¹H NMR (400 MHz, DMSO-d₆) δ ppm 8.39 (s, 1H), 7.89 (d, J=2.4 Hz, 1H), 7.85 (s, 1H), 6.59 (d, J=2.4 Hz, 1H), 4.06 (q, J=6.8 Hz, 2H), 1.37 (t, J=6.8 Hz, 3H), 1.29 (s, 9H).

5-(tert-butylsulfonyl)-6-ethoxypyrazolo[1,5-a]pyridine: To a solution of 5-(tert-butylthio)-6-ethoxypyrazolo[1,5-a]pyridine (60 mg, 215.69 μmol, 1 eq) in MeOH (3 mL) and H₂O (1 mL) was added Oxone© (198.90 mg, 323.54 mol, 1.5 eq). The mixture was stirred at 25° C. for 12 h before it was concentrated under reduced pressure to give the title compound, which was used to next step without further purification. [M+H]⁺=283.1.

5-(tert-butylsulfonyl)-6-ethoxy-3-iodopyrazolo[1,5-a]pyridine: To a solution of 5-(tert-butylsulfonyl)-6-ethoxypyrazolo[1,5-a]pyridine (50 mg, 177.08 mol, 1 eq) in H₂O (0.25 mL) was added NIS (59.76 mg, 265.62 mol, 1.5 eq). The mixture was stirred at 25° C. for 1 h before was added dropwise to saturated NaHCO₃ (10 mL) and extracted with DCM (3×20 mL). The combined organic extracts were dried over Na₂SO₄, filtered, and concentrated under reduced pressure to give a residue, which was purified by column chromatography (SiO₂, petroleum ether/EtOAc=100/0 to 80/20) to give the title compound. [M+H]⁺=408.8. ¹H NMR (400 MHz, DMSO-d₆) δ ppm 8.77 (s, 1H), 8.21 (s, 1H), 7.88 (s, 1H), 4.15 (q, J=6.8 Hz, 2H), 1.39-1.30 (m, 12H).

### Intermediate 5

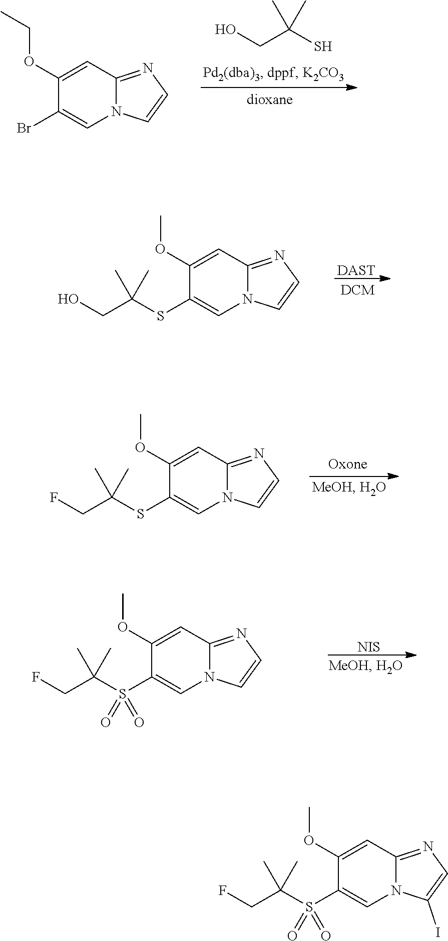

2-((7-methoxyimidazo[1,2-a]pyridin-6-yl)thio)-2-methylpropan-1-ol: A mixture of 6-bromo-7-methoxyimidazo[1,2-a]pyridine (50 mg, 198.19 μmol, 1 eq), K₂CO₃ (82.17 mg, 594.56 μmol, 3 eq), dppf (10.99 mg, 19.82 μmol, 0.1 eq) and Pd₂(dba)₃ (9.07 mg, 9.91 μmol, 0.05 eq) in dioxane (1 mL) was degassed and purged with N₂ (3×). 2-mercapto-2-methylpropan-1-ol (23.15 mg, 218.01 μmol, 1.1 eq) was added, and then the mixture was stirred at 100° C. for 12 hours under N₂ atmosphere. The reaction mixture was diluted with H₂O (10 mL) and extracted with EtOAc (3×10 mL). The combined organic layers were washed with brine (15 mL), dried over Na₂SO₄, filtered, and concentrated in vacuo. The resulting crude material was purified via silica gel chromatography (DCM/MeOH=100/0 to 10/1) to yield the title compound. LCMS [M+H]⁺=253.0. ¹H NMR (400 MHz, DMSO-d₆) δ ppm 8.70 (s, 1H), 7.76 (s, 1H), 7.41 (s, 1H), 6.96 (s, 1H), 4.78 (t, J=6.0 Hz, 1H), 3.85 (s, 3H), 3.27 (d, J=6.0 Hz, 2H), 1.13 (s, 6H).

6-((1-fluoro-2-methylpropan-2-yl)thio)-7-methoxyimidazo[1,2-a]pyridine: To a solution of 2-((7-methoxyimidazo[1,2-a]pyridin-6-yl)thio)-2-methylpropan-1-ol (0.58 g, 2.07 mmol, 1 eq) in DCM (5 mL) was added DAST (546.64 μL, 4.14 mmol, 2 eq) at 0° C. The mixture was stirred at 25° C. for 16 h under N₂ atmosphere. The reaction mixture was quenched by the addition of saturated NaHCO₃ at 0° C. until pH=8 and was then extracted with DCM (3×20 mL). The combined organic layers were washed with brine (30 mL), dried over Na₂SO₄, filtered, and concentrated in vacuo. The resulting crude material was purified via silica gel chromatography (DCM/MeOH=1/0 to 95/5) to yield the title compound. LCMS [M+H]⁺=255.0. ¹H NMR (400 MHz, DMSO-d₆) δ ppm 8.59 (s, 1H), 7.70 (s, 1H), 7.38 (s, 1H), 6.97 (s, 1H), 3.89 (s, 3H), 3.15 (d, J=18.8 Hz, 2H), 1.45 (s, 3H), 1.40 (s, 3H).

6-((1-fluoro-2-methylpropan-2-yl)sulfonyl)-7-methoxyimidazo[1,2-a]pyridine: To a solution of 6-((1-fluoro-2-methylpropan-2-yl)thio)-7-methoxyimidazo[1,2-a]pyridine (260 mg, 920.09 μmol, 1 eq) in H₂O (3 mL) and MeOH (9 mL) was added Oxone (2.26 g, 3.68 mmol, 4 eq), and the reaction mixture was stirred at 50° C. for 16 h. The title compound was obtained, which was used into the next step without further purification.

6-((1-fluoro-2-methylpropan-2-yl)sulfonyl)-3-iodo-7-methoxyimidazo[1,2-a]pyridine: To a solution of 6-((1-fluoro-2-methylpropan-2-yl)sulfonyl)-7-methoxyimidazo[1,2-a]pyridine (263.4 mg, 919.94 mol, 1 eq) in H₂O (3 mL) was added NIS (310.46 mg, 1.38 mmol, 1.5 eq) at 25° C. The mixture was stirred at 25° C. for 2 h. The reaction mixture was quenched by the addition saturated NaHCO₃ at 0° C. until pH=8 and extracted with DCM (3×20 mL). The combined organic layers were washed with brine (30 mL), dried over Na₂SO₄, filtered, and concentrated in vacuo. The resulting crude material was purified via silica gel chromatography (DCM/MeOH=1/0 to 10/1) to yield the title compound. LCMS [M+H]⁺=412.9. ¹H NMR (400 MHz, CDCl₃) δ ppm 8.74 (s, 1H), 7.65 (s, 1H), 7.03 (s, 1H), 4.05 (s, 3H), 3.78 (d, J=15.2 Hz, 2H), 1.65 (s, 3H), 1.60 (s, 3H).

### Intermediate 6

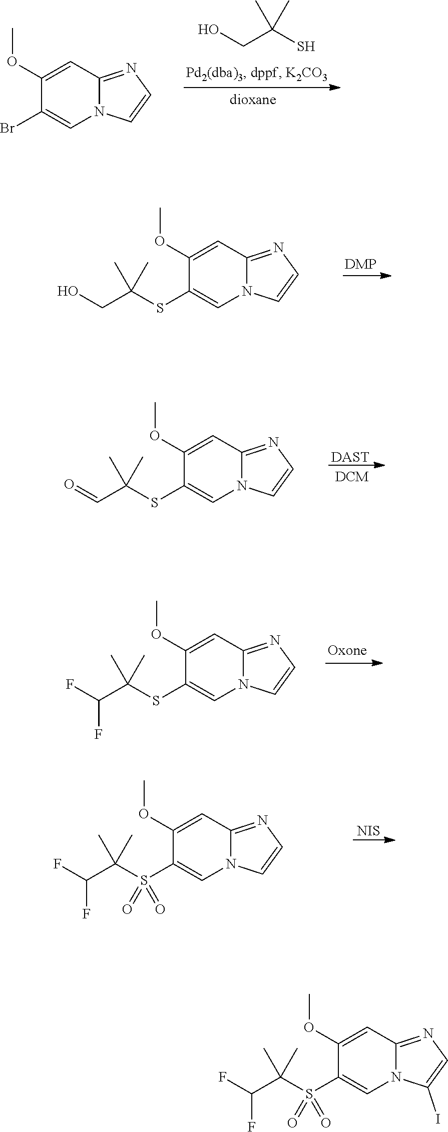

2-((7-methoxyimidazo[1,2-a]pyridin-6-yl)thio)-2-methylpropan-1-ol: A mixture of 6-bromo-7-methoxyimidazo[1,2-a]pyridine (50 mg, 198.19 μmol, 1 eq), K₂CO₃ (82.17 mg, 594.56 μmol, 3 eq), dppf (10.99 mg, 19.82 μmol, 0.1 eq) and Pd₂(dba)₃ (9.07 mg, 9.91 μmol, 0.05 eq) in dioxane (1 mL) was degassed and purged with N₂ (3×). 2-mercapto-2-methylpropan-1-ol (23.15 mg, 218.01 μmol, 1.1 eq) was added, and then the mixture was stirred at 100° C. for 12 hours under N₂ atmosphere. The reaction mixture was diluted with H₂O (10 mL) and extracted with EtOAc (3×10 mL). The combined organic layers were washed with brine (15 mL), dried over Na₂SO₄, filtered, and concentrated in vacuo. The resulting crude material was purified via silica gel chromatography (DCM/MeOH=100/0 to 10/1) to yield the title compound. LCMS [M+H]⁺=253.0. ¹H NMR (400 MHz, DMSO-d₆) δ ppm 8.70 (s, 1H), 7.76 (s, 1H), 7.41 (s, 1H), 6.96 (s, 1H), 4.78 (t, J=6.0 Hz, 1H), 3.85 (s, 3H), 3.27 (d, J=6.0 Hz, 2H), 1.13 (s, 6H).

2-((7-methoxyimidazo[1,2-a]pyridin-6-yl)thio)-2-methylpropanal: To a solution of 2-((7-methoxyimidazo[1,2-a]pyridin-6-yl)thio)-2-methylpropan-1-ol (2 g, 7.13 mmol, 1 eq) in DCM (30 mL) was added DMP (3.32 mL, 10.70 mmol, 1.5 eq). The mixture was stirred at 20° C. for 2 h. The reaction mixture was quenched by the addition of saturated NaHCO₃ (80 mL) and extracted with DCM (3×80 mL). The combined organic layers were dried over Na₂SO₄, filtered, and concentrated in vacuo. The resulting crude material was purified via silica gel chromatography (DCM/MeOH=100/0 to 93/7) to yield the title compound. ¹H NMR (400 MHz, DMSO-d₆) δ ppm 9.27 (s, 1H), 8.69 (s, 1H), 7.76 (s, 1H), 7.43 (d, J=0.8 Hz, 1H), 6.96 (s, 1H), 3.80 (s, 3H), 1.26 (s, 6H).

6-((1,1-difluoro-2-methylpropan-2-yl)thio)-7-methoxyimidazo[1,2-a]pyridine: To a mixture of 2-((7-methoxyimidazo[1,2-a]pyridin-6-yl)thio)-2-methylpropanal (300 mg, 1.08 mmol, 1 eq) in DCM (30 mL) was added DAST (855.07 μL, 6.47 mmol, 6 eq) at −65° C. The reaction mixture was stirred at 25° C. for 2 h under N₂ atmosphere. The reaction mixture was quenched by the addition of saturated NaHCO₃ at 0° C. until pH=8. The resulting mixture was extracted with DCM (3×40 mL), and the combined organic layers were dried over Na₂SO₄, filtered, and concentrated in vacuo. The resulting crude material was purified via silica gel chromatography (DCM/MeOH=1/0 to 4/1) to yield the title compound. LCMS [M+H]⁺=272.9. ¹H NMR (400 MHz, DMSO-d₆) δ ppm 8.76 (s, 1H), 7.83-7.72 (m, 1H), 7.43 (s, 1H), 6.99 (s, 1H), 6.18-5.82 (m, 1H), 3.88 (s, 3H), 1.22 (s, 6H).

6-((1,1-difluoro-2-methylpropan-2-yl)sulfonyl)-7-methoxyimidazo[1,2-a]pyridine: To a mixture of 6-((1,1-difluoro-2-methylpropan-2-yl)thio)-7-methoxyimidazo[1,2-a]pyridine (220 mg, 727.10 μmol, 1 eq) in MeOH (15 mL) and H₂O (5 mL) was added Oxone (4.47 g, 7.27 mmol, 10 eq). The mixture was stirred at 30° C. for 12 h under N2 atmosphere. The reaction mixture was filtered and concentrated in vacuo to yield the title compound, which was used in the next step without further purification. LCMS [M+H]⁺=305.0.

6-((1,1-difluoro-2-methylpropan-2-yl)sulfonyl)-3-iodo-7-methoxyimidazo[1,2-a]pyridine: To a mixture of 6-((1,1-difluoro-2-methylpropan-2-yl)sulfonyl)-7-methoxyimidazo[1,2-a]pyridine (200 mg, 591.50 μmol, 1 eq) in MeOH (15 mL) and H₂O (5 mL) was added NIS (159.69 mg, 709.80 μmol, 1.2 eq) at 0° C., and then the mixture was stirred at 0° C. for 2 h under N₂ atmosphere. The reaction mixture was diluted with H₂O (30 mL) and extracted with DCM (3×30 mL). The combined organic layers were dried over Na₂SO₄, filtered, and concentrated in vacuo. The resulting crude material was purified via silica gel chromatography (DCM/MeOH=1/0 to 10/1) to yield the title compound. LCMS [M+H]⁺=430.8.

### Intermediate 7

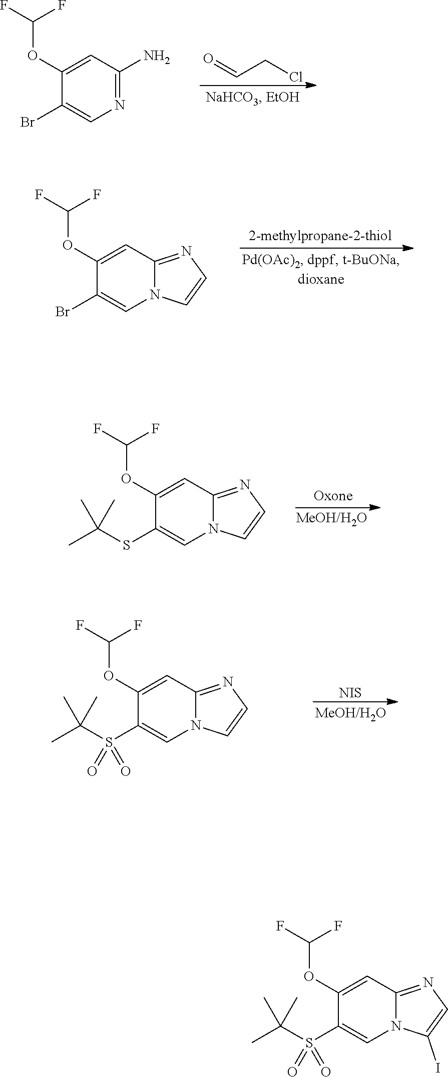

6-bromo-7-(difluoromethoxy)imidazo[1,2-a]pyridine: A mixture of 5-bromo-4-(difluoromethoxy)pyridin-2-amine (700 mg, 2.64 mmol, 1 eq), 2-chloroacetaldehyde (1.55 g, 7.91 mmol, 1.28 mL, 3 eq) and NaHCO₃ (664.27 mg, 7.91 mmol, 307.68 μL, 3 eq) in EtOH (10 mL) was stirred at 80° C. for 12 h under a N₂ atmosphere. The reaction mixture was concentrated under reduced pressure to give a residue, which was purified by column chromatography (SiO₂, petroleum ether/EtOAc=1/0 to 1/1) to give the title compound. [M+H]⁺=262.9. ¹H NMR (400 MHz, DMSO-d₆) δ ppm 9.46 (s, 1H), 8.25-8.05 (m, 2H), 7.94-7.50 (m, 2H).

6-(tert-butylthio)-7-(difluoromethoxy)imidazo[1,2-a]pyridine: A mixture of 6-bromo-7-(difluoromethoxy)imidazo[1,2-a]pyridine (700 mg, 2.40 mmol, 1 eq), 2-methylpropane-2-thiol (648.02 mg, 7.19 mmol, 809.01 μL, 3 eq), dppf (53.11 mg, 95.80 μmol, 0.04 eq), Pd(OAc)₂ (10.75 mg, 47.90 μmol, 0.02 eq) and t-BuONa (690.53 mg, 7.19 mmol, 3 eq) in dioxane (15 mL) was degassed and purged with N₂ 3 times, and then it was stirred at 90° C. for 12 h under a N₂ atmosphere. The reaction mixture was concentrated under reduced pressure to give a residue, which was purified by column chromatography (SiO₂, petroleum ether/EtOAc=1/0 to 4/1) to give the title compound. [M+H]⁺273.0. ¹H NMR (400 MHz, DMSO-d₆) δ ppm 8.89 (s, 1H), 7.94 (s, 1H), 7.60-7.20 (m, 3H), 1.27 (s, 9H).

6-(tert-butylsulfonyl)-7-(difluoromethoxy)imidazo[1,2-a]pyridine: A mixture of 6-(tert-butylthio)-7-(difluoromethoxy)imidazo[1,2-a]pyridine (30 mg, 99.15 mol, 1 eq) and Oxone® (304.77 mg, 495.75 μmol, 5 eq) in MeOH (3 mL) and H₂O (1 mL) was stirred at 25° C. for 2 h under a N₂ atmosphere. The reaction mixture was concentrated under reduced pressure to give the title compound. [M+H]⁺=304.9.

6-(tert-butylsulfonyl)-7-(difluoromethoxy)-3-iodoimidazo[1,2-a]pyridine: To a mixture of 6-(tert-butylsulfonyl)-7-(difluoromethoxy)imidazo[1,2-a]pyridine (30 mg, 98.58 μmol, 1 eq) in MeOH (3 mL) and H₂O (1 mL) was added NIS (26.62 mg, 118.30 mol, 1.2 eq) at 0° C., and the mixture stirred at 0° C. for 1 h under a N₂ atmosphere. The reaction mixture was quenched with sat. Na₂SO₃ (10 mL), and then diluted with H₂O (10 mL) and extracted with ethyl acetate (2×20 mL), dried over Na₂SO₄, filtered, and concentrated under reduced pressure to give the title compound. [M+H]⁺=430.8. ¹H NMR (400 MHz, DMSO-d₆) δ ppm 8.58 (s, 1H), 7.89 (s, 1H), 7.57 (s, 1H), 7.44-7.20 (m, 1H), 1.35 (s, 9H).

### Intermediate 8

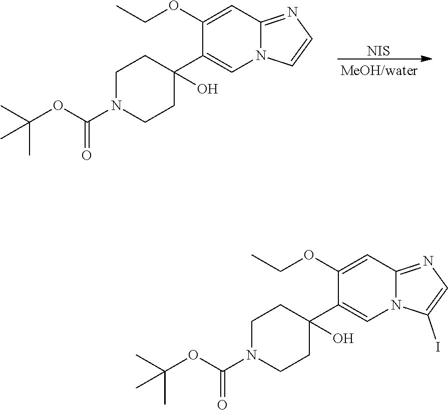

tert-Butyl 4-(7-ethoxy-3-iodoimidazo[1,2-a]pyridin-6-yl)-4-hydroxypiperidine-1-carboxylate: To tert-butyl 4-(7-ethoxyimidazo[1,2-a]pyridin-6-yl)-4-hydroxypiperidine-1-carboxylate (1.83 g, 5.06 mmol, prepared according to US20180072717 A1) in methanol (20 mL) and water (8 mL) was added N-iodosuccinimide (1.37 g, 6.08 mmol) and the mixture was stirred for 30 min at 20° C. Aqueous sodium thiosulfate-solution (10%) and water were added and the precipitate was collected by filtration, washed with water and dried to give the title compound, which was used without further purification. [M+H]⁺=488.5. ¹H NMR (400 MHz, DMSO-d₆) δ ppm 8.34 (s, 1H), 7.51 (s, 1H), 7.00 (s, 1H), 5.52 (s, 1H), 4.03-4.19 (m, 2H), 3.73-3.98 (m, 2H), 2.97-3.28 (m, 2H), 1.42 (s, 9H), 1.30-1.39 (m, 5H).

### Intermediate 9

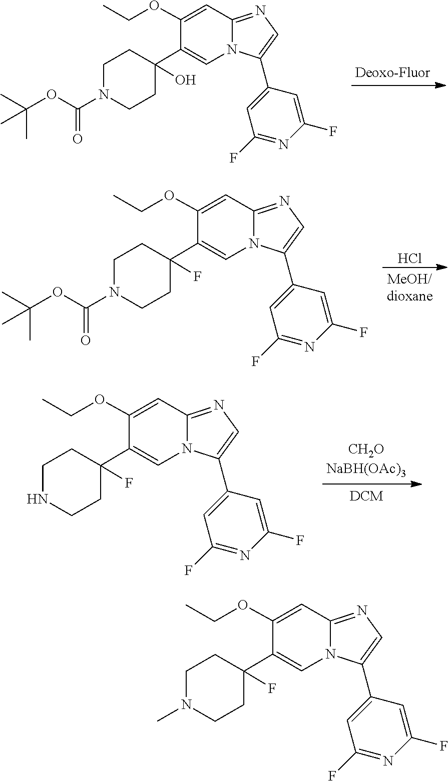

tert-Butyl 4-(3-(2,6-difluoropyridin-4-yl)-7-ethoxyimidazo[1,2-a]pyridin-6-yl)-4-fluoropiperidine-1-carboxylate: tert-Butyl 4-(3-(2,6-difluoropyridin-4-yl)-7-ethoxyimidazo[1,2-a]pyridin-6-yl)-4-hydroxypiperidine-1-carboxylate (500 mg, 1.05 mmol) was provided in CH₂Cl₂ (15 mL) and cooled to −8° C. Bis(2-methoxyethyl)aminosulfur trifluoride solution (2.33 g, 2.00 mL, 50% wt, 5.27 mmol) was added dropwise and the mixture was allowed to warm to −30° C. within 1 h. The mixture was cooled to −78° C., bis(2-methoxyethyl)aminosulfur trifluoride solution (2.33 g, 2.00 mL, 50% wt, 5.27 mmol) was added dropwise and the mixture was allowed to warm to rt overnight. The mixture was diluted with DCM and a saturated aqueous sodium bicarbonate solution at 0° C. and was stirred for 10 min. The organic phase was separated, dried and concentrated. The residue was purified by flash column chromatography on silica gel using a mixture of CH₂Cl₂/EtOAc/EtOH as eluent to give the title compound. [M+H]⁺=477.5 ¹H NMR (400 MHz, DMSO-d₆) δ ppm 8.49 (s, 1H), 8.09 (s, 1H), 7.52 (s, 2H), 7.23 (s, 1H), 4.13-4.24 (m, 2H), 3.90-4.09 (m, 2H), 2.93-3.24 (m, 2H), 2.34-2.56 (m, 2H), 1.88 (br t, J=12.42 Hz, 2H), 1.43 (s, 9H), 1.37 (t, J=6.97 Hz, 3H).

3-(2,6-Difluoropyridin-4-yl)-7-ethoxy-6-(4-fluoropiperidin-4-yl)imidazo[1,2-a]pyridine, hydrogen chloride (HC): tert-Butyl 4-(3-(2,6-difluoropyridin-4-yl)-7-ethoxyimidazo[1,2-a]pyridin-6-yl)-4-fluoropiperidine-1-carboxylate (270 mg, 567 mol) was provided in methanol (2 mL). Hydrogen chloride solution (207 mg, 1.42 mL, 4 M in 1,4-dioxane, 5.67 mmol) was added and the mixture was stirred at rt for 1 h. The mixture was concentrated to give the title compound, which was used without further purification. [M+H]⁺=377.4. ¹H NMR (400 MHz, DMSO-d₆) δ ppm 9.16-9.53 (m, 2H), 8.58 (s, 1H), 8.48 (s, 1H), 7.66 (s, 2H), 7.47 (s, 1H), 4.39 (q, J=6.84 Hz, 2H), 3.32-3.43 (m, 2H), 3.05-3.26 (m, 2H), 2.62-2.89 (m, 2H), 2.04-2.17 (m, 2H), 1.51 (t, J=6.97 Hz, 3H).

3-(2,6-Difluoropyridin-4-yl)-7-ethoxy-6-(4-fluoro-1-methylpiperidin-4-yl)imidazo[1,2-a]pyridine, hydrogen chloride (HCl): 3-(2,6-Difluoropyridin-4-yl)-7-ethoxy-6-(4-fluoropiperidin-4-yl)imidazo[1,2-a]pyridine, hydrogen chloride (HCl) (53 mg, 0.13 mmol) were provided in CH₂Cl₂ (2 mL). Formaldehyde (48 μL, 37 wt % in water, 0.64 mmol) was added, followed by sodium triacetoxyborohydride (140 mg, 0.64 mmol). The mixture was stirred at rt for 1 h. The mixture was diluted with CH₂Cl₂ and a saturated aqueous NaHCO₃ solution. The organic phase was separated, dried and concentrated. The residue was purified by flash column chromatography on silica gel using a mixture of EtOAc/EtOH as eluent to give the title compound. [M+H]⁺=391.5. ¹H NMR (400 MHz, DMSO-d₆) δ ppm 8.48 (s, 1H), 8.08 (s, 1H), 7.51 (s, 2H), 7.22 (s, 1H), 4.22 (q, J=6.8 Hz, 2H), 2.73 (br dd, J=10.6, 3.8 Hz, 2H), 2.52-2.66 (m, 2H), 2.23 (m, 3H), 2.20-2.29 (m, 2H), 1.80-1.90 (m, 2H), 1.41 (t, J=6.8 Hz, 3H).

### Intermediate 10

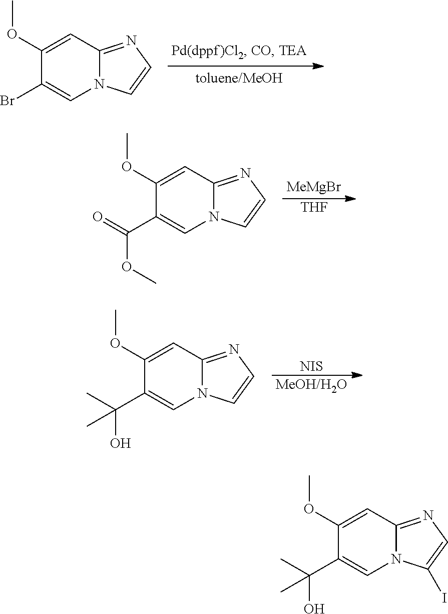

Methyl 7-methoxyimidazo[1,2-a]pyridine-6-carboxylate: A mixture of 6-bromo-7-methoxyimidazo[1,2-a]pyridine (1 g, 3.96 mmol, 1 eq) in MeOH (10 mL) and toluene (10 mL) was added TEA (602 mg, 5.95 mmol, 828 μL, 1.5 eq) and Pd(dppf)Cl₂ (290 mg, 396 μmol, 0.1 eq) under N₂. The mixture was stirred at 80° C. under CO (40 Psi) atmosphere for 16 h. The mixture was concentrated under reduced pressure to give a residue, which was purified by flash silica gel chromatography (from EtOAc/MeOH=100/1 to 94/6) to yield the title compound. [M+H]⁺=207.2.

2-(7-methoxyimidazo[1,2-a]pyridin-6-yl)propan-2-ol: To a solution of methyl 7-methoxyimidazo[1,2-a]pyridine-6-carboxylate (850 mg, 3.71 mmol, 1 eq) in THE (30 mL) was added MeMgBr (3 M, 6.18 mL, 5 eq) at 0° C. under N₂. The mixture was stirred at 20° C. for 2 h. The reaction mixture was quenched with H₂O (20 mL) at 0° C. and extracted with EtOAc (100 mL×3). The combined organic layers were washed with brine (50 mL), dried over Na₂SO₄, filtered, and concentrated under reduced pressure to give the title compound, which was used in the next step without further purification. ¹H NMR (400 MHz, DMSO-d₆) δ ppm: 8.44 (s, 1H), 7.79 (s, 1H), 7.34 (s, 1H), 6.90 (s, 1H), 5.23 (s, 1H), 3.85 (s, 3H), 1.51 (s, 6H).

2-(3-iodo-7-methoxyimidazo[1,2-a]pyridin-6-yl)propan-2-ol: To a solution of 2-(7-methoxyimidazo[1,2-a]pyridin-6-yl)propan-2-ol (700 mg, 3.05 mmol, 1 eq) in MeOH (18 mL) and H₂O (12 mL) was added NIS (893 mg, 3.97 mmol, 1.3 eq) at 0° C. The mixture was stirred at 20° C. for 2 h. The mixture was filtered, diluted with saturated Na₂SO₃ (20 mL) and extracted with DCM (100 mL×3). The combined organic layers were washed with saturated NaHCO₃ (100 mL), dried over Na₂SO₄, filtered, and concentrated under reduced pressure to give the title compound, which was used without further purification. ¹H NMR (400 MHz, DMSO-d₆) δ ppm: 8.29 (s, 1H), 7.50 (s, 1H), 7.02 (s, 1H), 5.44 (s, 1H), 3.89 (s, 3H), 1.52 (s, 6H).

### Intermediate 11

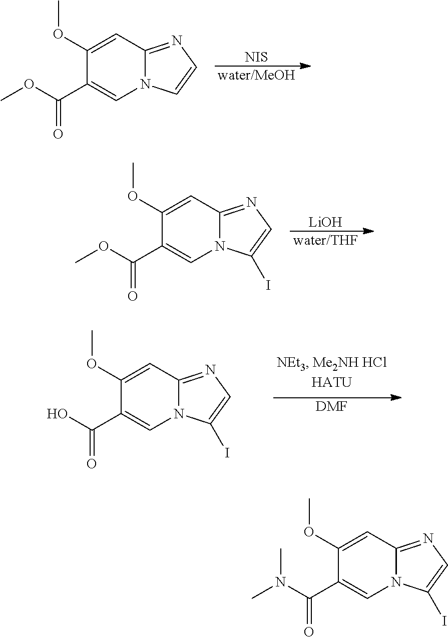

Methyl 3-iodo-7-methoxyimidazo[1,2-a]pyridine-6-carboxylate: To methyl 7-methoxyimidazo[1,2-a]pyridine-6-carboxylate (1.58 g, 7.64 mmol) in methanol (30.0 mL) and water (12.0 mL) was added N-iodosuccinimide (2.06 g, 9.17 mmol) and the mixture was stirred for 90 min at 20° C. Aqueous sodium thiosulfate solution (10%) and water were added, and the precipitate was collected by filtration, washed with water, and dried to give the title compound, which was used without further purification. [M+H]⁺=333.2. ¹H NMR (400 MHz, DMSO-d₆) δ ppm 8.56 (s, 1H), 7.64 (s, 1H), 7.15 (s, 1H), 3.89 (s, 3H), 3.85 (s, 3H).

3-Iodo-7-methoxyimidazo[1,2-a]pyridine-6-carboxylic acid: To methyl 3-iodo-7-methoxyimidazo[1,2-a]pyridine-6-carboxylate (2.05 g, 6.17 mmol) in THE (20 mL) and water (5 mL) was added lithium hydroxide (222 mg, 926 mmol) and the mixture was stirred for 22 h at 20° C. The suspension was heated to 60° C. and stirred without further heating for 1 h. The mixture was diluted with water, acidified with an aqueous 1M HCl solution and cooled to 0° C. The precipitate was collected by filtration, washed with water, and dried to give the title compound. [M+H]⁺=319.2. ¹H NMR (400 MHz, DMSO-d₆) δ ppm 8.37 (s, 1H), 7.56 (s, 1H), 7.03 (s, 1H), 3.85 (s, 3H).

3-Iodo-7-methoxy-N,N-dimethylimidazo[1,2-a]pyridine-6-carboxamide: 3-Iodo-7-methoxyimidazo[1,2-a]pyridine-6-carboxylic acid (500 mg, 1.57 mmol) was suspended in DMF (5 mL) and treated with triethylamine (159 mg, 1.57 mmol) and dimethylamine hydrochloride (128 mg, 1.57 mmol), followed by HATU (598 mg, 1.57 mmol) under an argon atmosphere. The mixture was stirred at rt for 19 h. The mixture was diluted with EtOAc and water. The organic phase was separated and dried. The solvent was evaporated, and the residue was purified by flash column chromatography on silica gel using a mixture of CH₂Cl₂/EtOH as eluent to give a product which was triturated with water and collected by filtration to give the title compound. [M+H]⁺=346.3. ¹H NMR (400 MHz, DMSO-d₆) δ ppm 8.37 (s, 1H), 7.89 (s, 1H), 7.23 (s, 1H), 3.95 (s, 3H), 3.00 (s, 3H), 2.81 (s, 3H).

### Intermediate 12

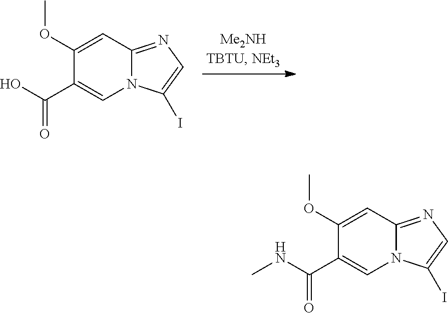 [SMILES: COc1cc2ncc(I)n2cc1C(=O)O ; CNC(=O)c1cn2c(I)cnc2cc1OC]

3-Iodo-7-methoxy-N-methylimidazo[1,2-a]pyridine-6-carboxamide: 3-Iodo-7-methoxyimidazo[1,2-a]pyridine-6-carboxylic acid (500 mg, 1.57 mmol) was suspended in CH₂Cl₂ (10 mL) before DIPEA (0.685 mL, 3.93 mmol), TBTU (757 mg, 2.36 mmol) and methyl amine (2 M in THF, 1.57 mL, 3.14 mmol) were added. The mixture was stirred at rt for 3 days. The mixture was diluted with water. An aqueous 1 M HCl solution was added, and the mixture was cooled to 0° C. The precipitate was collected by filtration and washed with water. The residue was purified by flash column chromatography on silica gel using a mixture of CH₂Cl₂/EtOH as eluent to give the title compound. [M+H]⁺=332.3. ¹H NMR (400 MHz, DMSO-d₆) δ ppm 8.54 (s, 1H), 8.32 (q, J=4.6 Hz, 1H), 7.62 (s, 1H), 7.15 (s, 1H), 3.95 (s, 3H), 2.83 (d, J=4.6 Hz, 3H).

### Intermediate 13

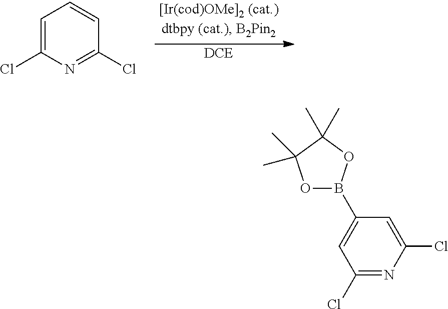 [SMILES: Clc1cccc(Cl)n1 ; "DCE" ; CC1(C)OB(c2cc(Cl)nc(Cl)c2)OC1(C)C]

2,6-dichloro-4-(4,4,5,5-tetramethyl-1,3,2-dioxaborolan-2-yl)pyridine: Bis(1,5-cyclooctadiene)dimethoxydiiridium (44.8 mg, 0.01 equiv, 67.6 gmol) and 4,4′-di-tert-butyl-2,2′-bipyridyl (36.3 mg, 0.02 equiv, 135.1 μmol) were dissolved in DCE (10 mL) under argon. The mixture was stirred at rt for 10 min. 2,6-Dichloropyridine (1.00 g, 1 equiv, 6.76 mmol) and 4,4,4′,4′,5,5,5′,5′-octamethyl-2,2′-bi(1,3,2-dioxaborolane) (1.97 g, 1.15 equiv, 7.77 mmol) were added and the mixture was stirred under argon at 100° C. for 4 h. The mixture was diluted with EtOAc, and brine was added. The organic phase was separated, washed with sat. NaHCO₃ solution, dried, and filtered. The solvent was evaporated to give a residue. Purification via biotage chromatography on silica gel using hexane/EtOAc as eluent afforded the title compound. [X+H]⁺=192.1 (X=(2,6-dichloropyridin-4-yl)boronic acid). ¹H NMR (400 MHz, DMSO-d₆) δ ppm 7.57 (s, 2H), 1.30 (s, 12H).

### Intermediate 14

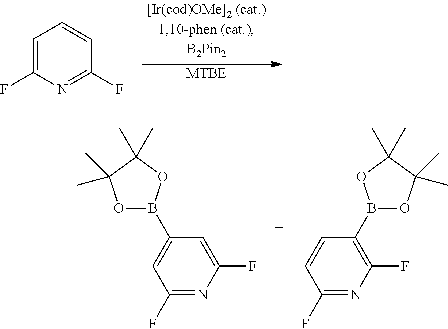

2,6-difluoro-4-(4,4,5,5-tetramethyl-1,3,2-dioxaborolan-2-yl)pyridine and 2,6-difluoro-3-(4,4,5,5-tetramethyl-1,3,2-dioxaborolan-2-yl)pyridine: To a stirred solution of 2,6-difluoropyridine (0.500 g, 4.34 mmol, 1 eq) in MTBE (5 mL) was added 4,4,4′,4′,5,5,5′,5′-octamethyl-2,2′-bis(1,3,2-dioxaborolane) (1.25 g, 4.91 mmol, 1.2 eq) followed by 1,10-phenanthroline (0.783 g, 0.043 mmol, 0.01 eq) and bis(1,5-cyclootadiene)dimethoxydiiridium (28.8 mg, 0.043 mmol, 0.01 eq) at rt. The reaction mixture was then heated at 70° C. for 4 h. After completion, the reaction mixture was concentrated under vacuum. The crude residue was purified by flash column chromatography over silica gel (100-200 mesh) using 5-10% ethyl acetate/petroleum ether to afford the title compound as a 1.6:1.0 mixture of 2,6-difluoro-4-(4,4,5,5-tetramethyl-1,3,2-dioxaborolan-2-yl)pyridine and 2,6-difluoro-3-(4,4,5,5-tetramethyl-1,3,2-dioxaborolan-2-yl)pyridine. [X+H]⁺=160.1 (X=(difluoropyridinyl)boronic acid).

### Intermediate 15

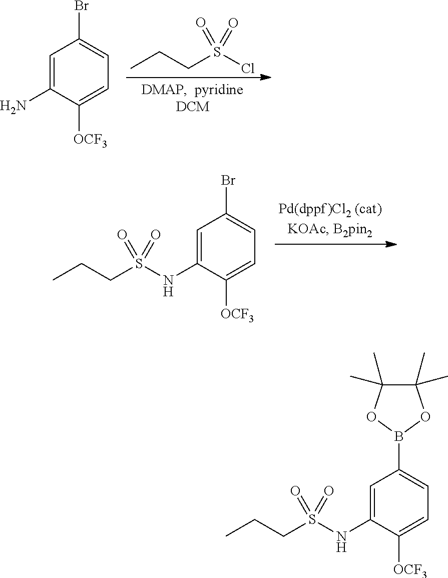

N-(5-Bromo-2-(trifluoromethoxy)phenyl)propane-1-sulfonamide: 5-Bromo-2-(trifluoromethoxy)aniline (600 mg, 2.34 mmol) was dissolved in DCM (40 mL), and then DMAP (28.6 mg, 234 mol), pyridine (371 mg, 378 μL, 4.69 mmol) and n-propylsulfonyl chloride (368 mg, 290 μL, 2.58 mmol) were added before the mixture was stirred for 3 days at 20° C. The mixture was concentrated, and the residue was purified by flash column chromatography on silica gel using a mixture of CH₂Cl₂/EtOAc as eluent to give the title compound. [M−H]⁻=362.2. ¹H NMR (400 MHz, DMSO-d₆) δ ppm 10.03 (s, 1H), 7.69 (d, J=2.28 Hz, 1H), 7.42-7.50 (m, 1H), 7.32-7.41 (m, 1H), 3.13-3.23 (m, 2H), 1.64-1.81 (m, 2H), 0.97 (t, J=7.48 Hz, 3H).

N-(5-(4,4,5,5-tetramethyl-1,3,2-dioxaborolan-2-yl)-2-(trifluoromethoxy)phenyl)propane-1-sulfonamide: To a solution of N-(5-bromo-2-(trifluoromethoxy)phenyl)propane-1-sulfonamide (245 mg, 1 eq, 676 μmol) and 4,4,4′,4′,5,5,5′,5′-octamethyl-2,2′-bi(1,3,2-dioxaborolane) (344 mg, 1.35 mmol) in DMSO (5 mL) was added potassium acetate (199 mg, 2.03 mmol) while the mixture was purged with argon. [1,1′-Bis(diphenylphosphino)ferrocene]dichloropalladium(II) dichloromethane complex (55.2 mg, 67.6 μmol) was added and the mixture was purged with argon for 5 min. The vial was sealed, and the mixture was stirred for 9 h at 90° C. The mixture was concentrated, and the residue was purified by flash column chromatography on silica gel using a mixture of CH₂Cl₂/EtOAc as eluent to give the title compound. [M−H]⁻=408.4. ¹H NMR (400 MHz, DMSO-d₆) δ ppm 9.74 (s, 1H), 7.77 (d, J=1.5 Hz, 1H), 7.57 (dd, J=8.4, 1.5 Hz, 1H), 7.41 (dd, J=8.4, 1.8 Hz, 1H), 3.03-3.10 (m, 2H), 1.68-1.80 (m, 2H), 1.30 (s, 12H), 0.96 (t, J=7.5 Hz, 3H).

### Intermediate 16

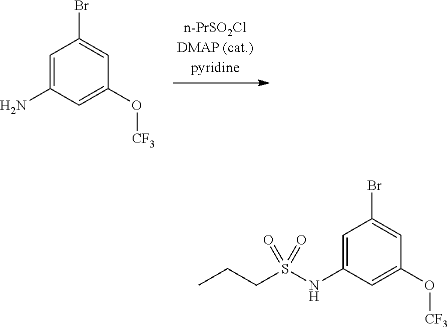 [SMILES: Nc1cc(Br)cc(OC(F)(F)F)c1 ; CCCS(=O)(=O)Nc1cc(Br)cc(OC(F)(F)F)c1]

N-(3-Bromo-5-(trifluoromethoxy)phenyl)propane-1-sulfonamide: 3-Bromo-5-(trifluoromethoxy)aniline (1.00 g, 3.91 mmol) was dissolved in DCM (40 mL), DMAP (47.7 mg, 391 μmol), pyridine (618 mg, 629 μL, 7.81 mmol) and n-propylsulfonyl chloride (613 mg, 483 μL, 4.30 mmol) were added and the mixture was stirred for 3 days at 20° C. The mixture was concentrated, and the residue was purified by flash column chromatography on silica gel using a mixture of CH₂Cl₂/EtOAc as eluent to give the title compound. [M−H]⁻=362.2. ¹H NMR (400 MHz, DMSO-d₆) δ ppm 10.40 (s, 1H), 7.36-7.38 (m, 1H), 7.33-7.37 (m, 1H), 7.16-7.19 (m, 1H), 3.17-3.24 (m, 2H), 1.62-1.73 (m, 2H), 0.95 (t, J=7.48 Hz, 3H).

The following compound was prepared following procedures analogous to that described for Intermediate 16.

| Int. | | | |
|---|---|---|---|
| No. | Structure | Name | LCMS, ¹H NMR |
| Int. 17 | 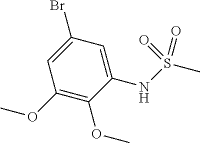 | N-(5-bromo-2,3- dimethoxyphenyl) propane-1-sulfonamide | [M + H]⁺ = 310.36 ¹H NMR (400 MHz, CDCl₃) δ ppm: 6.97 (s, 1H), 6.83 (d, J = 2.00 Hz, 1H), 3.87 (s, 6H), 3.03 (s, 3H) |

### Intermediate 18

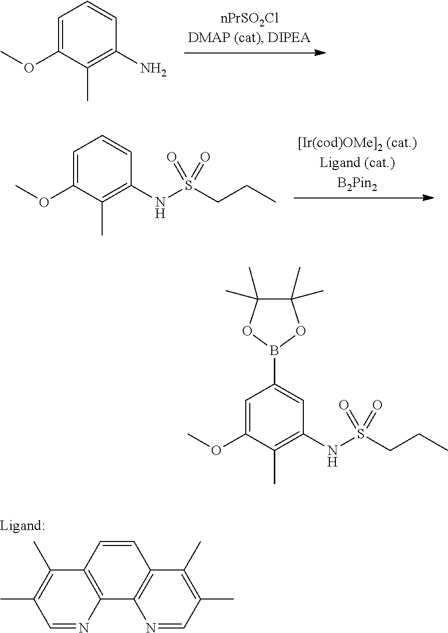

N-(3-Methoxy-2-methylphenyl)propane-1-sulfonamide: 3-Methoxy-2-methyl-aniline was dissolved in CH₂Cl₂ (20 mL) before DIPEA (1.56 mL, 8.92 mmol) was added, followed by n-propylsulfonyl chloride (0.40 mL, 3.57 mmol) and DMAP (43.6 mg, 0.36 mmol). The mixture was stirred at rt for 1 h. Additional DIPEA (1.56 mL, 8.92 mmol) was added, followed by n-propylsulfonyl chloride (0.40 mL, 3.57 mmol) and DMAP (43.6 mg, 0.36 mmol) and the mixture was stirred at rt for 88 h. The reaction was diluted with CH₂Cl₂ and water. The organic phase was separated and dried. The solvent was evaporated, and the residue was purified by flash column chromatography on silica gel to give crude title compound. The compound was used in the next step without any further purification. [M−H]⁻=242.3.

N-(3-Methoxy-2-methyl-5-(4,4,5,5-tetramethyl-1,3,2-dioxaborolan-2-yl)phenyl)propane-1-sulfonamide: Cyclooctadiene iridium methoxide dimer (13.6 mg, 0.021 mmol) and 3,4,7,8-tetramethyl-1,10-diazaphenanthrene (9.7 mg, 0.041 mmol) were dissolved in heptane (3 mL) and stirred at rt for 10 min. N-(3-methoxy-2-methylphenyl)propane-1-sulfonamide (200 mg, 0.622 mmol) and is(pinacolato)diborane (417 mg, 1.64 mmol) were added and the mixture was heated to 80° C. for 16 h. The mixture was cooled to rt and diluted with EtOAc and brine. The organic phase was separated and dried. The solvent was evaporated, and the residue was purified by flash column chromatography on silica gel to give the title compound. The compound was used in the next step without any further purification. [M−H]⁻=368.5.

The following compounds were prepared following procedures analogous to that described for Intermediate 18.

| Int. | | | |
|---|---|---|---|
| No. | Structure | Name | LCMS, ¹H NMR |
| Int. 19 | 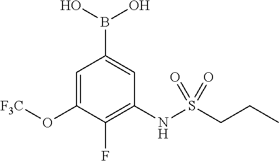 [SMILES: CCCS(=O)(=O)Nc1cc(B(O)O)cc(OC(F)(F)F)c1F] | (4-fluoro-3-(propylsulfonamido)-5- (trifluoromethoxy)phenyl)boronicacid | [M − H]⁻ = 344.3 |
| Int. 20 | 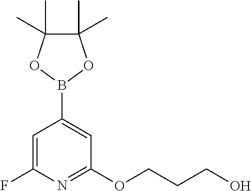 [SMILES: CC1(C)OB(c2cc(F)nc(OCCCO)c2)OC1(C)C] | 3-((6-fluoro-4-(4,4,5,5-tetramethyl- 1,3,2-dioxaborolan-2-yl)pyridin-2- yl)oxy)propan-1-ol | [M + H]⁺ = 298.4 |

### Intermediate 21

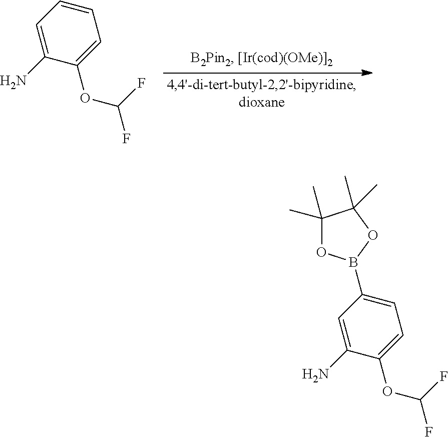 [SMILES: Nc1ccccc1OC(F)F ; CC1(C)OB(c2ccc(OC(F)F)c(N)c2)OC1(C)C]

2-(difluoromethoxy)-5-(4,4,5,5-tetramethyl-1,3,2-dioxaborolan-2-yl)aniline: A mixture of 2-(difluoromethoxy)aniline, 4,4,5,5-tetramethyl-2-(4,4,5,5-tetramethyl-1,3,2-dioxaborolan-2-yl)-1,3,2-dioxaborolane (718 mg, 2.83 mmol, 1.5 eq), (1Z,5Z)-cycloocta-1,5-diene;2,4-dimethyl-bicyclo[1.1.0]butane (125 mg, 189 μmol, 0.1 eq), 4-tert-butyl-2-(4-tert-butyl-2-pyridyl)pyridine (50.6 mg, 189 μmol, 0.1 eq) in dioxane (5 mL) was degassed and purged with N₂ three times, and then the mixture was stirred at 75° C. for 16 h under N₂. The reaction mixture was filtered and concentrated under reduced pressure to give a residue which was purified by prep-TLC (SiO₂, PE:EA=1:1) to give the title compound. ¹H NMR (400 MHz, CDCl₃) δ ppm: 7.47 (d, J=7.6 Hz, 1H), 7.42 (s, 1H), 6.82-6.65 (m, 2H), 1.32 (s, 12H).

### Intermediate 22

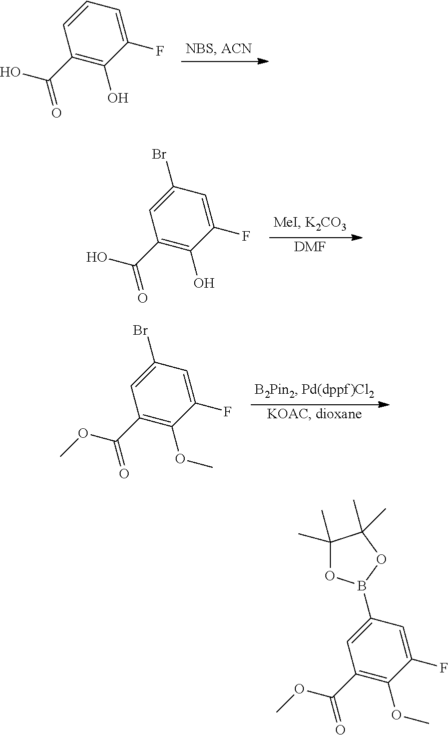

5-bromo-3-fluoro-2-hydroxybenzoic acid: To a mixture of 3-fluoro-2-hydroxybenzoic acid (200 mg, 1.28 mmol, 1 eq) in ACN (4 mL) was added NBS (250 mg, 1.41 mmol, 1.1 eq), and the mixture was stirred at 25° C. for 12 h under nitrogen. The reaction mixture was concentrated under reduced pressure to give a residue, which was purified by column chromatography (SiO₂, DCM/MeOH=1/0 to 10/1) to give the title compound. ¹H NMR (400 MHz, DMSO-d₆) δ ppm: 11.18-10.90 (m, 1H), 7.79 (dd, J=2.4, 10.4 Hz, 1H), 7.72-7.65 (m, 1H).

Methyl 5-bromo-3-fluoro-2-methoxybenzoate: To a mixture of 5-bromo-3-fluoro-2-hydroxybenzoic acid (300 mg, 1.15 mmol, 1 eq), and K₂CO₃ (874 mg, 6.32 mmol, 5.5 eq) in DMF (7 mL) was added Mel (718 mg, 5.06 mmol, 0.315 mL, 4.40 eq) at 0° C. The mixture was stirred at 0° C. for 1 h under a N₂ atmosphere, after which it was stirred at 25° C. for 12 h. The reaction mixture was quenched with H₂O (20 mL), extracted with DCM (20 mL×3), dried over Na₂SO₄, filtered, and concentrated under reduced pressure. The crude product was purified by flash silica gel chromatography (DCM/MeOH=1/0) to give the title compound. ¹H NMR (400 MHz, CDCl₃) δ ppm: 7.75-7.55 (m, 1H), 7.48-7.30 (m, 1H), 3.96 (s, 3H), 3.91 (s, 3H).

Methyl 3-fluoro-2-methoxy-5-(4,4,5,5-tetramethyl-1,3,2-dioxaborolan-2-yl)benzoate: To a mixture of methyl 5-bromo-3-fluoro-2-methoxybenzoate (250 mg, 855 μmol, 1 eq), B₂Pin₂ (434 mg, 1.71 mmol, 2 eq), and KOAc (252 mg, 2.57 mmol, 3 eq) in dioxane (3 mL) was added Pd(dppf)Cl₂ (63 mg, 86 μmol, 0.1 eq). The mixture was degassed and purged with N₂ three times, and then the mixture was stirred at 100° C. for 2 h under a N₂ atmosphere. The reaction mixture was concentrated under reduced pressure, and the resulting residue was purified by column chromatography (SiO₂, PE/EtOAc=1/0) to give the title compound. [M+H]⁺=311.1. ¹H NMR: (400 MHz, CDCl₃) δ ppm: 7.94 (s, 1H), 7.69-7.60 (m, 1H), 4.01 (s, 3H), 3.90 (s, 3H), 1.33 (s, 12H).

### Intermediate 23

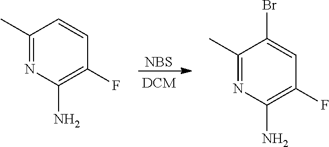

5-bromo-3-fluoro-6-methylpyridin-2-amine: To a solution of 3-fluoro-6-methylpyridin-2-amine (400 mg, 3.17 mmol, 1 eq) in DCM (4 mL) was added NBS (677.31 mg, 3.81 mmol, 1.2 eq) at 0° C. The mixture was stirred at 20° C. for 1 h. The reaction mixture was quenched by the addition of sat. Na₂SO₃ (10 mL) at 0° C., and then extracted with DCM (3×10 mL). The combined organic layers were dried over Na₂SO₄, filtered, and concentrated in vacuo. The resulting crude material was purified via silica gel chromatography (PE/EtOAc=90/10 to 70/30) to yield the title compound. ¹H NMR (400 MHz, DMSO-d₆) δ ppm 7.60 (d, J=10.4 Hz, 1H), 6.35 (s, 2H), 2.32 (s, 3H).

### Intermediate 24

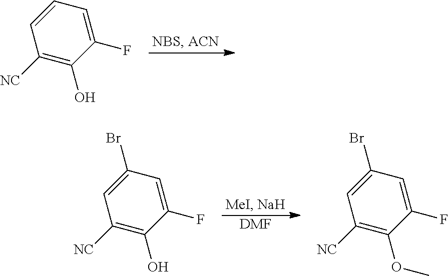

5-bromo-3-fluoro-2-hydroxybenzonitrile: To a solution of 3-fluoro-2-hydroxybenzonitrile (200 mg, 1.46 mmol, 1 eq) in ACN (10 mL) was added NBS (260 mg, 1.46 mmol, 1 eq). The mixture was stirred at 25° C. for 1 h. The reaction mixture was filtered and concentrated under reduced pressure to give a residue, which was purified by column chromatography (SiO₂, petroleum ether/ethyl acetate=1/0 to 9/1) to give the title compound. ¹H NMR (400 MHz, DMSO-d₆) δ ppm: 11.86 (br s, 1H), 7.87 (dd, J=2.4, 10.8 Hz, 1H), 7.82-7.73 (m, 1H).

5-bromo-3-fluoro-2-methoxybenzonitrile: To a solution of 5-bromo-3-fluoro-2-hydroxybenzonitrile (240 mg, 1.00 mmol, 1 eq) in DMF (4 mL) was added NaH (80 mg, 2.00 mmol, 60% purity, 2 eq). The mixture was stirred at 25° C. for 20 min, after which iodomethane (284 mg, 2.00 mmol, 125 μL, 2 eq) was added. The mixture was stirred at 25° C. for 12 h, after which the reaction was quenched by addition H₂O (10 mL). The resulting solid was collected by filtration and was used for directly without purification. [M+H]⁺=287.0. ¹H NMR (400 MHz, DMSO-d₆) δ ppm: 8.04 (dd, J=2.3, 11.7 Hz, 1H), 7.95 (d, J=1.5 Hz, 1H), 4.06 (d, J=2.8 Hz, 3H).

### Intermediate 25

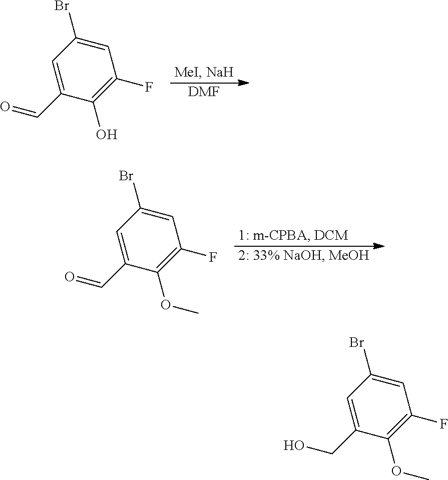

5-bromo-3-fluoro-2-methoxybenzaldehyde: To a solution of 5-bromo-3-fluoro-2-hydroxybenzaldehyde (1 g, 4.57 mmol, 1 eq) in DMF (15 mL) was added NaH (365 mg, 9.13 mmol, 60% purity, 2 eq) at 0° C. Then Mel (2.59 g, 18.3 mmol, 1.14 mL, 4 eq) was added to the reaction mixture. The resulting mixture was stirred at 25° C. for 12 h. The reaction mixture was quenched by addition H₂O (30 mL) at 0° C. and extracted with DCM (120 mL). The combined organic layers were washed with brine (60 mL), dried over Na₂SO₄, filtered, and concentrated under reduced pressure to give a residue, which was purified reversed-phase HPLC (H₂O/ACN=0-56%) to give the title compound. ¹H NMR: (400 MHz, DMSO-d₆) δ ppm: 10.20 (s, 1H), 7.99 (dd, J=2.4, 11.2 Hz, 1H), 7.63 (s, 1H), 4.03 (s, 3H).

5-bromo-3-fluoro-2-methoxyphenol: A mixture of 5-bromo-3-fluoro-2-methoxybenzaldehyde (500 mg, 1.93 mmol, 1 eq), m-CPBA (784 mg, 3.86 mmol, 85% purity, 2 eq) in DCM (20 mL) was degassed and purged with N₂ three times, and then the mixture was stirred at 40° C. for 12 h. The mixture was diluted with DCM (60 mL) and washed with saturated aqueous NaHCO₃ (30 mL), dried over Na₂SO₄, filtered, and concentrated under reduced pressure. Methanol (10 mL) was added, followed by NaOH (33% aqueous, 1 mL), and the mixture was stirred at 25° C. for 2 h. The pH was adjusted to pH=5 with HCl (1N, 10 mL) at 0° C., and the mixture was extracted with EtOAc (90 mL). The combined organic layers were dried over Na₂SO₄, filtered, and concentrated under reduced pressure to give the title compound, which was used without further purification. ¹H NMR (400 MHz, DMSO-d₆) δ ppm: 6.96 (dd, J=2.4, 10.0 Hz, 1H), 6.91-6.85 (m, 1H), 3.76 (s, 3H).

### Intermediate 26

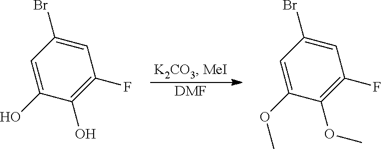

5-bromo-1-fluoro-2,3-dimethoxybenzene: To a solution of 5-bromo-3-fluorobenzene-1,2-diol (300 mg, 1.45 mmol, 1 eq) in DMF (2 mL) was added K₂CO₃ (600 mg, 4.35 mmol, 3 eq) and Mel (617 mg, 4.35 mmol, 270 μL, ³ eq). The mixture was stirred at 25° C. for 3 h. The mixture was concentrated, and then water (30 mL) was added. The mixture was extracted with EtOAc (30 mL×3). The combined organic layers were washed with brine (50 mL), dried over Na₂SO₄, filtered, and concentrated to give a residue, which was purified by flash silica gel chromatography (SiO₂, ethyl acetate/petroleum from 0% to 10%) to give the title compound. ¹H NMR: (400 MHz, CDCl₃) δ ppm: 6.93-6.86 (m, 1H), 6.82 (t, J=2.0 Hz, 1H), 3.90 (d, J=0.8 Hz, 3H), 3.86 (s, 3H).

### Intermediate 27

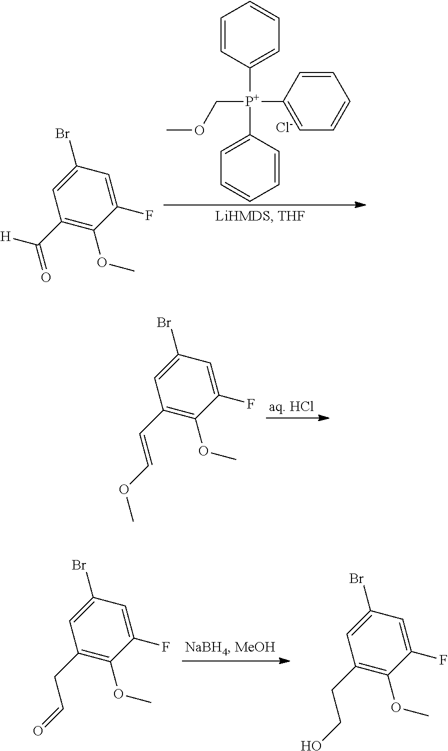 [SMILES: COc1c(F)cc(Br)cc1C=O ; COC[P+](c1ccccc1)(c1ccccc1)c1ccccc1 ; [Cl-] ; CO/C=C/c1cc(Br)cc(F)c1OC ; COc1c(F)cc(Br)cc1CC=O ; COc1c(F)cc(Br)cc1CCO]

(E)-5-bromo-1-fluoro-2-methoxy-3-(2-methoxyvinyl)benzene: To a solution of methoxymethyl)triphenylphosphonium chloride (1.10 g, 3.22 mmol, 1.5 eq) in THE (10 mL) was added LiHMDS (1 M, 3.43 mL, 1.6 eq) at 0° C. under N₂ and the reaction mixture was stirred at 0° C. 30 min, and then 5-bromo-3-fluoro-2-methoxybenzaldehyde (500 mg, 2.15 mmol, 1 eq) in THE (2 mL) was added to the reaction mixture and the mixture was stirred at 25° C. for 30 min. The reaction mixture was quenched by addition H₂O (20 mL) at 0° C. The mixture was concentrated under reduced pressure to give a residue, which was purified by flash silica gel chromatography (eluting with petroleum ether) to give the title compound, which was used without further purification.

2-(5-bromo-3-fluoro-2-methoxyphenyl)acetaldehyde: To a solution of (E)-5-bromo-1-fluoro-2-methoxy-3-(2-methoxyvinyl)benzene (300 mg, 1.15 mmol, 1 eq) in DCM (20 mL) was added HCl (1 mL, 12 N). The mixture was stirred at 25° C. for 12 h. The reaction mixture was diluted with H₂O (2 mL), adjust pH to 8 with saturated NaHCO₃ at 0° C., extracted with DCM (50 mL×3), dried over Na₂SO₄, filtered, and concentrated under reduced pressure to give a residue, which was purified by flash silica gel chromatography (from petroleum ether/ethyl acetate=100/1 to 4/1) to give the title compound. ¹H NMR (400 MHz, DMSO-d₆) δ ppm: 9.66 (s, 1H), 7.58-7.50 (m, 1H), 7.35-7.29 (m, 1H), 3.83 (d, J=1.6 Hz, 2H), 3.78 (d, J=2.0 Hz, 3H).

2-(5-bromo-3-fluoro-2-methoxyphenyl)ethan-1-ol: To a solution of 2-(5-bromo-3-fluoro-2-methoxyphenyl)acetaldehyde (150 mg, 607 mol, 1 eq) in MeOH (2 mL) was added NaBH₄ (41.4 mg, 1.09 mmol, 1.8 eq) at 0° C. The mixture was stirred at 25° C. for 2 h. The reaction mixture was quenched by addition H₂O (20 mL) at 0° C. and concentrated under reduced pressure to give a residue, which was purified by flash silica gel chromatography (from petroleum ether/ethyl acetate=100/1 to 3/1) to give the title compound. ¹H NMR (400 MHz, DMSO-d₆) δ ppm: 7.43 (dd, J=2.4, 11.2 Hz, 1H), 7.31-7.22 (m, 1H), 4.70 (t, J=5.6 Hz, 1H), 3.82 (d, J=1.6 Hz, 3H), 3.61-3.52 (m, 2H), 2.73 (t, J=6.8 Hz, 2H).

### Intermediate 28

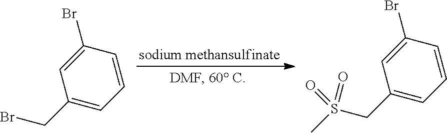

1-bromo-3-((methylsulfonyl)methyl)benzene: To a solution of 1-bromo-3-(bromomethyl)benzene (200 mg, 800 μmol, 1 eq) in DMF (2.5 mL) was added sodium methanesulfinate (245 mg, 2.40 mmol, 3 eq). The mixture was stirred at 60° C. for 1 h under N₂. The reaction mixture was diluted with H₂O (20 mL) and extracted with EtOAc (20 mL×3). The combined organic layers were washed with brine (30 mL), dried over Na₂SO₄, filtered, and concentrated under reduced pressure to give a residue, which was purified by flash silica gel chromatography (from petroleum ether/EtOAc=100/1 to 2/1) to give the title compound. ¹H NMR (400 MHz, CDCl₃) δ ppm: 7.64-7.49 (m, 2H), 7.40-7.34 (m, 1H), 7.34-7.27 (m, 1H), 4.21 (s, 2H), 2.80 (s, 3H).

### Intermediate 29

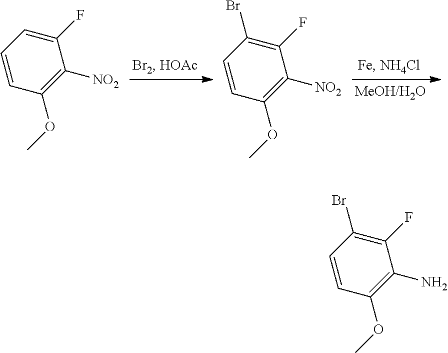

1-bromo-2-fluoro-4-methoxy-3-nitrobenzene: To a solution of 1-fluoro-3-methoxy-2-nitrobenzene (500 mg, 2.92 mmol, 1 eq) in AcOH (5.0 mL) was added Br₂ (1.87 g, 11.7 mmol, 602 μL, 4.0 eq). The mixture was stirred at 60° C. for 12 h. The reaction mixture was concentrated under reduced pressure to give a residue. The residue was diluted with H₂O (20 mL), the pH was adjusted to pH 8 with saturated aqueous NaHCO₃, and the mixture was extracted with EtOAc (10 mL×3). The combined organic layers were concentrated under reduced pressure to give the title compound, which was used without further purification. ¹H NMR (400 MHz, CDCl₃) δ ppm: 7.64-7.55 (m, 1H), 6.80-6.74 (m, 1H), 3.93 (s, 3H).

3-bromo-2-fluoro-6-methoxyaniline: To a solution of 1-bromo-2-fluoro-4-methoxy-3-nitrobenzene (600 mg, 2.04 mmol, 1 eq) in MeOH (6 mL) and H₂O (6 mL) was added Fe (1.14 g, 20 mmol, 10 eq) and NH₄Cl (1.09 g, 20 mmol, 10 eq). The mixture was stirred at 60° C. for 2 h. The reaction mixture was filtered under reduced pressure and concentrated. Then the mixture was adjusted pH=8 with saturated aqueous NaHCO₃, diluted with H₂O (10 mL) and extracted with EtOAc (20 mL×3). The combined organic layers were washed with brine (20 mL), dried over Na₂SO₄, filtered, and concentrated under reduced pressure to give the title compound, which was used without further purification. ¹H NMR (400 MHz, CDCl₃) δ ppm: 6.94-6.77 (m, 1H), 6.49 (dd, J=1.6, 8.8 Hz, 1H), 3.85 (s, 3H).

### Intermediate 30

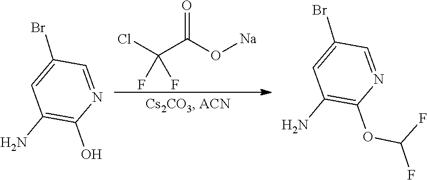

5-bromo-2-(difluoromethoxy)pyridin-3-amine: A mixture of 3-amino-5-bromopyridin-2-ol (50 mg, 265 μmol, 1 eq), compound sodium 2-chloro-2,2-difluoroacetate (121 mg, 794 mol, 3 eq) and Cs₂CO₃ (172 mg, 529 mol, 2 eq) in ACN (3 mL) was degassed and purged with N₂ three times, and then the mixture was stirred at 80° C. for 2 h under N₂. The reaction mixture was diluted with water (20 mL), extracted with EtOAc (20 mL×3). The combined organic layers were dried over Na₂SO₄, filtered, and concentrated under reduced pressure to give a residue, which was purified by column chromatography (SiO₂, petroleum ether/ethyl acetate=1/1) to give the title compound. [M+H]⁺=238.9). ¹H NMR (400 MHz, DMSO-d₆) δ ppm: 7.79-7.41 (m, 2H), 7.22 (d, J=2.0 Hz, 1H), 5.63 (s, 2H).

### Intermediate 31

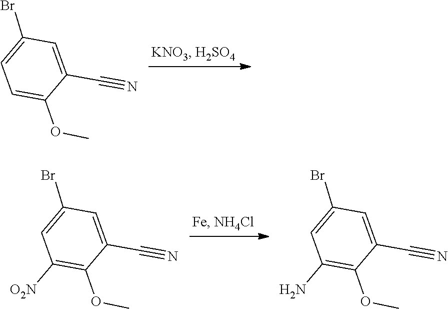

5-bromo-2-methoxy-3-nitrobenzonitrile: To a solution of 5-bromo-2-methoxybenzonitrile (500 mg, 2.36 mmol, 1 eq) in H₂SO₄ (5 mL) was added KNO₃ (358 mg, 3.54 mmol, 1.5 eq). The mixture was stirred at 25° C. for 2 h. The mixture was poured into ice, and saturated NaHCO₃ was added to adjust pH to 7, then 10 mL H₂O was added and extracted with ethyl acetate (10 mL×2), the combined organic layers were washed with brine (10 mL), dried over Na₂SO₄, filtered, and concentrated under reduced pressure to give a residue, which was purified by flash silica gel chromatography (from petroleum ether/ethyl acetate=100/0 to 67/33) to give the title compound. ¹H NMR (400 MHz, CDCl₃) δ ppm: 8.14 (d, J=2.4 Hz, 1H), 7.93 (d, J=2.4 Hz, 1H), 4.18 (s, 3H).

3-amino-5-bromo-2-methoxybenzonitrile: To a solution of 5-bromo-2-methoxy-3-nitrobenzonitrile (425 mg, 1.49 mmol, 1 eq) in EtOH (15 mL) and H₂O (5 mL) was added Fe (416 mg, 7.44 mmol, 5 eq) and NH₄Cl (398 mg, 7.44 mmol, 5 eq). The mixture was stirred at 80° C. for 2 h under N₂. The reaction solution was filtered, and the filter cake was washed with EtOH (20 mL). The filtrate was concentrated under reduced pressure, dissolved in water (20 mL) and extracted with ethyl acetate (20 mL×2), The combined organic layers were washed with brine (10 mL), dried over Na₂SO₄, filtered, and concentrated under reduced pressure to give a residue, which was purified by flash silica gel chromatography (from petroleum ether/ethyl acetate=5/1 to 1/1) to give the title compound. [M+H]⁺=227.1. H NMR (400 MHz, DMSO-d₆) d ppm: 7.10 (d, J=2.4 Hz, 1H), 7.03 (d, J=2.4 Hz, 1H), 5.79 (s, 2H), 3.79 (s, 3H).

### Intermediate 32

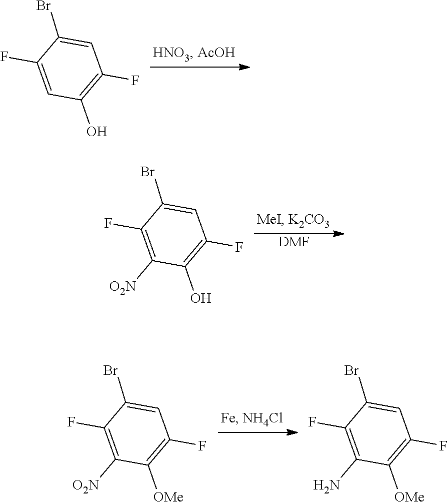

4-bromo-3,6-difluoro-2-nitrophenol: To a solution of 4-bromo-2,5-difluorophenol (0.6 g, 2.87 mmol, 1 eq) in AcOH (3 mL) was added HNO₃ (221 mg, 3.45 mmol, 158 μL, 1.2 eq). The mixture was stirred at 25° C. for 12 h under N₂. The reaction mixture was quenched by saturated aqueous NaHCO₃ at 0° C. and extracted with EtOAc (20 mL×3). The combined organic layers were washed with brine (20 mL), dried over Na₂SO₄, filtered, and concentrated under reduced pressure. The residue was purified by flash silica gel chromatography (from petroleum ether/ethyl acetate=100/1 to 0/1) to yield the title compound. ¹H NMR (400 MHz, DMSO-d₆) δ ppm: 8.02 (dd, J=6.8, 10.4 Hz, 1H).

1-bromo-2,5-difluoro-4-methoxy-3-nitrobenzene: To a solution of 4-bromo-3,6-difluoro-2-nitrophenol (230 mg, 815 μmol, 1 eq) in DMF (4 mL) was added K₂CO₃ (338 mg, 2.45 mmol, 3 eq) and iodomethane (231 mg, 1.63 mmol, 101 μL, 2 eq). The mixture was stirred at 25° C. for 2 h under N₂. The mixture was diluted with water (100 mL), extracted with EtOAc (100 mL×3), dried over Na₂SO₄, filtered, and concentrated under reduced pressure to give the title compound, which was used without further purification. ¹H NMR (400 MHz, DMSO-d₆) δ ppm: 8.25 (dd, J=6.8, 11.6 Hz, 1H), 4.05 (d, J=2.4 Hz, 3H).

3-bromo-2,5-difluoro-6-methoxyaniline: To a solution of 1-bromo-2,5-difluoro-4-methoxy-3-nitrobenzene (180 mg, 604 μmol, 1 eq) in EtOH (3 mL) and H₂O (1 mL) was added Fe (169 mg, 3.02 mmol, 5 eq) and NH₄Cl (162 mg, 3.02 mmol, 5 eq). The mixture was stirred at 80° C. for 2 h under N₂. The mixture was filtered, the filter cake was washed with EtOAc (30 mL×3). The filtrate was diluted with water (50 mL), extracted with EtOAc (50 mL×3), dried over Na₂SO₄, filtered, and concentrated under reduced pressure. The residue was purified by flash silica gel chromatography (from petroleum ether/ethyl acetate=100/1 to 4/1) to yield the title compound. ¹H NMR (400 MHz, DMSO-d₆) δ ppm: 6.77 (dd, J=6.0, 10.4 Hz, 1H), 5.58 (s, 2H), 3.76 (d, J=0.8 Hz, 3H).

### Intermediate 33

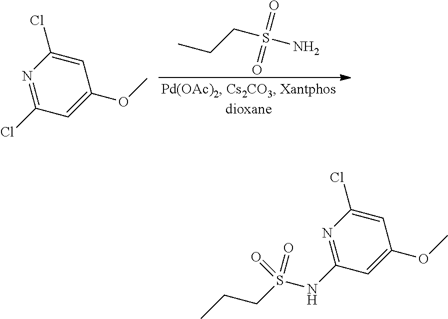

N-(6-chloro-4-methoxypyridin-2-yl)propane-1-sulfonamide: A mixture of 2,6-dichloro-4-methoxypyridine (500 mg, 2.81 mmol, 1 eq), propane-1-sulfonamide (415 mg, 3.37 mmol, 1.2 eq), Pd(OAc)₂ (63 mg, 281 mol, 0.1 eq), Cs₂CO₃ (1.83 g, 5.62 mmol, 2 eq) and Xantphos (195 mg, 337 mol, 0.12 eq) in 1,4-dioxane (10 mL) was degassed and purged with N₂ three times, and then the mixture was stirred at 100° C. for 16 h under N₂. The reaction mixture was concentrated under reduced pressure to remove 1,4-dioxane. The residue was purified by column chromatography (SiO₂, DCM/MeOH=100/0 to 100/3) to give the title compound. [M+H]⁺=265.0. ¹H NMR (400 MHz, DMSO-d₆) δ ppm: 10.71 (s, 1H), 6.81 (d, J=2.0 Hz, 1H), 6.49 (d, J=1.6 Hz, 1H), 3.83 (s, 3H), 3.45-3.36 (m, 2H), 1.77-1.62 (m, 2H), 0.96 (t, J=7.2 Hz, 3H).

### Intermediate 34

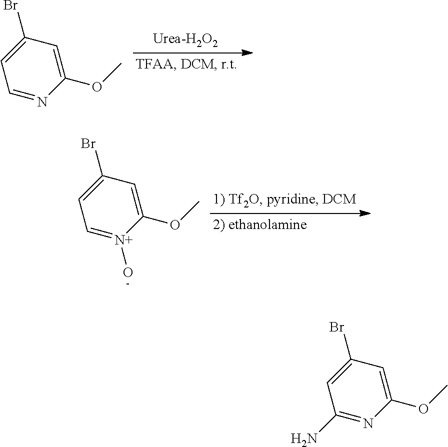

4-bromo-2-methoxypyridine 1-oxide: A mixture of 4-bromo-2-methoxypyridine (1 g, 5.32 mmol, 1 eq), hydrogen peroxide; urea (5.00 g, 53 mmol, 10 eq), and TFAA (3.35 g, 16 mmol, 2.22 mL, 3 eq) in DCM (3 mL) was degassed and purged with N₂ three times, and then the mixture was stirred at 25° C. for 4 h under N₂. The reaction mixture was quenched by saturated aqueous Na₂SO₃ solution (10 mL), diluted with H₂O (20 mL) and extracted with EtOAc (60 mL). The combined organic layers were washed with brine (30 mL), dried over Na₂SO₄, filtered, and concentrated under reduced pressure to give the title compound. ¹H NMR (400 MHz, CDCl₃) δ ppm: 8.12 (d, J=6.8 Hz, 1H), 7.29-7.00 (m, 2H), 4.08 (s, 3H).

4-bromo-6-methoxypyridin-2-amine: To a solution of 4-bromo-2-methoxypyridine 1-oxide (220 mg, 970 mol, 1 eq) in DCM (10 mL) was added dropwise pyridine (154 mg, 1.94 mmol, 157 μL, 2 eq) at 0° C. Then Tf₂O (301 mg, 1.07 mmol, 176 μL, 1.1 eq) in DCM (5 mL) was added dropwise to the mixture at 0° C. After addition, the mixture was stirred at 0° C. for 0.5 h, and then ethanolamine (301 mg, 1.07 mmol, 176 μL, 1.1 eq) was added dropwise at 0° C. The mixture was slowly warmed to 25° C. The resulting mixture was stirred at 25° C. for 14 h. The reaction mixture was quenched by addition H₂O (20 mL), and then extracted with DCM (20 mL×3). The combined organic layers were dried over Na₂SO₄, filtered, and concentrated under reduced pressure to give a residue, which was purified by flash silica gel chromatography (from petroleum ether/ethyl acetate=100/1 to 1/1) to yield the title compound. ¹H NMR (400 MHz, DMSO-d₆) δ ppm: 6.24-6.15 (m, 3H), 6.07 (d, J=1.2 Hz, 1H), 3.73 (s, 3H).

### Intermediate 35

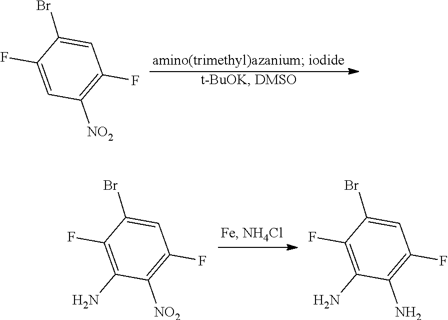

3-bromo-2,5-difluoro-6-nitroaniline: To a solution of 1-bromo-2,5-difluoro-4-nitrobenzene (2 g, 8.40 mmol, 1 eq) in DMSO (40 mL) was added amino(trimethyl)ammonium;iodide (2.04 g, 10.1 mmol, 1.2 eq) and t-BuOK (3.77 g, 33.6 mmol, 4 eq) in one portion at 15° C. After addition, the mixture was stirred at 15° C. for 15 min. The reaction mixture was quenched with 10% HCl (20 mL) to make pH=4-5 at 10° C., then the mixture was poured into water (150 mL) and extracted with EtOAc (60 mL×3). The combined organic layers were washed with water (50 mL×3), brine (50 mL×3), and dried over Na₂SO₄, filtered, and concentrated under reduced pressure. The residue was purified by flash silica gel chromatography (from petroleum ether/EtOAc=50/1 to 10/1) to yield the title compound. ¹H NMR (400 MHz, DMSO-d₆) δ ppm: 7.19 (s, 2H), 7.00 (dd, J=5.6, 11.2 Hz, 1H).

4-bromo-3,6-difluorobenzene-1,2-diamine: To a solution 3-bromo-2,5-difluoro-6-nitroaniline (250 mg, 889 μmol, 1 eq) in EtOH (5 mL), THE (5 mL) and H₂O (3 mL) was added Fe (500 mg, 8.89 mmol, 10 eq) and NH₄Cl (476 mg, 8.89 mmol, 10 eq). The reaction mixture was stirred at 55° C. for 2 h. The reaction mixture was concentrated under reduced pressure to give the title compound, which was used without further purification. [M+H]⁺=222.9. ¹H NMR (400 MHz, DMSO-d₆) δ ppm: 6.68-6.61 (m, 1H), 5.05 (s, 2H), 4.88 (s, 2H).

### Intermediate 36

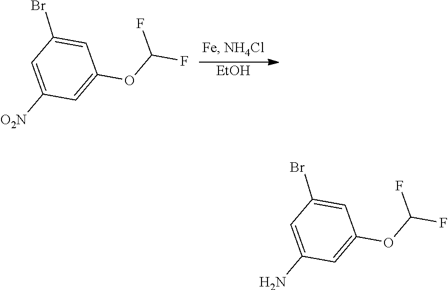 [SMILES: "EtOH" ; O=[N+]([O-])c1cc(Br)cc(OC(F)F)c1 ; Nc1cc(Br)cc(OC(F)F)c1]

3-bromo-5-(difluoromethoxy)aniline: A mixture of Fe (208 mg, 3.73 mmol, 10 eq), NH₄Cl (200 mg, 3.73 mmol, 10 eq) and 1-bromo-3-(difluoromethoxy)-5-nitrobenzene (100 mg, 373 μmol, 1 eq) in EtOH (5 mL) was stirred at 80° C. for 2 h, then the mixture was stirred at 90° C. for 1 h. The mixture was heated to 100° C. and stirred for 1 h under N₂. The reaction mixture was diluted with H₂O (20 mL) and extracted with EtOAc (20 mL×3), dried over Na₂SO₄, filtered, and concentrated under reduced pressure to give the title compound. ¹H NMR (400 MHz, DMSO-d₆) δ ppm: 7.15 (t, J=74 Hz, 1H), 6.61-6.57 (m, 1H), 6.43 (d, J=1.6 Hz, 1H), 6.38-6.22 (m, 1H), 5.71 (s, 2H).

### Intermediate 37

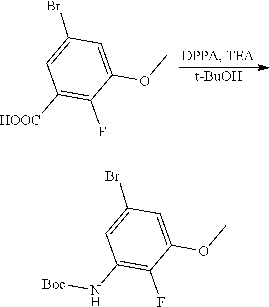 [SMILES: "t-BuOH" ; COc1cc(Br)cc(C(=O)O)c1F ; COc1cc(Br)cc(NC(=O)OC(C)(C)C)c1F]

tert-butyl (5-bromo-2-fluoro-3-methoxyphenyl)carbamate: A mixture of 5-bromo-2-fluoro-3-methoxybenzoic acid (200 mg, 803 μmol, 1 eq), Et₃N (163 mg, 1.61 mmol, 224 μL, 2 eq) and DPPA (265 mg, 964 mol, 209 μL, 1.2 eq) in t-BuOH (4 mL) was degassed and purged with N₂ three times, and then the mixture was stirred at 90° C. for 6 h under N₂. The reaction mixture was diluted with H₂O (30 mL) and extracted with EtOAc (30 mL×3). The combined organic layers were washed with brine (30 mL), dried over Na₂SO₄, filtered, and concentrated under reduced pressure to give a residue, which was purified by column chromatography (SiO₂, petroleum ether/ethyl acetate=100/0 to 95/5) to give the title compound. ¹H NMR (400 MHz, CDCl₃) δ ppm: 7.95 (d, J=4.8 Hz, 1H), 6.77 (dd, J=2.4, 7.6 Hz, 1H), 6.70 (s, 1H), 3.86 (s, 3H), 1.52 (s, 9H).

### Intermediate 38

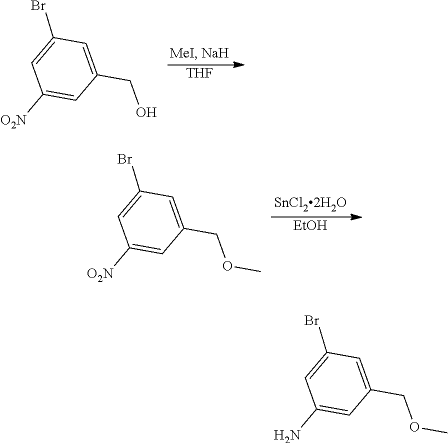

1-bromo-3-(methoxymethyl)-5-nitrobenzene: To a solution of (3-bromo-5-nitrophenyl)methanol (0.6 g, 2.59 mmol, 1 eq) in THF (10 mL) was added NaH (310 mg, 7.76 mmol, 60% purity, 3 eq) at 0° C. After addition, the mixture was stirred at 0° C. for 0.5 h, and then CH₃I (880 mg, 6.21 mmol, 386 μL, 2.4 eq) was added dropwise. The resulting mixture was stirred at 20° C. for 3 h. The reaction mixture was quenched by addition H₂O (10 mL), and the mixture was extracted with EtOAc 30 mL (10 mL×3). The combined organic layers were dried over Na₂SO₄, filtered, and concentrated under reduced pressure to give a residue. The residue was purified by flash silica gel chromatography (0-20% Ethyl acetate/petroleum ether) to give the title compound.

3-bromo-5-(methoxymethyl)aniline: To a solution of 1-bromo-3-(methoxymethyl)-5-nitrobenzene (300 mg, 1.22 mmol, 1 eq) in EtOH (20 mL) was added SnCl₂·2H₂O (2.20 g, 9.75 mmol, 8 eq). The mixture was stirred at 90° C. for 1 h under N₂. The mixture was concentrated, diluted with water (50 mL), extracted with EtOAc (50 mL×3). The combined organic layers were washed with brine (50 mL), dried over Na₂SO₄, filtered, and concentrated. The residue was purified by flash silica gel chromatography (0-50% ethyl acetate/petroleum ether gradient) to give the title compound. [M+H]⁺=217.6). ¹H NMR (400 MHz, DMSO-d₆) δ ppm: 6.64 (s, 1H), 6.57 (s, 1H), 6.49 (s, 1H), 4.24 (s, 2H), 3.25 (s, 3H).

### Intermediate 39

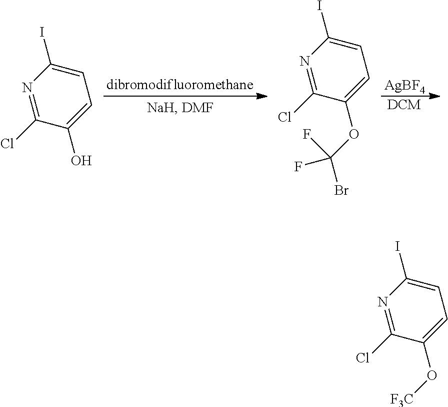

3-(bromodifluoromethoxy)-2-chloro-6-iodopyridine: To a solution of 2-chloro-6-iodopyridin-3-ol (2 g, 7.83 mmol, 1 eq) in DMF (15 mL) was added NaH (1.67 g, 41.7 mmol, 60% purity, 5.32 eq) and dibromodifluoromethane (9.86 g, 47.0 mmol, 4.34 mL, 6 eq) in DMF (5 mL). The mixture was stirred at 25° C. for 12 h. The reaction mixture was diluted with water (80 mL) and extracted with DCM (80 mL×3). The combined organic layers were washed with water (80 mL), dried over Na₂SO₄, filtered, and concentrated under reduced pressure to give a residue. The residue was purified by flash silica gel chromatography (from petroleum ether/ethyl acetate=100/0 to 99/1) to yield the title compound. ¹H NMR (400 MHz, DMSO-d₆) δ ppm: 8.02 (d, J=8.4 Hz, 1H), 7.80-7.72 (m, 1H).

2-chloro-6-iodo-3-(trifluoromethoxy)pyridine: To a mixture of 3-(bromodifluoromethoxy)-2-chloro-6-iodopyridine (3.5 g, 8.20 mmol, 1 eq) in DCM (20 mL) was added AgBF₄ (3.51 g, 18.0 mmol, 2.2 eq) at −78° C. under N₂, and then the mixture was stirred at 20° C. for 16 h under N₂. The mixture was filtered, and the filtrate was concentrated to give the title compound, which was used without further purification. [M+H]⁺=324.0. ¹H NMR (400 MHz, DMSO-d₆) δ ppm: 8.03 (d, J=8.4 Hz, 1H), 7.82 (dd, J=1.2, 8.4 Hz, 1H).

### Intermediate 40

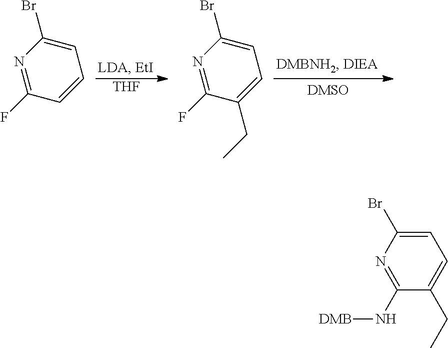

6-bromo-3-ethyl-2-fluoropyridine: To a solution of 2-bromo-6-fluoropyridine (1 g, 5.7 mmol, 1 eq) in THF (20 mL) was slowly added LDA (2 M, 5.68 mL, 2 eq) at −78° C., the mixture was stirred at −78° C. for 30 min. And to the mixture was slowly added EtI (2.66 g, 17.0 mmol, 1.36 mL, 3 eq), the mixture was stirred at −78° C. for 3 h, then it was stirred at 25° C. for 1 h under N₂. The reaction mixture was diluted with H₂O (20 mL) and extracted with EtOAc (20 mL×3), dried over Na₂SO₄, filtered, and concentrated under reduced pressure. The residue was purified by column chromatography (SiO₂, petroleum ether/ethyl acetate=1/0) to give the title compound. [M+H]⁺=203.8.

6-bromo-N-(2,4-dimethoxybenzyl)-3-ethylpyridin-2-amine: To a mixture of 6-bromo-3-ethyl-2-fluoropyridine (760 mg, 3.72 mmol, 1 eq), and DIEA (4.81 g, 37.3 mmol, 6.49 mL, 10 eq) in DMSO (20 mL) was added DMBNH₂ (6.23 g, 37.3 mmol, 5.61 mL, 10 eq), and then the mixture was degassed and purged with N₂ three times, the mixture was stirred at 80° C. for 12 h under N₂. The reaction mixture was diluted with H₂O (20 mL), extracted with DCM (20 mL×3), dried over Na₂SO₄, filtered, and concentrated under reduced pressure to give a residue. The residue was purified by column chromatography (SiO₂, petroleum ether/ethyl acetate=1/0 to 10/1) to give the title compound. [M+H]⁺=351.1. ¹H NMR (400 MHz, CDCl₃) δ ppm: 7.31 (d, J=8.0 Hz, 1H), 7.01 (d, J=7.2 Hz, 1H), 6.66 (d, J=7.6 Hz, 1H), 6.49-6.42 (m, 2H), 4.86-4.73 (m, 1H), 4.56 (d, J=5.6 Hz, 2H), 3.84 (s, 3H), 3.80 (s, 3H), 2.30 (q, J=7.6 Hz, 2H), 1.17 (t, J=7.6 Hz, 3H).

### Intermediate 41

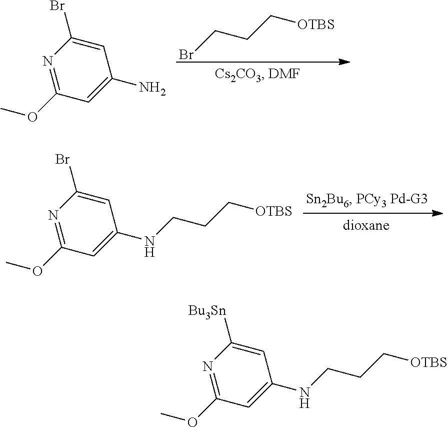

2-bromo-N-(3-((tert-butyldimethylsilyl)oxy)propyl)-6-methoxypyridin-4-amine: To a solution of 2-bromo-6-methoxypyridin-4-amine (1.25 g, 4.93 mmol, 6.41 μL, 2 eq) and (3-bromopropoxy)(tert-butyl)dimethylsilane (500 mg, 2.46 mmol, 1 eq) in DMF (15 mL) was added Cs₂CO₃ (4.01 g, 12.3 mmol, 5 eq). The mixture was stirred at 80° C. for 12 h. The reaction mixture was diluted with H₂O (30 mL) and extracted with EtOAc (50 mL×3). The combined organic layers were washed with brine (50 mL), dried over Na₂SO₄, filtered, and concentrated under reduced pressure. The residue was purified by column chromatography (SiO₂, petroleum ether/ethyl acetate=100/1 to 100/5) to give the title compound. [M+H]⁺=376.2. ¹H NMR (400 MHz, CDCl₃) δ ppm: 6.29 (d, J=1.6 Hz, 1H), 5.74 (d, J=1.6 Hz, 1H), 3.86 (s, 3H), 3.76 (t, J=5.2 Hz, 2H), 3.22 (t, J=6.4 Hz, 2H), 1.85-1.74 (m, 2H), 0.92 (s, 9H), 0.07 (s, 6H).

N-(3-((tert-butyldimethylsilyl)oxy)propyl)-2-methoxy-6-(tributylstannyl)pyridin-4-amine: A mixture of 2-bromo-N-(3-((tert-butyldimethylsilyl)oxy)propyl)-6-methoxypyridin-4-amine (620 mg, 1.49 mmol, 1 eq), Sn₂Bu₆ (2.59 g, 4.46 mmol, 2.23 mL, 3 eq) and PCy3 Pd-G3 (109 mg, 149 mol, 0.1 eq) in 1,4-dioxane (10 mL) was degassed and purged with N₂ three times, and then the mixture was stirred at 115° C. for 12 h under N₂. The reaction mixture was diluted with H₂O (30 mL) and extracted with EtOAc (50 mL×3). The combined organic layers were washed with brine (50 mL), dried over Na₂SO₄, filtered, and concentrated under reduced pressure to give a residue. The residue was purified by column chromatography (SiO₂, petroleum ether/ethyl acetate=100/0 to 100/15) to give the title compound.

### Intermediate 42

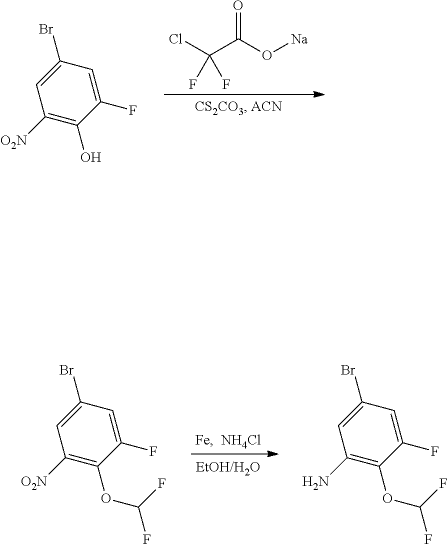

5-bromo-2-(difluoromethoxy)-1-fluoro-3-nitrobenzene: To a solution of 4-bromo-2-fluoro-6-nitrophenol (100 mg, 424 μmol, 1 eq) in ACN (10 mL) was added sodium 2-chloro-2,2-difluoroacetate (194 mg, 1.27 mmol, 3.0 eq) and Cs₂CO₃ (276 mg, 847 μmol, 2.0 eq). The mixture was stirred at 80° C. for 2 h. The reaction mixture was diluted with H₂O (20 mL) and extracted with EtOAc (10 mL×3). The combined organic layers were washed with brine (10 mL), dried over Na₂SO₄, filtered, and concentrated under reduced pressure to give a residue, which was purified by column chromatography (SiO₂, petroleum ether/ethyl acetate=1/0 to 10/1) to give the title compound. ¹H NMR (400 MHz, CDCl₃) δ ppm: 7.90 (t, J=2.0 Hz, 1H), 7.64 (dd, 1=2.4, 8.8 Hz, 1H), 6.88-6.43 (m, 1H).

5-bromo-2-(difluoromethoxy)-3-fluoroaniline: To a solution of 5-bromo-2-(difluoromethoxy)-1-fluoro-3-nitrobenzene (114 mg, 319 μmol, 1 eq) in EtOH (1.0 mL) and H₂O (1.0 mL) was added Fe (178 mg, 3.19 mmol, 10 eq) and NH₄Cl (171 mg, 3.19 mmol, 10 eq). The mixture was stirred at 80° C. for 1 h. The reaction mixture was filtered under reduced pressure and the filtrate was adjusted pH=8 with saturated aqueous NaHCO₃, diluted with H₂O (10 mL) and extracted with EtOAc (10 mL×3). The combined organic layers were washed with brine (10 mL), dried over Na₂SO₄, filtered, and concentrated under reduced pressure to give a residue, which was purified by prep-TLC (SiO₂, petroleum ether: ethyl acetate=10:1) to give the title compound.

### Intermediate 43

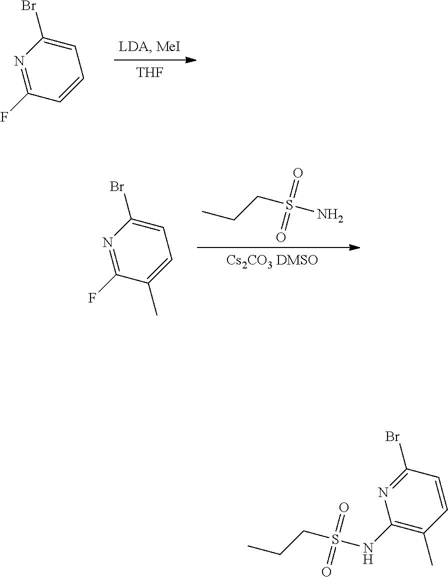

6-bromo-2-fluoro-3-methylpyridine: To a solution of 2-bromo-6-fluoropyridine (2.0 g, 11.36 mmol, 1 eq) in THE (32 mL) was added LDA (2.0 M, 8.52 mL, 1.5 eq). The mixture was stirred at −70° C. for 0.5 h. Then to the mixture was added Mel (9.68 g, 68.2 mmol, 4.24 mL, 6.0 eq) and the mixture was stirred at −70° C. for 3 h. The reaction mixture was diluted with H₂O (50 mL) and extracted with EtOAc (50 mL×3), dried over Na₂SO₄, filtered, and concentrated under reduced pressure. The residue was purified by column chromatography (SiO₂, petroleum ether/ethyl acetate=1/0 to 4/1) to give the title compound. [M+H]⁺=189.7. ¹H NMR (400 MHz, DMSO-d₆) δ ppm: 7.85-7.75 (m, 1H), 7.56-7.50 (m, 1H), 2.21 (s, 3H).

N-(6-bromo-3-methylpyridin-2-yl)propane-1-sulfonamide: To a mixture of 6-bromo-2-fluoro-3-methylpyridine (400 mg, 1.89 mmol, 1 eq) and Cs₂CO₃ (1.85 g, 5.68 mmol, 3 eq) in DMSO (8 mL) was added propane-1-sulfonamide (700 mg, 5.68 mmol, 3 eq) and then the mixture was degassed and purged with N₂ three times. The mixture was stirred at 120° C. for 3 h under microwave irradiation. The reaction mixture was diluted with H₂O (30 mL), extracted with EtOAc (30 mL×3), dried over Na₂SO₄, filtered, and concentrated under reduced pressure. The residue was purified by column chromatography (SiO₂, petroleum ether/ethyl acetate=1/0 to 4/1) to give the title compound. [M+H]⁺=294.8. H NMR (400 MHz, CDCl₃) δ ppm: 7.31 (d, J=8.0 Hz, 1H), 7.12 (d, J=8.0 Hz, 1H), 3.73-3.60 (m, 2H), 2.20 (s, 3H), 1.98-1.85 (m, 2H), 1.10 (t, J=7.6 Hz, 3H).

The following compounds were prepared following procedures analogous to that described for Intermediate 43.

| Int. | | | |
|---|---|---|---|
| No. | Structure | Name | LCMS, ¹H NMR |
| Int. 44 | 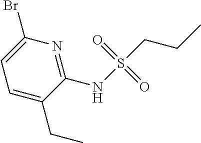 [SMILES: CCCS(=O)(=O)Nc1nc(Br)ccc1CC] | N-(6-bromo-3- ethylpyridin-2- yl)propane-1- sulfonamide | [M + H]⁺ = 308.0 ¹H NMR (400 MHz, CDCl₃) δ ppm: 7.33 (d, J = 8.0 Hz, 1H), 7.16 (d, J = 8.0 Hz, 1H), 3.75-3.55 (m, 2H), 2.51 (q, J = 7.6 Hz, 2H), 2.00-1.85 (m, 2H), 1.30- 1.20 (m, 3H), 1.11 (t, J = 7.6 Hz, 3H) |

### Intermediate 45

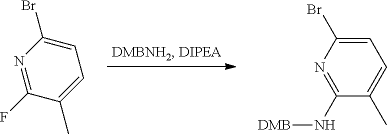

6-bromo-N-(2,4-dimethoxybenzyl)-3-methylpyridin-2-amine: A mixture of 6-bromo-2-fluoro-3-methylpyridine (1.5 g, 7.10 mmol, 1 eq), DMBNH₂ (11.9 g, 71.0 mmol, 10.7 mL, 10 eq), and DIEA (9.18 g, 71.0 mmol, 12.4 mL, 10 eq) in DMSO (30 mL) was degassed and purged with N₂ three times, and then the mixture was stirred at 80° C. for 12 h under N₂. The reaction mixture was diluted with H₂O (100 mL) and extracted with EtOAc (50 mL×3), dried over Na₂SO₄, filtered, and concentrated under reduced pressure to give a residue. The residue was purified by column chromatography (SiO₂, petroleum ether/ethyl acetate=1/0 to 5/1) to give the title compound. ¹H NMR (400 MHz, DMSO-d₆) δ ppm: 7.15 (d, J=7.2 Hz, 1H), 7.05 (d, J=8.4 Hz, 1H), 6.59 (d, J=7.2 Hz, 1H), 6.55 (d, J=2.0 Hz, 1H), 6.53-6.48 (m, 1H), 6.44 (dd, J=2.4, 8.4 Hz, 1H), 4.41 (d, J=6.0 Hz, 2H), 3.81 (s, 3H), 3.73 (s, 3H), 2.04 (s, 3H).

### Intermediate 46

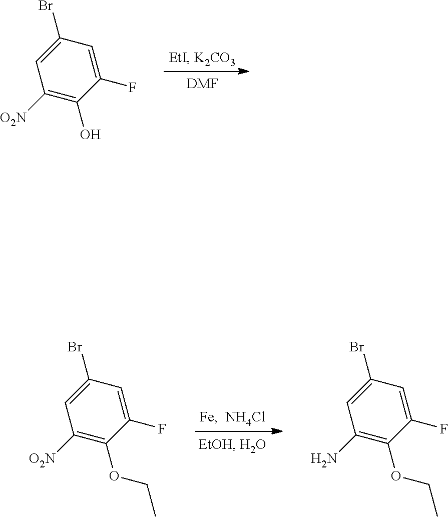

5-bromo-2-ethoxy-1-fluoro-3-nitrobenzene: To a solution of 4-bromo-2-fluoro-6-nitrophenol (150 mg, 636 μmol, 1 eq) in DMF (2 mL) was added K₂CO₃ (264 mg, 1.91 mmol, 3 eq). The mixture was stirred for 1 h at 25° C., and then EtI (496 mg, 3.18 mmol, 254 μL, 5 eq) was added to the reaction mixture. The mixture was stirred at 25° C. for 17 h. The reaction mixture was diluted with H₂O (10 mL) and extracted with EtOAc (10 mL×2). The combined organic layers were washed with brine (10 mL×2), dried over Na₂SO₄, filtered, and concentrated under reduced pressure to give the title compound, which was used without further purification. ¹H NMR (400 MHz, DMSO-d₆) δ ppm: 8.08-8.06 (m, 1H), 8.05-8.03 (m, 1H), 4.27-4.18 (m, 2H), 1.30 (t, J=6.8 Hz, 3H).

5-bromo-2-ethoxy-3-fluoroaniline: A mixture of 5-bromo-2-ethoxy-1-fluoro-3-nitrobenzene (150 mg, 568 mol, 1 eq), NH₄Cl (304 mg, 5.68 mmol, 10 eq), and Fe (317 mg, 5.68 mmol, 10 eq) in EtOH (10 mL) and H₂O (5 mL) was degassed and purged with N₂ three times, and then the mixture was stirred at 80° C. for 2 h under N₂. The reaction mixture was diluted with H₂O (10 mL) and extracted with EtOAc (10 mL×2). The combined organic layers were washed with brine (10 mL×2), dried over Na₂SO₄, filtered, and concentrated under reduced pressure to give the title compound, which was used without further purification. ¹H NMR (400 MHz, DMSO-d₆) δ ppm: 6.68-6.64 (m, 1H), 6.59-6.55 (m, 1H), 5.46 (s, 2H), 3.95-3.88 (m, 2H), 1.28 (t, J=7.2 Hz, 3H).

### Intermediate 47

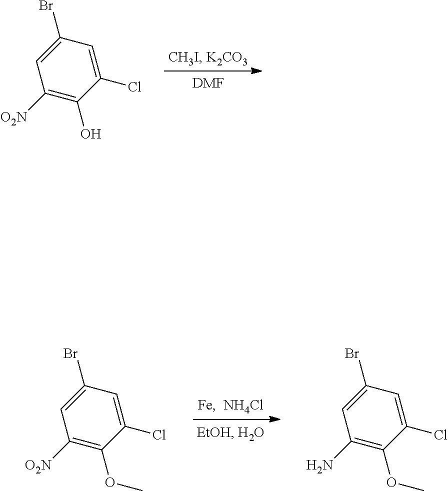

5-bromo-1-chloro-2-methoxy-3-nitrobenzene: To a solution of 4-bromo-2-chloro-6-nitrophenol (500 mg, 1.98 mmol, 1 eq) in DMF (15 mL) was added CH₃I (562 mg, 3.96 mmol, 247 μL, 2 eq) and K₂CO₃ (547 mg, 3.96 mmol, 2 eq). The mixture was stirred at 60° C. for 2 h. The reaction mixture was quenched by addition H₂O (5 mL), diluted with brine (100 mL) and extracted with ethyl acetate (100 mL×3). The combined organic layers were washed with brine (200 mL), dried over Na₂SO₄, filtered, and concentrated under reduced pressure to give the title compound, which was used without further purification. ¹H NMR (400 MHz, DMSO-d₆) δ ppm: 8.21 (s, 2H), 3.93 (s, 3H).

5-bromo-3-chloro-2-methoxyaniline: To a solution of 5-bromo-1-chloro-2-methoxy-3-nitrobenzene (200 mg, 675 μmol, 1 eq) in EtOH (2.5 mL) and H₂O (0.5 mL) was added Fe (377 mg, 6.75 mmol, 10 eq) and NH₄Cl (361 mg, 6.75 mmol, 10 eq). The mixture was stirred at 80° C. for 1 h. The reaction mixture was diluted with H₂O (10 mL) and extracted with ethyl acetate (20×3 mL). The combined organic layers were dried over Na₂SO₄, filtered, and concentrated under reduced pressure to give the title compound, which was used without further purification. ¹H NMR (400 MHz, DMSO-d₆) δ ppm: 6.80 (d, J=2.4 Hz, 1H), 6.72 (d, J=2.0 Hz, 1H), 5.59 (s, 2H), 3.67 (s, 3H).

### Intermediate 48

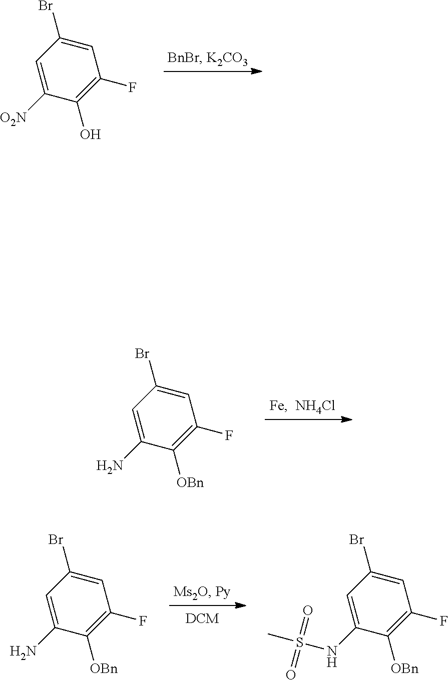

2-(benzyloxy)-5-bromo-3-fluoroaniline: To a solution of 4-bromo-2-fluoro-6-nitrophenol (500 mg, 2.12 mmol, 1 eq) in DMF (6 mL) was added K₂CO₃ (878 mg, 6.36 mmol, 3 eq) and BnBr (1.81 g, 10.6 mmol, 1.26 mL, 5 eq). The mixture was stirred at 25° C. for 12 h. The reaction mixture was diluted with H₂O (10 mL) and extracted with EtOAc (20 mL×3). The combined organic layers were washed with brine (15 mL), dried over Na₂SO₄, filtered, and concentrated under reduced pressure to give a residue, which was purified by flash silica gel chromatography (from petroleum ether/EtOAc=100/1 to 7/3) to give the title compound. ¹H NMR: (400 MHz, DMSO-d₆) δ ppm: 8.12-8.07 (m, 1H), 8.06-7.98 (m, 1H), 7.42-7.34 (m, 5H), 5.23 (d, J=0.8 Hz, 2H).

2-(benzyloxy)-5-bromo-3-fluoroaniline: To a solution of 2-(benzyloxy)-5-bromo-3-fluoroaniline (700 mg, 2.15 mmol, 1 eq) in EtOH (6 mL) and H₂O (6 mL) was added Fe (1.20 g, 21.5 mmol, 10 eq) and NH₄Cl (1.15 g, 21.5 mmol, 10 eq). The mixture was stirred at 80° C. for 1 h under N₂. The reaction mixture was diluted with H₂O (45 mL) and extracted with EtOAc (30 mL×3). The combined organic layers were washed with brine (20 mL), dried over Na₂SO₄, filtered, and concentrated under reduced pressure to give a residue, which was purified by flash silica gel chromatography (from petroleum ether/EtOAc=100/1 to 7/3) to give the title compound. ¹H NMR (400 MHz, DMSO-d₆) δ ppm: 7.51-7.43 (m, 2H), 7.42-7.33 (m, 3H), 6.69-6.65 (m, 1H), 6.62-6.53 (m, 1H), 5.46 (s, 2H), 4.94 (s, 2H).

N-(2-(benzyloxy)-5-bromo-3-fluorophenyl)methanesulfonamide: To a solution of 2-(benzyloxy)-5-bromo-3-fluoroaniline (114 mg, 346 μmol, 1 eq) in DCM (2 mL) was added pyridine (82 mg, 1.04 mmol, 84 μL, 3 eq) and Ms₂O (66.4 mg, 381 μmol, 1.1 eq). The mixture was stirred at 25° C. for 1 h under N₂. To the reaction mixture was added Ms₂O (60.4 mg, 346 mol, 1 eq). The mixture was stirred at 25° C. for 1 h under N₂. To the reaction mixture was added Ms₂O (60.4 mg, 346 μmol, 1 eq). The mixture was stirred at 25° C. for 1 h under N₂. The reaction mixture was diluted with H₂O (15 mL) and extracted with DCM (20 mL×3). The combined organic layers were washed with brine (20 mL), dried over Na₂SO₄, filtered, and concentrated under reduced pressure to give a residue, which was purified by flash silica gel chromatography (from petroleum ether/EtOAc=100/1 to 7/3) to give the title compound. ¹H NMR (400 MHz, DMSO-d₆) δ ppm: 9.53 (s, 1H), 7.55-7.46 (m, 2H), 7.45-7.28 (m, 5H), 5.11 (s, 2H), 3.04 (s, 3H).

### Intermediate 49

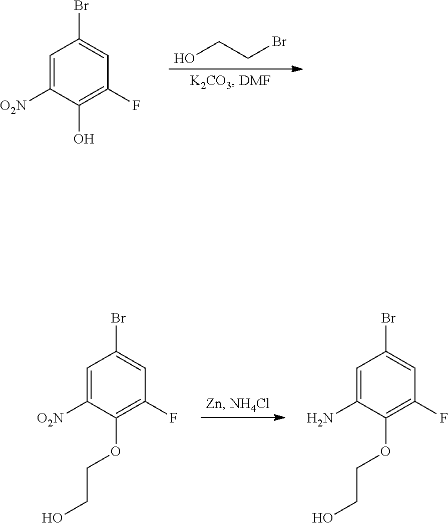

2-(4-bromo-2-fluoro-6-nitrophenoxy)ethan-1-ol: To a solution of 4-bromo-2-fluoro-6-nitrophenol (1 g, 4.24 mmol, 1 eq) in MeCN (10 mL) was added K₂CO₃ (1.17 g, 8.47 mmol, 2 eq) and 2-bromoethanol (1.20 mL, 16.95 mmol, 4 eq). The mixture was stirred at 80° C. for 12 h. The reaction mixture was concentrated in vacuo, and the resulting crude material was purified via silica gel chromatography (PE/EtOAc=1/0 to 3/1) to yield the title compound. ¹H NMR (400 MHz, CDCl₃) δ ppm 7.78 (t, J=2.4 Hz, 1H), 7.53 (dd, J=2.4, 10.0 Hz, 1H), 4.40-4.37 (m, 2H), 3.93-3.88 (m, 2H).

2-(2-amino-4-bromo-6-fluorophenoxy)ethan-1-ol: To a solution of 2-(4-bromo-2-fluoro-6-nitrophenoxy)ethan-1-ol (453 mg, 1.46 mmol, 1 eq) in EtOH (4 mL) and H₂O (2.4 mL) was added Zn (951.96 mg, 14.56 mmol, 10 eq) and NH₄Cl (778.72 mg, 14.56 mmol, 10 eq). The mixture was stirred at 80° C. for 10 m. The mixture was then stirred at 25° C. for 2 h. The reaction mixture was quenched by the addition of 1N HCl (1 mL). The reaction mixture was filtered and concentrated in vacuo. The resulting crude material was purified via silica gel chromatography (PE/EtOAc=8/1 to 7/2) to yield the title compound. LCMS [M+H]⁺=249.9. H NMR (400 MHz, CDCl₃) δ ppm 6.72-6.64 (m, 2H), 4.17-4.12 (m, 2H), 3.89-3.84 (m, 2H).

### Intermediate 50

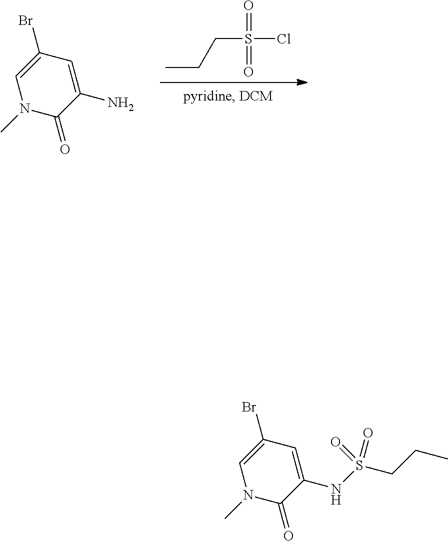

N-(5-bromo-1-methyl-2-oxo-1,2-dihydropyridin-3-yl)propane-1-sulfonamide: To a solution of 3-amino-5-bromo-1-methylpyridin-2(1H)-one (200 mg, 985 μmol, 1 eq) and propane-1-sulfonyl chloride (702 mg, 4.93 mmol, 554 μL, 5 eq) in DCM (10 mL) was added pyridine (389 mg, 4.93 mmol, 397 μL, 5 eq). The mixture was stirred at 25° C. for 2 h. The reaction mixture was filtered and concentrated under reduced pressure to give a residue, which was purified by column chromatography (SiO₂, petroleum ether/ethyl acetate=100/0 to 0/100) to give the title compound. ¹H NMR (400 MHz, CDCl₃) δ ppm 7.58 (d, J=2.0 Hz, 1H), 7.19 (d, J=2.0 Hz, 1H), 3.62 (s, 3H), 3.18-3.03 (m, 2H), 1.92-1.75 (m, 2H), 1.10-0.95 (m, 3H).

### Intermediate 51

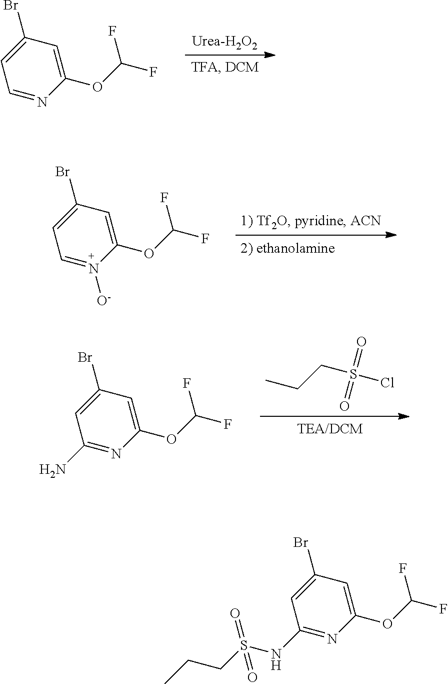

4-bromo-2-(difluoromethoxy)pyridine 1-oxide: The mixture of 4-bromo-2-(difluoromethoxy)pyridine (500 mg, 2.23 mmol, 1 eq), TFA (1.41 g, 6.70 mmol, 931 μL, 3 eq) in DCM (5 mL) was added Urea-H₂O₂ (1.68 g, 17.9 mmol, 8 eq) stirred at 40° C. for 12 h under N₂. The reaction mixture was quenched by addition of a saturated aqueous Na₂SO₃ solution (50 mL), diluted with H₂O (50 mL), and extracted with EtOAc (50 mL×3). The combined organic layers were washed with brine (30 mL), dried over Na₂SO₄, filtered, and concentrated under reduced pressure to give a residue, which was purified by column chromatography (SiO₂, petroleum ether/ethyl acetate=1/0 to 3/2) to give the title compound. ¹H NMR (400 MHz, CDCl₃) δ ppm: 8.10 (d, J=7.2 Hz, 1H), 7.70-7.34 (m, 2H), 7.32 (dd, J=2.8, 6.8 Hz, 1H).

4-bromo-6-(difluoromethoxy)pyridin-2-amine: To a mixture 4-bromo-2-(difluoromethoxy)pyridine 1-oxide (150 mg, 563 mol, 1 eq), pyridine (178 mg, 2.25 mmol, 182 μL, 4 eq) in acetonitrile (15 mL) was added Tf₂O (238 mg, 844 μmol, 139 μL, 1.5 eq) at 0° C., and then stirred at 25° C. for 3 h under N₂. To the mixture was added ethanolamine (344 mg, 5.62 mmol, 340 μL, 10 eq) at 0° C. and then stirred at 25° C. for 12 h under N₂. The reaction mixture was diluted with H₂O (20 mL) and extracted with EtOAc (20 mL×3). The combined organic layers were washed with brine (30 mL), dried over Na₂SO₄, filtered, and concentrated under reduced pressure to give a residue, which was purified by column chromatography (SiO₂, petroleum ether/ethyl acetate=1/0 to 1/1) to give the title compound. [M+H]⁺=238. ¹H NMR (400 MHz, CDCl₃) δ ppm: 7.52-7.28 (m, 1H), 6.42 (d, J=1.2 Hz, 1H), 6.41 (d, J=1.2 Hz, 1H).

N-(4-bromo-6-(difluoromethoxy)pyridin-2-yl)propane-1-sulfonamide: To the mixture of 4-bromo-6-(difluoromethoxy)pyridin-2-amine (100 mg, 377 μmol, 1 eq), TEA (191 mg, 1.88 mmol, 262 μL, 5 eq) in DCM (10 mL) was added propane-1-sulfonyl chloride (268 mg, 1.88 mmol, 211 μL, 5 eq), and then was degassed and purged with N₂ three times, and then the mixture was stirred at 40° C. for 12 h under N₂. The reaction mixture was diluted with H₂O (20 mL) and extracted with EtOAc (20 mL×3). The combined organic layers were washed with brine (30 mL), dried over Na₂SO₄, filtered, and concentrated under reduced pressure to give a residue, which was purified by column chromatography (SiO₂, petroleum ether/ethyl acetate=1/0 to 1/1) to give the title compound. ¹H NMR (400 MHz, CDCl₃) δ ppm: 7.28-7.20 (m, 1H), 7.14 (s, 1H), 7.05 (d, J=1.2 Hz, 1H), 6.81 (d, J=1.2 Hz, 1H), 3.40-3.30 (m, 2H), 1.95-1.84 (m, 2H), 1.08 (t, J=7.2 Hz, 3H).

### Intermediate 52

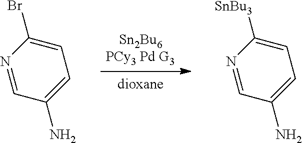

6-(tributylstannyl)pyridin-3-amine: A mixture of 6-bromopyridin-3-amine (200 mg, 1.16 mmol, 1 eq), Sn₂Bu₆ (2.68 g, 4.62 mmol, 2.31 mL, 4 eq) and PCy3 Pd G3 (84.98 mg, 115.60 μmol, 0.1 eq) in 1,4-dioxane (10 mL) was degassed and purged with N₂ 3 times, and then the mixture was stirred at 115° C. for 12 h under a N₂ atmosphere. The reaction mixture was diluted with sat. KF (10 mL) and extracted with EtOAc (3×20 mL). The combined organic layers were washed with brine (50 mL), dried over Na₂SO₄, filtered and concentrated under reduced pressure to give a residue. The residue was purified by column chromatography (SiO₂, 0-100% EtOAc/petroleum ether) to give the title compound. [M+H]⁺=382.8. ¹H NMR (400 MHz, CDCl₃) δ ppm 8.31 (d, J=2.4 Hz, 1H), 7.18 (d, J=8.0 Hz, 1H), 6.87 (dd, J=8.0, 2.8 Hz, 1H), 3.62 (br s, 2H), 1.58-1.47 (m, 6H), 1.37-1.25 (m, 9H), 1.12-1.05 (m, 3H), 0.90-0.85 (m, 9H).

### Intermediate 53

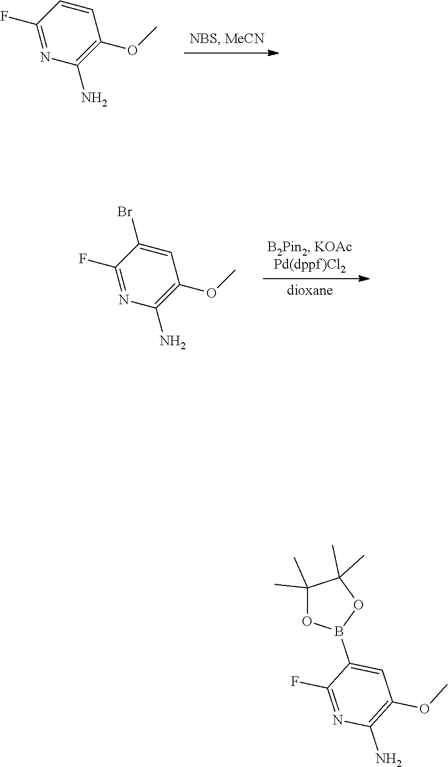

5-bromo-6-fluoro-3-methoxypyridin-2-amine: To a solution of 6-fluoro-3-methoxypyridin-2-amine (100 mg, 703.58 μmol, 1 eq) in MeCN (5 mL) was added NBS (130.23 mg, 731.72 mol, 1.04 eq) at 0° C. The mixture was stirred at 0° C. for 30 min and then the reaction mixture was warmed to 25° C. and stirred at 25° C. for 1 h before the mixture was concentrated under reduced pressure to give a residue. The residue was purified by column chromatography (SiO₂, petroleum ether/EtOAc=100/1 to 2/8) to yield the title compound. [M+H]⁺=220.9. ¹H NMR (400 MHz, CDCl₃) δ ppm 7.08 (d, J=6.8 Hz, 1H), 5.77-4.00 (m, 2H), 3.83 (s, 3H).

6-fluoro-3-methoxy-5-(4,4,5,5-tetramethyl-1,3,2-dioxaborolan-2-yl)pyridin-2-amine: A mixture of 5-bromo-6-fluoro-3-methoxypyridin-2-amine (80 mg, 325.75 μmol, 1 eq), B₂Pin₂ (248.16 mg, 977.26 μmol, 3 eq), KOAc (95.91 mg, 977.26 mol, 3 eq), Pd(dppf)Cl₂ (23.84 mg, 32.58 μmol, 0.1 eq) in 1,4-dioxane (8 mL) was degassed and purged with N₂ 3 times, and then the mixture was stirred at 100° C. for 12 h under a N₂ atmosphere. The reaction mixture was diluted with H₂O (30 mL) and extracted with EtOAc (3×30 mL). The combined organic layers were washed with brine (30 mL), dried over Na₂SO₄, filtered, and concentrated under reduced pressure to give a residue. The residue was purified by prep-TLC (petroleum ether/EtOAc=3/1) to give the title compound. [M+H]⁺=269.0.

### Intermediate 54

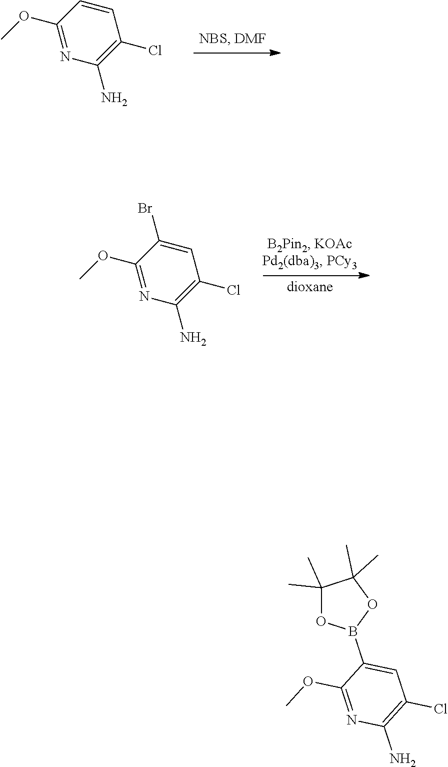

5-bromo-3-chloro-6-methoxypyridin-2-amine: To a solution of 3-chloro-6-methoxypyridin-2-amine (50 mg, 315.29 gmol, 1 eq) in DMF (2.5 mL) was added NBS (56.12 mg, 315.29 μmol, 1 eq). The mixture was stirred at 0° C. for 2 h. The reaction mixture was filtered and concentrated under reduced pressure to give a residue, which was purified by column chromatography (SiO₂, petroleum ether/EtOAc=100/0 to 100/15) to give the title compound. ¹H NMR (400 MHz, DMSO-d₆) δ ppm 7.74 (s, 1H), 6.45 (s, 2H), 3.81 (s, 3H).

3-chloro-6-methoxy-5-(4,4,5,5-tetramethyl-1,3,2-dioxaborolan-2-yl)pyridin-2-amine: A mixture of 5-bromo-3-chloro-6-methoxypyridin-2-amine (110 mg, 463.19 gmol, 1 eq), B₂Pin₂ (352.87 mg, 1.39 mmol, 3 eq), Pd₂(dba)₃ (42.42 mg, 46.32 μmol, 0.1 eq), KOAc (136.38 mg, 1.39 mmol, 3 eq) and PCy3 (12.99 mg, 46.32 μmol, 15.02 μL, 0.1 eq) in 1,4-dioxane (3 mL) was degassed and purged with N₂ 3 times, and then the mixture was stirred at 100° C. for 12 h under a N₂ atmosphere. The reaction mixture was filtered and concentrated under reduced pressure to give a residue, which was purified by column chromatography (SiO₂, petroleum ether/EtOAc=100/0 to 100/15) to give the title compound. ¹H NMR (400 MHz, CDCl₃) δ ppm 7.78 (s, 1H), 4.90 (s, 2H), 3.86 (s, 3H), 1.32 (s, 12H).

### Intermediate 55

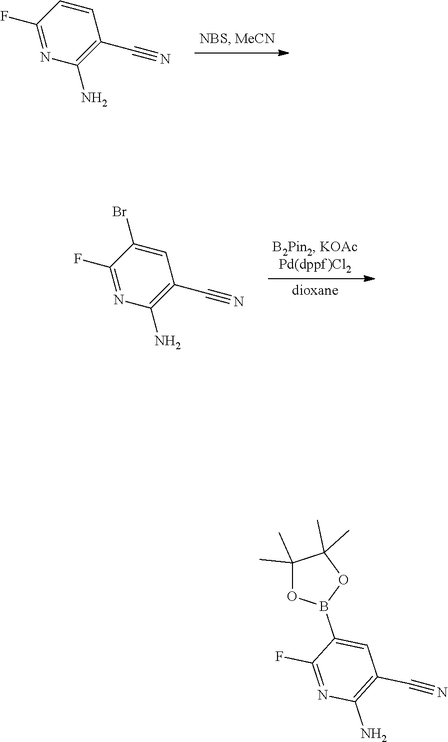 [SMILES: N#Cc1ccc(F)nc1N ; N#Cc1cc(Br)c(F)nc1N ; CC1(C)OB(c2cc(C#N)c(N)nc2F)OC1(C)C]

2-amino-5-bromo-6-fluoronicotinonitrile: To a solution of 2-amino-6-fluoronicotinonitrile (230 mg, 1.68 mmol, 1 eq) in MeCN (3.0 mL) was added NBS (597.11 mg, 3.35 mmol, 2.0 eq). The mixture was stirred at 70° C. for 12 h before it was filtered and concentrated under reduced pressure to give a residue, which was purified by column chromatography (SiO₂, petroleum ether/EtOAc=1/0 to 4/1) to give the title compound. ¹H NMR (400 MHz, CDCl₃) δ ppm 7.91 (d, J=8.0 Hz, 1H), 5.32 (s, 2H).

2-amino-6-fluoro-5-(4,4,5,5-tetramethyl-1,3,2-dioxaborolan-2-yl)nicotinonitrile: A mixture of 2-amino-5-bromo-6-fluoronicotinonitrile (80 mg, 333.32 mol, 1 eq), B₂Pin₂ (211.60 mg, 833.29 μmol, 2.5 eq), Pd(dppf)Cl₂ (24.39 mg, 33.33 mol, 0.1 eq), KOAc (81.78 mg, 833.29 μmol, 2.5 eq) in 1,4-dioxane (2.0 mL) was degassed and purged with N₂ 3 times, and then the mixture was stirred at 100° C. for 12 h under a N₂ atmosphere. The reaction mixture was filtered and concentrated under reduced pressure to give the title compound, which was used in the next step without further purification.

### Intermediate 56

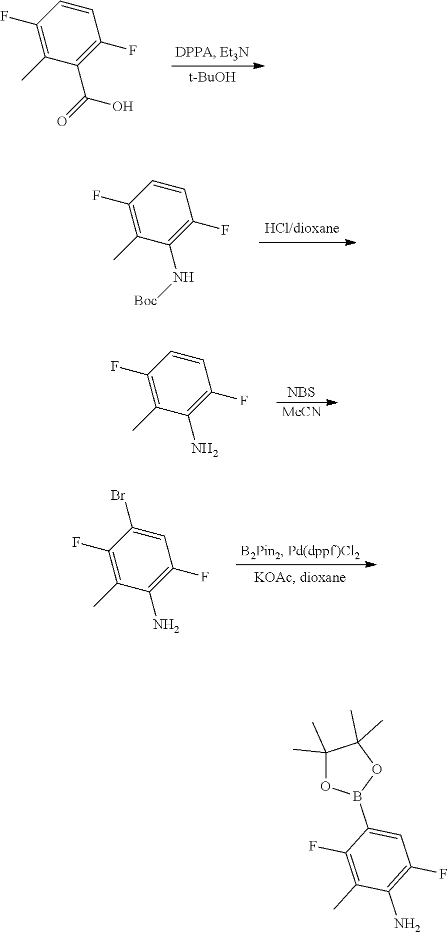

tert-butyl (3,6-difluoro-2-methylphenyl)carbamate: A mixture of 3,6-difluoro-2-methylbenzoic acid (790 mg, 4.13 mmol, 1 eq) and Et₃N (836 mg, 8.26 mmol, 1.15 mL, 2 eq) in t-BuOH (20 mL) was degassed and purged with N₂ 3 times, and then the mixture was heated to 40° C. before DPPA (1.25 g, 4.54 mmol, 980.73 μL, 1.1 eq) was added to the mixture. The mixture was stirred at 90° C. for 12 h under a N₂ atmosphere. The mixture was concentrated under reduced pressure to give a residue, which was purified by column chromatography (SiO₂, petroleum ether/EtOAc=100/0 to 3/1) to give the title compound. ¹H NMR (400 MHz, CDCl₃) δ ppm 6.93-6.85 (m, 2H), 5.99 (br s, 1H), 2.20 (d, J=2.4 Hz, 3H), 1.50 (s, 9H).

3,6-difluoro-2-methylaniline: A mixture of tert-butyl (3,6-difluoro-2-methylphenyl)carbamate (500 mg, 1.85 mmol, 1 eq) in 4 M HCl in dioxane (25 mL) was stirred at 25° C. for 1 h. The reaction mixture was filtered and concentrated under reduced pressure to give a residue, which was purified by column chromatography (SiO₂, petroleum ether/EtOAc=100/0 to 70/30) to give the title compound. ¹H NMR (400 MHz, DMSO-d₆) δ ppm 6.89-6.82 (m, 1H), 6.33-6.27 (m, 1H), 3.57 (s, 2H), 2.01 (d, J=2.0 Hz, 3H).

4-bromo-3,6-difluoro-2-methylaniline: To a solution of 3,6-difluoro-2-methylaniline (200 mg, 1.26 mmol, 1 eq) in MeCN (18 mL) was added NBS (224 mg, 1.26 mmol, 1 eq) and Et₃N (254.50 mg, 2.52 mmol, 350.07 μL, 2 eq). The mixture was stirred at 25° C. for 2 h before it was concentrated, diluted with water (20 mL) and extracted with EtOAc (3×60 mL). The combined organic layers were washed with brine (20 mL), dried over Na₂SO₄, filtered, and concentrated under reduced pressure to give a residue, which was purified by column chromatography (SiO₂, petroleum ether/EtOAc=100/0 to 90/10) to give the title compound. ¹H NMR (400 MHz, CDCl₃) δ ppm 7.10-7.01 (m, 1H), 3.78 (s, 2H), 2.13 (d, J=2.0, 3H).

3,6-difluoro-2-methyl-4-(4,4,5,5-tetramethyl-1,3,2-dioxaborolan-2-yl)aniline: A mixture of 4-bromo-3,6-difluoro-2-methylaniline (240 mg, 972.84 μmol, 1 eq), B₂Pin₂ (741.13 mg, 2.92 mmol, 3 eq), Pd(dppf)Cl₂ (71.18 mg, 97.28 μmol, 0.1 eq) and KOAc (286.43 mg, 2.92 mmol, 3 eq) in 1,4-dioxane (1 mL) was degassed and purged with N₂ 3 times, and then the mixture was stirred at 80° C. for 3 h under a N₂ atmosphere. The reaction mixture was filtered and then the filtrate was concentrated under reduced pressure to give a residue, which was purified by column chromatography (SiO₂, petroleum ether/EtOAc=100/0 to 70/30) to give the title compound. ¹H NMR (400 MHz, CDCl₃) δ ppm: 7.21-7.15 (m, 1H), 2.06 (d, J=1.6 Hz, 3H), 1.33 (s, 12H).

### Intermediate 57

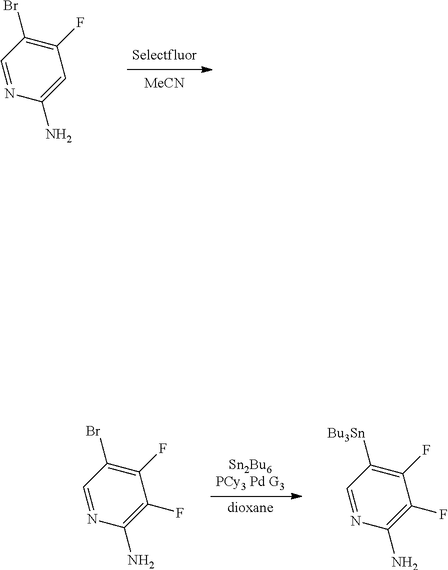

5-bromo-3,4-difluoropyridin-2-amine: To a solution of 5-bromo-4-fluoropyridin-2-amine (1 g, 5.24 mmol, 1 eq) in MeCN (40 mL) was added Selectfluor™ (11.13 g, 31.41 mmol, 6 eq). The mixture was stirred at 40° C. for 4 h under a N₂ atmosphere before it was diluted with H₂O (20 mL) and extracted with EtOAc (3×20 mL). The combined organic layers were washed with brine (60 mL), dried over Na₂SO₄, filtered, and concentrated under reduced pressure to give a residue, which was purified by flash column chromatography (SiO₂, petroleum ether/EtOAc=1/0 to 5/1) to give the title compound. [M+H]⁺=210.9. ¹H NMR (400 MHz, DMSO-d₆) δ ppm 7.94 (dd, J=8.4, 1.2 Hz, 1H), 6.79 (s, 2H).

3,4-difluoro-5-(tributylstannyl)pyridin-2-amine: To a solution of 5-bromo-3,4-difluoropyridin-2-amine (130 mg, 497.63 mol, 1 eq) in 1,4-dioxane (15 mL) was added PCy₃ Pd G₃ (36.58 mg, 49.76 mol, 0.1 eq) and Sn₂Bu₆ (3.79 g, 6.53 mmol, 3.27 mL, 13.13 eq). The mixture was stirred at 110° C. for 16 h under a N₂ atmosphere before it was cool to 25° C., diluted with water (15 mL), and extracted with EtOAc (3×10 mL). The combined organic layers were washed with brine (30 mL), dried over Na₂SO₄, filtered, and concentrated under reduced pressure to give a residue, which was purified by column chromatography (SiO₂, petroleum ether/EtOAc=1/0 to 3/1) to yield the title compound. [M+H]⁺=420.9. ¹H NMR (400 MHz, DMSO-d₆) δ ppm 7.56 (d, J=6.0 Hz, 1H), 6.46 (s, 2H), 1.52-1.45 (m, 6H), 1.33-1.24 (m, 6H), 1.12-1.05 (m, 6H), 0.89-0.79 (m, 9H).

### Intermediate 58

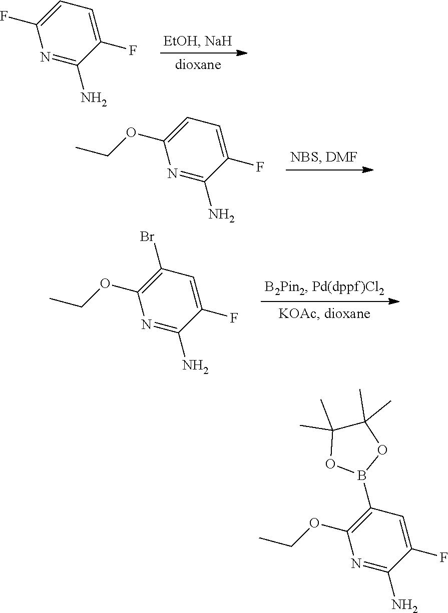 [SMILES: Nc1nc(F)ccc1F ; CCOc1ccc(F)c(N)n1 ; CCOc1nc(N)c(F)cc1Br ; CCOc1nc(N)c(F)cc1B1OC(C)(C)C(C)(C)O1]

6-ethoxy-3-fluoropyridin-2-amine: To a solution of 3,6-difluoropyridin-2-amine (200 mg, 1.38 mmol, 1 eq) and EtOH (127 mg, 2.77 mmol, 2 eq) in 1,4-dioxane (20 mL) was added NaH (221.35 mg, 5.53 mmol, 60% in mineral oil, 4 eq) under a N₂ atmosphere at 0° C. The mixture was stirred at 90° C. for 2 h before it was quenched with MeOH (4 mL) at 0° C., and then concentrated under reduced pressure. The residue was purified by column chromatography (SiO₂, petroleum ether/EtOAc=20/1 to 6/1) to give the title compound. ¹H NMR (400 MHz, CDCl₃) δ ppm 7.15 (t, J=9.2 Hz, 1H), 5.97 (dd, J=8.8, 2.0 Hz, 1H), 4.39 (s, 2H), 4.24-4.13 (m, 2H), 1.35 (t, J=7.2 Hz, 3H).

5-bromo-6-ethoxy-3-fluoropyridin-2-amine: To a solution of 6-ethoxy-3-fluoropyridin-2-amine (150 mg, 864.51 μmol, 1 eq) in DMF (4 mL) was added NBS (153.87 mg, 864.51 μmol, 1 eq). The mixture was stirred at 0° C. for 0.5 h before it was diluted with H₂O (20 mL) and extracted with EtOAc (2×20 mL). The combined organic layers were washed with brine (20 mL), dried over Na₂SO₄, filtered, and concentrated under reduced pressure to give a residue, which was purified by prep-TLC (SiO₂, petroleum ether/EtOAc=7/1) to give the title compound. ¹H NMR (400 MHz, CDCl₃) δ ppm 7.39 (d, J=8.8 Hz, 1H), 4.41 (s, 2H), 4.34-4.27 (m, 2H), 1.38 (t, J=7.2 Hz, 3H).

6-ethoxy-3-fluoro-5-(4,4,5,5-tetramethyl-1,3,2-dioxaborolan-2-yl)pyridin-2-amine: A mixture of 5-bromo-6-ethoxy-3-fluoropyridin-2-amine (100 mg, 382.89 μmol, 1 eq), B₂Pin₂ (291.69 mg, 1.15 mmol, 3 eq), Pd(dppf)Cl₂ (28.02 mg, 38.29 mol, 0.1 eq) and KOAc (112.73 mg, 1.15 mmol, 3 eq) in 1,4-dioxane (5 mL) was degassed and purged with N₂ 3 times, and then the mixture was stirred at 100° C. for 12 h under a N₂ atmosphere before it was diluted with H₂O (10 mL) and extracted with EtOAc (2×10 mL). The combined organic layers were washed with brine (20 mL), dried over Na₂SO₄, filtered, and concentrated under reduced pressure to give a residue, which was purified by column chromatography (SiO₂, petroleum ether/EtOAc=20/1 to 7/1) to give the title compound. ¹H NMR (400 MHz, CDCl₃) δ ppm 7.50 (d, J=10.4 Hz, 1H), 4.59 (s, 2H), 4.29-4.23 (m, 2H), 1.36-1.33 (m, 3H), 1.31 (s, 12H).

### Intermediate 59

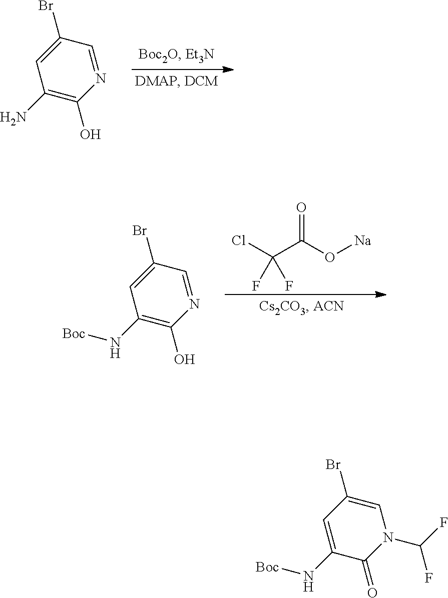

tert-butyl (5-bromo-2-hydroxypyridin-3-yl)carbamate: A mixture of 3-amino-5-bromopyridin-2-ol (200 mg, 1.06 mmol, 1 eq), Boc₂O (577 mg, 2.65 mmol, 608 μL, 2.5 eq), DMAP (12.9 mg, 106 mol, 0.1 eq) and Et₃N (107 mg, 1.06 mmol, 147 μL, 1 eq) in DCM (5 mL) was degassed and purged with N₂ three times, and then the mixture was stirred at 25° C. for 1 h under N₂. The reaction mixture was diluted with water (20 mL), extracted with EtOAc (20 mL×3). The combined organic layers were dried over Na₂SO₄, filtered, and concentrated under reduced pressure to give a residue, which was purified by column chromatography (SiO₂, petroleum ether/ethyl acetate=1/1) to give the title compound. ¹H NMR (400 MHz, DMSO-d₆) δ ppm: 12.21 (s, 1H), 7.85 (d, J=2.4 Hz, 2H), 7.30 (d, J=2.4 Hz, 1H), 1.46 (s, 9H).

tert-butyl (5-bromo-1-(difluoromethyl)-2-oxo-1,2-dihydropyridin-3-yl)carbamate: A mixture of tert-butyl (5-bromo-2-hydroxypyridin-3-yl)carbamate (115 mg, 358 mol, 1 eq), sodium 2-chloro-2,2-difluoroacetate (164 mg, 1.07 mmol, 3 eq), Cs₂CO₃ (233 mg, 716 μmol, 2 eq) in ACN (5 mL) was degassed and purged with N₂ three times, and then the mixture was stirred at 80° C. for 1 h under N₂. The reaction mixture was diluted with water (20 mL), extracted with EtOAc (20 mL×3). The combined organic layers were dried over Na₂SO₄, filtered, and concentrated under reduced pressure to give a residue, which was purified by column chromatography (SiO₂, petroleum ether/ethyl acetate=5/1) to give the title compound. ¹H NMR (400 MHz, DMSO-d₆) δ ppm: 8.34 (s, 1H), 8.03-7.73 (m, 3H), 1.47 (s, 9H).

### Intermediate 60

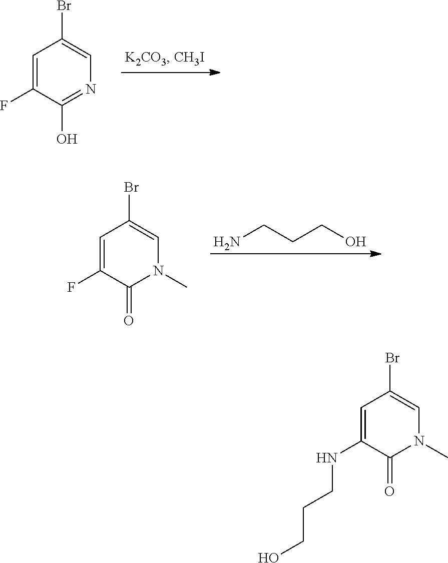

5-bromo-3-fluoro-1-methylpyridin-2(1H)-one: To a solution of 5-bromo-3-fluoropyridin-2-ol (500 mg, 2.60 mmol, 1 eq) in DMF (5 mL) was added K₂CO₃ (720 mg, 5.21 mmol, 2 eq) and CH₃I (407 mg, 2.86 mmol, 178 μL, 1.1 eq). The reaction mixture was concentrated under reduced pressure to remove solvent. The residue was diluted with H₂O (30 mL) and extracted with EtOAc 90 mL (30 mL×3). The combined organic layers were dried over Na₂SO₄, filtered, and concentrated under reduced pressure. The residue was purified by column chromatography (SiO₂, petroleum ether/ethyl acetate=1/0 to 1/1) to give the title compound. [M+H]⁺=207.7). ¹H NMR (400 MHz, DMSO-d₆) δ ppm: 7.96-7.91 (m, 1H), 7.74-7.65 (m, 1H), 3.47 (s, 3H).

5-bromo-3-((3-hydroxypropyl)amino)-1-methylpyridin-2(1H)-one: To a solution of 5-bromo-3-fluoro-1-methylpyridin-2(1H)-one (310 mg, 1.35 mmol, 1 eq) in 3-aminopropan-1-ol (3 mL) was stirred at 90° C. for 12 h. The reaction mixture was diluted with H₂O (50 mL) and extracted with EtOAc (50 mL×3). The combined organic layers were dried over Na₂SO₄, filtered, and concentrated under reduced pressure. The residue was purified by column chromatography (SiO₂, petroleum ether/ethyl acetate=1/0 to 0/1) to give the title compound. [M+H]⁺=260.7.

¹H NMR (400 MHz, DMSO-d₆) δ ppm: 7.13 (d, J=2.0 Hz, 1H), 6.20 (d, J=2.4 Hz, 1H), 5.85-5.78 (m, 1H), 4.55 (t, J=5.2 Hz, 1H), 3.51-3.42 (m, 2H), 3.41 (s, 3H), 3.12-3.05 (m, 2H), 1.73-1.62 (m, 2H).

### Intermediate 61

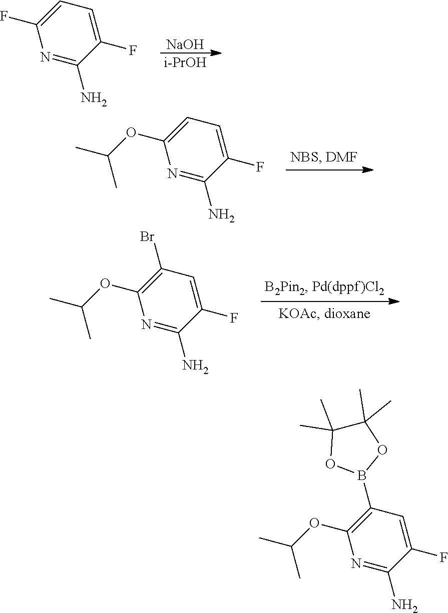

3-fluoro-6-isopropoxypyridin-2-amine: To a solution of 3,6-difluoropyridin-2-amine (200 mg, 1.38 mmol, 1 eq) in i-PrOH (20 mL) was added NaOH (138.35 mg, 3.46 mmol, 2.5 eq). The mixture was stirred at 150° C. for 6 h before it was diluted with H₂O (20 mL) and extracted with EtOAc (3×10 mL). The combined organic layers were washed with brine (10 mL), dried over Na₂SO₄, filtered, and concentrated under reduced pressure to give a residue, which was purified by column chromatography (SiO₂, petroleum ether/EtOAc=1/0 to 5/1) to give the title compound. ¹H NMR (400 MHz, CDCl₃) δ ppm 7.14 (t, J=8.4 Hz, 1H), 5.95 (dd, J=8.8, 2.4 Hz, 1H), 5.10-4.96 (m, 1H), 4.39 (s, 2H), 1.30 (d, J=6.4 Hz, 6H).

5-bromo-3-fluoro-6-isopropoxypyridin-2-amine: To a solution of 3-fluoro-6-isopropoxypyridin-2-amine (54 mg, 285.57 mol, 1 eq) in DMF (1.0 mL) was added NBS (60.99 mg, 342.69 mol, 1.2 eq) at 0° C. The mixture was stirred at 0° C. for 2 h before it was diluted with H₂O (10 mL) and extracted with EtOAc (3×10 mL). The combined organic layers were washed with brine (10 mL), dried over Na₂SO₄, filtered, and concentrated under reduced pressure to give a residue, which was purified by prep-TLC (SiO₂, petroleum ether/Ethyl acetate=10/1) to give the title compound. ¹H NMR (400 MHz, CDCl₃) δ ppm 7.38 (d, J=9.2 Hz, 1H), 5.21-5.08 (m, 1H), 4.39 (br s, 2H), 1.33 (d, J=6.4 Hz, 6H).

3-fluoro-6-isopropoxy-5-(4,4,5,5-tetramethyl-1,3,2-dioxaborolan-2-yl)pyridin-2-amine: To a solution of 5-bromo-3-fluoro-6-isopropoxypyridin-2-amine (34 mg, 122.85 μmol, 1 eq) and B₂Pin₂ (62.39 mg, 245.70 μmol, 2.0 eq) in 1,4-dioxane (1.0 mL) was added Pd(dppf)Cl₂ (8.99 mg, 12.29 mol, 0.1 eq) and KOAc (30.14 mg, 307.13 μmol, 2.5 eq). The mixture was stirred at 100° C. for 12 h under N₂ atmosphere before it was diluted with H₂O (10 mL) and extracted with EtOAc (3×10 mL). The combined organic layers were washed with brine (10 mL), dried over Na₂SO₄, filtered, and concentrated under reduced pressure to give the title compound. [M+H]⁺=297.0.

### Intermediate 62

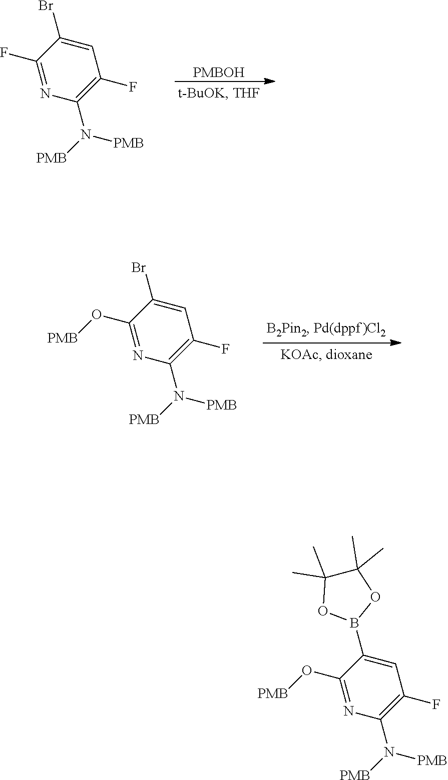

5-bromo-3-fluoro-N,N-bis(4-methoxybenzyl)-6-((4-methoxybenzyl)oxy)pyridin-2-amine: To a solution of (4-methoxyphenyl)methanol (83.03 mg, 600.95 μmol, 74.93 μL, 2 eq) in THE (0.5 mL) was added t-BuOK (1 M, 901.43 μL, 3 eq) dropwise at 25° C. After addition, the mixture was stirred at this temperature for 0.5 h, and then a solution of 5-bromo-3,6-difluoro-N,N-bis(4-methoxybenzyl)pyridin-2-amine (150 mg, 300.48 μmol, 1 eq) in THF (1.5 mL) was added dropwise at 25° C. The resulting mixture was stirred at 90° C. for 11.5 h before it was diluted with water (50 mL) and extracted with EtOAc (3×50 mL). The combined organic extracts were dried over Na₂SO₄, filtered, and concentrated under reduced pressure to give a residue, which was purified by column chromatography (SiO₂, petroleum ether/EtOAc=100/1 to 5/1) to give the title compound. ¹H NMR (400 MHz, DMSO-d₆) δ ppm 7.79 (d, J=12.4 Hz, 1H), 7.21-7.12 (m, 6H), 6.90-6.83 (m, 6H), 5.12 (s, 2H), 4.59 (s, 4H), 3.75-3.72 (m, 9H).

5-bromo-3-fluoro-N,N-bis(4-methoxybenzyl)-6-((4-methoxybenzyl)oxy)pyridin-2-amine: A mixture of 5-bromo-3-fluoro-N,N-bis(4-methoxybenzyl)-6-((4-methoxybenzyl)oxy)pyridin-2-amine (110 mg, 174.47 μmol, 1 eq), B₂Pin₂ (132.91 mg, 523.40 μmol, 3 eq), KOAc (51.37 mg, 523.40 μmol, 3 eq) and Pd(dppf)Cl₂ (12.77 mg, 17.45 μmol, 0.1 eq) in 1,4-dioxane (2 mL) was degassed and purged with N₂ 3 times, and then the mixture was stirred at 100° C. for 2 h under a N₂ atmosphere. The solids were removed by filtration and the filter cake was washed with 1,4-dioxane (3×5 mL). The filtrate was concentrated under reduced pressure to give a residue, which was purified by column chromatography (SiO₂, petroleum ether/EtOAc=100/1 to 5/1) to give the title compound. ¹H NMR (400 MHz, DMSO-d₆) δ ppm 7.41 (d, J=14.0 Hz, 1H), 7.29 (d, J=8.8 Hz, 2H), 7.15 (d, J=8.4 Hz, 4H), 6.88-6.82 (m, 6H), 5.11 (s, 2H), 4.65 (s, 4H), 3.74-3.70 (m, 9H), 1.26 (s, 12H).

### Intermediate 63

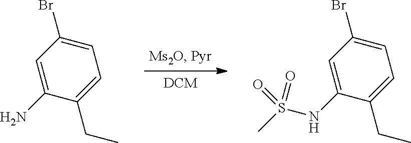

N-(5-bromo-2-ethylphenyl)methanesulfonamide: To a mixture of 5-bromo-2-ethylaniline (100 mg, 500 μmol, 1 eq) and Ms₂O (174 mg, 1 mmol, 2 eq) in DCM (3 mL) was added pyridine (119 mg, 1.50 mmol, 121 μL, 3 eq). The mixture was stirred at 20° C. for 5 h. The reaction mixture was concentrated under reduced pressure to remove DCM and give a residue, which was purified by column chromatography (SiO₂, petroleum ether/ethyl acetate=1/0 to 3/1) to give the title compound. ¹H NMR (400 MHz, DMSO-d₆) δ ppm: 9.24 (s, 1H), 7.45 (d, J=2.0 Hz, 1H), 7.41-7.37 (m, 1H), 7.23 (d, J=8.4 Hz, 1H), 3.03 (s, 3H), 2.70-2.61 (m, 2H), 1.13 (t, J=8.8 Hz, 3H).

### Intermediate 64

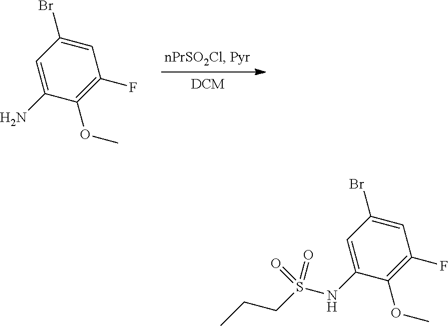 [SMILES: COc1c(N)cc(Br)cc1F ; CCCS(=O)(=O)Nc1cc(Br)cc(F)c1OC]

N-(5-bromo-3-fluoro-2-methoxyphenyl)propane-1-sulfonamide: To a solution of 5-bromo-3-fluoro-2-methoxyaniline (200 mg, 909 mol, 1 eq) in DCM (5 mL) was added propane-1-sulfonyl chloride (1.30 g, 9.09 mmol, 1.02 mL, 10 eq) and pyridine (719 mg, 9.09 mmol, 734 μL, 10 eq). The mixture was stirred at 25° C. for 3 h. The reaction mixture was filtered and concentrated under reduced pressure to give a residue, which was purified by flash silica gel chromatography (SiO₂, ethyl acetate/petroleum from 0 to 10%) to give the title compound. ¹H NMR (400 MHz, CDCl₃) δ ppm: 7.49 (t, J=2.0 Hz, 1H), 7.03 (dd, J=2.4, 10.8 Hz, 1H), 6.92 (s, 1H), 3.99 (d, J=2.4 Hz, 3H), 3.18-3.02 (m, 2H), 1.96-1.77 (m, 2H), 1.05 (t, J=7.2 Hz, 3H).

### Intermediate 65

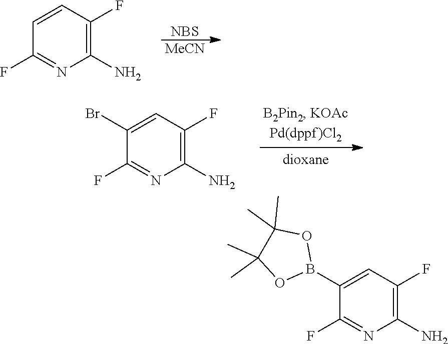

5-bromo-3,6-difluoropyridin-2-amine: To a mixture of 3,6-difluoropyridin-2-amine (130 mg, 999.27 μmol, 1 eq) in MeCN (15 mL) was added NBS (124.50 mg, 699.49 μmol, 0.7 eq). The mixture was stirred in absence of light at 25° C. for 30 min, and then a solution of additional NBS (124.50 mg, 699.49 mol, 0.7 eq) in MeCN (5 mL) was added. The mixture was stirred in absence of light at 25° C. for another 12 hours under a N₂ atmosphere before it was concentrated under reduced pressure. The residue was purified by column chromatography (SiO₂, 0-30% EtOAc/petroleum ether) to give the title compound. ¹H NMR (400 MHz, CDCl₃) δ ppm 7.58-7.34 (m, 1H), 4.98-4.37 (m, 2H).

3,6-difluoro-5-(4,4,5,5-tetramethyl-1,3,2-dioxaborolan-2-yl)pyridin-2-amine: To a mixture of 5-bromo-3,6-difluoropyridin-2-amine (140 mg, 602.90 μmol, 1 eq), B₂Pin₂ (459.29 mg, 1.81 mmol, 3 eq) and KOAc (177.50 mg, 1.81 mmol, 3 eq) in dioxane (10 mL) was added Pd(dppf)Cl₂ (44.11 mg, 60.29 μmol, 0.1 eq). The mixture was degassed and purged with N₂ 3 times, and then it was stirred at 100° C. for 12 hours under a N₂ atmosphere. The reaction mixture concentrated under reduced pressure to give a residue, which was purified by column chromatography (SiO₂, 0-30% EtOAc/petroleum ether) to give the title compound. ¹H NMR (400 MHz, CDCl₃) δ ppm 7.63-7.55 (m, 1H), 4.89 (s, 2H), 1.33 (s, 12H).

### Intermediate 66

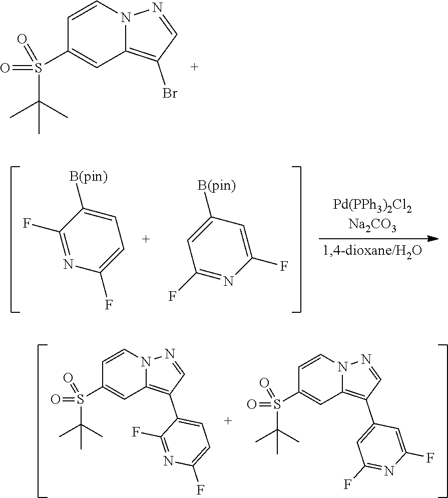

5-(tert-butylsulfonyl)-3-(2,6-difluoropyridin-3-yl)pyrazolo[1,5-a]pyridine and 5-(tert-butylsulfonyl)-3-(2,6-difluoropyridin-4-yl)pyrazolo[1,5-a]pyridine: A stirred solution of 3-bromo-5-(tert-butylsulfonyl)pyrazolo[1,5-a]pyridine (0.200 g, 0.631 mmol, 1 eq, prepared as described in U.S. Published Application No. US 2018/0072717, which is incorporated by reference herein in its entirety) and a 1.6:1.0 mixture of 2,6-difluoro-4-(4,4,5,5-tetramethyl-1,3,2-dioxaborolan-2-yl)pyridine and 2,6-difluoro-3-(4,4,5,5-tetramethyl-1,3,2-dioxaborolan-2-yl)pyridine (0.226 g, 0.946 mmol, 1.2 eq) in 1,4-dioxane (1.6 mL) was sparged with N₂. Bis(triphenylphosphine)palladium(II) dichloride (0.046 g, 0.063 mmol, 0.1 eq), sodium carbonate (0.133 g, 1.26 mmol, 2.0 eq) and water (0.4 mL) were added into the reaction mixture. The reaction mixture was stirred at 100° C. for 1 h, after which it was diluted with water (10 mL) and extracted with EtOAc (2×15 mL). The combined organic layer was dried over anhydrous Mg₂SO₄ and concentrated under reduced pressure. The crude product was purified through flash column chromatography over silica gel (100-200 mesh) using 5-15% ethyl acetate/petroleum ether as eluent to obtain a mixture of regioisomers 5-(tert-butylsulfonyl)-3-(2,6-difluoropyridin-4-yl)pyrazolo[1,5-a]pyridine and 5-(tert-butylsulfonyl)-3-(2,6-difluoropyridin-3-yl)pyrazolo[1,5-a]pyridine in a 3:1 ratio.

### Intermediate 67

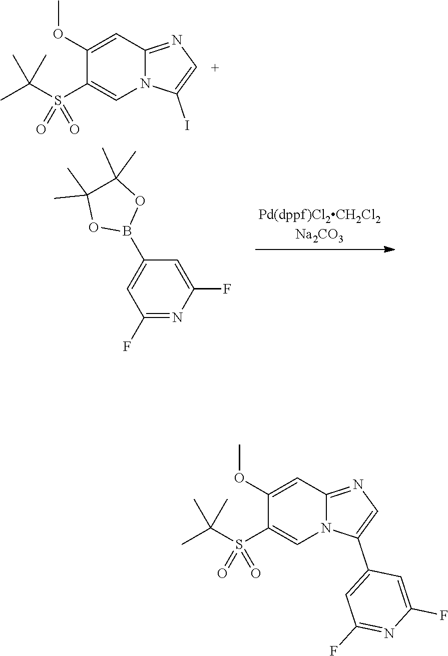

6-(tert-butylsulfonyl)-3-(2,6-difluoropyridin-4-yl)-7-methoxyimidazo[1,2-a]pyridine: In a first batch 2,6-difluoro-4-(4,4,5,5-tetramethyl-1,3,2-dioxaborolan-2-yl)pyridine (612 mg, 2.54 mmol), 6-(tert-butylsulfonyl)-3-iodo-7-methoxyimidazo[1,2-a]pyridine (1.00 g, 1.95 mmol, prepared as described in ACS Med. Chem. Lett. 2017, 8, 1048, which is incorporated by reference herein in its entirety) and an aqueous solution of sodium carbonate (2.9 mL, 2 M, 5.86 mmol) were combined in a mixture of ethanol (9 mL) and 1,4-dioxane (9 mL), and argon was bubbled through the mixture for 2 min. [1,1′-Bis(diphenylphosphino)ferrocene]dichloropalladium(II) dichloromethane complex (160 mg, 195 μmol) was added. The mixture was stirred in a microwave oven for 60 min at 100° C. A second batch was performed in the same way but with [1,1′-bis(diphenylphosphino)ferrocene]dichloropalladium(II) dichloromethane complex (120 mg, 146 μmol). The two mixtures were combined and filtered through a celite pad. Water and a saturated aqueous solution of sodium chloride were added. The phases were separated, and the aqueous phase was extracted with ethyl acetate. The combined organic phases were filtered, concentrated, purified by flash chromatography (silica gel, hexane/ethyl acetate/ethanol gradient) to give the title compound. [M+H]⁺=382.6. ¹H NMR (400 MHz, DMSO-d₆) δ ppm 8.89 (s, 1H), 8.18 (s, 1H), 7.48 (s, 2H), 7.37 (s, 1H), 3.96 (s, 3H), 1.33 (s, 9H).

### Exemplary Compounds of Formula (I)

### Example 1

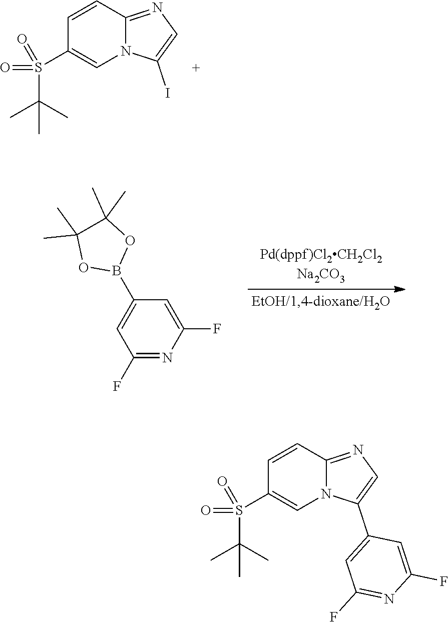 [SMILES: CC(C)(C)S(=O)(=O)c1ccc2ncc(I)n2c1 ; CC1(C)OB(c2cc(F)nc(F)c2)OC1(C)C ; CC(C)(C)S(=O)(=O)c1ccc2ncc(-c3cc(F)nc(F)c3)n2c1]

6-(tert-butylsulfonyl)-3-(2,6-difluoropyridin-4-yl)imidazo[1,2-a]pyridine: 2,6-difluoro-4-(4,4,5,5-tetramethyl-1,3,2-dioxaborolan-2-yl)pyridine (430 mg, 1.78 mmol), 6-(tert-butylsulfonyl)-3-iodoimidazo[1,2-a]pyridine (500 mg, 1.37 mmol) and sodium carbonate (437 mg, 4.12 mmol) were combined in a mixture of ethanol (4 mL), 1,4-dioxane (4 mL) and water (2 mL), and argon was bubbled through the mixture for 3 min. [1,1′-bis(diphenylphosphino)ferrocene]dichloropalladium(II) dichloromethane complex (112 mg, 137 μmol) was added and argon was bubbled through the mixture for another 2 min. The mixture was stirred in a microwave oven for 90 min at 100° C. The mixture was filtered and concentrated, and the crude product was purified by flash chromatography (silica gel, dichloromethane/ethanol gradient) to give the title compound. [M+H]=352.4. ¹H NMR (400 MHz, DMSO-d₆) δ ppm 8.95 (dd, J=1.6, 0.9 Hz, 1H), 8.31 (s, 1H), 7.93 (dd, J=9.4, 0.8 Hz, 1H), 7.66 (dd, J=9.4, 1.8 Hz, 1H), 7.56 (s, 2H), 1.33 (s, 9H).

### Example 2

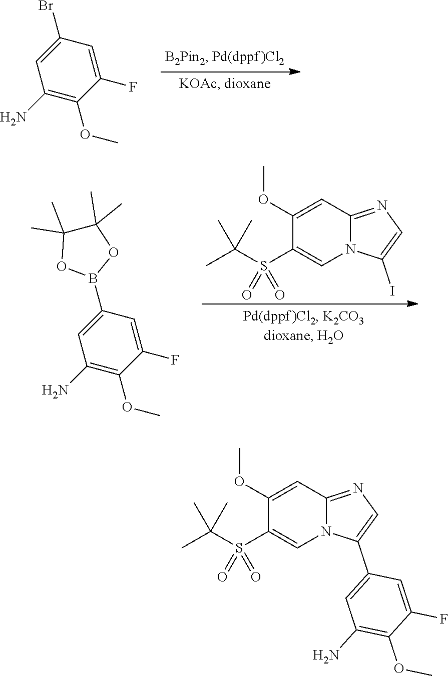 [SMILES: COc1c(N)cc(Br)cc1F ; COc1c(N)cc(B2OC(C)(C)C(C)(C)O2)cc1F ; COc1cc2ncc(I)n2cc1S(=O)(=O)C(C)(C)C ; COc1cc2ncc(-c3cc(N)c(OC)c(F)c3)n2cc1S(=O)(=O)C(C)(C)C]

3-fluoro-2-methoxy-5-(4,4,5,5-tetramethyl-1,3,2-dioxaborolan-2-yl)aniline: A mixture of 5-bromo-3-fluoro-2-methoxyaniline (240 mg, 1.09 mmol, 1 eq), B₂Pin₂ (831 mg, 3.27 mmol, 3 eq), KOAc (321 mg, 3.27 mmol, 3 eq), and Pd(dppf)Cl₂ (80 mg, 109 μmol, 0.1 eq) in 1,4-dioxane (3 mL) was degassed and purged with N₂ three times, and then the mixture was stirred at 100° C. for 12 h under N₂. The reaction mixture was diluted with H₂O (30 mL) and extracted with EtOAc (30 mL×3). The combined organic layers were washed with brine (30 mL), dried over Na₂SO₄, filtered, and concentrated under reduced pressure to give a residue, which was purified by flash silica gel chromatography (from PE/EtOAc=100/1 to 3/1) to afford the title compound. [M+H]⁺=268.3. ¹H NMR (400 MHz, DMSO-d₆) δ ppm 6.87 (s, 1H), 6.58-6.51 (m, 1H), 5.19 (s, 2H), 3.75 (s, 3H), 1.26 (s, 12H).

5-(6-(tert-butylsulfonyl)-7-methoxyimidazo[1,2-a]pyridin-3-yl)-3-fluoro-2-methoxyaniline: A mixture of 3-fluoro-2-methoxy-5-(4,4,5,5-tetramethyl-1,3,2-dioxaborolan-2-yl)aniline (150 mg, 505 μmol, 1 eq), 6-(tert-butylsulfonyl)-3-iodo-7-methoxyimidazo[1,2-a]pyridine (332 mg, 758 μmol, 1.5 eq), K₂CO₃ (210 mg, 1.52 mmol, 3 eq), Pd(dppf)Cl₂ (37 mg, 51 gmol, 0.1 eq) in 1,4-dioxane (2 mL) and H₂O (0.5 mL) was degassed and purged with N₂ three times, and then the mixture was stirred at 90° C. for 12 h under N₂. The reaction mixture was diluted with H₂O (20 mL) and extracted with EtOAc (20 mL×3). The combined organic layers were washed with brine (20 mL), dried over Na₂SO₄, filtered and concentrated under reduced pressure to give a residue, which was purified by prep-HPLC to give the title compound. [M+H]⁺=408.0. ¹H NMR (400 MHz, DMSO-d₆) δ ppm 8.69 (s, 1H), 764 (s, 1H), 7.24 (s, 1H), 6.67-6.60 (m, 2H), 5.54 (s, 2H), 3.92 (s, 3H), 3.81 (s, 3H), 1.31 (s, 9H).

The following compounds were prepared following procedures analogous to that described for Example 1 and Example 2.

| Ex. | | | |
|---|---|---|---|
| No. | Structure | Name | LCMS, ¹H NMR |
| 3 | 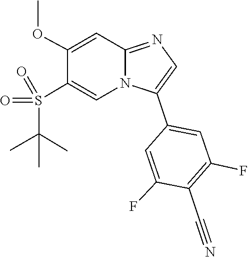 [SMILES: COc1cc2ncc(-c3cc(F)c(C#N)c(F)c3)n2cc1S(=O)(=O)C(C)(C)C] | 4-(6-(tert- butylsulfonyl)-7- methoxyimidazo[1,2- a]pyridin-3-yl)-2,6- difluorobenzonitrile | [M + H]⁺ = 405.9 ¹H NMR (400 MHz, DMSO-d₆) δ ppm: 8.82 (s, 1H), 8.08 (s, 1H), 7.80-7.74 (m, 2H), 7.35 (s, 1H), 3.96 (s, 3H), 1.32 (s, 9H) |
| 4 | 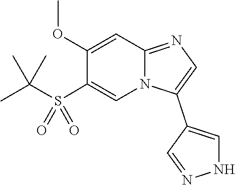 [SMILES: COc1cc2ncc(-c3cn[nH]c3)n2cc1S(=O)(=O)C(C)(C)C] | 6-(tert-butylsulfonyl)- 7-methoxy-3-(1H- pyrazol-4- yl)imidazo[1,2- a]pyridine | [M + H]⁺ = 335.1 ¹H NMR (400 MHz, DMSO-d₆) δ ppm: 13.33 (br s, 1H), 8.53 (s, 1H), 8.18 (br s, 1H), 7.83 (br s, 1H), 7.64 (s, 1H), 7.23 (s, 1H), 3.92 (s, 3H), 1.31 (s, 9H) |
| 5 | 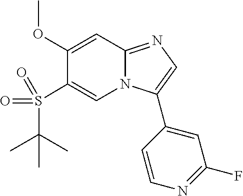 [SMILES: COc1cc2ncc(-c3ccnc(F)c3)n2cc1S(=O)(=O)C(C)(C)C] | 6-(tert-butylsulfonyl)- 3-(2-fluoropyridin-4- yl)-7- methoxyimidazo[1,2- a]pyridine | [M + H]⁺ = 364.1 ¹H NMR (400 MHz, DMSO-d₆) δ ppm 8.87 (s, 1H), 8.40 (d, J = 5.1 Hz, 1H), 8.12 (s, 1H), 7.62 (d, J = 5.3 Hz, 1H), 7.47 (s, 1H), 7.35 (s, 1H), 3.96 (s, 3H), 1.33 (s, 9H) |
| 6 | 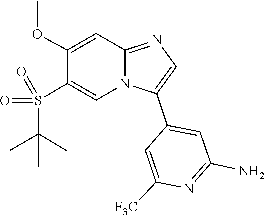 [SMILES: COc1cc2ncc(-c3cc(N)nc(C(F)(F)F)c3)n2cc1S(=O)(=O)C(C)(C)C] | 4-(6-(tert- butylsulfonyl)-7- methoxyimidazo[1,2- a]pyridin-3-yl)-6- (trifluoromethyl) pyridin-2-amine | [M + H]⁺ = 429.1 ¹H NMR (400 MHz, DMSO-d₆) δ ppm 8.79 (s, 1H), 7.98 (s, 1H), 7.31 (s, 1H), 7.14 (d, J = 1.0 Hz, 1H), 6.85 (s, 2H), 6.82 (s, 1H), 3.95 (s, 3H), 1.33 (s, 9H) |
| 7 | 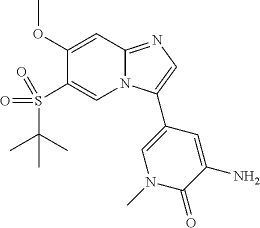 [SMILES: COc1cc2ncc(-c3cc(N)c(=O)n(C)c3)n2cc1S(=O)(=O)C(C)(C)C] | 3-amino-5-(6-(tert- butylsulfonyl)-7- methoxyimidazo[1,2- a]pyridin-3-yl)-1- methylpyridin-2(1H)- one | [M + H]⁺ = 391.5 ¹H NMR (400 MHz, DMSO-d₆) δ ppm 8.53 (s, 1H), 7.55 (s, 1H), 7.20−7.25 (m, 2H), 6.54 (d, J = 2.3 Hz, 1H), 5.47 (s, 2H), 3.92 (s, 3H), 3.51 (s, 3H), 1.30 (s, 9H) |
| 8 | 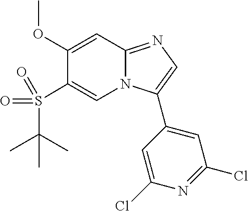 [SMILES: COc1cc2ncc(-c3cc(Cl)nc(Cl)c3)n2cc1S(=O)(=O)C(C)(C)C] | 6-(tert-butylsulfonyl)- 3-(2,6-dichloropyridin- 4-yl)-7- methoxyimidazo[1,2- a]pyridine | [M + H]⁺ = 414.3 ¹H NMR (400 MHz, DMSO-d₆) δ ppm 8.87 (s, 1H), 8.18 (s, 1H), 7.85 (s, 2H), 7.35 (s, 1H), 3.96 (s, 3H), 1.33 (s, 9H) |
| 9 | 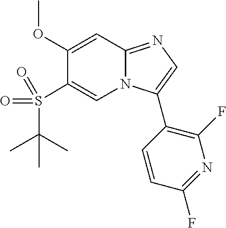 [SMILES: COc1cc2ncc(-c3ccc(F)nc3F)n2cc1S(=O)(=O)C(C)(C)C] | 6-(tert-butylsulfonyl)- 3-(2,6-difluoropyridin- 3-yl)-7- methoxyimidazo[1,2- a]pyridine | [M + H]⁺ = 382.4 ¹H NMR (400 MHz, DMSO-d₆) δ ppm 8.55 (d, J = 2.5 Hz, 1H), 8.38−8.48 (m, 1H), 7.85 (s, 1H), 7.43 (dd, J = 8.1, 2.3 Hz, 1H), 7.33 (s, 1H), 3.95 (s, 3H), 1.30 (s, 9H) |
| 10 | 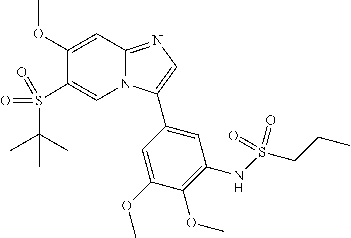 [SMILES: CCCS(=O)(=O)Nc1cc(-c2cnc3cc(OC)c(S(=O)(=O)C(C)(C)C)cn23)cc(OC)c1OC] | N-(5-(6-(tert- butylsulfonyl)-7- methoxyimidazo[1,2- a]pyridin-3-yl)-2,3- dimethoxyphenyl) propane-1-sulfonamide | [M + H]⁺ =526.5 ¹H NMR (400 MHz, DMSO-d₆) δ ppm 9.30 (s, 1H), 8.75 (s, 1H), 7.80 (s, 1H), 7.29 (s, 1H), 7.21 (d, J = 1.8 Hz, 1H), 7.12 (d, J = 2.0 Hz, 1H), 3.94 (s, 3H), 3.86 (s, 3H), 3.82 (s, 3H), 3.10−3.18 (m, 2H), 1.69−1.81 (m, 2H), 1.33 (s, 9H), 0.96 (t, J = 7.5 Hz, 3H) |
| Int. 68 | 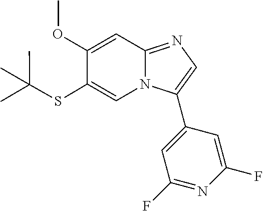 [SMILES: COc1cc2ncc(-c3cc(F)nc(F)c3)n2cc1SC(C)(C)C] | 6-(tert-butylthio)-3- (2,6-difluoropyridin-4- yl)-7- methoxyimidazo[1,2- a]pyridine | [M + H]⁺ = 350.5 ¹H NMR (400 MHz, DMSO-d₆) δ ppm 8.69 (s, 1H), 8.10 (s, 1H), 7.47 (s, 2H), 7.20 (s, 1H), 3.92 (s, 3H), 1.27 (s, 9H) |
| Int. 69 | 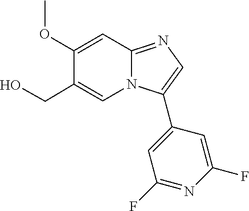 [SMILES: COc1cc2ncc(-c3cc(F)nc(F)c3)n2cc1CO] | (3-(2,6-difluoropyridin- 4-yl)-7- methoxyimidazo[1,2- a]pyridin-6- yl)methanol | [M + H]⁺ =292.4 ¹H NMR (400 MHz, DMSO-d₆) δ ppm 8.56 (s, 1 H), 8.10 (s, 1 H), 7.49 (s, 2 H), 7.14 (s, 1 H), 5.27−5.31 (m, 1 H), 4.56 (dd, J = 5.7, 1.1 Hz, 2 H), 3.91 (s, 3H). |
| 11 | 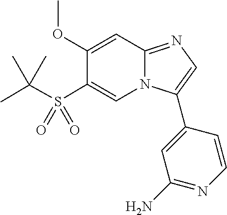 [SMILES: COc1cc2ncc(-c3ccnc(N)c3)n2cc1S(=O)(=O)C(C)(C)C] | 4-[7-methoxy-6-(2- methylpropane-2- sulfonyl)imidazo[1,2- a]pyridin-3-yl]pyridin- 2-amine | [M + H]⁺ = 361.4 ¹H NMR (400 MHz, DMSO-d₆) δ ppm 8.80 (s, 1 H), 8.00−8.10 (m, 1 H), 7.86 (s, 1 H), 7.30 (s, 1 H), 6.68−6.75 (m, 1 H), 6.28 (br s, 2 H), 3.94 (s, 3 H), 1.32 (s, 9 H) |
| 12 | 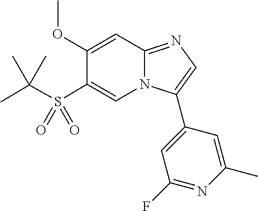 [SMILES: COc1cc2ncc(-c3cc(C)nc(F)c3)n2cc1S(=O)(=O)C(C)(C)C] | 2-fluoro-4-[7-methoxy- 6-(2-methylpropane-2- sulfonyl)imidazo[1,2- a]pyridin-3-yl]-6- methylpyridine | [M + H]⁺ = 378.4 ¹H NMR (400 MHz, DMSO-d₆) δ ppm 8.85 (s, 1 H), 8.07 (s, 1 H), 7.48 (s, 1 H), 7.34 (s, 1 H), 7.25 (s, 1 H), 3.95 (s, 3 H), 1.32 (s, 9 H) |
| 13 | 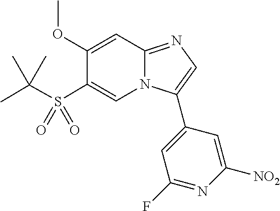 [SMILES: COc1cc2ncc(-c3cc(F)nc([N+](=O)[O-])c3)n2cc1S(=O)(=O)C(C)(C)C] | 3-(3-fluoro-5- nitrophenyl)-7- methoxy-6-(2- methylpropane-2- sulfonyl)imidazo[1,2- a]pyridine | [M + H]⁺ = 408.5 ¹H NMR (400 MHz, DMSO-d₆) δ ppm 8.78 (s, 1 H), 8.32−8.35 (m, 1 H), 8.17 (dt, J = 8.62, 2.15 Hz, 1 H), 8.09 (dt, J = 9.38, 1.90 Hz, 1 H), 8.01 (s, 1 H), 7.33 (s, 1 H), 3.95 (s, 3 H), 1.33 (s, 9 H) |
| 14 | 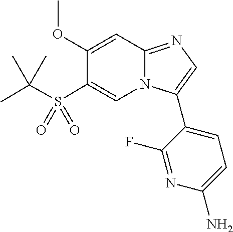 [SMILES: COc1cc2ncc(-c3ccc(N)nc3F)n2cc1S(=O)(=O)C(C)(C)C] | 5-(6-(tert- butylsulfonyl)-7- methoxyimidazo[1,2- a]pyridin-3-yl)-6- fluoropyridin-2-amine | [M + H]⁺ = 379.0 ¹H NMR (400 MHz, DMSO-d₆) δ ppm 8.30 (d, J = 2.4 Hz, 1H), 7.67 (dd, J = 8.4, 10.0 Hz, 1H), 7.63 (s, 1H), 7.25 (s, 1H), 6.79 (s, 2H), 6.49 (dd, J = 2.0, 8.0 Hz, 1H), 3.93 (s, 3H), 1.29 (s, 9H) |
| 15 | 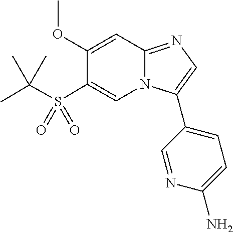 [SMILES: COc1cc2ncc(-c3ccc(N)nc3)n2cc1S(=O)(=O)C(C)(C)C] | 5-(6-(tert- butylsulfonyl)-7- methoxyimidazo[1,2- a]pyridin-3-yl)pyridin- 2-amine | [M + H]⁺ = 360.9 ¹H NMR (400 MHz, DMSO-d₆) δ ppm 8.50 (s, 1H), 8.10 (d, J = 2.0 Hz, 1H), 7.64-7.56 (m, 2H), 7.22 (s, 1H), 6.61 (d, J = 8.4 Hz, 1H), 6.32 (s, 2H), 3.91 (s, 3H), 1.29 (s, 9H) |
| 16 | 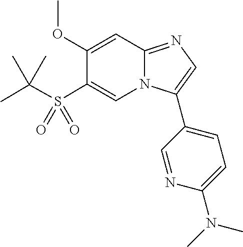 [SMILES: COc1cc2ncc(-c3ccc(N(C)C)nc3)n2cc1S(=O)(=O)C(C)(C)C] | 5-(6-(tert- butylsulfonyl)-7- methoxyimidazo[1,2- a]pyridin-3-yl)-N,N- dimethylpyridin-2- amine | [M + H]⁺ = 389.0 ¹H NMR (400 MHz, DMSO-d₆) δ ppm 8.52 (s, 1H), 8.28 (d, J = 2.4 Hz, 1H), 7.76-7.70 (m, 1H), 7.63 (s, 1H), 7.24 (s, 1H), 6.86- 6.81 (m, 1H), 3.92 (s, 3H), 3.10 (s, 6H), 1.30 (s, 9H) |
| 17 | 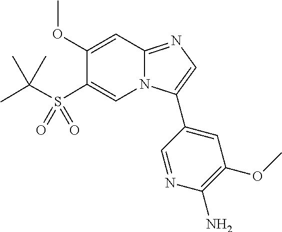 [SMILES: COc1cc2ncc(-c3cnc(N)c(OC)c3)n2cc1S(=O)(=O)C(C)(C)C] | 5-(6-(tert- butylsulfonyl)-7- methoxyimidazo[1,2- a]pyridin-3-yl)-3- methoxypyridin-2- amine | [M + H]⁺ = 390.9 ¹H NMR (400 MHz, DMSO-d₆) δ ppm 8.59 (s, 1H), 7.72 (d, J = 1.6 Hz, 1H), 7.64 (s, 1H), 7.26 (d, J = 1.6 Hz, 1H), 7.22 (s, 1H), 6.11 (s, 2H), 3.92 (s, 3H), 3.81 (s, 3H), 1.31 (s, 9H) |
| 18 | 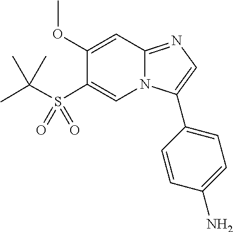 [SMILES: COc1cc2ncc(-c3ccc(N)cc3)n2cc1S(=O)(=O)C(C)(C)C] | 4-(6-(tert- butylsulfonyl)-7- methoxyimidazo[1,2- a]pyridin-3-yl)aniline | [M + H]⁺ = 360.0 ¹H NMR (400 MHz, DMSO-d₆) δ ppm 8.59 (s, 1H), 7.53 (s, 1H), 7.26-7.17 (m, 3H), 6.72 (d, J = 8.0 Hz, 2H), 5.46 (s, 2H), 3.91 (s, 3H), 1.30 (s, 9H). |
| 19 | 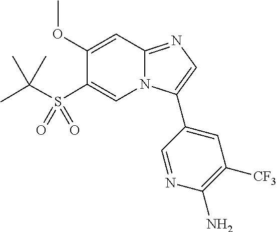 [SMILES: COc1cc2ncc(-c3cnc(N)c(C(F)(F)F)c3)n2cc1S(=O)(=O)C(C)(C)C] | 5-(6-(tert- butylsulfonyl)-7- methoxyimidazo[1,2- a]pyridin-3-yl)-3- (trifluoromethyl)pyridin- 2-amine | [M + H]⁺ = 428.8 ¹H NMR (400 MHz, DMSO-d₆) δ ppm 8.49 (s, 1H), 8.42 (d, J = 1.6 1H), 7.99 (d, J = 2.0 Hz, 1H), 7.71 (s, 1H), 7.23 (s, 1H), 6.84 (s, 2H), 3.92 (s, 3H), 1.30 (s, 9H) |
| 20 | 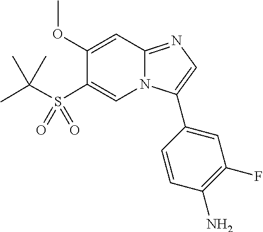 [SMILES: COc1cc2ncc(-c3ccc(N)c(F)c3)n2cc1S(=O)(=O)C(C)(C)C] | 4-(6-(tert- butylsulfonyl)-7- methoxyimidazo[1,2- a]pyridin-3-yl)-2- fluoroaniline | [M + H]⁺ = 377.9 ¹H NMR (400 MHz, DMSO-d₆) δ ppm 8.60 (s, 1H), 7.60 (s, 1H), 7.29-7.19 (m, 1H), 7.22 (s, 1H), 7.10 (dd, J = 2.0, 8.4 Hz, 1H), 6.96-6.90 (m, 1H), 5.52 (s, 2H), 3.92 (s, 3H), 1.30 (s, 9H) |
| 21 | 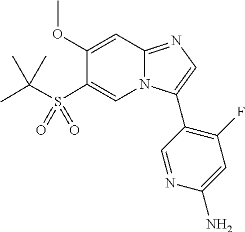 [SMILES: COc1cc2ncc(-c3cnc(N)cc3F)n2cc1S(=O)(=O)C(C)(C)C] | 5-(6-(tert- butylsulfonyl)-7- methoxyimidazo[1,2- a]pyridin-3-yl)-4- fluoropyridin-2-amine | [M + H]⁺ = 378.8 ¹H NMR (400 MHz, DMSO-d₆) δ ppm 8.29 (d, J = 2.4 Hz, 1H), 8.10 (d, J = 11.6 Hz, 1H), 7.66 (s, 1H), 7.26 (s, 1H), 6.69 (s, 2H), 6.41 (d, J = 13.2 Hz, 1H), 3.93 (s, 3H), 1.29 (s, 9H) |
| 22 | 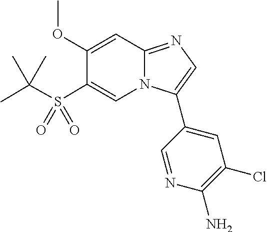 [SMILES: COc1cc2ncc(-c3cnc(N)c(Cl)c3)n2cc1S(=O)(=O)C(C)(C)C] | 5-(6-(tert- butylsulfonyl)-7- methoxyimidazo[1,2- a]pyridin-3-yl)-3- chloropyridin-2-amine | [M + H]⁺ = 395.0 ¹H NMR (400 MHz, DMSO-d₆) δ ppm 8.51 (s, 1H), 8.13 (d, J = 1.6 Hz, 1H), 7.87 (d, J = 1.6 Hz, 1H), 7.67 (s, 1H), 7.23 (s, 1H), 6.69 (s, 2H), 3.92 (s, 3H), 1.30 (s, 9H) |
| 23 | 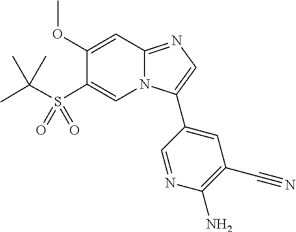 [SMILES: COc1cc2ncc(-c3cnc(N)c(C#N)c3)n2cc1S(=O)(=O)C(C)(C)C] | 2-amino-5-(6-(tert- butylsulfonyl)-7- methoxyimidazo[1,2- a]pyridin-3- yl)nicotinonitrile | [M + H]⁺ = 386.0 ¹H NMR (400 MHz, DMSO-d₆) δ ppm 8.49 (s, 1H), 8.42 (d, J = 2.0 Hz, 1H), 8.18 (d, J = 2.4 Hz, 1H), 7.68 (s, 1H), 7.28 (s, 2H), 7.24 (s, 1H), 3.92 (s, 3H), 1.30 (s, 9H) |
| 24 | 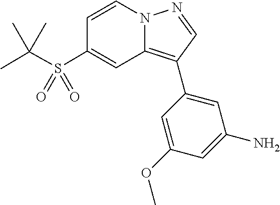 [SMILES: COc1cc(N)cc(-c2cnn3ccc(S(=O)(=O)C(C)(C)C)cc23)c1] | 3-(5-(tert- butylsulfonyl)pyrazolo [1,5-a]pyridin-3-yl)-5- methoxyaniline | [M + H]⁺ = 360.34 ¹H NMR (400 MHz, DMSO-d₆) δ ppm 8.93 (dd, J = 7.4, 0.6 Hz, 1H), 8.47 (s, 1H), 8.18 (d, J = 1.2 Hz, 1H), 7.17 (dd, J = 7.6, 2.0 Hz, 1H), 6.44 (t, J = 1.6 Hz, 1H), 6.35 (t, J = 1.8 Hz, 1H), 6.16 (t, J = 2.0 Hz, 1H). |
| 25 | 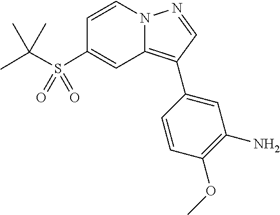 [SMILES: COc1ccc(-c2cnn3ccc(S(=O)(=O)C(C)(C)C)cc23)cc1N] | 5-(5-(tert- butylsulfonyl)pyrazolo [1,5-a]pyridin-3-yl)-2- methoxyaniline | [M + H]⁺ = 360.28 ¹H NMR (400 MHz, DMSO-d₆) δ ppm 8.91 (d, J = 7.2 Hz, 1H), 8.40 (s, 1H), 8.16 (d, J = 1.2 Hz, 1H), 7.14 (dd, J = 7.2, 2.0 Hz, 1H), 6.93 (q, J = 5.2 Hz, 2H), 6.80 (dd, J = 8.2, 2.2 Hz, 1H), 4.94 (s, 2H), 3.81 (s, 3H), 1.32 (s, 9H) |
| 26 | 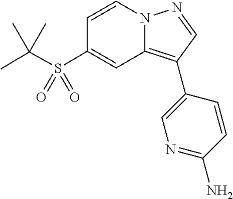 [SMILES: CC(C)(C)S(=O)(=O)c1ccn2ncc(-c3ccc(N)nc3)c2c1] | 5-(5-(tert- butylsulfonyl)pyrazolo [1,5-a]pyridin-3- yl)pyridin-2-amine | [M + H]⁺ = 331.3. ¹H NMR (400 MHz, DMSO-d₆) δ ppm 8.91 (dd, J = 7.2, 0.8 Hz, 1H), 8.46 (s, 1H), 8.22 (dd, J = 2.4, 0.4 Hz, 1H), 8.11-8.10 (m, 1H), 7.68 (dd, J = 8.6, 2.6 Hz, 1H), 7.14 (dd, J = 7.2, 2.0 Hz, 1H), 6.60 (dd, J = 8.6, 0.6 Hz, 1H), 6.12 (s, 2H), 1.32 (s, 9H). |
| Int. 70 | 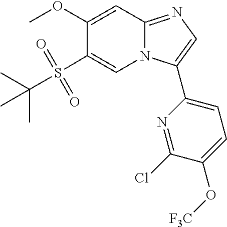 [SMILES: COc1cc2ncc(-c3ccc(OC(F)(F)F)c(Cl)n3)n2cc1S(=O)(=O)C(C)(C)C] | 6-(tert-butylsulfonyl)- 3-(6-chloro-5- (trifluoromethoxy) pyridin-2-yl)-7- methoxyimidazo[1,2- a]pyridine | [M + H]⁺ = 463.7 ¹H NMR (400 MHz, DMSO-d₆) δ ppm 10.23 (s, 1H), 8.59 (s, 1H), 8.24-8.12 (m, 2H), 7.38 (s, 1H), 3.97 (s, 3H), 1.34 (s, 9H). |
| 27 | 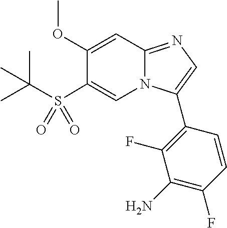 [SMILES: COc1cc2ncc(-c3ccc(F)c(N)c3F)n2cc1S(=O)(=O)C(C)(C)C] | 3-(6-(tert- butylsulfonyl)-7- methoxyimidazo[1,2- a]pyridin-3-yl)-2,6- difluoroaniline | [M + H]⁺ = 396.0 ¹H NMR (400 MHz, DMSO-d₆) δ ppm 8.37 (d, J = 2.8 Hz, 1H), 7.71 (s, 1H), 7.30 (s, 1H), 7.17- 7.08 (m, 1H), 6.79-6.69 (m, 1H), 5.54 (s, 2H), 3.94 (s, 3H), 1.30 (s, 9H). |
| Int. 71 | 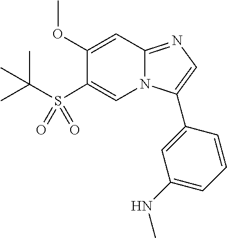 | 3-(6-(tert- butylsulfonyl)-7- methoxyimidazo[1,2- a]pyridin-3-yl)-N- methylaniline | [M + H]⁺ = 466.1 ¹H NMR (400 MHz, DMSO-d₆) δ ppm 9.59 (s, 1H), 9.12 (s, 1H), 8.79 (s, 1H), 7.68 (s, 1H), 7.26-7.24 (m, 2H), 6.74-6.63 (m, 3H), 3.92 (s, 3H), 2.70 (d, J = 4.8 Hz, 3H), 1.31-1.30 (m, 9H). |
| 28 | 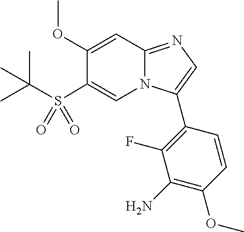 [SMILES: COc1cc2ncc(-c3ccc(OC)c(N)c3F)n2cc1S(=O)(=O)C(C)(C)C] | 3-(6-(tert- butylsulfonyl)-7- methoxyimidazo[1,2- a]pyridin-3-yl)-2- fluoro-6- methoxyaniline | [M + H]⁺ = 408.1 ¹H NMR (400 MHz, DMSO-d₆) δ ppm 8.38 (d, J = 2.4 Hz, 1H), 7.65 (s, 1H), 7.27 (s, 1H), 6.89 (d, J = 8.8 Hz, 1H), 6.73 (t, J = 8.0 Hz, 1H), 4.95 (br s, 2H), 3.93 (s, 3H), 3.88 (s, 3H), 1.30 (s, 9H). |
| 29 | 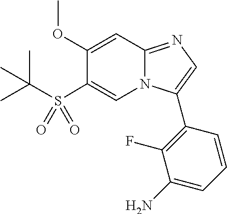 [SMILES: COc1cc2ncc(-c3cccc(N)c3F)n2cc1S(=O)(=O)C(C)(C)C] | 3-(6-(tert- butylsulfonyl)-7- methoxyimidazo[1,2- a]pyridin-3-yl)-2- fluoroaniline | [M + H]⁺ = 378.1 ¹H NMR (400 MHz, DMSO-d₆) δ ppm 8.40 (d, J = 2.8 Hz, 1H), 7.72 (s, 1H), 7.29 (s, 1H), 7.10- 7.01 (m, 1H), 6.97-6.86 (m, 1H), 6.69 (t, J = 6.4 Hz, 1H), 5.43 (s, 2H), 3.94 (s, 3H), 1.30 (s, 9H). |
| Int. 72 | 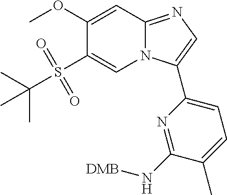 | 6-(6-(tert- butylsulfonyl)-7- methoxyimidazo[1,2- a]pyridin-3-yl)-N-(2,4- dimethoxybenzyl)-3- methylpyridin-2-amine | [M + H]⁺ = 525.0 |
| 30 | 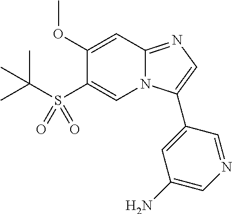 [SMILES: COc1cc2ncc(-c3cncc(N)c3)n2cc1S(=O)(=O)C(C)(C)C] | 5-(6-(tert- butylsulfonyl)-7- methoxyimidazo[1,2- a]pyridin-3-yl)pyridin- 3-amine | [M + H]⁺ = 360.9 ¹H NMR (400 MHz, DMSO-d₆) δ ppm 8.67 (s, 1H), 8.00 (d, J = 2.4 Hz, 1H), 7.95 (d, J = 2.0 Hz, 1H), 7.75 (s, 1H), 7.27 (s, 1H), 7.06 (t, J = 2.4 Hz, 1H), 5.64 (s, 2H), 3.93 (s, 3H), 1.31 (s, 9H) |
| 31 | 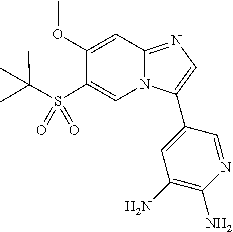 [SMILES: COc1cc2ncc(-c3cnc(N)c(N)c3)n2cc1S(=O)(=O)C(C)(C)C] | 5-(6-(tert- butylsulfonyl)-7- methoxyimidazo[1,2- a]pyridin-3- yl)pyridine-2,3-diamine | [M + H]⁺ = 375.9 ¹H NMR (400 MHz, DMSO-d₆) δ ppm 8.59 (s, 1H), 7.54 (s, 1H), 7.46 (d, J = 1.6 Hz, 1H), 7.21 (s, 1H), 6.82 (d, J = 2.0 Hz, 1H), 5.79 (s, 2H), 4.99 (s, 2H), 3.91 (s, 3H), 1.30 (s, 9H). |
| 32 | 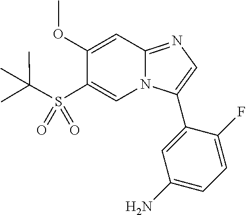 [SMILES: COc1cc2ncc(-c3cc(N)ccc3F)n2cc1S(=O)(=O)C(C)(C)C] | 3-(6-(tert- butylsulfonyl)-7- methoxyimidazo[1,2- a]pyridin-3-yl)-4- fluoroaniline | [M + H]⁺ = 377.9 ¹H NMR (400 MHz, DMSO-d₆) δ ppm 8.36 (d, J = 3.6 Hz, 1H), 7.70 (s, 1H), 7.28 (s, 1H), 7.16- 7.06 (m, 1H), 6.74-6.66 (m, 2H), 5.25 (br s, 2H), 3.94 (s, 3H), 1.30 (s, 9H). |
| 33 | 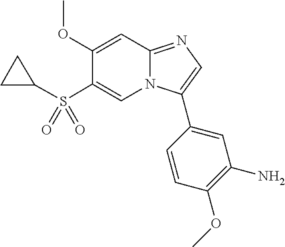 [SMILES: COc1ccc(-c2cnc3cc(OC)c(S(=O)(=O)C4CC4)cn23)cc1N] | 5-(6- (cyclopropylsulfonyl)- 7-methoxyimidazo[1,2- a]pyridin-3-yl)-2- methoxyaniline | [M + H]⁺ = 374.26 ¹H NMR (400 MHz, DMSO-d₆) δ ppm 8.67 (s, 1H), 7.55 (s, 1H), 7.26 (s, 1H), 6.98 (d, J = 8.0 Hz, 1H), 6.76 (t, J = 11.2 Hz, 2H), 4.99 (s, 2H), 4.01 (s, 3H), 3.83 (s, 3H), 3.06 (d, J = 4.8 Hz, 1H), 1.06 (t, J = 12.8 Hz, 4H). |
| 34 | 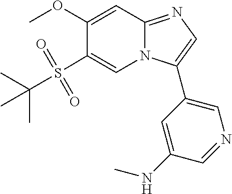 [SMILES: CNc1cncc(-c2cnc3cc(OC)c(S(=O)(=O)C(C)(C)C)cn23)c1] | 5-(6-(tert- butylsulfonyl)-7- methoxyimidazo[1,2- a]pyridin-3-yl)-N- methylpyridin-3-amine | [M + H]⁺ = 375.0 ¹H NMR (400 MHz, DMSO-d₆) δ ppm 8.73 (s, 1H), 8.02 (d, J = 2.4 Hz, 1H), 7.98 (s, 1H), 7.79 (s, 1H), 7.28 (s, 1H), 7.04 (t, J = 2.0 Hz, 1H), 6.30-6.20 (m, 1H), 3.94 (s, 3H), 2.73 (d, J = 4.8 Hz, 3H), 1.32 (s, 9H). |
| 35 | 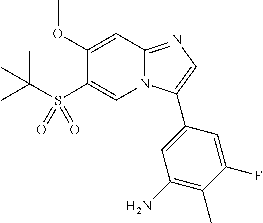 [SMILES: COc1cc2ncc(-c3cc(N)c(C)c(F)c3)n2cc1S(=O)(=O)C(C)(C)C] | 5-(6-(tert- butylsulfonyl)-7- methoxyimidazo[1,2- a]pyridin-3-yl)-3- fluoro-2-methylaniline | [M + H]⁺ = 392.0. ¹H NMR (400 MHz, DMSO-d₆) δ ppm 8.71 (s, 1H), 7.65 (s, 1H), 7.24 (s, 1H), 6.61 (s, 1H), 6.55 (d, J = 10.0 Hz, 1H), 5.51 (s, 2H), 3.93 (s, 3H), 2.02 (s, 3H), 1.31 (s, 9H). |
| Int. 73 | 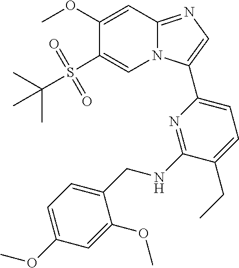 [SMILES: CCc1ccc(-c2cnc3cc(OC)c(S(=O)(=O)C(C)(C)C)cn23)nc1NCc1ccc(OC)cc1OC] | 6-(6-(tert- butylsulfonyl)-7- methoxyimidazo[1,2- a]pyridin-3-yl)-N-(2,4- dimethoxybenzyl)-3- ethylpyridin-2-amine | [M + H]⁺ = 539.3 |
| 36 | 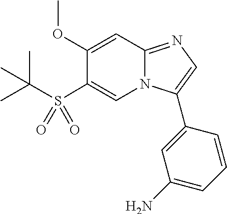 [SMILES: COc1cc2ncc(-c3cccc(N)c3)n2cc1S(=O)(=O)C(C)(C)C] | 3-(6-(tert- butylsulfonyl)-7- methoxyimidazo[1,2- a]pyridin-3-yl)aniline | [M + H]⁺ = 360.1 ¹H NMR (400 MHz, DMSO-d₆) δ ppm 8.73 (s, 1H), 7.64 (s, 1H), 7.24 (s, 1H), 7.20 (t, J = 8.0 Hz, 1H), 6.72 (d, J = 2.0 Hz, 1H), 6.70-6.63 (m, 2H), 5.37 (s, 2H), 3.93 (s, 3H), 1.31 (s, 9H). |
| 37 | 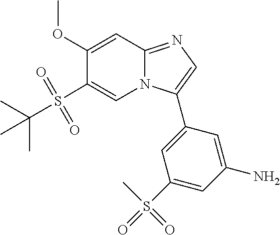 [SMILES: COc1cc2ncc(-c3cc(N)cc(S(C)(=O)=O)c3)n2cc1S(=O)(=O)C(C)(C)C] | 3-(6-(tert- butylsulfonyl)-7- methoxyimidazo[1,2- a]pyridin-3-yl)-5- (methylsulfonyl)aniline | [M + H]⁺ = 438.0 ¹H NMR (400 MHz, DMSO-d₆) δ ppm 8.72 (s, 1H), 8.40 (br s, 0.5H), 7.77 (s, 1H), 7.27 (s, 1H), 7.18 (s, 1H), 7.15 (d, J = 2.0 Hz, 1H), 6.99 (s, 1H), 6.01 (s, 2H), 3.94 (s, 3H), 3.17 (s, 3H), 1.32 (s, 9H). |
| Int. 74 | 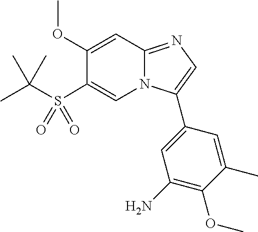 [SMILES: COc1cc2ncc(-c3cc(C)c(OC)c(N)c3)n2cc1S(=O)(=O)C(C)(C)C] | 5-(6-(tert- butylsulfonyl)-7- methoxyimidazo[1,2- a]pyridin-3-yl)-2- methoxy-3- methylaniline | ¹H NMR (400 MHz, DMSO-d₆) δ ppm 8.71 (s, 1H), 7.58 (s, 1H), 7.23 (s, 1H), 6.70 (d, J = 2.0 Hz, 1H), 6.54 (d, J = 2.0 Hz, 1H), 5.15 (s, 2H), 3.93 (s, 3H), 3.68 (s, 3H), 2.22 (s, 3H), 1.31 (s, 9H). |
| 38 | 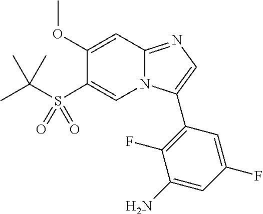 [SMILES: COc1cc2ncc(-c3cc(F)cc(N)c3F)n2cc1S(=O)(=O)C(C)(C)C] | 3-(6-(tert- butylsulfonyl)-7- methoxyimidazo[1,2- a]pyridin-3-yl)-2,5- difluoroaniline | [M + H]⁺ = 395.9 ¹H NMR (400 MHz, DMSO-d₆) δ ppm 8.41 (d, J = 3.2 Hz, 1H), 7.77 (s, 1H), 7.30 (s, 1H), 6.70- 6.63 (m, 1H), 6.56-6.49 (m, 1H), 5.79 (s, 2H), 3.94 (s, 3H), 1.31 (s, 9H). |
| 39 | 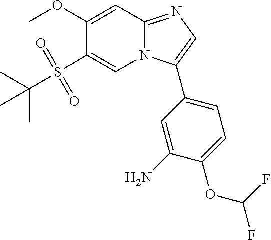 [SMILES: COc1cc2ncc(-c3ccc(OC(F)F)c(N)c3)n2cc1S(=O)(=O)C(C)(C)C] | 5-(6-(tert- butylsulfonyl)-7- methoxyimidazo[1,2- a]pyridin-3-yl)-2- (difluoromethoxy)aniline | [M + H]⁺ = 426.0 ¹H NMR (400 MHz, DMSO-d₆) δ ppm 8.69 (s, 1H), 7.64 (s, 1H), 7.32-6.93 (m, 4H), 6.74 (d, J = 2.0, 8.3 Hz, 1H), 5.39 (s, 2H), 3.93 (s, 3H), 1.31 (s, 9H). |
| 40 | 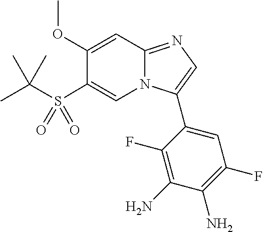 [SMILES: COc1cc2ncc(-c3cc(F)c(N)c(N)c3F)n2cc1S(=O)(=O)C(C)(C)C] | 4-(6-(tert- butylsulfonyl)-7- methoxyimidazo[1,2- a]pyridin-3-yl)-3,6- difluorobenzene-1,2- diamine | [M + H]⁺ = 411.0 ¹H NMR (400 MHz, DMSO-d₆) δ ppm 8.35 (d, J = 2.4 Hz, 1H), 7.61 (s, 1H), 7.25 (s, 1H), 6.57 (dd, J = 6.0, 10.8 Hz, 1H), 5.15 (s, 2H), 5.04 (s, 2H), 3.93 (s, 3H), 1.30 (s, 9H). |
| 41 | 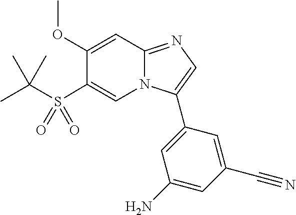 [SMILES: COc1cc2ncc(-c3cc(N)cc(C#N)c3)n2cc1S(=O)(=O)C(C)(C)C] | 3-amino-5-(6-(tert- butylsulfonyl)-7- methoxyimidazo[1,2- a]pyridin-3- yl)benzonitrile | [M + H]⁺ = 384.9 ¹H NMR (400 MHz, DMSO-d₆) δ ppm 8.67 (s, 1H), 7.75 (s, 1H), 7.27 (s, 1H), 7.12 (t, J = 1.6 Hz, 1H), 7.01 (t, J = 1.6 Hz, 1H), 6.97-6.90 (m, 1H), 5.92 (s, 2H), 3.93 (s, 3H), 1.31 (s, 9H). |
| 42 | 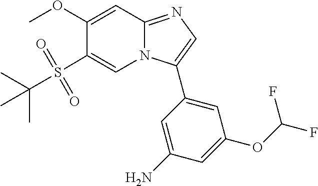 [SMILES: COc1cc2ncc(-c3cc(N)cc(OC(F)F)c3)n2cc1S(=O)(=O)C(C)(C)C] | 3-(6-(tert- butylsulfonyl)-7- methoxyimidazo[1,2- a]pyridin-3-yl)-5- (difluoromethoxy)aniline | [M + H]⁺ = 426.0 ¹H NMR (400 MHz, DMSO-d₆) δ ppm 8.72 (s, 1H), 7.69 (s, 1H), 7.39-6.92 (m, 2H), 6.59 (s, 1H), 6.48 (s, 1H), 6.42 (s, 1H), 5.71 (s, 2H), 3.93 (s, 3H), 1.31 (s, 9H). |
| 43 | 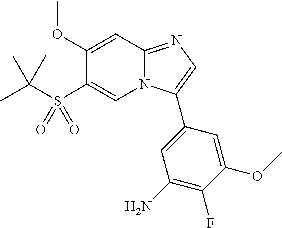 [SMILES: COc1cc2ncc(-c3cc(N)c(F)c(OC)c3)n2cc1S(=O)(=O)C(C)(C)C] | 5-(6-(tert- butylsulfonyl)-7- methoxyimidazo[1,2- a]pyridin-3-yl)-2- fluoro-3- methoxyaniline | [M + H]⁺ = 408.0 ¹H NMR (400 MHz, DMSO-d₆) δ ppm 8.74 (s, 1H), 7.71 (s, 1H), 7.27 (s, 1H), 6.56 (d, J = 7.2 Hz, 2H), 5.72-5.07 (m, 2H), 3.95 (s, 3H), 3.79 (s, 3H), 1.31 (s, 9H). |
| 44 | 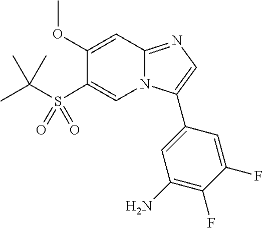 [SMILES: COc1cc2ncc(-c3cc(N)c(F)c(F)c3)n2cc1S(=O)(=O)C(C)(C)C] | 5-(6-(tert- butylsulfonyl)-7- methoxyimidazo[1,2- a]pyridin-3-yl)-2,3- difluoroaniline | [M + H]⁺ = 396.0 ¹H NMR (400 MHz, DMSO-d₆) δ ppm 8.66 (s, 1H), 7.67 (s, 1H), 7.25 (s, 1H), 6.80-6.71 (m, 2H), 5.84 (s, 2H), 3.93 (s, 3H), 1.31 (s, 9H). |
| 45 | 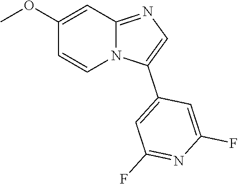 [SMILES: COc1ccn2c(-c3cc(F)nc(F)c3)cnc2c1] | 3-(2,6-difluoropyridin- 4-yl)-7- methoxyimidazo[1,2- a]pyridine | [M + H]⁺ = 262.3. ¹H NMR (400 MHz, DMSO-d₆) δ ppm 8.74 (d, J = 2.5 Hz, 1 H), 8.31 (s, 1 H), 7.50 (s, 2 H), 7.14 (d, J = 2.5 Hz, 1 H), 6.78 (dd, J = 7.5 Hz, 2.7 Hz, 1 H), 3.89 (s, 3 H). |
| 46 | 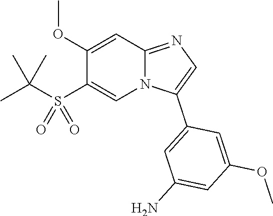 [SMILES: COc1cc(N)cc(-c2cnc3cc(OC)c(S(=O)(=O)C(C)(C)C)cn23)c1] | 3-(6-(tert- butylsulfonyl)-7- methoxyimidazo[1,2- a]pyridin-3-yl)-5- methoxyaniline | [M + H]⁺ = 389.9 ¹H NMR (400 MHz, DMSO-d₆) δ ppm 8.78 (s, 1H), 7.64 (s, 1H), 7.23 (s, 1H), 6.35-6.32 (m, 1H), 6.3-6.28 (m, 1H), 6.25- 6.22 (m, 1H), 5.40 (s, 2H), 3.92 (s, 3H), 3.71 (s, 3H), 1.31 (s, 9H). |
| 47 | 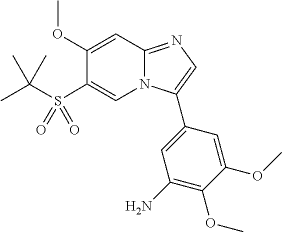 [SMILES: COc1cc2ncc(-c3cc(N)c(OC)c(OC)c3)n2cc1S(=O)(=O)C(C)(C)C] | 5-(6-(tert- butylsulfonyl)-7- methoxyimidazo[1,2- a]pyridin-3-yl)-2,3- dimethoxyaniline | [M + H]⁺ = 420.1 ¹H NMR (400 MHz, DMSO-d₆) δ ppm 8.78 (s, 1H), 7.62 (s, 1H), 7.23 (s, 1H), 6.50 (d, J = 2.0 Hz, 1H), 6.45 (d, J = 1.6 Hz, 1H), 5.16 (s, 2H), 3.93 (s, 3H), 3.77 (s, 3H), 3.70 (s, 3H), 1.32 (s, 9H). |
| 48 | 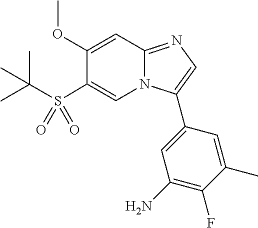 [SMILES: COc1cc2ncc(-c3cc(C)c(F)c(N)c3)n2cc1S(=O)(=O)C(C)(C)C] | 5-(6-(tert- butylsulfonyl)-7- methoxyimidazo[1,2- a]pyridin-3-yl)-2- fluoro-3-methylaniline | [M + H]⁺ = 392.1 ¹H NMR (400 MHz, DMSO-d₆) δ ppm 8.67 (s, 1H), 7.60 (s, 1H), 7.23 (s, 1H), 6.75 (dd, J = 2.0, 8.0 Hz, 1H), 6.61-6.56 (m, 1 H), 5.37 (s, 2H), 3.92 (s, 3H), 2.22 (d, J = 2.0 Hz, 3 H), 1.30 (s, 9H). |
| 49 | 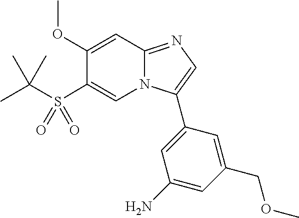 [SMILES: COCc1cc(N)cc(-c2cnc3cc(OC)c(S(=O)(=O)C(C)(C)C)cn23)c1] | 3-(6-(tert- butylsulfonyl)-7- methoxyimidazo[1,2- a]pyridin-3-yl)-5- (methoxymethyl)aniline | [M + H]⁺ = 403.9 ¹H NMR (400 MHz, DMSO-d₆) δ ppm 8.96-8.55 (m, 1H), 7.78- 7.54 (m, 1H), 7.38-7.13 (m, 1H), 6.70-6.55 (m, 3H), 5.59- 5.15 (m, 2H), 4.43-4.24 (m, 2H), 3.97-3.86 (m, 3H), 3.30- 3.29 (m, 3H), 1.36-1.24 (m, 9H). |
| 50 | 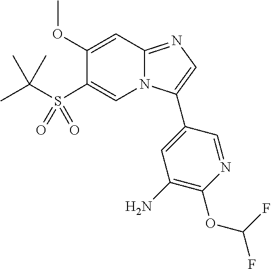 [SMILES: COc1cc2ncc(-c3cnc(OC(F)F)c(N)c3)n2cc1S(=O)(=O)C(C)(C)C] | 5-(6-(tert- butylsulfonyl)-7- methoxyimidazo[1,2-a] pyridin-3-yl)-2- (difluoromethoxy)pyridin- 3-amine | [M + H]⁺ = 427.0 ¹H NMR (400 MHz, DMSO-d₆) δ ppm 8.63 (s, 1H), 7.93-7.52 (m, 3H), 7.27 (s, 1H), 7.23 (d, J = 2.0 Hz, 1H), 5.63 (s, 2H), 3.93 (s, 3H), 1.31 (s, 9H). |
| 51 | 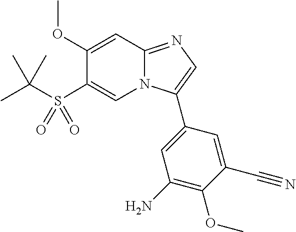 [SMILES: COc1cc2ncc(-c3cc(N)c(OC)c(C#N)c3)n2cc1S(=O)(=O)C(C)(C)C] | 3-amino-5-(6-(tert- butylsulfonyl)-7- methoxyimidazo[1,2- a]pyridin-3-yl)-2- methoxybenzonitrile | [M + H]⁺ = 415.1 ¹H NMR (400 MHz, DMSO-d₆) δ ppm 8.64 (s, 1H), 7.70 (s, 1H), 7.26 (s, 1H), 7.15-7.06 (m, 2H), 5.78 (s, 2H), 3.93 (s, 3H), 3.89 (s, 3H), 1.31 (s, 9H). |
| 52 | 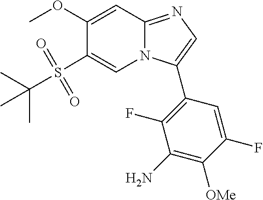 [SMILES: COc1cc2ncc(-c3cc(F)c(OC)c(N)c3F)n2cc1S(=O)(=O)C(C)(C)C] | 3-(6-(tert- butylsulfonyl)-7- methoxyimidazo[1,2- a]pyridin-3-yl)-2,5- difluoro-6- methoxyaniline | [M + H]⁺ = 426.0 ¹H NMR (400 MHz, DMSO-d₆) δ ppm 8.39 (d, J = 2.4 Hz, 1H), 7.72 (s, 1H), 7.29 (s, 1H), 6.72- 6.60 (m, 1H), 5.56 (s, 2H), 3.94 (s, 3H), 3.86 (s, 3H), 1.31 (s, 9H) |
| 53 | 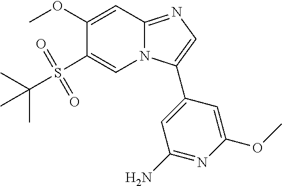 [SMILES: COc1cc(-c2cnc3cc(OC)c(S(=O)(=O)C(C)(C)C)cn23)cc(N)n1] | 4-(6-(tert- butylsulfonyl)-7- methoxyimidazo[1,2- a]pyridin-3-yl)-6- methoxypyridin-2- amine | [M + H]⁺ = 391.0 ¹H NMR (400 MHz, DMSO-d₆) δ ppm 8.81 (s, 1H), 8.42 (s, 0.2H), 7.81 (s, 1H), 7.28 (s, 1H), 6.22 (s, 2H), 6.17 (s, 1H), 6.09 (s, 1H), 3.93 (s, 3H), 3.79 (s, 3H), 1.32 (s, 9H). |
| 54 | 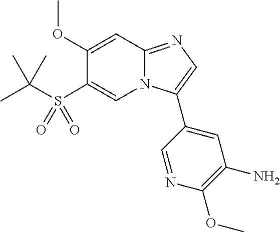 [SMILES: COc1cc2ncc(-c3cnc(OC)c(N)c3)n2cc1S(=O)(=O)C(C)(C)C] | 5-(6-(tert- butylsulfonyl)-7- methoxyimidazo[1,2- a]pyridin-3-yl)-2- methoxypyridin-3- amine | [M + H]⁺ = 390.9 ¹H NMR (400 MHz, DMSO-d₆) δ ppm 8.62 (s, 1H), 7.64 (s, 1H), 7.57 (d, J = 2.0 Hz, 1H), 7.25 (s, 1H), 7.01 (d, J = 2.0 Hz, 1H), 5.30 (s, 2H), 3.96-3.90 (m, 6H), 1.31 (s, 9H). |
| Int. 75 | 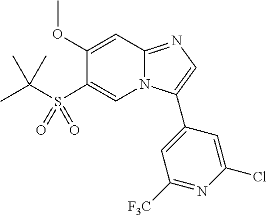 [SMILES: COc1cc2ncc(-c3cc(Cl)nc(C(F)(F)F)c3)n2cc1S(=O)(=O)C(C)(C)C] | 6-(tert-butylsulfonyl)- 3-(2-chloro-6- (trifluoromethyl)pyridin- 4-yl)-7- methoxyimidazo[1,2- a]pyridine | [M + H]⁺ = 448.4 |
| 55 | 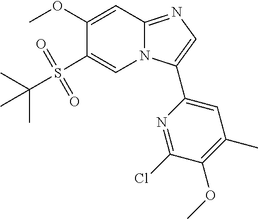 [SMILES: COc1cc2ncc(-c3cc(C)c(OC)c(Cl)n3)n2cc1S(=O)(=O)C(C)(C)C] | 6-(tert-butylsulfonyl)- 3-(6-chloro-5-methoxy- 4-methylpyridin-2-yl)- 7-methoxyimidazo[1,2- a]pyridine | [M + H]⁺ = 424.0 ¹H NMR (400 MHz, DMSO-d₆) δ ppm: 10.25 (s, 1H), 8.38 (s, 1H), 7.97 (s, 1H), 7.31 (s, 1H), 3.95 (s, 3H), 3.83 (s, 3H), 2.39 (s, 3H), 1.34 (s, 9H). |
| 56 | 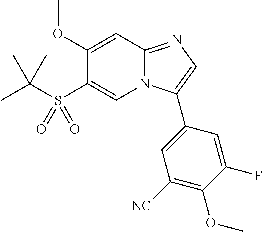 [SMILES: COc1cc2ncc(-c3cc(F)c(OC)c(C#N)c3)n2cc1S(=O)(=O)C(C)(C)C] | 5-(6-(tert- butylsulfonyl)-7- methoxyimidazo[1,2- a]pyridin-3-yl)-3- fluoro-2- methoxybenzonitrile | [M + H]⁺ = 417.9 ¹H NMR (400 MHz, DMSO-d₆) δ ppm: 8.65 (s, 1H), 8.01 (dd, J = 1.8, 13.1 Hz, 1H), 7.94 (s, 1H), 7.83 (s, 1H), 7.29 (s, 1H), 4.15 (d, J = 2.8 Hz, 3H), 3.94 (s, 3H), 1.31 (s, 9H). |
| 57 | 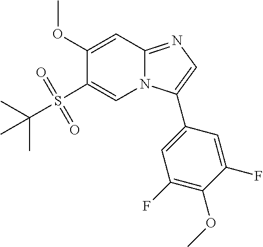 [SMILES: COc1cc2ncc(-c3cc(F)c(OC)c(F)c3)n2cc1S(=O)(=O)C(C)(C)C] | 6-(tert-butylsulfonyl)- 3-(3,5-difluoro-4- methoxyphenyl)-7- methoxyimidazo[1,2- a]pyridine | [M + H]⁺ = 411.1 ¹H NMR (400 MHz, DMSO-d₆) δ ppm: 8.68 (s, 1H), 7.80 (s, 1H), 7.53-7.45 (m, 2H), 7.28 (s, 1H), 4.00 (s, 3H), 3.94 (s, 3H), 1.32 (s, 9H). |
| 58 | 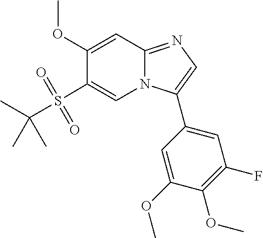 [SMILES: COc1cc2ncc(-c3cc(F)c(OC)c(OC)c3)n2cc1S(=O)(=O)C(C)(C)C] | 6-(tert-butylsulfonyl)- 3-(3-fluoro-4,5- dimethoxyphenyl)-7- methoxyimidazo[1,2- a]pyridine | [M + H]⁺ = 422.9 ¹H NMR (400 MHz, DMSO-d₆) δ ppm: 8.74 (s, 1H), 7.78 (s, 1H), 7.27 (s, 1H), 7.25-7.10 (m, 2H), 3.93 (s, 3H), 3.89-3.84 (m, 6H), 1.32 (s, 9H). |
| Int. 76 | 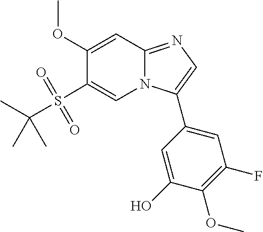 [SMILES: COc1cc2ncc(-c3cc(O)c(OC)c(F)c3)n2cc1S(=O)(=O)C(C)(C)C] | 5-(6-(tert- butylsulfonyl)-7- methoxyimidazo[1,2- a]pyridin-3-yl)-3- fluoro-2- methoxyphenol | ¹H NMR (400 MHz, DMSO-d₆) δ ppm: 10.21 (s, 1H), 8.69 (s, 1H), 7.71 (s, 1H), 7.26 (s, 1H), 7.02-6.93 (m, 1H), 6.89 (s, 1H), 3.93 (s, 3H), 3.86 (s, 3H), 1.32 (s, 9H). |
| 59 | 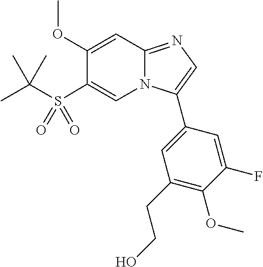 [SMILES: COc1cc2ncc(-c3cc(F)c(OC)c(CCO)c3)n2cc1S(=O)(=O)C(C)(C)C] | 2-(5-(6-(tert- butylsulfonyl)-7- methoxyimidazo[1,2- a]pyridin-3-yl)-3- fluoro-2- methoxyphenyl)ethan- 1-ol | [M + H]⁺ = 436.9 ¹H NMR (400 MHz, DMSO-d₆) δ ppm: 8.69 (s, 1H), 7.74 (s, 1H), 7.46-7.38 (m, 1H), 7.31 (s, 1H), 7.26 (s, 1H), 4.75 (t, J = 5.2 Hz, 1H), 3.93 (s, 3H), 3.91 (d, J = 2.0 Hz, 3H), 3.68-3.57 (m, 2H), 2.82 (t, J = 6.8 Hz, 2H), 1.32 (s, 9H). |
| 60 | 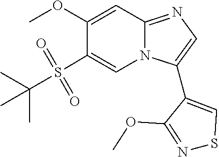 [SMILES: COc1cc2ncc(-c3csnc3OC)n2cc1S(=O)(=O)C(C)(C)C] | 4-(6-(tert- butylsulfonyl)-7- methoxyimidazo[1,2- a]pyridin-3-yl)-3- methoxyisothiazole | [M + H]⁺ = 382.0 |
| 61 | 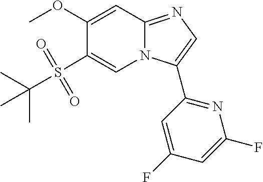 [SMILES: COc1cc2ncc(-c3cc(F)cc(F)n3)n2cc1S(=O)(=O)C(C)(C)C] | 6-(tert-butylsulfonyl)- 3-(4,6-difluoropyridin- 2-yl)-7- methoxyimidazo[1,2- a]pyridine | [M + H]⁺ = 382.0 ¹H NMR (400 MHz, DMSO-d₆) δ ppm: 10.26 (s, 1H), 8.70 (s, 1H), 8.04 (dd, J = 1.6, 9.6 Hz, 1H), 7.42 (s, 1H), 7.22 (d, J = 8.8 Hz, 1H), 4.00 (s, 3H), 1.34 (s, 9H). |
| 62 | 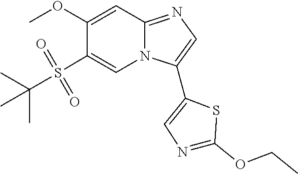 [SMILES: CCOc1ncc(-c2cnc3cc(OC)c(S(=O)(=O)C(C)(C)C)cn23)s1] | 5-(6-(tert- butylsulfonyl)-7- methoxyimidazo[1,2- a]pyridin-3-yl)-2- ethoxythiazole | [M + H]⁺ = 396.1 |
| 63 | 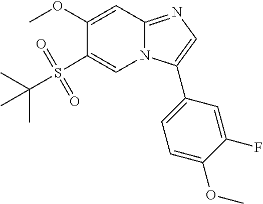 [SMILES: COc1ccc(-c2cnc3cc(OC)c(S(=O)(=O)C(C)(C)C)cn23)cc1F] | 6-(tert-butylsulfonyl)- 3-(3-fluoro-4- methoxyphenyl)-7- methoxyimidazo[1,2- a]pyridine | [M + H]⁺ = 393.0 ¹H NMR (400 MHz, DMSO-d₆) δ ppm: 8.64 (s, 1H), 8.43-8.36 (m, 0.19H), 7.72 (s, 1H), 7.56- 7.49 (m, 1H), 7.42-7.36 (m, 2H), 7.26 (s, 1H), 3.92 (d, J = 3.6 Hz, 6H), 1.31 (s, 9H). |
| 64 | 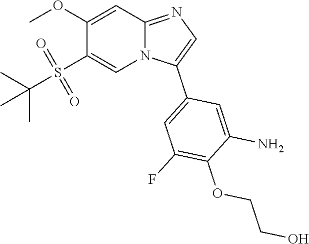 [SMILES: COc1cc2ncc(-c3cc(N)c(OCCO)c(F)c3)n2cc1S(=O)(=O)C(C)(C)C] | 2-(2-amino-4-(6-(tert- butylsulfonyl)-7- methoxyimidazo[1,2- a]pyridin-3-yl)-6- fluorophenoxy)ethan-1- ol | [M + H]⁺ = 438.0 ¹H NMR (400 MHz, DMSO-d₆) δ ppm: 8.70 (s, 1H), 7.65 (s, 1H), 7.25 (s, 1H), 6.66 (s, 1H), 6.62 (dd, J = 2.0, 11.6 Hz, 1H), 5.60 (s, 2H), 5.09 (t, J = 5.6 Hz, 1H), 3.99 (t, J = 4.4 Hz, 2H), 3.93 (s, 3H), 3.710-3.65 (m, 2H), 1.31 (s, 9H). |
| 65 | 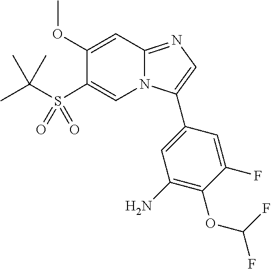 [SMILES: COc1cc2ncc(-c3cc(N)c(OC(F)F)c(F)c3)n2cc1S(=O)(=O)C(C)(C)C] | 5-(6-(tert- butylsulfonyl)-7- methoxyimidazo[1,2- a]pyridin-3-yl)-2- (difluoromethoxy)-3- fluoroaniline | [M + H]⁺ = 444.2 ¹H NMR (400 MHz, DMSO-d₆) δ ppm 8.70 (s, 1H), 7.70 (s, 1H), 7.26 (s, 1H), 7.24-6.83 (m, 1H), 6.77-6.74 (m, 1H), 6.74- 6.70 (m, 1H), 5.78 (s, 2H), 3.93 (s, 3H), 1.31 (s, 9H). |
| 66 | 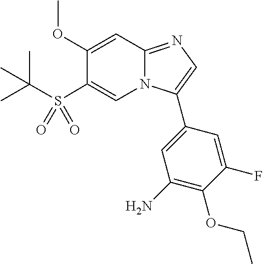 [SMILES: CCOc1c(N)cc(-c2cnc3cc(OC)c(S(=O)(=O)C(C)(C)C)cn23)cc1F] | 5-(6-(tert- butylsulfonyl)-7- methoxyimidazo[1,2- a]pyridin-3-yl)-2- ethoxy-3-fluoroaniline | [M + H]⁺ = 422.1 ¹H NMR (400 MHz, DMSO-d₆) δ ppm 8.70 (s, 1H), 7.64 (s, 1H), 7.25 (s, 1H), 6.68-6.57 (m, 2H), 5.48 (s, 2H), 4.06-3.97 (m, 2H), 3.92 (s, 3H), 1.37-1.32 (m, 3H), 1.31 (s, 9H). |
| 67 | 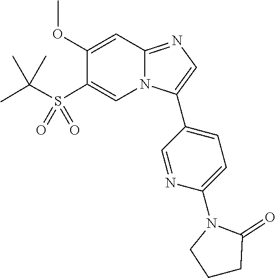 [SMILES: COc1cc2ncc(-c3ccc(N4CCCC4=O)nc3)n2cc1S(=O)(=O)C(C)(C)C] | 1-(5-(6-(tert- butylsulfonyl)-7- methoxyimidazo[1,2- a]pyridin-3-yl)pyridin- 2-yl)pyrrolidin-2-one | [M + H]⁺ = 428.9 ¹H NMR (400 MHz, DMSO-d₆) δ ppm 8.65 (d, J = 2.0 Hz, 1H), 8.62 (s, 1H), 8.48 (d, J = 8.8 Hz, 1H), 8.11-8.04 (m, 1H), 7.81 (s, 1H), 7.29 (s, 1H), 4.06 (t, J = 6.8 Hz, 2H), 3.94 (s, 3H), 2.62 (t, J = 8.4 Hz, 2H), 2.14- 2.02 (m, 2H), 1.31 (s, 9H). |
| 68 | 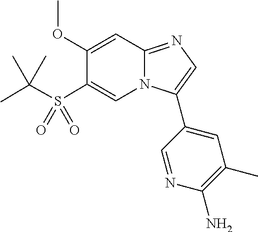 [SMILES: COc1cc2ncc(-c3cnc(N)c(C)c3)n2cc1S(=O)(=O)C(C)(C)C] | 5-(6-(tert- butylsulfonyl)-7- methoxyimidazo[1,2- a]pyridin-3-yl)-3- methylpyridin-2-amine | [M + H]⁺ = 374.9 ¹H NMR (400 MHz, DMSO-d₆) δ ppm 8.53 (s, 1H), 8.00 (d, J = 2.0 Hz, 1H), 7.59 (s, 1H), 7.45 (d, J = 1.2 Hz, 1H), 7.22 (s, 1H), 6.12 (s, 2H), 3.92 (s, 3H), 2.11 (s, 3H), 130 (s, 9H). |
| 69 | 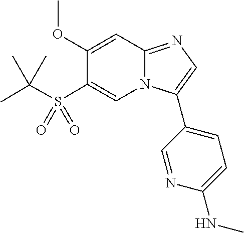 [SMILES: CNc1ccc(-c2cnc3cc(OC)c(S(=O)(=O)C(C)(C)C)cn23)cn1] | 5-(6-(tert- butylsulfonyl)-7- methoxyimidazo[1,2- a]pyridin-3-yl)-N- methylpyridin-2-amine | [M + H]⁺ = 375.0 ¹H NMR (400 MHz, DMSO-d₆) δ ppm 8.51 (s, 1H), 8.19 (d, J = 2.0 Hz, 1H), 7.63-7.56 (m, 2H), 7.23 (s, 1H), 6.93-6.85 (m, 1H), 6.65-6.59 (m, 1H), 3.92 (s, 3H), 2.83 (d, J = 4.8 Hz, 3H), 1.30 (s, 9H). |
| 70 | 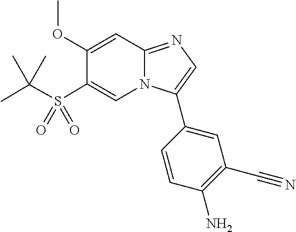 [SMILES: COc1cc2ncc(-c3ccc(N)c(C#N)c3)n2cc1S(=O)(=O)C(C)(C)C] | 2-amino-5-(6-(tert- butylsulfonyl)-7- methoxyimidazo[1,2- a]pyridin-3- yl)benzonitrile | [M + H]⁺ = 385.0 ¹H NMR (400 MHz, DMSO-d₆) δ ppm 8.52 (s, 1H), 7.64 (d, J = 2.0 Hz, 1H), 7.62 (s, 1H), 7.52 (dd, J = 8.8, 2.4 Hz, 1H), 7.23 (s, 1H), 6.96 (d, J = 8.4 Hz, 1H), 6.43 (s, 2H), 3.92 (s, 3H), 1.30 (s, 9H). |
| 71 | 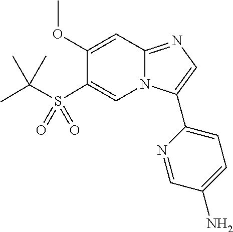 [SMILES: COc1cc2ncc(-c3ccc(N)cn3)n2cc1S(=O)(=O)C(C)(C)C] | 6-(6-(tert- butylsulfonyl)-7- methoxyimidazo[1,2- a]pyridin-3-yl)pyridin- 3-amine | [M + H]⁺ = 361.1 ¹H NMR (400 MHz, DMSO-d₆) δ ppm 10.29 (s, 1H), 8.05 (s, 1H), 8.00 (d, J = 2.8 Hz, 1H), 7.66 (d, J = 8.8 Hz, 1H), 7.22 (s, 1H), 7.07 (dd, J = 8.4, 2.8 Hz, 1H), 5.55 (s, 2H), 3.92 (s, 3H), 1.31 (s, 9H). |
| 72 | 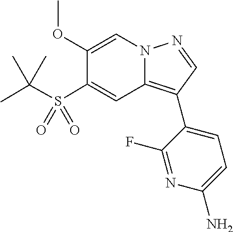 [SMILES: COc1cn2ncc(-c3ccc(N)nc3F)c2cc1S(=O)(=O)C(C)(C)C] | 5-(5-(tert- butylsulfonyl)-6- methoxypyrazolo[1,5- a]pyridin-3-yl)-6- fluoropyridin-2-amine | [M + H]⁺ = 379.2 ¹H NMR (400 MHz, DMSO-d₆) δ ppm 8.69 (s, 1H), 8.20 (s, 1H), 8.07 (d, J = 1.2 Hz, 1H), 7.78-7.73 (m, 1H), 6.53-6.51 (dd, J = 8.1, 2.0 Hz, 1H), 6.53 (brs, 2H), 3.9 (s, 3H), 1.3 (s, 9H). |
| 73 | 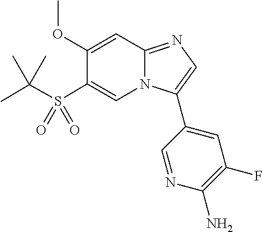 [SMILES: COc1cc2ncc(-c3cnc(N)c(F)c3)n2cc1S(=O)(=O)C(C)(C)C] | 5-(6-(tert- butylsulfonyl)-7- methoxyimidazo[1,2- a]pyridin-3-yl)-3- fluoropyridin-2-amine | [M + H]⁺ = 379.1 ¹H NMR (400 MHz, DMSO-d₆) δ ppm 8.52 (s, 1H), 7.98 (s, 1H), 7.71-7.63 (m, 2H), 7.22 (s, 1H), 6.61 (s, 2H), 3.92 (s, 3H), 1.30 (s, 9H). |
| 74 | 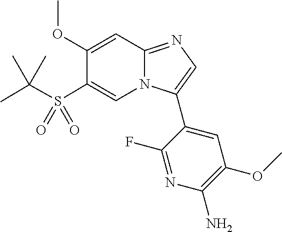 [SMILES: COc1cc2ncc(-c3cc(OC)c(N)nc3F)n2cc1S(=O)(=O)C(C)(C)C] | 5-(6-(tert- butylsulfonyl)-7- methoxyimidazo[1,2- a]pyridin-3-yl)-6- fluoro-3- methoxypyridin-2- amine | [M + H]⁺ = 409.0 ¹H NMR (400 MHz, DMSO-d₆) δ ppm 8.35 (d, J = 2.4 Hz, 1H), 7.67 (s, 1H), 7.32 (d, J = 7.6 Hz, 1H), 7.26 (s, 1H), 6.61 (s, 2H), 3.93 (s, 3H), 3.81 (s, 3H), 1.30 (s, 9H). |
| 75 | 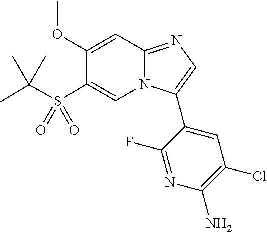 [SMILES: COc1cc2ncc(-c3cc(Cl)c(N)nc3F)n2cc1S(=O)(=O)C(C)(C)C] | 5-(6-(tert- butylsulfonyl)-7- methoxyimidazo[1,2- a]pyridin-3-yl)-3- chloro-6-fluoropyridin- 2-amine | [M + H]⁺ = 413.0 ¹H NMR (400 MHz, DMSO-d₆) δ ppm 8.36 (d, J = 2.4 Hz, 1H), 7.94 (d, J = 8.4 Hz, 1H), 7.69 (s, 1H), 7.26 (s, 1H), 7.13 (s, 2H), 3.93 (s, 3H), 1.30 (s, 9H). |
| 76 | 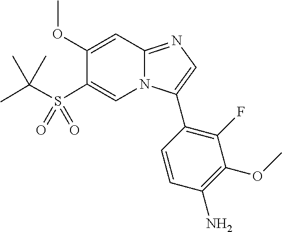 [SMILES: COc1cc2ncc(-c3ccc(N)c(OC)c3F)n2cc1S(=O)(=O)C(C)(C)C] | 4-(6-(tert- butylsulfonyl)-7- methoxyimidazo[1,2- a]pyridin-3-yl)-3- fluoro-2- methoxyaniline | [M + H]⁺ = 408.0 ¹H NMR (400 MHz, DMSO-d₆) δ ppm 8.32 (d, J = 2.8 Hz, 1H), 7.60 (s, 1H), 7.25 (s, 1H), 6.98 (t, J = 8.0 Hz, 1H), 6.65 (d, J = 7.2 Hz, 1H), 5.65 (s, 2H), 3.93 (s, 3H), 3.78 (s, 3H), 1.30 (s, 9H). |
| 77 | 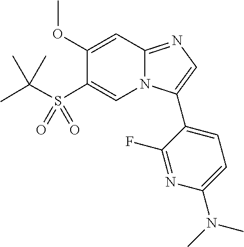 [SMILES: COc1cc2ncc(-c3ccc(N(C)C)nc3F)n2cc1S(=O)(=O)C(C)(C)C] | 5-(6-(tert- butylsulfonyl)-7- methoxyimidazo[1,2- a]pyridin-3-yl)-6- fluoro-N,N- dimethylpyridin-2- amine | [M + H]⁺ = 407.1 ¹H NMR (400 MHz, DMSO-d₆) δ ppm 8.32 (d, J = 2.4 Hz, 1H), 7.85-7.75 (m, 1H), 7.66 (s, 1H), 7.27 (s, 1H), 6.75-6.66 (m, 1H), 3.93 (s, 3H), 3.09 (s, 6H), 1.30 (s, 9H). |
| 78 | 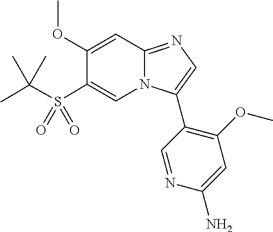 [SMILES: COc1cc(N)ncc1-c1cnc2cc(OC)c(S(=O)(=O)C(C)(C)C)cn12] | 5-(6-(tert- butylsulfonyl)-7- methoxyimidazo[1,2- a]pyridin-3-yl)-4- methoxypyridin-2- amine | [M + H]⁺ = 391.0 ¹H NMR (400 MHz, DMSO-d₆) δ ppm 8.13 (s, 1H), 7.83 (s, 1H), 7.53 (s, 1H), 7.21 (s, 1H), 6.32 (s, 2H), 6.20 (s, 1H), 3.92 (s, 3H), 3.69 (s, 3H), 1.30 (s, 9H). |
| 79 | 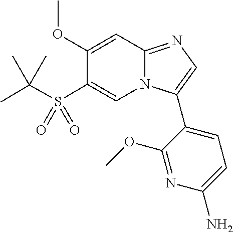 [SMILES: COc1cc2ncc(-c3ccc(N)nc3OC)n2cc1S(=O)(=O)C(C)(C)C] | 5-(6-(tert- butylsulfonyl)-7- methoxyimidazo[1,2- a]pyridin-3-yl)-6- methoxypyridin-2- amine | [M + H]⁺ = 391.1 ¹H NMR (400 MHz, DMSO-d₆) δ ppm 8.16 (s, 1H), 7.50 (s, 1H), 7.43 (d, J = 8.0 Hz, 1H), 7.21 (s, 1H), 6.36 (s, 2H), 6.18 (d, J = 8.4 Hz, 1H), 3.92 (s, 3H), 3.72 (s, 3H), 1.31 (s, 9H). |
| 80 | 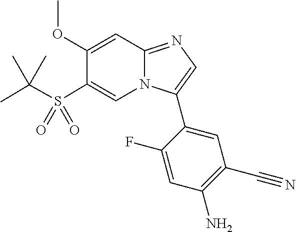 [SMILES: COc1cc2ncc(-c3cc(C#N)c(N)cc3F)n2cc1S(=O)(=O)C(C)(C)C] | 2-amino-5-(6-(tert- butylsulfonyl)-7- methoxyimidazo[1,2- a]pyridin-3-yl)-4- fluorobenzonitrile | [M + H]⁺ = 403.0 ¹H NMR (400 MHz, DMSO-d₆) δ ppm 8.29 (d, J = 2.8 Hz, 1H), 7.74 (d, J = 8.0 Hz, 1H), 7.67 (s, 1H) 7.26 (s, 1H), 6.79-6.41 (m, 3H), 3.93 (s, 3H), 1.29 (s, 9H). |
| 81 | 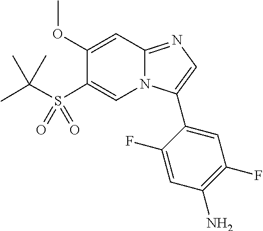 [SMILES: COc1cc2ncc(-c3cc(F)c(N)cc3F)n2cc1S(=O)(=O)C(C)(C)C] | 4-(6-(tert- butylsulfonyl)-7- methoxyimidazo[1,2- a]pyridin-3-yl)-2,5- difluoroaniline | [M + H]⁺ = 396.0 ¹H NMR (400 MHz, DMSO-d₆) δ ppm 8.28 (d, J = 2.8 Hz, 1H), 7.64 (s, 1H), 7.40-7.13 (m, 2H), 6.87-6.49 (m, 1H), 5.90 (s, 2H), 3.92 (s, 3H), 1.29 (s, 9H). |
| 82 | 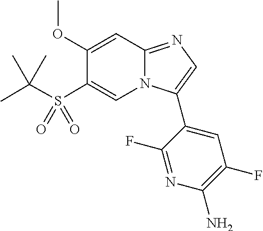 [SMILES: COc1cc2ncc(-c3cc(F)c(N)nc3F)n2cc1S(=O)(=O)C(C)(C)C] | 5-(6-(tert- butylsulfonyl)-7- methoxyimidazo[1,2- a]pyridin-3-yl)-3,6- difluoropyridin-2- amine | [M + H]⁺ = 397.1 ¹H NMR (400 MHz, DMSO-d₆) δ ppm 8.36 (d, J = 2.4 Hz, 1H), 7.77 (dd, J = 7.6, 2.8 Hz, 1H), 7.67 (s, 1H), 7.26 (s, 1H), 7.05 (s, 2H), 3.93 (s, 3H), 1.30 (s, 9H). |
| 83 | 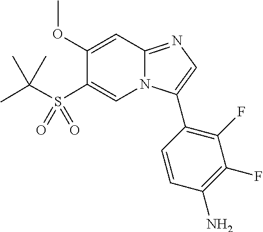 [SMILES: COc1cc2ncc(-c3ccc(N)c(F)c3F)n2cc1S(=O)(=O)C(C)(C)C] | 4-(6-(tert- butylsulfonyl)-7- methoxyimidazo[1,2- a]pyridin-3-yl)-2,3- difluoroaniline | [M + H]⁺ = 396.0 ¹H NMR (400 MHz, DMSO-d₆) δ ppm 8.35 (d, J = 2.4 Hz, 1H), 7.64 (s, 1H), 7.26 (s, 1H), 7.07 (t, J = 7.6 Hz, 1H), 6.73 (t, J = 7.6 Hz, 1H), 5.92 (s, 2H), 3.93 (s, 3H), 1.30 (s, 9H). |
| 84 | 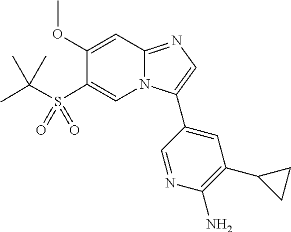 [SMILES: COc1cc2ncc(-c3cnc(N)c(C4CC4)c3)n2cc1S(=O)(=O)C(C)(C)C] | 5-(6-(tert- butylsulfonyl)-7- methoxyimidazo[1,2- a]pyridin-3-yl)-3- cyclopropylpyridin-2- amine | [M + H]⁺ = 401.1 ¹H NMR (400 MHz, DMSO-d₆) δ ppm 8.61 (s, 1H), 8.36 (br s, 2H), 8.19 (d, J = 2.0 Hz, 1H), 8.14 (s, 1H), 7.81 (s, 1H), 7.47 (s, 1H), 4.06 (s, 3H), 1.94-1.84 (m, 1H), 1.33 (s, 9H), 1.08-1.00 (m, 2H), 0.76-0.65 (m, 2H) |
| 85 | 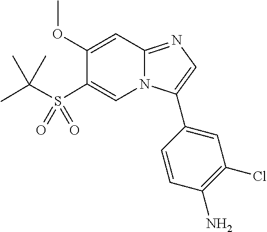 [SMILES: COc1cc2ncc(-c3ccc(N)c(Cl)c3)n2cc1S(=O)(=O)C(C)(C)C] | 4-(6-(tert- butylsulfonyl)-7- methoxyimidazo[1,2- a]pyridin-3-yl)-2- chloroaniline | [M + H]⁺ = 394.0 ¹H NMR (400 MHz, DMSO-d₆) δ ppm 8.58 (s, 1H), 7.66 (s, 1H), 7.44 (d, J = 1.6 Hz, 1H), 7.28-7.22 (m, 2H), 6.99-6.91 (m, 1H), 5.75 (br s, 2H), 3.93 (s, 3H), 1.30 (s, 9H). |
| 86 | 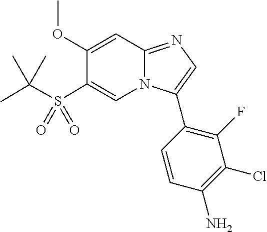 [SMILES: COc1cc2ncc(-c3ccc(N)c(Cl)c3F)n2cc1S(=O)(=O)C(C)(C)C] | 4-(6-(tert- butylsulfonyl)-7- methoxyimidazo[1,2- a]pyridin-3-yl)-2- chloro-3-fluoroaniline | [M + H]⁺ = 412.0 ¹H NMR (400 MHz, DMSO-d₆) δ ppm 8.31 (d, J = 2.8 Hz, 1H), 7.64 (s, 1H), 7.28-7.20 (m, 2H), 6.80-6.75 (m, 1H), 6.12 (s, 2H), 3.93 (s, 3H), 1.30 (s, 9H). |
| 87 | 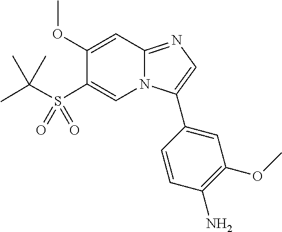 [SMILES: COc1cc(-c2cnc3cc(OC)c(S(=O)(=O)C(C)(C)C)cn23)ccc1N] | 4-(6-(tert- butylsulfonyl)-7- methoxyimidazo[1,2- a]pyridin-3-yl)-2- methoxyaniline | [M + H]⁺ = 390.0 ¹H NMR (400 MHz, DMSO-d₆) δ ppm 8.68 (s, 1H), 7.58 (s, 1H), 7.20 (s, 1H), 7.03-7.00 (m, 1H), 6.90 (dd, J = 8.0, 2.0 Hz, 1H), 6.81-6.76 (m, 1H), 5.08 (s, 2H), 3.92 (s, 3H), 3.80 (s, 3H), 1.31 (s, 9H). |
| 88 | 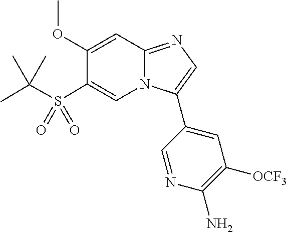 [SMILES: COc1cc2ncc(-c3cnc(N)c(OC(F)(F)F)c3)n2cc1S(=O)(=O)C(C)(C)C] | 5-(6-(tert- butylsulfonyl)-7- methoxyimidazo[1,2- a]pyridin-3-yl)-3- (trifluoromethoxy) pyridin-2-amine | [M + H]⁺ = 445.0 ¹H NMR (400 MHz, DMSO-d₆) δ ppm 8.49 (s, 1H), 8.15 (d, J = 1.6 Hz, 1H), 7.82 (s, 1H), 7.68 (s, 1H), 7.23 (s, 1H), 6.82 (s, 2H), 3.92 (s, 3H), 1.30 (s, 9H). |
| 89 | 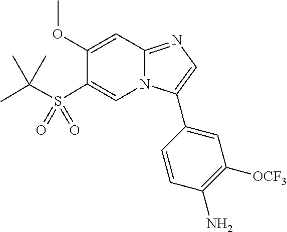 [SMILES: COc1cc2ncc(-c3ccc(N)c(OC(F)(F)F)c3)n2cc1S(=O)(=O)C(C)(C)C] | 4-(6-(tert- butylsulfonyl)-7- methoxyimidazo[1,2- a]pyridin-3-yl)-2- (trifluoromethoxy)aniline | [M + H]⁺ = 444.0 ¹H NMR (400 MHz, DMSO-d₆) δ ppm 8.59 (s, 1H), 7.62 (s, 1H), 7.36 (s, 1H), 7.27 (dd, J = 8.4, 1.6 Hz, 1H), 7.22 (s, 1H), 6.98 (d, J = 8.4 Hz, 1H), 5.78 (s, 2H), 3.92 (s, 3H), 1.31 (s, 9H). |
| 90 | 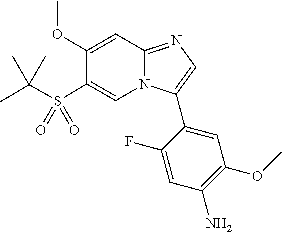 [SMILES: COc1cc(-c2cnc3cc(OC)c(S(=O)(=O)C(C)(C)C)cn23)c(F)cc1N] | 4-(6-(tert- butylsulfonyl)-7- methoxyimidazo[1,2- a]pyridin-3-yl)-5- fluoro-2- methoxyaniline | [M + H]⁺ = 408.1 ¹H NMR (400 MHz, DMSO-d₆) δ ppm 8.36 (d, J = 2.8 Hz, 1H), 7.63 (s, 1H), 7.24 (s, 1H), 6.93 (d, J = 6.8 Hz, 1H), 6.61 (d, J = 12.0 Hz, 1H), 5.47 (s, 2H), 3.93 (s, 3H), 3.79 (s, 3H), 1.30 (s, 9H). |
| 91 | 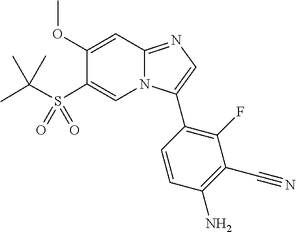 [SMILES: COc1cc2ncc(-c3ccc(N)c(C#N)c3F)n2cc1S(=O)(=O)C(C)(C)C] | 6-amino-3-(6-(tert- butylsulfonyl)-7- methoxyimidazo[1,2- a]pyridin-3-yl)-2- fluorobenzonitrile | [M + H]⁺ = 403.1 ¹H NMR (400 MHz, DMSO-d₆) δ ppm 8.32 (d, J = 1.6 Hz, 1H), 7.66 (s, 1H), 7.51 (t, J = 8.4 Hz, 1H), 7.27 (s, 1H), 6.87 (s, 2H), 6.77 (d, J = 8.4 Hz, 1H), 3.93 (s, 3H), 1.30 (s, 9H). |
| 92 | 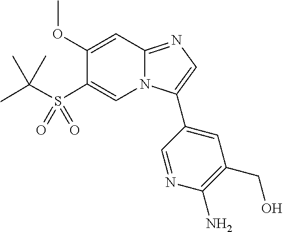 [SMILES: COc1cc2ncc(-c3cnc(N)c(CO)c3)n2cc1S(=O)(=O)C(C)(C)C] | (2-amino-5-(6-(tert- butylsulfonyl)-7- methoxyimidazo[1,2- a]pyridin-3-yl)pyridin- 3-yl)methanol | [M + H]⁺ = 391.0 ¹H NMR (400 MHz, DMSO-d₆) δ ppm 8.54 (s, 1H), 8.07 (d, J = 2.4 Hz, 1H), 7.67-7.53 (m, 2H), 7.23 (s, 1H), 6.10 (s, 2H), 5.42- 5.17 (m, 1H), 4.40 (s, 2H), 3.92 (s, 3H), 1.30 (s, 9H). |
| 93 | 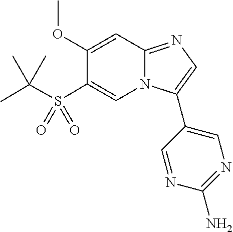 [SMILES: COc1cc2ncc(-c3cnc(N)nc3)n2cc1S(=O)(=O)C(C)(C)C] | 5-(6-(tert- butylsulfonyl)-7- methoxyimidazo[1,2- a]pyridin-3- yl)pyrimidin-2-amine | [M + H]⁺ = 362.1 ¹H NMR (400 MHz, DMSO-d₆) δ ppm 8.48 (s, 1H), 8.46 (s, 2H), 7.67 (s, 1H), 7.23 (s, 1H), 7.04 (s, 2H), 3.92 (s, 3H), 1.30 (s, 9H). |
| 94 | 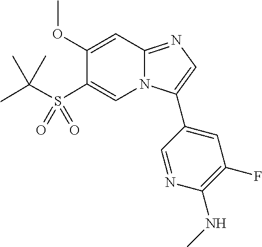 [SMILES: CNc1ncc(-c2cnc3cc(OC)c(S(=O)(=O)C(C)(C)C)cn23)cc1F] | 5-(6-(tert- butylsulfonyl)-7- methoxyimidazo[1,2- a]pyridin-3-yl)-3- fluoro-N- methylpyridin-2-amine | [M + H]⁺ = 393.1 ¹H NMR (400 MHz, DMSO-d₆) δ ppm 8.54 (s, 1H), 8.08 (s, 1H), 7.69-7.62 (m, 2H), 7.24 (s, 1H), 7.06-6.98 (m, 1H), 3.92 (s, 3H), 2.91 (d, J = 4.4 Hz, 3H), 1.30 (s, 9H). |
| 95 | 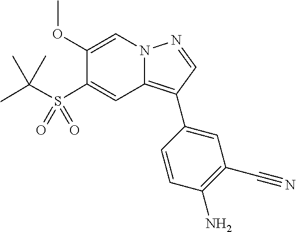 [SMILES: COc1cn2ncc(-c3ccc(N)c(C#N)c3)c2cc1S(=O)(=O)C(C)(C)C] | 2-amino-5-(5-(tert- butylsulfonyl)-6- methoxypyrazolo[1,5- a]pyridin-3- yl)benzonitrile | [M + H]⁺ = 385.3 ¹H NMR (400 MHz, DMSO-d₆) δ ppm 8.69 (s, 1H), 8.33 (s, 1H), 8.14 (s, 1H), 7.65 (d, J = 2.0 Hz, 1H), 7.58 (dd, J = 8.8, 2.4 Hz, 1H), 6.94 (d, J = 8.8 Hz, 1H), 6.22 (brs, 2H), 3.91 (s, 3H), 1.31 (s, 9H). |
| 96 | 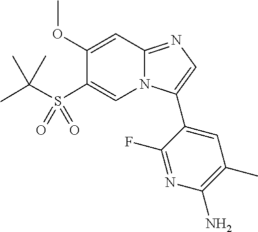 [SMILES: COc1cc2ncc(-c3cc(C)c(N)nc3F)n2cc1S(=O)(=O)C(C)(C)C] | 5-(6-(tert- butylsulfonyl)-7- methoxyimidazo[1,2- a]pyridin-3-yl)-6- fluoro-3- methylpyridin-2-amine | [M + H]⁺ = 393.2 ¹H NMR (400 MHz, DMSO-d₆) δ ppm 8.30 (d, J = 2.8 Hz, 1H), 7.63 (s, 1H), 7.59-7.50 (m, 1H), 7.25 (s, 1H), 6.57 (s, 2H), 3.93 (s, 3H), 2.09 (s, 3H), 1.30 (s, 9H). |
| 97 | 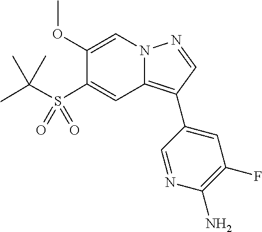 [SMILES: COc1cn2ncc(-c3cnc(N)c(F)c3)c2cc1S(=O)(=O)C(C)(C)C] | 5-(5-(tert- butylsulfonyl)-6- methoxypyrazolo[1,5- a]pyridin-3-yl)-3- fluoropyridin-2-amine | [M + H]⁺ = 379.2 ¹H NMR (400 MHz, DMSO-d₆) δ ppm 8.71 (s, 1H), 8.36 (s, 1H), 8.16 (s, 1H), 8.05 (s, 1H), 7.68 (dd, J = 12.40, 1.60 Hz, 1H), 6.37 (brs, 2H), 3.91 (s, 3H), 1.32 (s, 9H). |
| 98 | 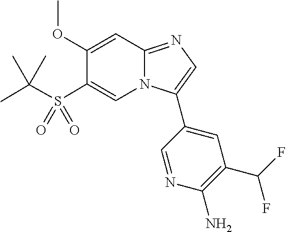 [SMILES: COc1cc2ncc(-c3cnc(N)c(C(F)F)c3)n2cc1S(=O)(=O)C(C)(C)C] | 5-(6-(tert- butylsulfonyl)-7- methoxyimidazo[1,2- a]pyridin-3-yl)-3- (difluoromethyl)pyridin- 2-amine | [M + H]⁺ = 411.0 ¹H NMR (400 MHz, DMSO-d₆) δ ppm 8.49 (s, 1H), 8.31 (s, 1H), 7.84 (s, 1H), 7.67 (s, 1H), 7.27-6.93 (m, 2H), 6.64 (s, 2H), 3.92 (s, 3H), 1.30 (s, 9H). |
| 99 | 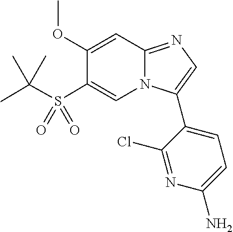 [SMILES: COc1cc2ncc(-c3ccc(N)nc3Cl)n2cc1S(=O)(=O)C(C)(C)C] | 5-(6-(tert- butylsulfonyl)-7- methoxyimidazo[1,2- a]pyridin-3-yl)-6- chloropyridin-2-amine | [M + H]⁺ = 395.0 ¹H NMR (400 MHz, DMSO-d₆) δ ppm 8.17 (s, 1H), 7.61 (s, 1H), 7.54 (d, J = 8.4 Hz, 1H), 7.27 (s, 1H), 6.84 (s, 2H), 6.55 (d, J = 8.0 Hz, 1H), 3.93 (s, 3H), 1.30 (s, 9H). |
| 100 | 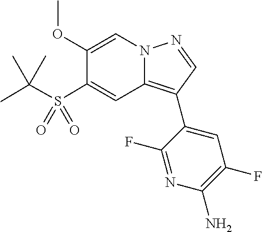 [SMILES: COc1cn2ncc(-c3cc(F)c(N)nc3F)c2cc1S(=O)(=O)C(C)(C)C] | 5-(5-(tert- butylsulfonyl)-6- methoxypyrazolo[1,5- a]pyridin-3-yl)-3,6- difluoropyridin-2- amine | [M + H]⁺ = 397.3 ¹H NMR (400 MHz, DMSO-d₆) δ ppm 8.71 (s, 1H), 8.27 (s, 1H), 8.10 (d, J = 1.6 Hz, 1H), 7.81 (dd, J = 10.8, 7.6 Hz, 1H), 6.79 (brs, 2H), 3.91 (s, 3H), 1.30 (s, 9H). |
| 101 | 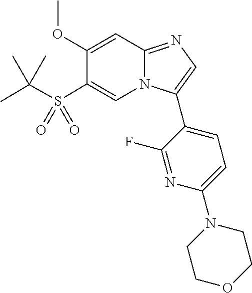 [SMILES: COc1cc2ncc(-c3ccc(N4CCOCC4)nc3F)n2cc1S(=O)(=O)C(C)(C)C] | 4-(5-(6-(tert- butylsulfonyl)-7- methoxyimidazo[1,2- a]pyridin-3-yl)-6- fluoropyridin-2- yl)morpholine | [M + H]⁺ = 449.1 ¹H NMR (400 MHz, DMSO-d₆) δ ppm 8.35 (d, J = 2.8 Hz, 1H), 7.95-7.82 (m, 1H), 7.69 (s, 1H), 7.28 (s, 1H), 6.93 (dd, J = 8.0, 2.0 Hz, 1H), 3.93 (s, 3H), 3.74- 3.67 (m, 4H), 3.58-3.52 (m, 4H), 1.30 (s, 9H). |
| 102 | 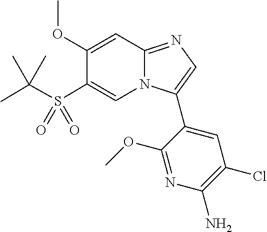 [SMILES: COc1cc2ncc(-c3cc(Cl)c(N)nc3OC)n2cc1S(=O)(=O)C(C)(C)C] | 5-(6-(tert- butylsulfonyl)-7- methoxyimidazo[1,2- a]pyridin-3-yl)-3- chloro-6- methoxypyridin-2- amine | [M + H]⁺ = 425.0 ¹H NMR (400 MHz, DMSO-d₆) δ ppm 8.18 (s, 1H), 7.64 (s, 1H), 7.58 (s, 1H), 7.23 (s, 1H), 6.70 (s, 2H), 3.92 (s, 3H), 3.74 (s, 3H), 1.31 (s, 9H). |
| 103 | 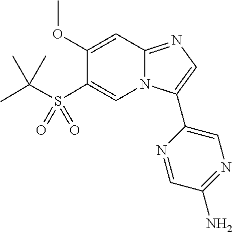 [SMILES: COc1cc2ncc(-c3cnc(N)cn3)n2cc1S(=O)(=O)C(C)(C)C] | 5-(6-(tert- butylsulfonyl)-7- methoxyimidazo[1,2- a]pyridin-3-yl)pyrazin- 2-amine | [M + H]⁺ = 362.1 ¹H NMR (400 MHz, DMSO-d₆) δ ppm 9.92 (s, 1H), 8.56 (s, 1H), 8.13 (s, 1H), 7.94 (s, 1H), 7.25 (s, 1H), 6.64 (s, 2H), 3.93 (s, 3H), 1.31 (s, 9H). |
| 104 | 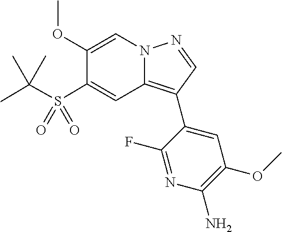 [SMILES: COc1cn2ncc(-c3cc(OC)c(N)nc3F)c2cc1S(=O)(=O)C(C)(C)C] | 5-(5-(tert- butylsulfonyl)-6- methoxypyrazolo[1,5- a]pyridin-3-yl)-6- fluoro-3- methoxypyridin-2- amine | [M + H]⁺ = 409.2 ¹H NMR (400 MHz, DMSO-d₆) δ ppm 8.70 (s, 1H), 8.29 (s, 1H), 8.10 (d, J = 1.6 Hz, 1H), 7.34 (d, J = 8.0 Hz, 1H), 6.33 (brs, 2H), 3.91 (s, 3H), 3.84 (s, 3H), 1.30 (s, 9H). |
| 105 | 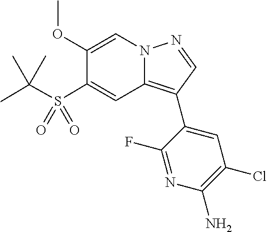 [SMILES: COc1cn2ncc(-c3cc(Cl)c(N)nc3F)c2cc1S(=O)(=O)C(C)(C)C] | 5-(5-(tert- butylsulfonyl)-6- methoxypyrazolo[1,5- a]pyridin-3-yl)-3- chloro-6-fluoropyridin- 2-amine | [M + H]⁺ = 413.0 ¹H NMR (400 MHz, DMSO-d₆) δ ppm 8.71 (s, 1H), 8.29 (s, 1H), 8.09 (s, 1H), 7.97 (d, J = 8.4 Hz, 1H), 6.87 (s, 2H), 3.91 (s, 3H), 1.30 (s, 9H). |
| 106 | 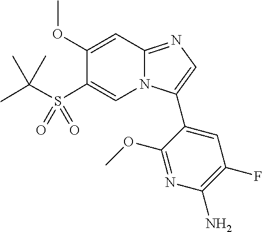 [SMILES: COc1cc2ncc(-c3cc(F)c(N)nc3OC)n2cc1S(=O)(=O)C(C)(C)C] | 5-(6-(tert- butylsulfonyl)-7- methoxyimidazo[1,2- a]pyridin-3-yl)-3- fluoro-6- methoxypyridin-2- amine | [M + H]⁺ = 409.0 ¹H NMR (400 MHz, DMSO-d₆) δ ppm 8.19 (s, 1H), 7.55 (s, 1H), 7.51 (d, J = 10.4 Hz, 1H), 7.22 (s, 1H), 6.62 (s, 2H), 3.92 (s, 3H), 3.72 (s, 3H), 1.31 (s, 9H). |
| 107 | 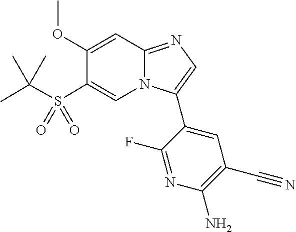 [SMILES: COc1cc2ncc(-c3cc(C#N)c(N)nc3F)n2cc1S(=O)(=O)C(C)(C)C] | 2-amino-5-(6-(tert- butylsulfonyl)-7- methoxyimidazo[1,2- a]pyridin-3-yl)-6- fluoronicotinonitrile | [M + H]⁺ = 404.0 ¹H NMR (400 MHz, DMSO-d₆) δ ppm 8.43 (d, J = 2.4 Hz, 1H), 8.29 (d, J = 9.2 Hz, 1H), 7.76- 7.70 (m, 2H), 7.69 (s, 1H), 7.26 (s, 1H), 3.93 (s, 3H), 1.29 (s, 9H). |
| 108 | 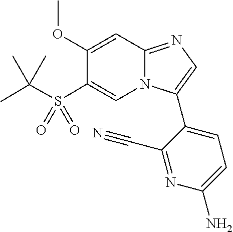 [SMILES: COc1cc2ncc(-c3ccc(N)nc3C#N)n2cc1S(=O)(=O)C(C)(C)C] | 6-amino-3-(6-(tert- butylsulfonyl)-7- methoxyimidazo[1,2- a]pyridin-3- yl)picolinonitrile | [M + H]⁺ = 386.1 ¹H NMR (400 MHz, DMSO-d₆) δ ppm 8.37 (s, 1H), 7.76 (s, 1H), 7.73 (d, J = 8.8 Hz, 1H), 7.30 (s, 1H), 6.98 (s, 2H), 6.88 (d, J = 8.8 Hz, 1H), 3.94 (s, 3H), 1.30 (s, 9H). |
| 109 | 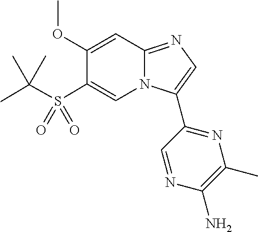 [SMILES: COc1cc2ncc(-c3cnc(N)c(C)n3)n2cc1S(=O)(=O)C(C)(C)C] | 5-(6-(tert- butylsulfonyl)-7- methoxyimidazo[1,2- a]pyridin-3-yl)-3- methylpyrazin-2-amine | [M + H]⁺ = 376.1 ¹H NMR (400 MHz, DMSO-d₆) δ ppm 10.17 (d, J = 2.4 Hz, 1H), 8.44 (s, 1H), 8.11 (d, J = 2.4 Hz, 1H), 7.23 (s, 1H), 6.42 (s, 2H), 3.93 (s, 3H), 2.39 (s, 3H), 1.33 (s, 9H). |
| 110 | 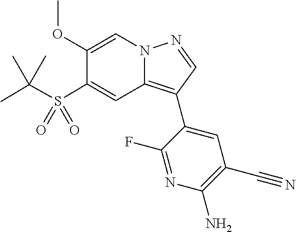 [SMILES: COc1cn2ncc(-c3cc(C#N)c(N)nc3F)c2cc1S(=O)(=O)C(C)(C)C] | 2-amino-5-(5-(tert- butylsulfonyl)-6- methoxypyrazolo[1,5- a]pyridin-3-yl)-6- fluoronicotinonitrile | [M + H]⁺ = 404.0 ¹H NMR (400 MHz, DMSO-d₆) δ ppm 8.71 (s, 1H), 8.33-8.29 (m, 1H), 8.29 (s, 1H), 8.12 (s, 1H), 7.49 (s, 2H), 3.91 (s, 3H), 1.30 (s, 9H). |
| 111 | 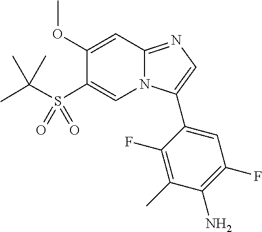 [SMILES: COc1cc2ncc(-c3cc(F)c(N)c(C)c3F)n2cc1S(=O)(=O)C(C)(C)C] | 4-(6-(tert- butylsulfonyl)-7- methoxyimidazo[1,2- a]pyridin-3-yl)-3,6- difluoro-2- methylaniline | [M + H]⁺ = 410.1 ¹H NMR (400 MHz, DMSO-d₆) δ ppm 8.30 (d, J = 2.8 Hz, 1H), 7.62 (s, 1H), 7.25 (s, 1H), 7.19- 7.11 (m, 1 H), 5.65 (s, 2H), 3.93 (s, 3H), 2.10 (d, J = 2.0 Hz, 3H), 1.29 (s, 9H). |
| 112 | 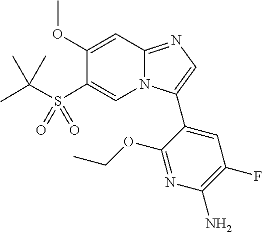 [SMILES: CCOc1nc(N)c(F)cc1-c1cnc2cc(OC)c(S(=O)(=O)C(C)(C)C)cn12] | 5-(6-(tert- butylsulfonyl)-7- methoxyimidazo[1,2- a]pyridin-3-yl)-6- ethoxy-3-fluoropyridin- 2-amine | [M + H]⁺ = 423.1 ¹H NMR (400 MHz, DMSO-d₆) δ ppm 8.26 (s, 1H), 7.55 (s, 1H), 7.48 (d, J = 10.8 Hz, 1H), 7.22 (s, 1H), 6.57 (s, 2H), 4.26- 4.19 (m, 2H), 3.92 (s, 3H), 1.29 (s, 9H), 1.16 (t, J = 6.8 Hz, 3H). |
| 113 | 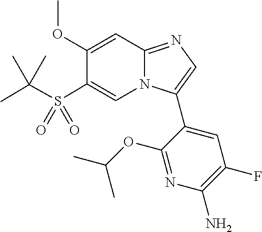 [SMILES: COc1cc2ncc(-c3cc(F)c(N)nc3OC(C)C)n2cc1S(=O)(=O)C(C)(C)C] | 5-(6-(tert- butylsulfonyl)-7- methoxyimidazo[1,2- a]pyridin-3-yl)-3- fluoro-6- isopropoxypyridin-2- amine | [M + H]⁺ = 437.2 ¹H NMR (400 MHz, DMSO-d₆) δ ppm 8.29 (s, 1H), 7.53 (s, 1H), 7.45 (d, J = 10.8 Hz, 1H), 7.22 (s, 1H), 6.54 (s, 2H), 5.18- 5.08 (m, 1H), 3.92 (s, 3H), 1.28 (s, 9H), 1.17 (d, J = 6.4 Hz, 6H). |
| 114 | 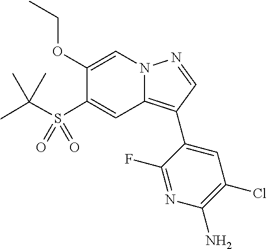 [SMILES: CCOc1cn2ncc(-c3cc(Cl)c(N)nc3F)c2cc1S(=O)(=O)C(C)(C)C] | 5-(5-(tert- butylsulfonyl)-6- ethoxypyrazolo[1,5- a]pyridin-3-yl)-3- chloro-6-fluoropyridin- 2-amine | [M + H]⁺ = 426.9 ¹H NMR (400 MHz, DMSO-d₆) δ ppm 8.69 (s, 1H), 8.28 (s, 1H), 8.09 (d, J = 1.2 Hz, 1H), 7.97 (d, J = 8.8 Hz, 1H), 6.88 (s, 2H), 4.16 (q, J = 6.8 Hz, 2H), 1.40-1.33 (m, 3H), 1.32 (s, 9H). |
| 115 | 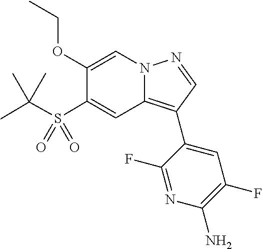 [SMILES: CCOc1cn2ncc(-c3cc(F)c(N)nc3F)c2cc1S(=O)(=O)C(C)(C)C] | 5-(5-(tert- butylsulfonyl)-6- ethoxypyrazolo[1,5- a]pyridin-3-yl)-3,6- difluoropyridin-2- amine | [M + H]⁺ = 410.9 ¹H NMR (400 MHz, DMSO-d₆) δ ppm 8.69 (s, 1H), 8.26 (s, 1H), 8.11 (s, 1H), 7.85-7.76 (m, 1H), 6.80 (s, 2H), 4.16 (q, J = 6.8 Hz, 2H), 1.37 (t, J = 7.2 Hz, 3H), 1.32 (s, 9H). |
| 116 | 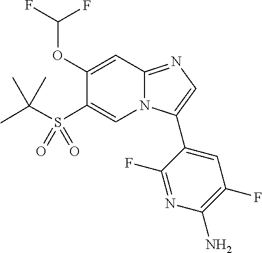 [SMILES: CC(C)(C)S(=O)(=O)c1cn2c(-c3cc(F)c(N)nc3F)cnc2cc1OC(F)F] | 5-(6-(tert- butylsulfonyl)-7- (difluoromethoxy)imid azo[1,2-a]pyridin-3- yl)-3,6-difluoropyridin- 2-amine | [M + H]⁺ = 432.8 ¹H NMR (400 MHz, DMSO-d₆) δ ppm 8.49 (d, J = 2.0 Hz, 1H), 7.84 (s, 1H), 7.82-7.71 (dd, J= 10.4, 7.6 Hz, 1H), 7.65-7.20 (m, 2H), 7.11 (s, 2H), 1.33 (s, 9H). |
| 117 | 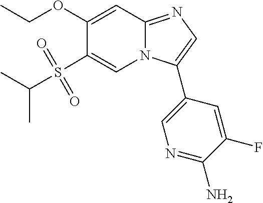 [SMILES: CCOc1cc2ncc(-c3cnc(N)c(F)c3)n2cc1S(=O)(=O)C(C)C] | 5-[6-ethoxy-5- (isopropylsulfonyl)- 1,3a-diaza-3-indenyl]- 3-fluoro-2- pyridylamine | [M + H]⁺ = 379.0 ¹H NMR (400 MHz, DMSO-d₆) δ ppm 8.32 (d, J = 2.0 Hz, 1H), 8.21 (s, 0.12H), 7.72-7.64 (m, 1H), 7.61 (s, 1H), 7.27 (s, 1H), 6.79 (s, 2H), 6.49 (dd, J = 2.0, 8.4 Hz, 1H), 4.26 (q, J = 6.8 Hz, 2H), 3.78-3.65 (m, 1H), 1.40 (t, J = 6.8 Hz, 3H), 1.21 (d, J = 6.8 Hz, 6H). |
| 118 | 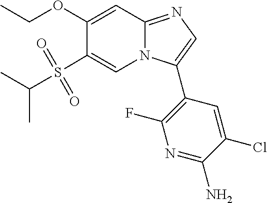 [SMILES: CCOc1cc2ncc(-c3cc(Cl)c(N)nc3F)n2cc1S(=O)(=O)C(C)C] | 3-chloro-5-[6-ethoxy- 5-(isopropylsulfonyl)- 1,3a-diaza-3-indenyl]- 6-fluoro-2- pyridylamine | [M + H]⁺ = 413.0 ¹H NMR (400 MHz, DMSO-d₆) δ ppm 8.38 (d, J = 2.4 Hz, 1H), 7.97 (d, J = 8.4 Hz, 1H), 7.67 (s, 1H), 7.27 (s, 1H), 7.12 (br s, 2H), 4.27 (q, J = 7.2 Hz, 2H), 3.74-3.67 (m, 1H), 1.40 (t, J = 7.2 Hz, 3H), 1.21 (d, J = 6.8 Hz, 6H |
| 119 | 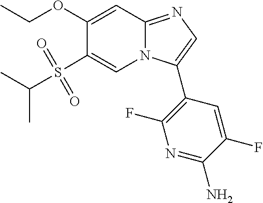 [SMILES: CCOc1cc2ncc(-c3cc(F)c(N)nc3F)n2cc1S(=O)(=O)C(C)C] | 5-[6-ethoxy-5- (isopropylsulfonyl)- 1,3a-diaza-3-indenyl]- 3,6-difluoro-2- pyridylamine | [M + H]⁺ = 397.0 ¹H NMR (400 MHz, DMSO-d₆) δ ppm 8.38 (s, 1H), 7.85-7.76 (m, 1H), 7.65 (s, 1H), 7.28 (s, 1H), 7.05 (s, 2H), 4.32-4.20 (m, 2H), 3.81-3.60 (m, 1H), 1.40 (t, J = 6.8 Hz, 3H), 1.21 (d, J = 6.8 Hz, 6H). |
| 120 | 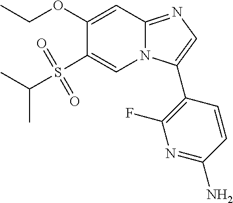 [SMILES: CCOc1cc2ncc(-c3ccc(N)nc3F)n2cc1S(=O)(=O)C(C)C] | 5-[6-ethoxy-5- (isopropylsulfonyl)- 1,3a-diaza-3-indenyl]- 6-fluoro-2- pyridylamine | [M + H]⁺ = 379.0 ¹H NMR (400 MHz, DMSO-d₆) δ ppm 8.32 (d, J = 2.0 Hz, 1H), 8.21 (s, 0.12H), 7.72-7.64 (m, 1H), 7.61 (s, 1H), 7.27 (s, 1H), 6.79 (s, 2H), 6.49 (dd, J = 2.0, 8.4 Hz, 1H), 4.26 (q, J = 6.8 Hz, 2H), 3.78-3.65 (m, 1H), 1.40 (t, J = 6.8 Hz, 3H), 1.21 (d, J = 6.8 Hz, 6H). |
| 121 | 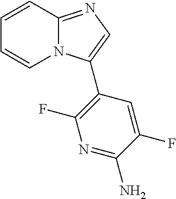 [SMILES: Nc1nc(F)c(-c2cnc3ccccn23)cc1F] | 5-(1,3a-diaza-3- indenyl)-3,6-difluoro- 2-pyridylamine | [M + H]⁺ = 246.9 ¹H NMR (400 MHz, DMSO-d₆) δ ppm 8.20 (d, J = 6.0 Hz, 1H), 7.80-7.72 (m, 1H), 7.65 (s, 2H), 7.36-7.27 (m, 1H), 7.01-6.90 (m, 3H). |
| 122 | 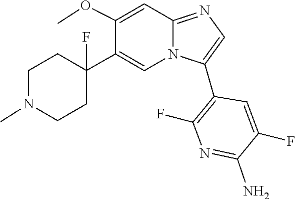 [SMILES: COc1cc2ncc(-c3cc(F)c(N)nc3F)n2cc1C1(F)CCN(C)CC1] | 3,6-difluoro-5-[5-(4- fluoro-1-methyl-4- piperidyl)-6-methoxy- 1,3a-diaza-3-indenyl]- 2-pyridylamine | [M + H]⁺ = 392.0 ¹H NMR (400 MHz, DMSO-d₆) δ ppm 7.88 (s, 1H), 7.82-7.67 (m, 1H), 7.50 (s, 1H), 7.12 (s, 1H), 6.97 (s, 2H), 3.91 (s, 3H), 2.72-2.64 (m, 2H), 2.63-2.55 (m, 2H), 2.28-2.12 (m, 5H), 1.81-1.69 (m, 2H). |
| 123 | 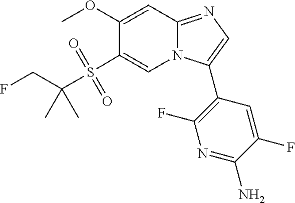 [SMILES: COc1cc2ncc(-c3cc(F)c(N)nc3F)n2cc1S(=O)(=O)C(C)(C)CF] | 3,6-difluoro-5-[5-(2- fluoro-1,1- dimethylethylsulfonyl)- 6-methoxy-1,3a-diaza- 3-indenyl]-2- pyridylamine | [M + H]⁺ = 415.0 ¹H NMR (400 MHz, DMSO-d₆) δ ppm 8.41 (d, J = 2.0 Hz, 1H), 7.80 (dd, J = 7.6, 10.4 Hz, 1H), 7.66 (s, 1H), 7.29 (s, 1H), 7.07 (s, 2H), 4.01 (s, 3H), 3.95 (d, J = 17.6 Hz, 2H), 1.50 (s, 3H), 1.44 (s, 3H). |
| Int. 77 | 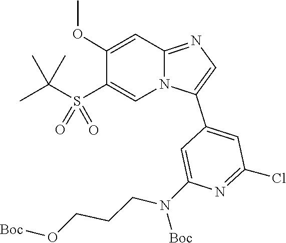 [SMILES: COc1cc2ncc(-c3cc(Cl)nc(N(CCCOC(=O)OC(C)(C)C)C(=O)OC(C)(C)C)c3)n2cc1S(=O)(=O)C(C)(C)C] | tert-butyl(3-((tert- butoxycarbonyl)oxy) propyl)(4-(6-(tert- butylsulfonyl)-7- methoxyimidazo[1,2- a]pyridin-3-yl)-6- chloropyridin-2- yl)carbamate | [M + H]⁺ = 653.7 |
| 124 | 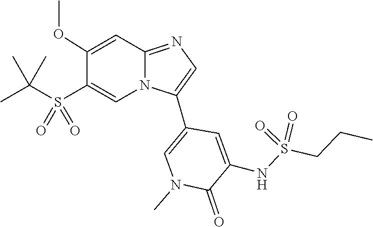 [SMILES: CCCS(=O)(=O)Nc1cc(-c2cnc3cc(OC)c(S(=O)(=O)C(C)(C)C)cn23)cn(C)c1=O] | N-(5-(6-(tert- butylsulfonyl)-7- methoxyimidazo[1,2- a]pyridin-3-yl)-1- methyl-2-oxo-1,2- dihydropyridin-3- yl)propane-1- sulfonamide | [M + H]⁺ = 497.1 ¹H NMR (400 MHz, DMSO-d₆) δ ppm 8.51 (s, 1H), 7.81 (s, 1H), 7.62 (s, 1H), 7.51 (s, 1H), 7.24 (s, 1H), 3.92 (s, 3H), 3.57 (s, 3H), 3.23-3.15 (m, 2H), 1.77-1.65 (m, 2H), 1.31 (s, 9H), 0.96 (t, J =7.2 Hz, 3H). |
| 125 | 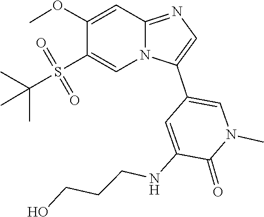 [SMILES: COc1cc2ncc(-c3cc(NCCCO)c(=O)n(C)c3)n2cc1S(=O)(=O)C(C)(C)C] | 5-(6-(tert- butylsulfonyl)-7- methoxyimidazo[1,2- a]pyridin-3-yl)-3-((3- hydroxypropyl)amino)- 1-methylpyridin-2(1H)- one | [M + H]⁺ = 449.0 ¹H NMR (400 MHz, DMSO-d₆) δ ppm 8.57 (s, 1H), 7.58 (s, 1H), 7.24-7.18 (m, 2H), 6.36 (d, J = 2.0 Hz, 1H), 5.79 (t, J = 5.6 Hz, 1H), 4.47 (t, J = 5.2 Hz, 1H), 3.92 (s, 3H), 3.52 (s, 3H), 3.49-3.41 (m, 2H), 3.14-3.04 (m, 2H), 1.75-1.635 (m, 2H), 1.31 (s, 9H). |
| 126 | 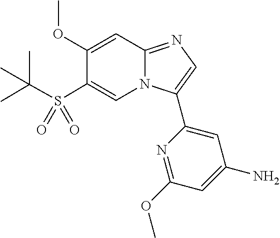 [SMILES: COc1cc(N)cc(-c2cnc3cc(OC)c(S(=O)(=O)C(C)(C)C)cn23)n1] | 2-(6-(tert- butylsulfonyl)-7- methoxyimidazo[1,2- a]pyridin-3-yl)-6- methoxypyridin-4- amine | [M + H]⁺ = 391.0 ¹H NMR (400 MHz, DMSO-d₆) δ ppm 10.53 (s, 1H), 8.08 (s, 1H), 7.26 (s, 1H), 6.74 (s, 1H), 6.10 (s, 2H), 5.82 (d, J = 1.6 Hz, 1H), 3.93 (s, 3H), 3.90 (s, 3H), 1.31 (s, 9H). |
| 127 | 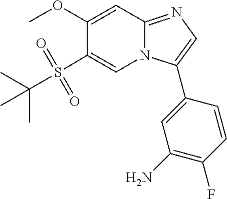 [SMILES: COc1cc2ncc(-c3ccc(F)c(N)c3)n2cc1S(=O)(=O)C(C)(C)C] | 5-(6-(tert- butylsulfonyl)-7- methoxyimidazo[1,2- a]pyridin-3-yl)-2- fluoroaniline | [M + H]⁺ = 378.0 ¹H NMR (400 MHz, DMSO-d₆) δ ppm 8.66 (s, 1H), 7.62 (s, 1H), 7.24 (s, 1H), 7.22-7.17 (m, 1H), 6.92 (dd, J = 2.4, 8.8 Hz, 1H), 6.74-6.66 (m, 1H), 5.44 (s, 2H), 3.92 (s, 3H), 1.30 (s, 9H). |
| 128 | 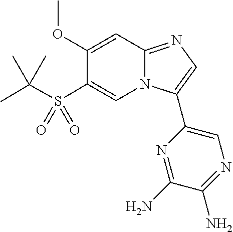 [SMILES: COc1cc2ncc(-c3cnc(N)c(N)n3)n2cc1S(=O)(=O)C(C)(C)C] | 5-(6-(tert- butylsulfonyl)-7- methoxyimidazo[1,2- a]pyridin-3- yl)pyrazine-2,3- diamine | [M + H]⁺ = 377.1 ¹H NMR (400 MHz, DMSO-d₆) δ ppm 9.75 (s, 1H), 8.20 (s, 0.51H), 7.88 (s, 1H), 7.71 (s, 1H), 7.20 (s, 1H), 6.21 (s, 2H), 6.01 (s, 2H), 3.91 (s, 3H), 1.33 (s, 9H). |
| 129 | 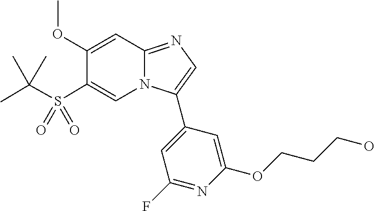 [SMILES: COc1cc2ncc(-c3cc(F)nc(OCCCO)c3)n2cc1S(=O)(=O)C(C)(C)C] | 3-({6-fluoro-4-[7- methoxy-6-(2- methylpropane-2- sulfonyl)imidazo[1,2- a]pyridin-3-yl]pyridin- 2-yl}oxy)propan-1-ol | [M + H]⁺ = 438.2 ¹H NMR (400 MHz, DMSO- d₆) δ ppm 8.84 (s, 1 H), 8.08 (s, 1 H), 7.34 (s, 1 H), 6.85−7.06 (m, 2 H), 4.54−4.64 (m, 1 H), 4.27−4.37 (m, 2 H), 3.92−3.99 (m, 3 H), 3.49−3.60 (m, 2 H), 1.82−1.95 (m, 2 H), 1.19−1.39 (m, 9 H) |
| 130 | 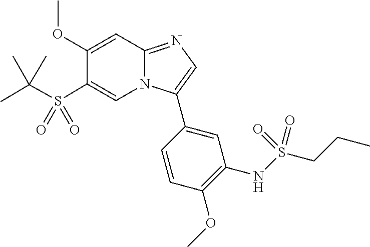 [SMILES: CCCS(=O)(=O)Nc1cc(-c2cnc3cc(OC)c(S(=O)(=O)C(C)(C)C)cn23)ccc1OC] | N-{2-methoxy-5-[7- methoxy-6-(2- methylpropane-2- sulfonyl)imidazo[1,2- a]pyridin-3- yl]phenyl}propane-1- sulfonamide | [M + H]⁺ = 496.7 ¹H NMR (400 MHz, DMSO-d₆) δ ppm 9.13 (s, 1 H), 8.65 (s, 1 H), 7.68 (s, 1 H), 7.49 (d, J = 2.28 Hz, 1 H), 7.41 (dd, J = 8.36, 2.03 Hz, 1 H), 7.28 (d, J = 8.62 Hz, 1 H), 7.26 (s, 1 H), 3.93 (m, 3 H), 3.90 (m, 3 H), 3.04−3.11 (m, 2 H), 1.69−1.81 (m, 2 H), 1.32 (s, 9 H), 0.96 (t, J = 7.35 Hz, 3 H) |
| 131 | 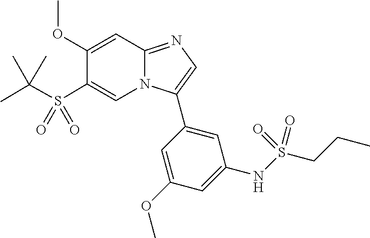 [SMILES: CCCS(=O)(=O)Nc1cc(OC)cc(-c2cnc3cc(OC)c(S(=O)(=O)C(C)(C)C)cn23)c1] | N-{3-methoxy-5-[7- methoxy-6-(2- methylpropane-2- sulfonyl)imidazo[1,2- a]pyridin-3- yl]phenyl}propane-1- sulfonamide | [M + H]⁺ = 496.5 ¹H NMR (400 MHz, DMSO-d₆) δ ppm 10.07 (s, 1 H), 8.76 (s, 1 H), 7.78 (s, 1 H), 7.28 (s, 1 H), 7.02 (t, J = 1.65 Hz, 1 H), 6.91 (dd, J = 2.28, 1.27 Hz, 1 H), 6.84 (t, J = 2.15 Hz, 1 H), 3.93 (s, 3 H), 3.79 (s, 3 H), 3.13- 3.19 (m, 2 H), 1.71 (sxt, J = 7.50 Hz, 2 H), 1.32 (s, 9 H), 0.96 (t, J = 7.48 Hz, 3 H) |
| 132 | 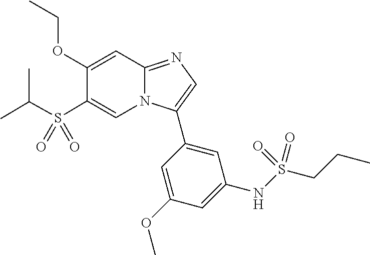 [SMILES: CCCS(=O)(=O)Nc1cc(OC)cc(-c2cnc3cc(OCC)c(S(=O)(=O)C(C)C)cn23)c1] | N-{3-[7-ethoxy-6- (propane-2- sulfonyl)imidazo[1,2- a]pyridin-3-yl]-5- methoxyphenyl}propane- 1-sulfonamide | [M + H]⁺ = 496.5 ¹H NMR (400 MHz, DMSO-d₆) δ ppm 10.08 (br s, 1 H), 8.77 (s, 1 H), 7.76 (s, 1 H), 7.29 (s, 1 H), 7.01 (s, 1 H), 6.94 (s, 1 H), 6.85 (t, J = 2.03 Hz, 1 H), 4.19- 4.35 (m, 2 H), 3.80 (s, 3 H), 3.62−3.78 (m, 1 H), 3.11−3.22 (m, 2 H), 1.71 (sxt, J = 7.50 Hz, 2 H), 1.40 (t, J = 6.97 Hz, 3 H), 1.22 (d, J = 6.84 Hz, 6 H), 0.96 (t, J = 7.35 Hz, 3 H) |
| 133 | 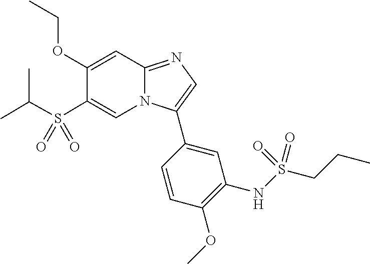 [SMILES: CCCS(=O)(=O)Nc1cc(-c2cnc3cc(OCC)c(S(=O)(=O)C(C)C)cn23)ccc1OC] | N-{5-[7-ethoxy-6- (propane-2- sulfonyl)imidazo[1,2- a]pyridin-3-yl]-2- methoxyphenyl}propane- 1-sulfonamide | [M + H]⁺ = 496.7 ¹H NMR (400 MHz, DMSO-d₆) δ ppm 9.14 (s, 1 H), 8.66 (s, 1 H), 7.67 (s, 1 H), 7.43−7.50 (m, 2 H), 7.26−7.30 (m, 2 H), 4.27 (q, J = 7.10 Hz, 2 H), 3.90 (s, 3 H), 3.71 (sept, J = 6.84 Hz, 1 H), 3.04−3.11 (m, 2 H), 1.70 − 1.82 (m, 2 H), 1.40 (t, J = 6.97 Hz, 3 H), 1.22 (d, J = 6.84 Hz, 6 H), 0.97 (t, J = 7.48 Hz, 3 H) |
| 134 | 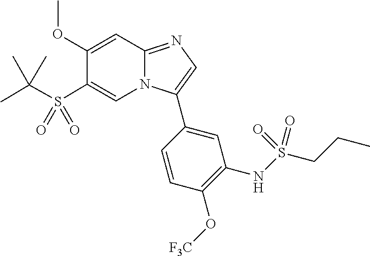 [SMILES: CCCS(=O)(=O)Nc1cc(-c2cnc3cc(OC)c(S(=O)(=O)C(C)(C)C)cn23)ccc1OC(F)(F)F] | N-{5-[7-methoxy-6-(2- methylpropane-2- sulfonyl)imidazo[1,2- a]pyridin-3-yl]-2- (trifluoromethoxy) phenyl}propane-1- sulfonamide | [M + H]⁺ = 550.5 ¹H NMR (400 MHz, DMSO- d₆) δ ppm 10.00 (s, 1H), 8.72 (s, 1 H), 7.82 (s, 1H), 7.75 (d, J = 2.03 Hz, 1H), 7.61 (dd, J = 8.49, 1.39 Hz, 1H), 7.49 (dd, J = 8.49, 1.65 Hz, 1H), 7.30 (s, 1H), 3.94 (s, 3H), 3.17−3.24 (m, 2H), 1.70−1.82 (m, 2H), 1.33 (s, 9H), 0.97 (t, J = 7.35 Hz, 3H) |
| 135 | 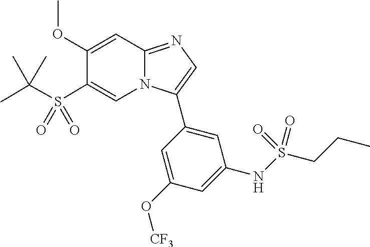 [SMILES: CCCS(=O)(=O)Nc1cc(OC(F)(F)F)cc(-c2cnc3cc(OC)c(S(=O)(=O)C(C)(C)C)cn23)c1] | N-{3-[7-methoxy-6-(2- methylpropane-2- sulfonyl)imidazo[1,2- a]pyridin-3-yl]-5- (trifluoromethoxy) phenyl}propane-1- sulfonamide | [M + H]⁺ = 550.5 ¹H NMR (400 MHz, DMSO-d₆) δ ppm 10.44 (s, 1H), 8.73 (s, 1H), 7.86 (s, 1H), 7.43 (t, J = 1.52 Hz, 1H), 7.34 (s, 1H), 7.30 (s, 1H), 7.19 (s, 1H), 3.94 (s, 3H), 3.21−3.27 (m, 2H), 1.72 (sxt, J = 7.50 Hz, 2H), 1.33 (s, 9H), 0.96 (t, J = 7.35 Hz, 3H) |
| 136 | 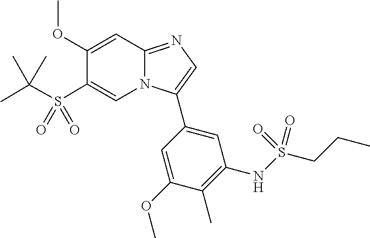 [SMILES: CCCS(=O)(=O)Nc1cc(-c2cnc3cc(OC)c(S(=O)(=O)C(C)(C)C)cn23)cc(OC)c1C] | N-(5-(6-(tert- Butylsulfonyl)-7- methoxyimidazo[1,2- a]pyridin-3-yl)-3- methoxy-2- methylphenyl)propane- 1-sulfonamide | [M + H]⁺ = 510.4 ¹H NMR (400 MHz, DMSO-d₆) δ ppm 9.27 (br s, 1 H), 8.77 (s, 1 H), 7.79 (s, 1 H), 7.27 (s, 1 H), 7.14−7.11 (m, 2 H), 3.93 (s, 3 H), 3.84 (s, 3 H), 3.07- 3.13 (m, 2 H), 2.20 (s, 3 H), 1.68−1.79 (m, 2 H), 1.32 (s, 9 H), 0.94−1.01 (m, 3 H) |
| 137 | 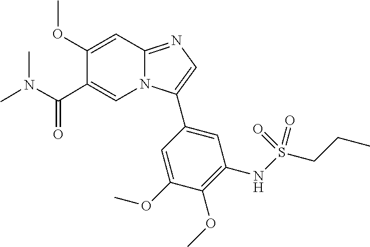 [SMILES: CCCS(=O)(=O)Nc1cc(-c2cnc3cc(OC)c(C(=O)N(C)C)cn23)cc(OC)c1OC] | 3-[3,4-dimethoxy-5- (propane-1- sulfonamido)phenyl]-7- methoxy-N,N- dimethylimidazo[1,2- a]pyridine-6- carboxamide | [M + H]⁺ = 477.5 ¹H NMR (400 MHz, DMSO-d₆) δ ppm 9.27 (s, 1 H), 8.31 (s, 1 H), 7.62 (s, 1 H), 7.13 (s, 2 H), 7.04 (d, J = 1.77 Hz, 1 H), 3.89 (s, 3 H), 3.88 (s, 3 H), 3.80 (s, 3 H), 3.08−3.17 (m, 2 H), 2.96 (s, 3 H), 2.84 (s, 3 H), 1.66- 1.80 (m, 2 H), 0.96 (t, J = 7.48 Hz, 3 H) |
| 138 | 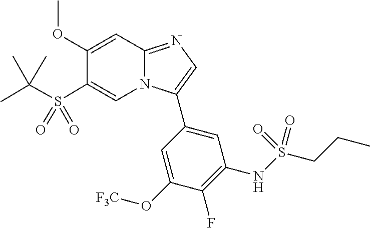 [SMILES: CCCS(=O)(=O)Nc1cc(-c2cnc3cc(OC)c(S(=O)(=O)C(C)(C)C)cn23)cc(OC(F)(F)F)c1F] | N-{2-fluoro-5-[7- methoxy-6-(2- methylpropane-2- sulfonyl)imidazo[1,2- a]pyridin-3-yl]-3- (trifluoromethoxy) phenyl}propane-1- sulfonamide | [M + H]⁺ = 568.1 ¹H NMR (400 MHz, DMSO-d₆) δ ppm 10.25 (s, 1 H), 8.68 (s, 1 H), 7.84 (s, 1 H), 7.70−7.65 (m, 2 H), 7.29 (s, 1 H), 3.94 (s, 3 H), 3.19−3.29 (m, 2 H), 1.69 − 1.83 (m, 2 H), 1.33 (s, 9 H), 0.98 (t, J = 7.35 Hz, 3 H) |
| 139 | 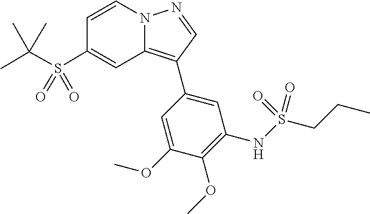 [SMILES: CCCS(=O)(=O)Nc1cc(-c2cnn3ccc(S(=O)(=O)C(C)(C)C)cc23)cc(OC)c1OC] | N-(5-(5-(tert- butylsulfonyl)pyrazolo [1,5-a]pyridin-3-yl)-2,3- dimethoxyphenyl) propane-1-sulfonamide | [M + H]⁺ = 496.33 ¹H NMR (400 MHz, DMSO-d₆) δ ppm 9.20 (brs, 1H), 8.94 (dd, J = 7.25, 0.75 Hz, 1H), 8.58 (s, 1H), 8.25 (d, J = 1.00 Hz, 1H), 7.28 (d, J = 2.00 Hz, 1H), 7.18 (dd, J = 7.38, 1.88 Hz, 1H), 7.06 (brs, 1H), 3.89 (s, 3H), 3.78 (s, 3H), 3.07 (t, J = 7.20 Hz, 2H), 1.77-1.71 (m, 2H), 1.33 (s, 9H), 0.95 (t, J = 7.50 Hz, 3H) |
| 140 | 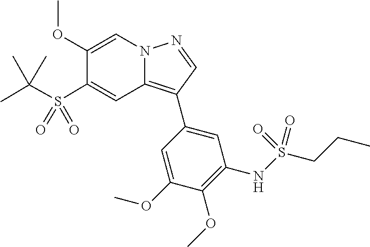 [SMILES: CCCS(=O)(=O)Nc1cc(-c2cnn3cc(OC)c(S(=O)(=O)C(C)(C)C)cc23)cc(OC)c1OC] | N-(5-(5-(tert- butylsulfonyl)-6- methoxypyrazolo[1,5- a]pyridin-3-yl)-2,3- dimethoxyphenyl) propane-1-sulfonamide | [M + H]⁺ = 526.3 ¹H NMR (400 MHz, DMSO-d₆) δ ppm 9.20 (brs, 1H), 8.71 (s, 1H), 8.36 (s, 1H), 8.28 (s, 1H), 7.23 (d, J = 2.00 Hz, 1H), 6.85 (brs, 1H), 3.91 (s, 3H), 3.84 (s, 3H), 3.73 (s, 3H), 2.98 (brs, 2H), 1.71 (q, J = 7.60 Hz, 2H), 1.32 (s, 9H), 0.94 (t, J = 7.60 Hz, 3H) |
| 141 | 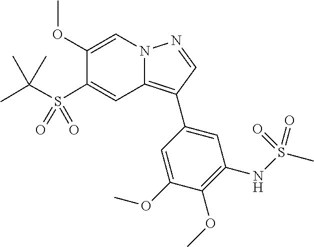 [SMILES: COc1cn2ncc(-c3cc(NS(C)(=O)=O)c(OC)c(OC)c3)c2cc1S(=O)(=O)C(C)(C)C] | N-(5-(5-(tert- butylsulfonyl)-6- methoxypyrazolo[1,5- a]pyridin-3-yl)-2,3- dimethoxyphenyl) methanesulfonamide | [M − H]⁻ = 496.46 ¹H NMR (400 MHz, DMSO-d₆) δ ppm 8.66 (s, 1H), 8.30 (s, 1H), 8.23 (s, 1H), 7.19 (d, J = 2.00 Hz, 1H), 6.42 (s, 1H), 3.90 (s, 3H), 3.75 (s, 3H), 3.64 (s, 3H), 2.60 (s, 3H), 1.32 (s, 9H) |
| 142 | 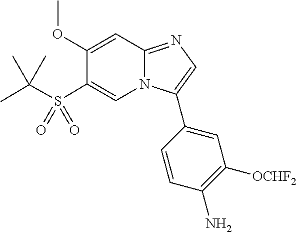 [SMILES: COc1cc2ncc(-c3ccc(N)c(OC(F)F)c3)n2cc1S(=O)(=O)C(C)(C)C] | 4-(6-(tert- butylsulfonyl)-7- methoxyimidazo[1,2- a]pyridin-3-yl)-2- (difluoromethoxy)aniline | [M + H]⁺ = 426.0 ¹H NMR (400 MHz, DMSO-d₆) δ ppm 8.61 (s, 1H), 7.59 (s, 1H), 7.29-6.89 (m, 5H), 5.48 (s, 2H), 3.92 (s, 3H), 1.30 (s, 9H). |
| 143 | 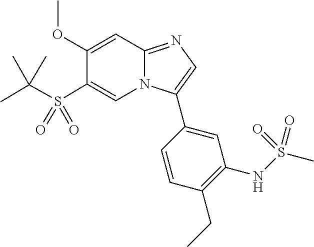 [SMILES: CCc1ccc(-c2cnc3cc(OC)c(S(=O)(=O)C(C)(C)C)cn23)cc1NS(C)(=O)=O] | N-(5-(6-(tert- butylsulfonyl)-7- methoxyimidazo[1,2-a] pyridin-3-yl)-2- ethylphenyl) methanesulfonamide | [M + H]⁺ = 466.1 ¹H NMR (400 MHz, DMSO-d₆) δ ppm 9.34-9.25 (m, 1H), 8.72 (s, 1H), 7.75 (s, 1H), 7.51-7.47 (m, 1H), 7.45 (s, 1H), 7.44-7.42 (m, 1H), 7.27 (s, 1H), 3.93 (s, 3H), 3.07 (s, 3H), 2.81-2.75 (m, 2H), 1.32 (s, 9H), 1.21(t, J = 7.6 Hz, 3H). |
| 144 | 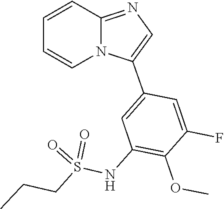 [SMILES: CCCS(=O)(=O)Nc1cc(-c2cnc3ccccn23)cc(F)c1OC] | N-(3-fluoro-5- (imidazo[1,2-a]pyridin- 3-yl)-2- methoxyphenyl) propane-1-sulfonamide | [M + H]⁺ = 363.8 ¹H NMR (400 MHz, DMSO-d₆) δ ppm 9.59 (s, 1H), 8.53 (d, J = 6.8 Hz, 1H), 7.78 (s, 1H), 7.67 (d, J = 8.8 Hz, 1H), 7.49-7.37 (m, 2H), 7.39-7.25 (m, 1H), 7.00 (t, J = 6.8 Hz, 1H), 3.93 (s, 3H), 3.25-3.11 (m, 2H), 1.83- 1.68 (m, 2H), 0.98 (t, J = 7.6 Hz, 3H). |
| 145 | 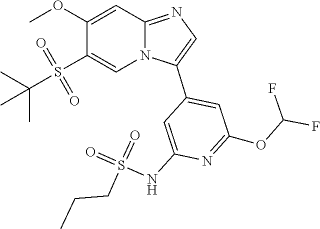 [SMILES: CCCS(=O)(=O)Nc1cc(-c2cnc3cc(OC)c(S(=O)(=O)C(C)(C)C)cn23)cc(OC(F)F)n1] | N-(4-(6-(tert- butylsulfonyl)-7- methoxyimidazo[1,2- a]pyridin-3-yl)-6- (difluoromethoxy) pyridin-2-yl)propane-1- sulfonamide | [M + H]⁺ = 533.2 ¹H NMR (400 MHz, DMSO-d₆) δ ppm 11.13-11.04 (m, 1H), 8.84 (s, 1H), 8.04 (s, 1H), 7.56 (t, J = 73.2 Hz, 1H), 7.34 (s, 1H), 6.99-6.93 (m, 2H), 3.95 (s, 3H), 3.53-3.42 (m, 2H), 1.82- 1.68 (m, 2H), 1.34 (s, 9H), 1.00 (t, J = 7.2 Hz, 3H). |
| 146 | 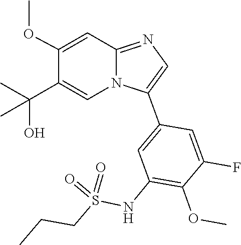 [SMILES: CCCS(=O)(=O)Nc1cc(-c2cnc3cc(OC)c(C(C)(C)O)cn23)cc(F)c1OC] | N-(3-fluoro-5-(6-(2- hydroxypropan-2-yl)-7- methoxyimidazo[1,2- a]pyridin-3-yl)-2- methoxyphenyl)propane- 1-sulfonamide | [M + H]⁺ = 452.2 ¹H NMR (400 MHz, DMSO-d₆) δ ppm 9.56 (s, 1H), 8.50 (s, 1H), 7.59 (s, 1H), 7.41-7.34 (m, 2H), 7.04 (s, 1H), 5.29 (s, 1H), 3.95-3.89 (m, 6H), 3.22-3.13 (m, 2H), 1.84-1.69 (m, 2H), 1.51 (s, 6H), 0.99 (t, J = 7.6 Hz, 3H). |
| 147 | 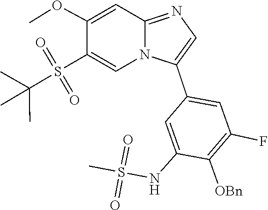 [SMILES: COc1cc2ncc(-c3cc(F)c(OCc4ccccc4)c(NS(C)(=O)=O)c3)n2cc1S(=O)(=O)C(C)(C)C] | N-(2-(benzyloxy)-5-(6- (tert-butylsulfonyl)-7- methoxyimidazo[1,2- a]pyridin-3-yl)-3- fluorophenyl) methanesulfonamide | [M + H]⁺ = 562.1 ¹H NMR (400 MHz, DMSO-d₆) δ ppm 9.70-9.36 (m, 1H), 8.71 (s, 1H), 7.78 (s, 1H), 7.58-7.52 (m, 2H), 7.46-7.33 (m, 5H), 7.28 (s, 1H), 5.20 (s, 2H), 3.93 (s, 3H), 3.05 (s, 3H), 1.33 (s, 9H). |
| 148 | 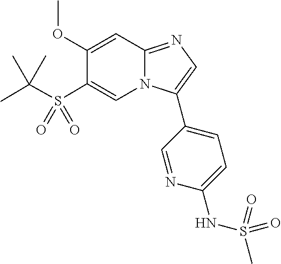 [SMILES: COc1cc2ncc(-c3ccc(NS(C)(=O)=O)nc3)n2cc1S(=O)(=O)C(C)(C)C] | N-(5-(6-(tert- butylsulfonyl)-7- methoxyimidazo[1,2- a]pyridin-3-yl)pyridin- 2- yl)methanesulfonamide | [M + H]⁺ = 439.1 ¹H NMR (400 MHz, DMSO-d₆) δ ppm 8.58 (s, 1H), 8.48 (d, J = 1.6 Hz, 1H), 7.95 (dd, J = 8.4, 2.4 Hz, 1H), 7.75 (s, 1H), 7.27 (s, 1H), 7.09 (d, J = 8.4 Hz, 1H), 3.93 (s, 3H), 3.31 (br s, 3H), 1.31 (s, 9H). |
| 149 | 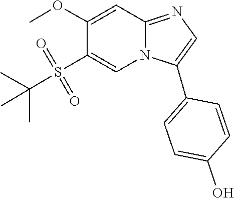 [SMILES: COc1cc2ncc(-c3ccc(O)cc3)n2cc1S(=O)(=O)C(C)(C)C] | 4-(6-(tert- butylsulfonyl)-7- methoxyimidazo[1,2- a]pyridin-3-yl)phenol | [M + H]⁺ = 360.9 ¹H NMR (400 MHz, DMSO-d₆) δ ppm: 9.86 (s, 1H), 8.60 (s, 1H), 7.61 (s, 1H), 7.40 (d, J = 8.4 Hz, 2H), 7.22 (s, 1H), 6.96 (d, J = 8.4 Hz, 2H), 3.92 (s, 3H), 1.30 (s, 9H). |
| 150 | 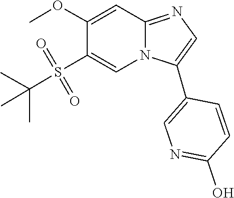 [SMILES: COc1cc2ncc(-c3ccc(O)nc3)n2cc1S(=O)(=O)C(C)(C)C] | 5-(6-(tert- butylsulfonyl)-7- methoxyimidazo[1,2- a]pyridin-3-yl)pyridin- 2-ol | [M + H]⁺ = 362.1 ¹H NMR (400 MHz, DMSO-d₆) δ ppm: 12.09-11.65 (m, 1H), 8.44 (s, 1H), 7.71-7.60 (m, 3H), 7.23 (s, 1H), 6.52 (d, J = 9.6 Hz, 1H), 3.92 (s, 3H), 1.30 (s, 9H). |
| 151 | 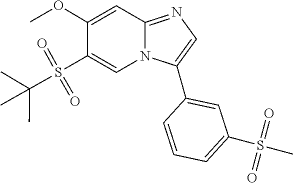 [SMILES: COc1cc2ncc(-c3cccc(S(C)(=O)=O)c3)n2cc1S(=O)(=O)C(C)(C)C] | 6-(tert-butylsulfonyl)- 7-methoxy-3-(3- (methylsulfonyl)phenyl) imidazo[1,2- a]pyridine | [M + H]⁺ = 423.0 ¹H NMR (400 MHz, DMSO-d₆) δ ppm: 8.72 (s, 1H), 8.15 (s, 1H), 8.03-7.95 (m, 2H), 7.92 (s, 1H), 7.89-7.84 (m, 1H), 7.31 (s, 1H), 3.95 (s, 3H), 3.29 (s, 3H), 1.32 (s, 9H). |
| 152 | 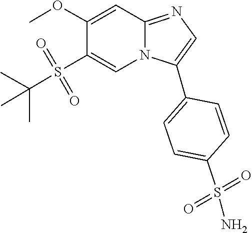 [SMILES: COc1cc2ncc(-c3ccc(S(N)(=O)=O)cc3)n2cc1S(=O)(=O)C(C)(C)C] | 4-(6-(tert- butylsulfonyl)-7- methoxyimidazo[1,2- a]pyridin-3- yl)benzenesulfonamide | [M + H]⁺ = 424.1 ¹H NMR (400 MHz, DMSO-d₆) δ ppm: 8.75 (s, 1H), 8.3-7.95 (m, 2H), 7.89 (s, 1H), 7.86-7.81 (m, 2H), 7.45 (s, 2H), 7.31 (s, 1H), 3.95 (s, 3H), 1.32 (s, 9H). |
| 153 | 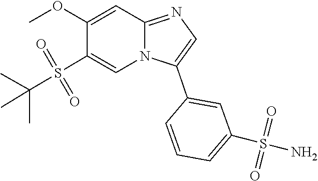 [SMILES: COc1cc2ncc(-c3cccc(S(N)(=O)=O)c3)n2cc1S(=O)(=O)C(C)(C)C] | 3-(6-(tert- butylsulfonyl)-7- methoxyimidazo[1,2- a]pyridin-3- yl)benzenesulfonamide | [M + H]⁺ = 424.0 ¹H NMR (400 MHz, DMSO-d₆) δ ppm: 8.70 (s, 1H), 8.02 (s, 1H), 7.95-7.76 (m, 2H), 7.47 (s, 2H), 7.30 (s, 1H), 3.95 (s, 3H), 1.32 (s, 9H). |
| Int. 78 | 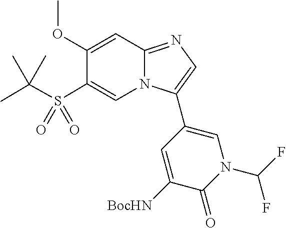 | tert-butyl(5-(6-(tert- butylsulfonyl)-7- methoxyimidazo[1,2- a]pyridin-3-yl)-1- (difluoromethyl)-2- oxo-1,2- dihydropyridin-3- yl)carbamate | [M + H]⁺ = 424.0 ¹H NMR (400 MHz, CDCl₃) δ ppm: 8.84-8.75 (m, 2H), 8.16- 8.09 (m, 2H), 7.67 (s, 1H), 7.52 (s, 1H), 7.00 (s, 1H), 4.18 (s, 3H), 1.48 (s, 9H), 0.07 (s, 9H). |
| Int. 79 | 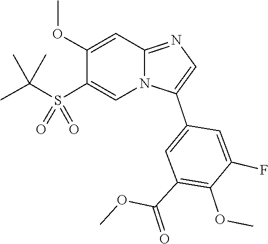 [SMILES: COC(=O)c1cc(-c2cnc3cc(OC)c(S(=O)(=O)C(C)(C)C)cn23)cc(F)c1OC] | methyl5-(6-(tert- butylsulfonyl)-7- methoxyimidazo[1,2- a]pyridin-3-yl)-3- fluoro-2- methoxybenzoate | [M + H]⁺ = 451.1 |
| 154 | 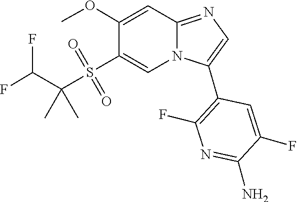 [SMILES: COc1cc2ncc(-c3cc(F)c(N)nc3F)n2cc1S(=O)(=O)C(C)(C)C(F)F] | 5-(6-((1,1-difluoro-2- methylpropan-2- yl)sulfonyl)-7- methoxyimidazo[1,2- a]pyridin-3-yl)-3,6- difluoropyridin-2- amine | [M + H]⁺ = 432.9 ¹H NMR (400 MHz, DMSO-d₆) δ ppm: 8.42 (d, J = 2.0 Hz, 1H), 7.89-7.71 (m, 1H), 7.69 (s, 1H), 7.29 (s, 1H), 7.07 (s, 2H), 6.34 (t, J = 14.8 Hz, 1H), 3.96 (s, 3H), 1.38 (s, 6H). |
| 155 | 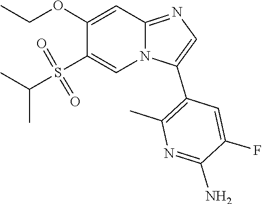 [SMILES: CCOc1cc2ncc(-c3cc(F)c(N)nc3C)n2cc1S(=O)(=O)C(C)C] | 5-(7-ethoxy-6- (isopropylsulfonyl) imidazo [1,2-a]pyridin-3- yl)-3-fluoro-6- methylpyridin-2-amine | [M + H]⁺ = 393.1 ¹H NMR (400 MHz, DMSO-d₆) δ ppm: 8.39 (s, 0.1H), 8.14 (s, 1H), 7.59 (s, 1H), 7.47 (d, J = 11.6 Hz, 1H), 7.27 (s, 1H), 6.57 (s, 2H), 4.30-4.22 (m, 2H), 3.80-3.65 (m, 1H), 2.05 (s, 3H), 1.40 (t, J = 6.8 Hz, 3H), 1.20 (d, J = 7.2 Hz, 6H). |
| 156 | 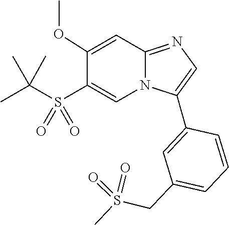 [SMILES: COc1cc2ncc(-c3cccc(CS(C)(=O)=O)c3)n2cc1S(=O)(=O)C(C)(C)C] | 6-(tert-butylsulfonyl)- 7-methoxy-3-(3- ((methylsulfonyl)methyl) phenyl)imidazo[1,2- a]pyridine | [M + H]⁺ = 437.0 ¹H NMR (400 MHz, DMSO-d₆) δ ppm: 8.71 (s, 1H), 8.38 (s, 0.1H), 7.76 (s, 1H), 7.68-7.61 (m, 3H), 7.54-7.48 (m, 1H), 7.28 (s, 1H), 4.58 (s, 2H), 3.94 (s, 3H), 2.96 (s, 3H), 1.31 (s, 9H). |
| 157 | 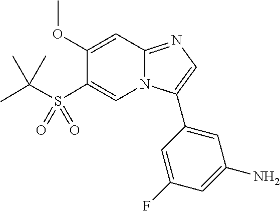 [SMILES: COc1cc2ncc(-c3cc(N)cc(F)c3)n2cc1S(=O)(=O)C(C)(C)C] | 3-(6-(tert- butylsulfonyl)-7- methoxyimidazo[1,2- a]pyridin-3-yl)-5- fluoroaniline | [M + H]⁺ = 378.4 ¹H NMR (400 MHz, DMSO-d₆) δ ppm 8.72 (s, 1 H), 7.72 (s, 1 H), 7.27 (s, 1 H), 6.53−6.48 (m, 1H), 653−6.48 (m, 1H), 6.41 (dt, J = 11.7, 2.2 Hz, 1H), 5.76 (br s, 2H), 3.93 (s, 3H), 1.31 (s, 9H). |
| Int. 80 | 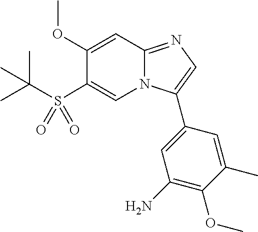 [SMILES: COc1cc2ncc(-c3cc(C)c(OC)c(N)c3)n2cc1S(=O)(=O)C(C)(C)C] | 5-(6-(tert- butylsulfonyl)-7- methoxyimidazo[1,2- a]pyridin-3-yl)-2- methoxy-3- methylaniline | ¹H NMR (400 MHz, DMSO-d₆) δ ppm 8.71 (s, 1H), 7.58 (s, 1H), 7.23 (s, 1H), 6.70 (d, J = 2.0 Hz, 1H), 6.54 (d, J = 2.0 Hz, 1H), 5.15 (s, 2H), 3.93 (s, 3H), 3.68 (s, 3H), 2.22 (s, 3H), 1.31 (s, 9H). |
| Int. 81 | 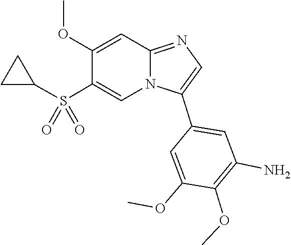 [SMILES: COc1cc2ncc(-c3cc(N)c(OC)c(OC)c3)n2cc1S(=O)(=O)C1CC1] | 5-(6- (cyclopropylsulfonyl)- 7-methoxyimidazo[1,2- a]pyridin-3-yl)-2,3- dimethoxyaniline | [M + H]⁺ = 404.0 ¹H NMR (400 MHz, CDCl₃) δ ppm: 8.87 (s, 1H), 7.55 (s, 1H), 7.01 (s, 1H), 6.60-6.26 (m, 2H), 4.03 (s, 3H), 3.85-3.83 (m, 6H), 3.06-2.88 (m, 1H), 1.37-1.26 (m, 2H), 1.12-0.96 (m, 2H). |
| Int. 82 | 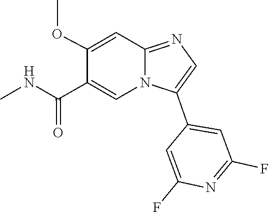 [SMILES: CNC(=O)c1cn2c(-c3cc(F)nc(F)c3)cnc2cc1OC] | 3-(2,6-difluoropyridin- 4-yl)-7-methoxy-N- methylimidazo[1,2- a]pyridine-6- carboxamide | [M + H]⁺ = 319.3 ¹H NMR (400 MHz, DMSO-d₆) δ ppm 8.94 (s, 1H), 8.33 (d, J = 4.6 Hz, 1H), 8.15 (s, 1H), 7.55 (s, 2H), 7.24 (s, 1H), 3.96 (s, 3H), 2.82 (d, J = 4.6 Hz, 3H). |
| Int. 83 | 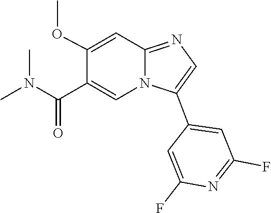 [SMILES: COc1cc2ncc(-c3cc(F)nc(F)c3)n2cc1C(=O)N(C)C] | 3-(2,6-difluoropyridin- 4-yl)-7-methoxy-N,N- dimethylimidazo[1,2- a]pyridine-6- carboxamide | [M + H]⁺ = 333.4 ¹H NMR (400 MHz, DMSO-d₆) δ ppm 8.74 (s, 1H), 8.15 (s, 1H), 7.51 (s, 2H), 7.25 (s, 1H), 3.92 (s, 3H), 3.01 (s, 3H), 2.84 (s, 3H). |
| 158 | 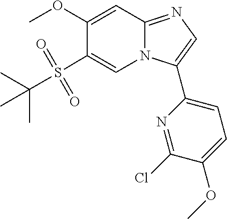 [SMILES: COc1cc2ncc(-c3ccc(OC)c(Cl)n3)n2cc1S(=O)(=O)C(C)(C)C] | 6-(tert-butylsulfonyl)- 3-(6-chloro-5- methoxypyridin-2-yl)- 7-methoxyimidazo[1,2- a]pyridine | [M + H]⁺ = 409.9 ¹H NMR (400 MHz, DMSO-d₆) δ ppm 10.19 (s, 1H), 8.35 (s, 1H), 8.02 (d, J = 8.4 Hz, 1H), 7.76 (d, J = 8.8 Hz, 1H), 7.30 (s, 1H), 3.97-3.92 (m, 6H), 1.33 (s, 9H). |

### Example 159

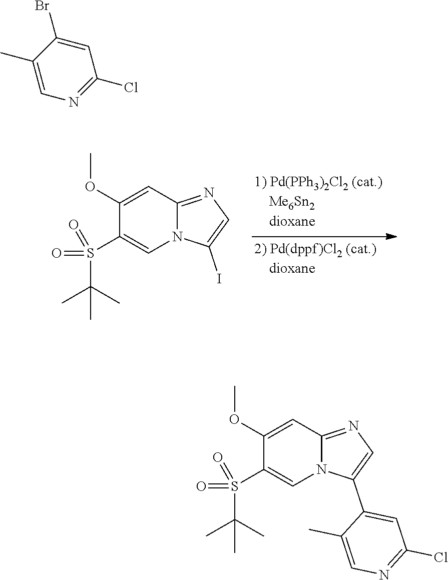

6-(tert-Butylsulfonyl)-3-(2-chloro-5-methylpyridin-4-yl)-7-methoxyimidazo[1,2-a]pyridine: 4-Bromo-2-chloro-5-methylpyridine (157 mg, 0.761 mmol) was dissolved in dioxane (4 mL). Bis(triphenylphosphine)palladium(II) dichloride (53.4 mg, 76.1 mol) and 1,1,1,2,2,2-hexamethyldistannane (333 μL, 1.60 mmol) were added and the mixture was heated to 100° C. for 0.5 h. 6-(tert-butylsulfonyl)-3-iodo-7-methoxyimidazo[1,2-a]pyridine (300 mg, 761 μmol) and [1,1′-Bis(diphenylphosphino)ferrocene]dichloropalladium(II) dichloromethane complex (62.1 mg, 76.1 μmol) were added and the mixture was heated to 100° C. for 12 h. The mixture was diluted with EtOAc and brine. The organic phase was separated and dried. The residue was purified by flash column chromatography on silica gel using CH₂Cl₂/EtOH as eluent to give the title compound. [M+H]⁺=394.4. ¹H NMR (400 MHz, DMSO-d₆) δ ppm 8.49 (s, 1H), 8.42 (s, 1H), 7.91 (s, 1H), 7.67 (s, 1H), 7.34 (s, 1H), 3.95 (s, 3H), 2.24 (s, 3H), 1.31 (s, 9H).

The following compounds were prepared following procedures analogous to that described for Example 159.

| Ex. | | | |
|---|---|---|---|
| No. | Structure | Name | LCMS, ¹H NMR |
| Int. 83 | 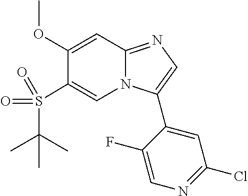 [SMILES: COc1cc2ncc(-c3cc(Cl)ncc3F)n2cc1S(=O)(=O)C(C)(C)C] | 6-(tert- butylsulfonyl)-3-(2- chloro-5- fluoropyridin-4-yl)-7- methoxyimidazo[1,2- al]pyridine | [M + H]+ = 398.4 |
| 160 | 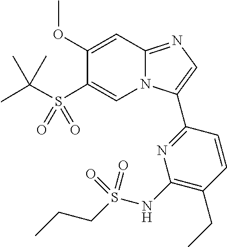 [SMILES: CCCS(=O)(=O)Nc1nc(-c2cnc3cc(OC)c(S(=O)(=O)C(C)(C)C)cn23)ccc1CC] | N-(6-(6-(tert- butylsulfonyl)-7- methoxyimidazo[1,2- a]pyridin-3-yl)-3- ethylpyridin-2- yl)propane-1- sulfonamide | [M + H]+ = 495.1 ¹H NMR (400 MHz, DMSO-d₆) δ ppm 9.85 (s, 1H), 9.84-9.65 (m, 1H), 8.27 (s, 1H), 7.84-7.73 (m, 1H), 7.72-7.68 (m, 1H), 7.32 (s, 1H), 3.94 (s, 3H), 3.50-3.43 (m, 2H), 2.73 (q, J = 7.6 Hz, 2H), 1.85-1.70 (m, 2H), 1.32 (s, 9H), 1.18 (t, J = 7.6 Hz, 3H), 0.94 (t, J = 7.2 Hz, 3H). |
| 161 | 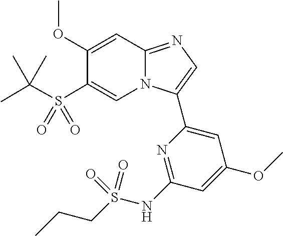 [SMILES: CCCS(=O)(=O)Nc1cc(OC)cc(-c2cnc3cc(OC)c(S(=O)(=O)C(C)(C)C)cn23)n1] | N-(6-(6-(tert- butylsulfonyl)-7- methoxyimidazo[1,2- a]pyridin-3-yl)-4- methoxypyridin-2- yl)propane-1- sulfonamide | [M + H]+ = 497.1 ¹H NMR (400 MHz, DMSO-d₆) δ ppm 9.87 (s, 1H), 8.35 (br s, 0.4H), 8.33 (s, 1H), 7.30 (s, 1H), 7.18 (br s, 1H), 6.45 (d, J = 2.0 Hz, 1H), 3.94 (s, 3H), 3.87 (s, 3H), 3.39 (br s, 2H), 1.77-1.69 (m, 2H), 1.33 (s, 9H), 0.94 (t, J = 7.6 Hz, 3H). |
| Int. 84 | 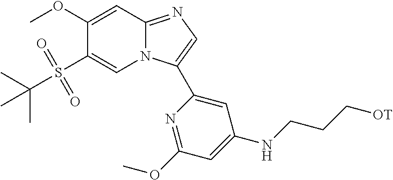 | N-(3-((tert- butyldimethylsilyl) oxy)propyl)-2-(6-(tert- butylsulfonyl)-7- methoxyimidazo[1,2- a]pyridin-3-yl)-6- methoxypyridin-4- amine | [M + H]⁺ = 563.2 ¹H NMR (400 MHz, CDCl₃) δ ppm: 10.66 (s, 1H), 7.93 (br s, 1H), 7.06 (br s, 1H), 6.54 (s, 1H), 5.80 (s, 1H), 4.89 (br s, 1H), 4.04 (s, 3H), 3.98 (s, 3H), 3.80 (t, J = 4.4 Hz, 2H), 3.30 (d, J = 5.2 Hz, 2H), 1.92-1.80 (m, 2H), 1.41 (s, 9H), 0.94 (s, 9H), 0.09 (s, 6H). |
| 162 | 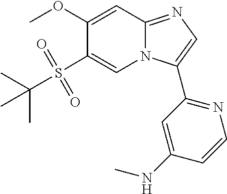 [SMILES: CNc1ccnc(-c2cnc3cc(OC)c(S(=O)(=O)C(C)(C)C)cn23)c1] | 2-(6-(tert- butylsulfonyl)-7- methoxyimidazo[1,2- a]pyridin-3-yl)-N- methylpyridin-4- amine | [M + H]⁺ = 375.1 ¹H NMR (400 MHz, CDCl₃) δ ppm: 10.50 (s, 1H), 8.25 (s, 1H), 8.21 (s, 0.6H), 8.11 (d, J = 5.6 Hz, 1H), 7.25 (s, 1H), 6.97 (d, J = 2.0 Hz, 1H), 6.68 (q, J = 4.8 Hz, 1H), 6.45 (dd, J = 2.4, 6.0 Hz, 1H), 3.93 (s, 3H), 2.78 (d, J = 4.8 Hz, 3H), 1.31 (s, 9H). |
| 163 | 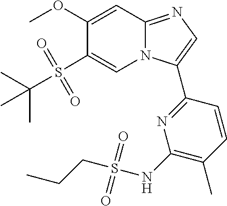 [SMILES: CCCS(=O)(=O)Nc1nc(-c2cnc3cc(OC)c(S(=O)(=O)C(C)(C)C)cn23)ccc1C] | N-(6-(6-(tert- butylsulfonyl)-7- methoxyimidazo[1,2- a]pyridin-3-yl)-3- methylpyridin-2- yl)propane-1- sulfonamide | [M + H]⁺ = 481.1 ¹H NMR (400 MHz, CDCl₃) δ ppm: 9.85 (s, 1H), 9.75 (s, 1H), 8.27 (s, 1H), 7.80-7.71 (m, 1H), 7.70-7.63 (m, 1H), 7.32 (s, 1H), 3.94 (s, 3H), 3.52- 3.43 (m, 2H), 2.32 (s, 3H), 1.82-1.71 (m, 2H), 1.32 (s, 9H), 0.95 (t, J = 7.6 Hz, 3H) |
| 164 | 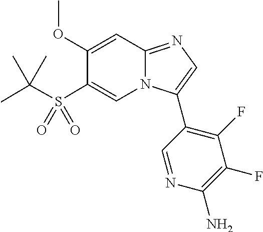 [SMILES: COc1cc2ncc(-c3cnc(N)c(F)c3F)n2cc1S(=O)(=O)C(C)(C)C] | 5-(6-(tert- butylsulfonyl)-7- methoxyimidazo[1,2- a]pyridin-3-yl)-3,4- difluoropyridin-2- amine | [M + H]⁺ = 397.0 ¹H NMR (400 MHz, DMSO-d₆) δ ppm 8.43 (d, J = 2.4 Hz, 1H), 7.97 (d, J = 8.8 Hz, 1H), 7.71 (s, 1H), 7.28 (s, 1H), 6.97 (s, 2H), 3.93 (s, 3H), 1.30 (s, 9H). |

### Example 165

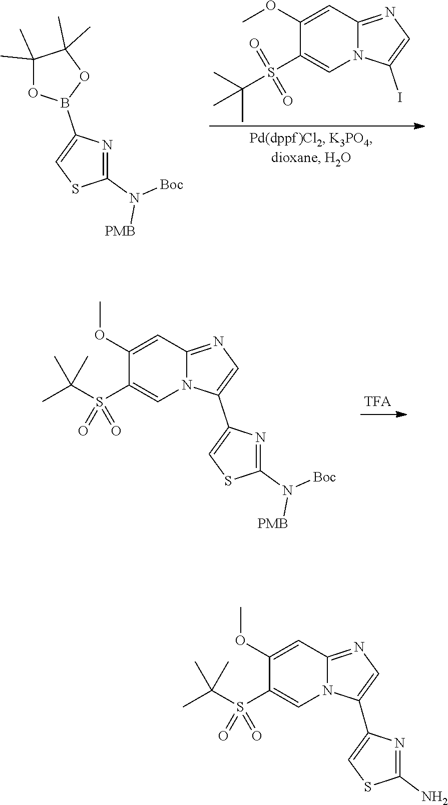

tert-butyl (4-(6-(tert-butylsulfonyl)-7-methoxyimidazo[1,2-a]pyridin-3-yl)thiazol-2-yl)(4-methoxybenzyl)carbamate: A mixture of 6-(tert-butylsulfonyl)-3-iodo-7-methoxyimidazo[1,2-a]pyridine (117 mg, 267 mol, 0.7 eq), tert-butyl (4-methoxybenzyl)(4-(4,4,5,5-tetramethyl-1,3,2-dioxaborolan-2-yl)thiazol-2-yl)carbamate (170 mg, 381 mol, 1 eq), K₃PO₄ (243 mg, 1.14 mmol, 3 eq) and Pd(dppf)Cl₂ (27.9 mg, 38.1 mol, 0.1 eq) in 1,4-dioxane (4 mL) and H₂O (1 mL) was degassed and purged with N₂ three times, and then the mixture was stirred at 80° C. for 12 h under a N₂ atmosphere. The reaction mixture was filtered and concentrated under reduced pressure to give the title compound, which was used in the next step without further purification.

4-(6-(tert-butylsulfonyl)-7-methoxyimidazo[1,2-a]pyridin-3-yl)thiazol-2-amine: A mixture of tert-butyl (4-(6-(tert-butylsulfonyl)-7-methoxyimidazo[1,2-a]pyridin-3-yl)thiazol-2-yl)(4-methoxybenzyl)carbamate (220 mg, 375 μmol, 1 eq) in TFA (10 mL) was stirred at 70° C. for 2 h. The mixture was filtered and concentrated under reduced pressure to give a residue, which was purified by RP-HPLC to give the title compound. [M+H]⁺=367.0. ¹H NMR (400 MHz, DMSO-d₆) δ ppm 9.65 (s, 1H), 8.21 (s, 0.14H), 7.89 (s, 1H), 7.21 (d, J=3.2 Hz, 3H), 6.94 (s, 1H), 3.92 (s, 3H), 1.32 (s, 9H).

The following compounds were prepared following procedures analogous to that described for Example 165.

| Ex. | | | |
|---|---|---|---|
| No. | Structure | Name | LCMS, ¹H NMR |
| 166 | 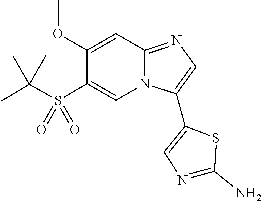 [SMILES: COc1cc2ncc(-c3cnc(N)s3)n2cc1S(=O)(=O)C(C)(C)C] | 5-(6-(tert- butylsulfonyl)-7- methoxyimidazo[1,2- a]pyridin-3- yl)thiazol-2-amine | [M + H]⁺ = 367.0 ¹H NMR (400 MHz, DMSO-d₆) δ ppm 8.59 (s, 1H), 7.66 (s, 1H), 7.36 (s, 2H), 7.24 (s, 1H), 7.21 (s, 1H), 3.92 (s, 3H), 1.31 (s, 9H). |
| 167 | 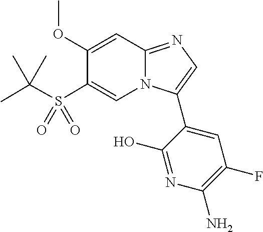 [SMILES: COc1cc2ncc(-c3cc(F)c(N)nc3O)n2cc1S(=O)(=O)C(C)(C)C] | 6-amino-3-(6-(tert- butylsulfonyl)-7- methoxyimidazo[1,2- a]pyridin-3-yl)-5- fluoropyridin-2-ol | [M + H]⁺ = 395.1. ¹H NMR (400 MHz, DMSO-d₆) δ ppm 8.35 (s, 1H), 8.13 (s, 1H), 7.53-7.45 (m, 2H), 7.19 (s, 1H), 6.41 (s, 2H), 3.91 (s, 3H), 1.29 (s, 9H). |

### Example 168

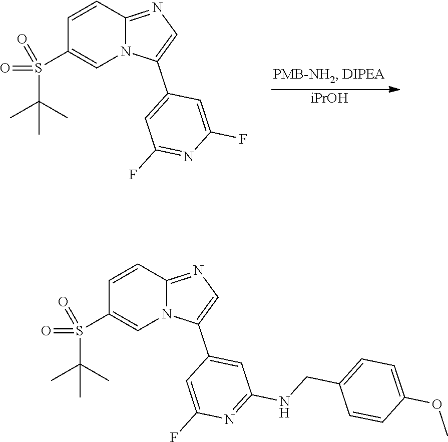 [SMILES: "iPrOH" ; CC(C)(C)S(=O)(=O)c1ccc2ncc(-c3cc(F)nc(F)c3)n2c1 ; COc1ccc(CNc2cc(-c3cnc4ccc(S(=O)(=O)C(C)(C)C)cn34)cc(F)n2)cc1]

4-(6-(tert-butylsulfonyl)imidazo[1,2-a]pyridin-3-yl)-6-fluoro-N-(4-methoxybenzyl)pyridin-2-amine: 6-(tert-butylsulfonyl)-3-(2,6-difluoropyridin-4-yl)imidazo[1,2-a]pyridine (180 mg, 92% wt, 1 equiv, 471 μmol) and (4-methoxyphenyl)methanamine (129 mg, 123 μL, 2 equiv, 943 mol) were dissolved in 2-propanol (4.6 mL). DIPEA (91.4 mg, 123 μL, 1.5 equiv, 707 μmol) was added, and the resulting mixture was heated to 120° C. for 2 h. The solvent was evaporated. A partial amount of the residue was dissolved in DMSO (2 mL) and submitted to HPLC purification (basic method) to give the title compound. [M+H]⁺=469.4. ¹H NMR (400 MHz, DMSO-d₆) δ ppm 8.82 (dd, J=1.8, 0.8 Hz, 1H), 8.09 (s, 1H), 7.88 (dd, J=9.4, 0.8 Hz, 1H), 7.78 (br t, J=5.7 Hz, 1H), 7.58 (dd, J=9.4, 1.8 Hz, 1H), 7.29 (d, J=8.6 Hz, 2H), 6.84-6.94 (m, 2H), 6.63 (s, 1H), 6.46 (s, 1H), 4.40 (d, J=5.8 Hz, 2H), 3.72 (s, 3H), 1.32 (s, 9H).

### Examples 169 and 170

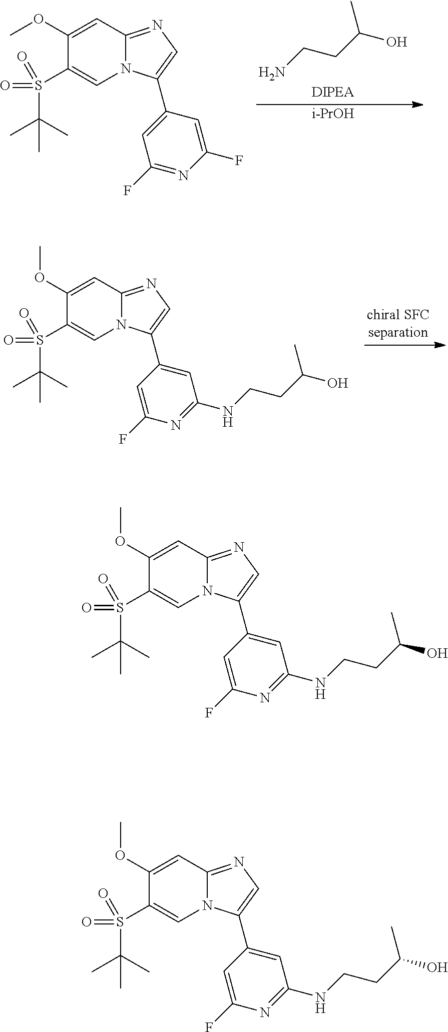

4-((4-(6-(tert-butylsulfonyl)-7-methoxyimidazo[1,2-a]pyridin-3-yl)-6-fluoropyridin-2-yl)amino)butan-2-ol: 6-(tert-butylsulfonyl)-3-(2,6-difluoropyridin-4-yl)-7-methoxyimidazo[1,2-a]pyridine (110 mg, 98% wt, 1 equiv, 280 μmol) and 4-aminobutan-2-ol (52.4 mg, 56 μL, 2.1 equiv, 588 gmol) were dissolved in 2-propanol (4 mL) and DIPEA (54.2 mg, 73.1 μL, 1.5 equiv, 420 μmol) was added. The vial was sealed and heated to 120° C. for 4 h. The reaction mixture was cooled to rt. The solvent was evaporated under reduced pressure. The residue was redissolved in DMSO (2.5 mL) and submitted to HPLC purification on a chiral stationary column to give the two resolved enantiomers, with stereochemistry assigned arbitrarily.

(R)-4-((4-(6-(tert-butylsulfonyl)-7-methoxyimidazo[1,2-a]pyridin-3-yl)-6-fluoropyridin-2-yl)amino)butan-2-ol [M+H]⁺=451.5. ¹H NMR (400 MHz, DMSO-d₆) δ ppm 8.81 (s, 1H), 7.92 (s, 1H), 7.31 (s, 1H), 7.21 (br s, 1H), 6.50 (s, 1H), 6.35 (s, 1H), 4.50 (d, J=4.8 Hz, 1H), 3.94 (s, 3H), 3.66-3.75 (m, 1H), 3.19-3.30 (m, 2H), 1.51-1.67 (m, 2H), 1.32 (s, 9H), 1.09 (d, J=6.1 Hz, 3H).

(S)-4-((4-(6-(tert-butylsulfonyl)-7-methoxyimidazo[1,2-a]pyridin-3-yl)-6-fluoropyridin-2-yl)amino)butan-2-ol [M+H]⁺=451.5. H NMR (400 MHz, DMSO-d₆) δ ppm 8.81 (s, 1H), 7.92 (s, 1H), 7.31 (s, 1H), 7.14-7.27 (i, 1H), 6.50 (s, 1H), 6.35 (s, H), 4.50 (d, J=4.8 Hz, H), 3.94 (s, 3H), 3.64-3.76 (m, 1H), 3.20-3.30 (m, 2H), 1.54-1.69 (m, 2H), 1.32 (s, 9H), 1.09 (d, J=6.1 Hz, 4H).

The following compounds were prepared following procedures analogous to those described for Example 168, 169, and 170.

| Ex. | | | |
|---|---|---|---|
| No. | Structure | Name | LCMS, ¹H NMR |
| 171 | 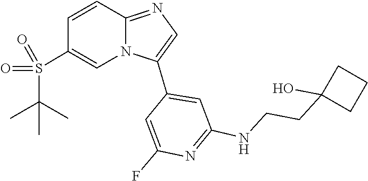 [SMILES: CC(C)(C)S(=O)(=O)c1ccc2ncc(-c3cc(F)nc(NCCC4(O)CCC4)c3)n2c1] | 1-(2-((4-(6-(tert- butylsulfonyl)imidazo [1,2-a]pyridin-3- yl)-6-fluoropyridin- 2- yl)amino)ethyl)cyclo- butan-1-ol | [M + H]⁺ = 447.5 ¹H NMR (400 MHz, DMSO- d₆) δ ppm 8.80-8.84 (m, 1H), 8.10 (s, 1H), 7.88 (dd, J = 9.4, 0.8 Hz, 1H), 7.55-7.61 (m, 1H), 7.20 (br s, 1H), 6.56 (s, 1H), 6.42 (s, 1H), 5.00 (s, 1H), 3.22-3.31 (m, 2H), 1.93-2.04 (m, 4H), 1.76-1.84 (m, 2H), 1.57-1.70 (m, 1H), 1.42-1.54 (m, 1H), 1.33 (s, 9H) |
| 172 | 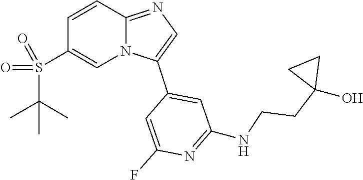 [SMILES: CC(C)(C)S(=O)(=O)c1ccc2ncc(-c3cc(F)nc(NCCC4(O)CC4)c3)n2c1] | 1-(2-((4-(6-(tert- butylsulfonyl)imidazo [1,2-a]pyridin-3- yl)-6-fluoropyridin- 2- yl)amino)ethyl)cyclo- propan-1-ol | [M + H]⁺ = 433.5 ¹H NMR (400 MHz, DMSO- d₆) δ ppm 8.82 (d, J = 0.8 Hz, 1H), 8.10 (s, 1H), 7.88 (dd, J = 9.6, 0.8 Hz, 1H), 7.58 (dd, J = 9.5, 1.9 Hz, 1H), 7.18-7.26 (m, 1H), 6.57 (s, 1H), 6.42 (s, 1H), 5.16 (s, 1H), 3.36-3.45 (m, 2H), 1.68-1.77 (m, 2H), 1.33 (s, 9H), 0.52-0.58 (m, 2H), 0.35-0.41 (m, 2H) |
| 173 | 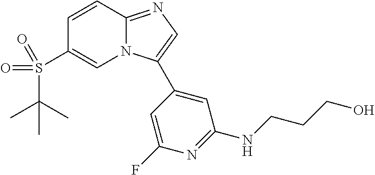 [SMILES: CC(C)(C)S(=O)(=O)c1ccc2ncc(-c3cc(F)nc(NCCCO)c3)n2c1] | 3-((4-(6-(tert- butylsulfonyl)imidazo [1,2-a]pyridin-3- yl)-6-fluoropyridin- 2-yl)amino)propan- 1-ol | [M + H]⁺ = 407.5 ¹H NMR (400 MHz, DMSO- d₆) δ ppm 8.80-8.84 (m, 1H), 8.10 (s, 1H), 7.88 (dd, J = 9.6, 0.8 Hz, 1H), 7.58 (dd, J = 9.4, 1.8 Hz, 1H), 7.28 (br s, 1H), 6.58 (s, 1H), 6.43 (s, 1H), 4.49 (t, J = 5.1 Hz, 1H), 3.49 (q, J = 6.1 Hz, 2H), 3.23-3.31 (m, 2H), 1.70 (quin, J = 6.6 Hz, 2H), 1.33 (s, 9H) |
| 174 | 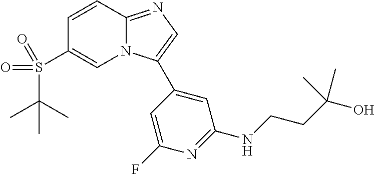 [SMILES: CC(C)(O)CCNc1cc(-c2cnc3ccc(S(=O)(=O)C(C)(C)C)cn23)cc(F)n1] | 4-((4-(6-(tert- butylsulfonyl)imidazo [1,2-a]pyridin-3- yl)-6-fluoropyridin- 2-yl)amino)-2- methylbutan-2-ol | [M + H]⁺ = 435.5 ¹H NMR (400 MHz, DMSO- d₆) δ ppm 8.79-8.85 (m, 1H), 8.10 (s, 1H), 7.88 (dd, J = 9.5, 0.9 Hz, 1H), 7.58 (dd, J = 9.4, 1.8 Hz, 1H), 7.20 (br d, J = 3.0 Hz, 1H), 6.55 (d, J = 0.8 Hz, 1H), 6.42 (s, 1H), 4.34 (s, 1H), 3.19-3.31 (m, 2H), 1.61-1.72 (m, 2H), 1.33 (s, 9H), 1.14 (s, 6H) |
| 175 | 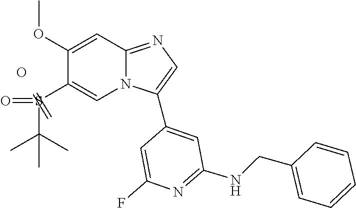 | N-benzyl-4-(6-(tert- butylsulfonyl)-7- methoxyimidazo[1,2- a]pyridin-3-yl)-6- fluoropyridin-2- amine | [M + H]⁺ = 469.2 ¹H NMR (400 MHz, DMSO- d₆) δ ppm 8.81 (s, 1H), 7.92 (s, 1H), 7.76-7.86 (m, 1H), 7.32- 7.38 (m, 4H), 7.31 (s, 1H), 7.21-7.27 (m, 1H), 6.52-6.62 (m, 1H), 6.41 (s, 1H), 4.48 (d, J = 6.1 Hz, 2H), 3.94 (s, 3H), 1.32 (s, 9H) |
| 176 | 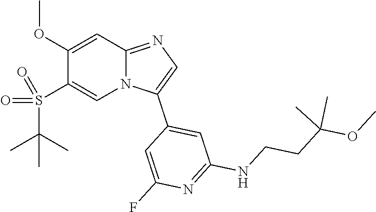 [SMILES: COc1cc2ncc(-c3cc(F)nc(NCCC(C)(C)OC)c3)n2cc1S(=O)(=O)C(C)(C)C] | 4-(6-(tert- butylsulfonyl)-7- methoxyimidazo[1,2- a]pyridin-3-yl)-6- fluoro-N-(3- methoxy-3- methylbutyl)pyridin- 2-amine | [M + H]⁺ = 479.6 ¹H NMR (400 MHz, DMSO- d₆) δ ppm 8.81 (s, 1H), 7.92 (s, 1H), 7.31 (s, 1H), 7.19 (br s, 1H), 6.48 (s, 1H), 6.36 (s, 1H), 3.94 (s, 3H), 3.19-3.27 (m, 2H), 3.12 (s, 3H), 1.68-1.78 (m, 2H), 1.31-1.35 (m, 9H), 1.12-1.15 (m, 6H) |
| 177 | 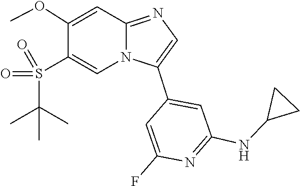 [SMILES: COc1cc2ncc(-c3cc(F)nc(NC4CC4)c3)n2cc1S(=O)(=O)C(C)(C)C] | 4-(6-(tert- butylsulfonyl)-7- methoxyimidazo[1,2- a]pyridin-3-yl)-N- cyclopropyl-6- fluoropyridin-2- amine | [M + H]⁺ = 419.5 ¹H NMR (400 MHz, DMSO- d₆) δ ppm 8.91 (s, 1H), 7.99 (s, 1H), 7.44-7.50 (m, 1H), 7.32 (s, 1H), 6.69 (s, 1H), 6.49 (s, 1H), 3.95 (s, 3H), 1.32 (s, 9H), 0.70-0.81 (m, 2H), 0.44-0.54 (m, 2H) |
| 178 | 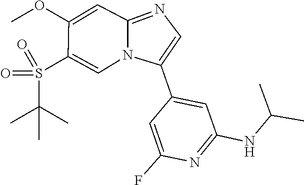 [SMILES: COc1cc2ncc(-c3cc(F)nc(NC(C)C)c3)n2cc1S(=O)(=O)C(C)(C)C] | 4-(6-(tert- butylsulfonyl)-7- methoxyimidazo[1,2- a]pyridin-3-yl)-6- fluoro-N- isopropylpyridin-2- amine | [M + H]⁺ = 421.2 ¹H NMR (400 MHz, DMSO- d₆) δ ppm 8.80 (s, 1H), 7.91 (s, 1H), 7.31 (s, 1H), 7.13 (br d, J = 7.6 Hz, 1H), 6.48 (s, 1H), 6.34 (s, 1H), 3.94 (s, 3H), 3.85- 3.93 (m, 1H), 1.32 (s, 9H), 1.17 (d, J = 6.6 Hz, 6H) |
| 179 | 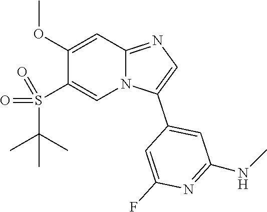 [SMILES: CNc1cc(-c2cnc3cc(OC)c(S(=O)(=O)C(C)(C)C)cn23)cc(F)n1] | 4-(6-(tert- butylsulfonyl)-7- methoxyimidazo[1,2- a]pyridin-3-yl)-6- fluoro-N- methylpyridin-2- amine | [M + H]⁺ = 393.2 1H NMR (400 MHz, DMSO- d₆) δ ppm 8.83 (s, 1H), 7.94 (s, 1H), 7.31 (s, 1H), 7.14-7.25 (m, 1H), 6.49 (s, 1H), 6.38 (s, 1H), 3.94 (s, 3H), 2.78 (d, J = 4.8 Hz, 3H), 1.32 (s, 9H) |
| 180 | 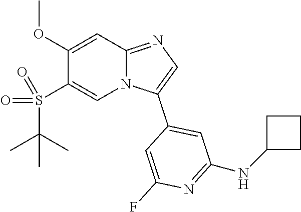 [SMILES: COc1cc2ncc(-c3cc(F)nc(NC4CCC4)c3)n2cc1S(=O)(=O)C(C)(C)C] | 4-(6-(tert- butylsulfonyl)-7- methoxyimidazo[1,2- a]pyridin-3-yl)-N- cyclobutyl-6- fluoropyridin-2- amine | [M + H]⁺ = 433.5 ¹H NMR (400 MHz, DMSO- d₆) δ ppm 8.82 (s, 1H), 7.93 (s, 1H), 7.51 (br d, J = 6.6 Hz, 1H), 7.31 (s, 1H), 6.44 (s, 1H), 6.39 (s, 1H), 4.08-4.22 (m, 1H), 3.94 (s, 3H), 2.26-2.37 (m, 2H), 1.86-1.98 (m, 2H), 1.59-1.76 (m, 2H), 1.33 (s, 9H) |
| 181 | 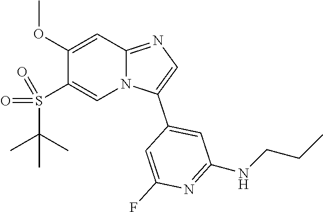 [SMILES: CCCNc1cc(-c2cnc3cc(OC)c(S(=O)(=O)C(C)(C)C)cn23)cc(F)n1] | 4-(6-(tert- butylsulfonyl)-7- methoxyimidazo[1,2- a]pyridin-3-yl)-6- fluoro-N- propylpyridin-2- amine | [M + H]⁺ = 421.2 ¹H NMR (400 MHz, DMSO- d₆) δ ppm 8.81 (s, 1H), 7.92 (s, 1H), 7.31 (s, 1H), 7.28 (br t, J = 5.1 Hz, 1H), 6.50 (s, 1H), 6.35 (s, 1H), 3.94 (s, 3H), 3.17 (q, J = 6.4 Hz, 2H), 1.56 (sxt, J = 7.3 Hz, 2H), 0.92 (t, J = 7.4 Hz, 3H) |
| 182 | 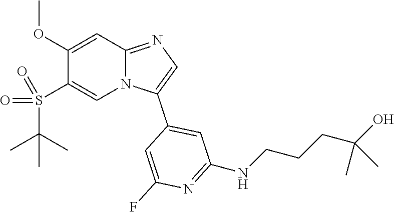 [SMILES: COc1cc2ncc(-c3cc(F)nc(NCCCC(C)(C)O)c3)n2cc1S(=O)(=O)C(C)(C)C] | 5-((4-(6-(tert- butylsulfonyl)-7- methoxyimidazo[1,2- a]pyridin-3-yl)-6- fluoropyridin-2- yl)amino)-2- methylpentan-2-ol | [M + H]⁺ = 479.6 ¹H NMR (400 MHz, DMSO- d₆) δ ppm 8.81 (s, 1H), 7.92 (s, 1H), 7.31 (s, 1H), 7.19-7.28 (m, 1H), 6.50 (s, 1H), 6.35 (s, 1H), 4.14 (s, 1H), 3.94 (s, 3H), 3.19 (q, J = 6.7 Hz, 2H), 1.52- 1.66 (m, 2H), 1.37-1.45 (m, 2H), 1.32 (s, 9H), 1.08 (s, 6H) |
| 183 | 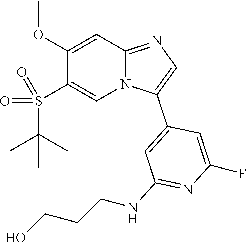 [SMILES: COc1cc2ncc(-c3cc(F)nc(NCCCO)c3)n2cc1S(=O)(=O)C(C)(C)C] | 3-((4-(6-(tert- butylsulfonyl)-7- methoxyimidazo[1,2- a]pyridin-3-yl)-6- fluoropyridin-2- yl)amino)propan-1- ol | [M + H]⁺ = 437.2 ¹H NMR (400 MHz, DMSO- d₆) δ ppm 8.81 (s, 1H), 7.92 (s, 1H), 7.31 (s, 1H), 7.19-7.26 (m, 1H), 6.51 (s, 1H), 6.36 (s, 1H), 4.48 (t, J = 5.1 Hz, 1H), 3.94 (s, 3H), 3.45-3.55 (m, 2H), 3.26 (q, J = 6.6 Hz, 2H), 1.70 (quin, J = 6.6 Hz, 2H), 1.32 (s, 9H) |
| 184 | 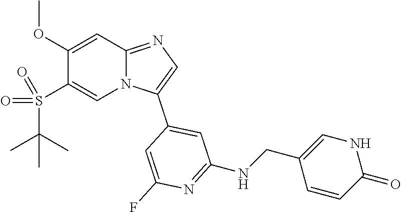 [SMILES: COc1cc2ncc(-c3cc(F)nc(NCc4ccc(=O)[nH]c4)c3)n2cc1S(=O)(=O)C(C)(C)C] | 5-(((4-(6-(tert- butylsulfonyl)-7- methoxyimidazo[1,2- a]pyridin-3-yl)-6- fluoropyridin-2- yl)amino)methyl) pyridin-2(1H)-one | [M + H]⁺ = 486.2 ¹H NMR (400 MHz, DMSO- d₆) δ ppm 10.92-11.65 (m, 1H), 8.80 (s, 1H), 7.92 (s, 1H), 7.63 (br t, J = 5.6 Hz, 1H), 7.45 (dd, J = 9.5, 2.7 Hz, 1H), 7.33 (d, J = 2.3 Hz, 1H), 7.31 (s, 1H), 6.57 (s, 1H), 6.42 (s, 1H), 6.31 (d, J = 9.4 Hz, 1H), 4.18 (d, J = 5.8 Hz, 2H), 3.94 (s, 3H), 1.32 (s, 9H) |
| 185 | 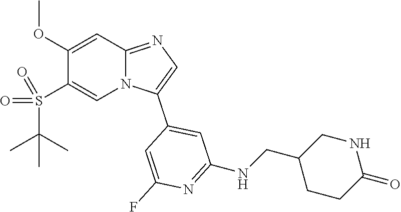 [SMILES: COc1cc2ncc(-c3cc(F)nc(NCC4CCC(=O)NC4)c3)n2cc1S(=O)(=O)C(C)(C)C] | 5-(((4-(6-(tert- butylsulfonyl)-7- methoxyimidazo[1,2- a]pyridin-3-yl)-6- fluoropyridin-2- yl)amino)methyl) piperidin-2-one | [M + H]⁺ = 490.4 ¹H NMR (400 MHz, DMSO- d₆) δ ppm 8.81 (s, 1H), 7.93 (s, 1H), 7.36-7.48 (m, 2H), 7.31 (s, 1H), 6.55 (s, 1H), 6.39 (s, 1H), 3.94 (s, 3H), 3.14-3.28 (m, 3H), 2.88 (t, J = 10.9 Hz, 1H), 2.11-2.25 (m, 2H), 1.94- 2.08 (m, 1H), 1.75-1.89 (m, 1H), 1.41-1.52 (m, 1H), 1.32 (s, 9H) |
| 186 | 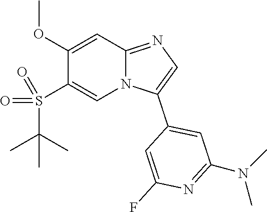 [SMILES: COc1cc2ncc(-c3cc(F)nc(N(C)C)c3)n2cc1S(=O)(=O)C(C)(C)C] | 4-(6-(tert- butylsulfonyl)-7- methoxyimidazo[1,2- a]pyridin-3-yl)-6- fluoro-N,N- dimethylpyridin-2- amine | [M + H]⁺ = 407.2 ¹H NMR (400 MHz, DMSO- d₆) δ ppm 8.90 (s, 1H), 7.99 (s, 1H), 7.31 (s, 1H), 6.71 (d, J = 1.3 Hz, 1H), 6.47 (s, 1H), 3.94 (s, 3H), 3.06 (s, 6H), 1.32 (s, 9H) |
| 187 | 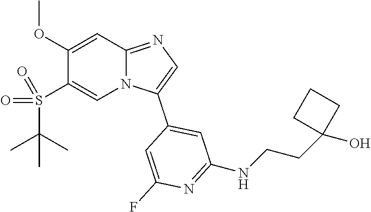 [SMILES: COc1cc2ncc(-c3cc(F)nc(NCCC4(O)CCC4)c3)n2cc1S(=O)(=O)C(C)(C)C] | 1-(2-((4-(6-(tert- butylsulfonyl)-7- methoxyimidazo[1,2- a]pyridin-3-yl)-6- fluoropyridin-2- yl)amino)ethyl)cyclo- butan-1-ol | [M + H]⁺ = 477.3 ¹H NMR (400 MHz, DMSO- d₆) δ ppm 8.81 (s, 1H), 7.92 (s, 1H), 7.31 (s, 1H), 7.10-7.20 (m, 1H), 6.49 (s, 1H), 6.36 (s, 1H), 4.99 (s, 1H), 3.94 (s, 3H), 3.21-3.31 (m, 2H), 1.97 (s, 4H), 1.76-1.84 (m, 2H), 1.59- 1.70 (m, 1H), 1.43-1.54 (m, 1H), 1.32 (s, 9H) |
| 188 | 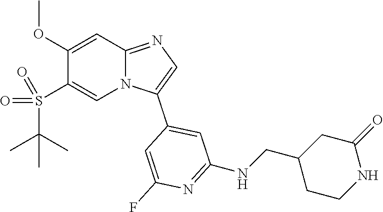 [SMILES: COc1cc2ncc(-c3cc(F)nc(NCC4CCNC(=O)C4)c3)n2cc1S(=O)(=O)C(C)(C)C] | 4-(((4-(6-(tert- butylsulfonyl)-7- methoxyimidazo[1,2- a]pyridin-3-yl)-6- fluoropyridin-2- yl)amino)methyl) piperidin-2-one | [M + H]⁺ = 490.2 ¹H NMR (400 MHz, DMSO- d₆) δ ppm 8.81 (s, 1H), 7.93 (s, 1H), 7.44-7.53 (m, 1H), 7.24- 7.42 (m, 2H), 6.47-6.59 (m, 1H), 6.35-6.40 (m, 1H), 3.94 (s, 3H), 3.34-3.40 (m, 1H), 3.15-3.26 (m, 2.33H), 2.87- 2.94 (m, 0.66H), 2.65-2.69 (m, 0.33H), 2.37-2.46 (m, 0.66H), 2.21-2.35 (m, 1.33H), 1.99-2.13 (m, 0.33H), 1.81- 1.93 (m, 1.33H), 1.62-1.72 (m, 1H), 1.32 (s, 9H) |
| 189 | 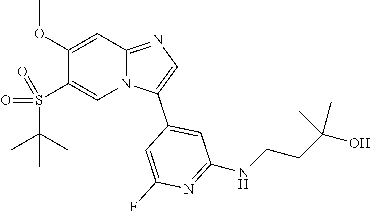 [SMILES: COc1cc2ncc(-c3cc(F)nc(NCCC(C)(C)O)c3)n2cc1S(=O)(=O)C(C)(C)C] | 4-((4-(6-(tert- butylsulfonyl)-7- methoxyimidazo[1,2- a]pyridin-3-yl)-6- fluoropyridin-2- yl)amino)-2- methylbutan-2-ol | [M + H]⁺ = 465.2 ¹H NMR (400 MHz, DMSO- d₆) δ ppm 8.81 (s, 1H), 7.92 (s, 1H), 7.31 (s, 1H), 7.15 (br s, 1H), 6.48 (s, 1H), 6.35 (s, 1H), 4.33 (s, 1H), 3.94 (s, 3H), 3.22- 3.31 (m, 2H), 1.61-1.75 (m, 2H), 1.32 (s, 9H), 1.14 (s, 6H) |
| 190 | 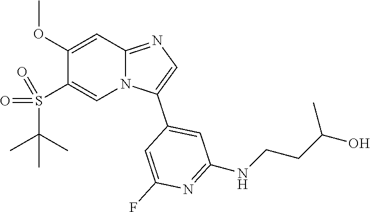 [SMILES: COc1cc2ncc(-c3cc(F)nc(NCCC(C)O)c3)n2cc1S(=O)(=O)C(C)(C)C] | rac-4-((4-(6-(tert- butylsulfonyl)-7- methoxyimidazo[1,2- a]pyridin-3-yl)-6- fluoropyridin-2- yl)amino)butan-2-ol | [M + H]⁺ = 451.2 ¹H NMR (400 MHz, DMSO- d₆) δ ppm 8.81 (s, 1H), 7.92 (s, 1H), 7.31 (s, 1H), 7.14-7.25 (m, 1H), 6.50 (s, 1H), 6.35 (s, 1H), 4.50 (d, J = 4.8 Hz, 1H), 3.94 (s, 3H), 3.67-3.76 (m, 1H), 3.19-3.31 (m, 2H), 1.54- 1.68 (m, 2H), 1.32 (s, 9H), 1.09 (d, J = 6.3 Hz, 3H) |
| 191 | 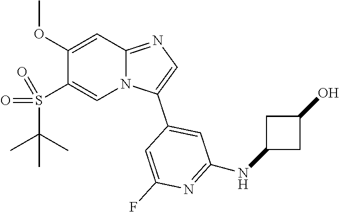 | (1s,3s)-3-((4-(6- (tert-butylsulfonyl)- 7- methoxyimidazo[1,2- a]pyridin-3-yl)-6- fluoropyridin-2- yl)amino)cyclobutan- 1-ol | [M + H]⁺ = 449.2 ¹H NMR (400 MHz, DMSO- d₆) δ ppm 8.81 (s, 1H), 7.93 (s, 1H), 7.47 (br d, J = 6.3 Hz, 1H), 7.31 (s, 1H), 6.44 (s, 1H), 6.38 (s, 1H), 5.11 (d, J = 6.1 Hz, 1H), 3.94 (s, 3H), 3.78- 3.89 (m, 1H), 3.52-3.72 (m, 1H), 2.61-2.72 (m, 2H), 1.67- 1.82 (m, 2H), 1.33 (s, 9H) |
| 192 | 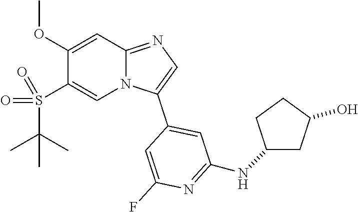 | rac-(1R,3S)-3-((4-(6- (tert-butylsulfonyl)- 7- methoxyimidazo[1,2- a]pyridin-3-yl)-6- fluoropyridin-2- yl)amino)cyclopentan- 1-ol | [M + H]⁺ = 463.2 ¹H NMR (400 MHz, DMSO- d₆) δ ppm 8.81 (s, 1H), 7.91 (s, 1H), 7.31 (s, 1H), 7.20-7.28 (m, 1H), 6.52 (s, 1H), 6.35 (s, 1H), 4.66 (d, J = 3.8 Hz, 1H), 4.08-4.15 (m, 1H), 3.96-4.04 (m, 1H), 3.94 (s, 3H), 2.19- 2.30 (m, 1H), 1.90-2.01 (m, 1H), 1.56-1.76 (m, 3H), 1.38- 1.46 (m, 1H), 1.32 (s, 9H) |
| 193 | 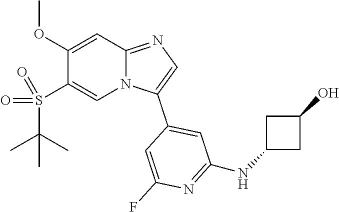 [SMILES: COc1cc2ncc(-c3cc(F)nc(N[C@H]4C[C@H](O)C4)c3)n2cc1S(=O)(=O)C(C)(C)C] | (1r,3r)-3-((4-(6- (tert-butylsulfonyl)- 7- methoxyimidazo[1,2- a]pyridin-3-yl)-6- fluoropyridin-2- yl)amino)cyclobutan- 1-ol | [M + H]⁺ = 449.2 ¹H NMR (400 MHz, DMSO- d₆) δ ppm 8.81 (s, 1H), 7.93 (s, 1H), 7.51 (br d, J = 5.8 Hz, 1H), 7.31 (s, 1H), 6.40 (s, 2H), 5.06 (d, J = 5.3 Hz, 1H), 4.26- 4.34 (m, 1H), 4.02-4.20 (m, 1H), 3.94 (s, 3H), 2.18 (t, J = 6.1 Hz, 4H), 1.33 (s, 9H) |
| 194 | 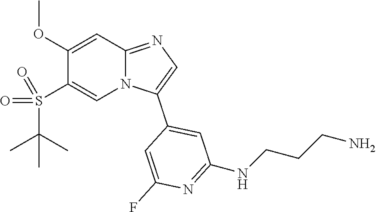 [SMILES: COc1cc2ncc(-c3cc(F)nc(NCCCN)c3)n2cc1S(=O)(=O)C(C)(C)C] | N1-(4-(6-(tert- butylsulfonyl)-7- methoxyimidazo[1,2- a]pyridin-3-yl)-6- fluoropyridin-2- yl)propane-1,3- diamine | [M + H]⁺ = 436.4 ¹H NMR (400 MHz, CDCl₃) δ ppm 8.99 (s, 1H), 7.77 (s, 1H), 7.04 (s, 1H), 6.32 (s, 1H), 6.23 (s, 1H), 5.57 (br d, J = 5.1 Hz, 1H), 3.99 (s, 3H), 3.42 (br d, J = 5.1 Hz, 2H), 2.89 (br t, J = 6.0 Hz, 2H), 1.79 (br t, J = 6.3 Hz, 2H), 1.42 (s, 9H), 0.79- 0.87 (m, 2H) |
| 195 | 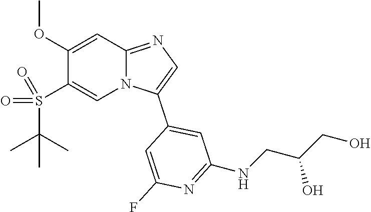 [SMILES: COc1cc2ncc(-c3cc(F)nc(NC[C@@H](O)CO)c3)n2cc1S(=O)(=O)C(C)(C)C] | (R)-3-((4-(6-(tert- butylsulfonyl)-7- methoxyimidazo[1,2- a]pyridin-3-yl)-6- fluoropyridin-2- yl)amino)propane- 1,2-diol | [M + H]⁺ = 453.2 ¹H NMR (400 MHz, DMSO- d₆) δ ppm 8.81 (s, 1H), 7.91 (s, 1H), 7.31 (s, 1H), 7.19 (br s, 1H), 6.61 (s, 1H), 6.36 (s, 1H), 4.82 (d, J = 5.1 Hz, 1H), 4.60 (t, J = 5.7 Hz, 1H), 3.94 (s, 3H), 3.58-3.71 (m, 1H), 3.36- 3.39 (m, 2H), 3.08-3.20 (m, 1H), 1.32 (s, 9H) |
| 196 | 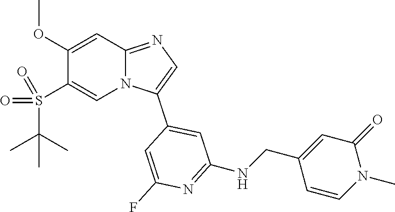 [SMILES: COc1cc2ncc(-c3cc(F)nc(NCc4ccn(C)c(=O)c4)c3)n2cc1S(=O)(=O)C(C)(C)C] | 4-(((4-(6-(tert- butylsulfonyl)-7- methoxyimidazo[1,2- a]pyridin-3-yl)-6- fluoropyridin-2- yl)amino)methyl)-1- methylpyridin- 2(1H)-one | [M + H]⁺ = 500.4 ¹H NMR (400 MHz, DMSO- d₆) δ ppm 8.81 (s, 1H), 7.95 (s, 1H), 7.76-7.86 (m, 1H), 7.62 (d, J = 7.1 Hz, 1H), 7.31 (s, 1H), 6.60 (s, 1H), 6.45 (s, 1H), 6.24-6.26 (m, 1H), 6.17 (dd, J = 7.0, 1.9 Hz, 1H), 4.31 (d, J = 6.1 Hz, 2H), 3.94 (s, 3H), 3.37 (s, 3H), 1.32 (s, 9H) |
| 197 | 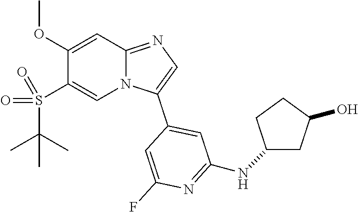 | rac-(1R,3R)-3-((4- (6-(tert- butylsulfonyl)-7- methoxyimidazo[1,2- a]pyridin-3-yl)-6- fluoropyridin-2- yl)amino)cyclopentan- 1-ol | [M + H]⁺ = 463.2 ¹H NMR (400 MHz, DMSO- d₆) δ ppm 8.80 (s, 1H), 7.91 (s, 1H), 7.31 (s, 1H), 7.25 (br d, J = 6.8 Hz, 1H), 6.47 (s, 1H), 6.35 (s, 1H), 4.52 (d, J = 3.8 Hz, 1H), 4.14-4.30 (m, 2H), 3.94 (s, 3H), 2.05-2.20 (m, 1H), 1.86-1.96 (m, 2H), 1.58- 1.67 (m, 1H), 1.37-1.52 (m, 2H), 1.32 (s, 9H) |
| 198 | 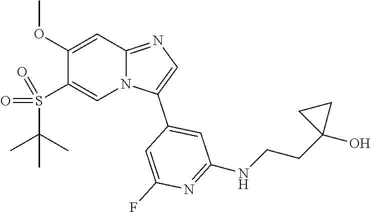 [SMILES: COc1cc2ncc(-c3cc(F)nc(NCCC4(O)CC4)c3)n2cc1S(=O)(=O)C(C)(C)C] | 1-(2-((4-(6-(tert- butylsulfonyl)-7- methoxyimidazo[1,2- a]pyridin-3-yl)-6- fluoropyridin-2- yl)amino)ethyl)cyclo- propan-1-ol | [M + H]⁺ = 463.5 ¹H NMR (400 MHz, DMSO- d₆) δ ppm 8.81 (s, 1H), 7.92 (s, 1H), 7.31 (s, 1H), 7.18 (br s, 1H), 6.50 (s, 1H), 6.35 (s, 1H), 5.16 (s, 1H), 3.94 (s, 3H), 3.36- 3.47 (m, 2H), 1.69-1.77 (m, 2H), 0.52-0.58 (m, 2H), 0.36- 0.40 (m, 2H) |
| 199 | 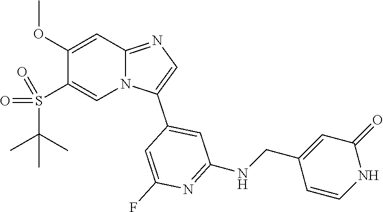 [SMILES: COc1cc2ncc(-c3cc(F)nc(NCc4cc[nH]c(=O)c4)c3)n2cc1S(=O)(=O)C(C)(C)C] | 4-(((4-(6-(tert- butylsulfonyl)-7- methoxyimidazo[1,2- a]pyridin-3-yl)-6- fluoropyridin-2- yl)amino)methyl) pyridin-2(1H)-one | [M + H]⁺ = 486.4 ¹H NMR (400 MHz, DMSO- d₆) δ ppm 11.41 (br s, 1H), 8.82 (s, 1H), 7.95 (s, 1H), 7.75- 7.85 (m, 1H), 7.27-7.36 (m, 2H), 6.61 (s, 1H), 6.45 (s, 1H), 6.18-6.21 (m, 1H), 6.13 (dd, J = 6.7, 1.6 Hz, 1H), 4.31 (d, J = 5.8 Hz, 2H), 3.94 (s, 3H), 1.32 (s, 9H) |
| 200 | 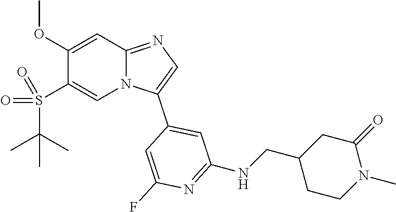 [SMILES: COc1cc2ncc(-c3cc(F)nc(NCC4CCN(C)C(=O)C4)c3)n2cc1S(=O)(=O)C(C)(C)C] | 4-(((4-(6-(tert- butylsulfonyl)-7- methoxyimidazo[1,2- a]pyridin-3-yl)-6- fluoropyridin-2- yl)amino)methyl)-1- methylpiperidin-2- one | [M + H]⁺ = 504.4 ¹H NMR (400 MHz, DMSO- d₆) δ ppm 8.81 (s, 1H), 7.93 (s, 1H), 7.35-7.43 (m, 1H), 7.31 (s, 1H), 6.56 (s, 1H), 6.38 (s, 1H), 3.94 (s, 3H), 3.22-3.31 (m, 2H), 3.16 (br t, J = 6.1 Hz, 2H), 2.79 (s, 3H), 2.30-2.39 (m, 1H), 2.09 (br s, 1H), 1.88- 2.00 (m, 2H), 1.42-1.54 (m, 1H), 1.32 (s, 9H) |
| 201 | 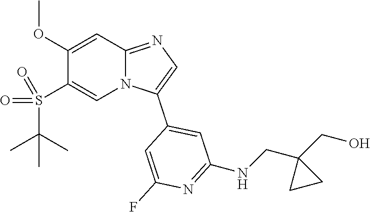 [SMILES: COc1cc2ncc(-c3cc(F)nc(NCC4(CO)CC4)c3)n2cc1S(=O)(=O)C(C)(C)C] | (1-(((4-(6-(tert- butylsulfonyl)-7- methoxyimidazo[1,2- a]pyridin-3-yl)-6- fluoropyridin-2- yl)amino)methyl)cyclo- propyl)methanol | [M + H]⁺ = 463.6 ¹H NMR (400 MHz, DMSO- d₆) δ ppm 8.80 (s, 1H), 7.91 (s, 1H), 7.31 (s, 1H), 7.15-7.26 (m, 1H), 6.59 (s, 1H), 6.35 (s, 1H), 4.58 (t, J = 5.6 Hz, 1H), 3.94 (s, 3H), 3.32 (br s, 2H), 3.27 (d, J = 5.6 Hz, 2H), 1.32 (s, 9H), 0.34-0.50 (m, 4H) |
| 202 | 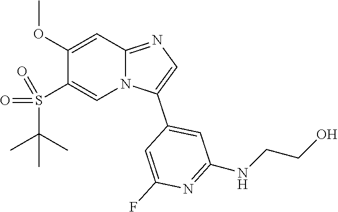 [SMILES: COc1cc2ncc(-c3cc(F)nc(NCCO)c3)n2cc1S(=O)(=O)C(C)(C)C] | 2-((4-(6-(tert- butylsulfonyl)-7- methoxyimidazo[1,2- a]pyridin-3-yl)-6- fluoropyridin-2- yl)amino)ethan-1-ol | [M + H]⁺ = 423.4 ¹H NMR (400 MHz, DMSO- d₆) δ ppm 8.81 (s, 1H), 7.92 (s, 1H), 7.31 (s, 1H), 7.24-7.30 (m, 1H), 6.56 (s, 1H), 6.37 (s, 1H), 4.74 (t, J = 5.4 Hz, 1H), 3.94 (s, 3H), 3.54 (q, J = 5.9 Hz, 2H), 3.27-3.33 (m, 2H), 1.33 (s, 9H) |
| 203 | 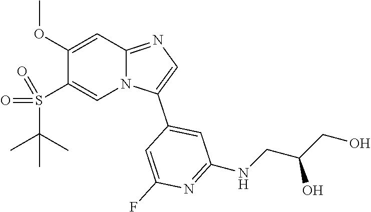 [SMILES: COc1cc2ncc(-c3cc(F)nc(NC[C@H](O)CO)c3)n2cc1S(=O)(=O)C(C)(C)C] | (S)-3-((4-(6-(tert- butylsulfonyl)-7- methoxyimidazo[1,2- a]pyridin-3-yl)-6- fluoropyridin-2- yl)amino)propane- 1,2-diol | [M + H]⁺ = 453.2 ¹H NMR (400 MHz, DMSO- d₆) δ ppm 8.81 (s, 1H), 7.91 (s, 1H), 7.31 (s, 1H), 7.14-7.26 (m, 1H), 6.61 (s, 1H), 6.36 (s, 1H), 4.83 (d, J = 5.1 Hz, 1H), 4.61 (t, J = 5.7 Hz, 1H), 3.94 (s, 3H), 3.60-3.70 (m, 1H), 3.36-3.39 (m, 2H), 3.07-3.20 (m, 1H), 1.32 (s, 9H) |
| 204 | 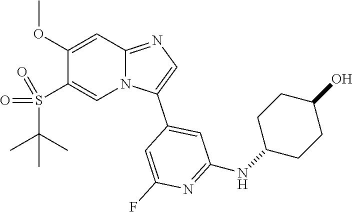 | (1r,4r)-4-((4-(6- (tert-butylsulfonyl)- 7- methoxyimidazo[1,2- a]pyridin-3-yl)-6- fluoropyridin-2- yl)amino)cyclohexan- 1-ol | [M + H]⁺ = 477.3 ¹H NMR (400 MHz, DMSO- d₆) δ ppm 8.79 (s, 1H), 7.90 (s, 1H), 7.31 (s, 1H), 7.11 (br d, J = 7.4 Hz, 1H), 6.49 (s, 1H), 6.33 (s, 1H), 4.57 (d, J = 4.3 Hz, 1H), 3.94 (s, 3H), 3.38- 3.60 (m, 2H), 1.78-1.99 (m, 4H), 1.32 (s, 9H), 1.17-1.28 (m, 4H). |
| 205 | 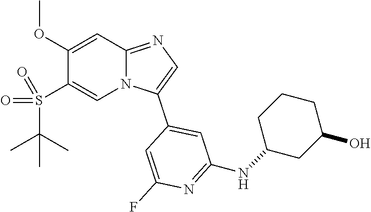 | rac-(1R,3R)-3-((4- (6-(tert- butylsulfonyl)-7- methoxyimidazo[1,2- a]pyridin-3-yl)-6- fluoropyridin-2- yl)amino)cyclohexan- 1-ol | [M + H]⁺ = 477.3 ¹H NMR (400 MHz, DMSO- d₆) δ ppm 8.79 (s, 1H), 7.89 (s, 1H), 7.30 (s, 1H), 7.09 (br d, J = 8.1 Hz, 1H), 6.51 (s, 1H), 6.32 (s, 1H), 4.46 (d, J = 3.5 Hz, 1H), 3.97-4.11 (m, 1H), 3.90-3.97 (m, 4H), 1.40-1.87 (m, 7H), 1.32 (s, 9H), 1.19- 1.29 (m, 1H). |
| 206 | 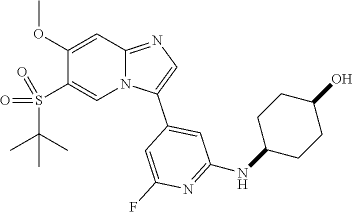 | (1s,4s)-4-((4-(6- (tert-butylsulfonyl)- 7- methoxyimidazo[1,2- a]pyridin-3-yl)-6- fluoropyridin-2- yl)amino)cyclohexan- 1-ol | [M + H]⁺= 477.2 ¹H NMR (400 MHz, DMSO- d₆) δ ppm 8.80 (s, 1H), 7.89 (s, 1H), 7.31 (s, 1H), 7.17 (br d, J = 7.1 Hz, 1H), 6.54 (s, 1H), 6.32 (s, 1H), 4.41 (d, J = 2.8 Hz, 1H), 3.94 (s, 3H), 3.58- 3.77 (m, 2H), 1.57-1.74 (m, 6H), 1.45-1.57 (m, 2H), 1.32 (s, 9H) |
| 207 | 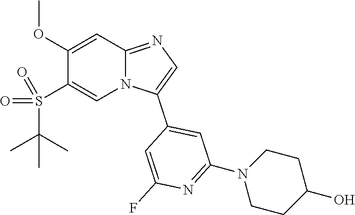 [SMILES: COc1cc2ncc(-c3cc(F)nc(N4CCC(O)CC4)c3)n2cc1S(=O)(=O)C(C)(C)C] | 1-(4-(6-(tert- butylsulfonyl)-7- methoxyimidazo[1,2- a]pyridin-3-yl)-6- fluoropyridin-2- yl)piperidin-4-ol | [M + H]⁺= 463.2 ¹H NMR (400 MHz, DMSO- d₆) δ ppm 8.84 (s, 1H), 7.98 (s, 1H), 7.30 (s, 1H), 6.94 (s, 1H), 6.47 (s, 1H), 4.76 (d, J = 4.1 Hz, 1H), 3.90-4.02 (m, 5H), 3.68-3.82 (m, 1H), 3.15-3.28 (m, 2H), 1.72-1.85 (m, 2H), 1.35-1.43 (m, 2H), 1.33 (s, 9H) |
| 208 | 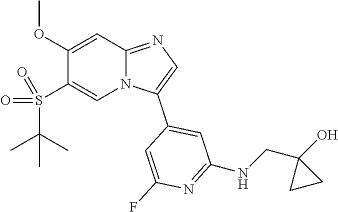 [SMILES: COc1cc2ncc(-c3cc(F)nc(NCC4(O)CC4)c3)n2cc1S(=O)(=O)C(C)(C)C] | 1-(((4-(6-(tert- butylsulfonyl)-7- methoxyimidazo[1,2- a]pyridin-3-yl)-6- fluoropyridin-2- yl)amino)methyl) cyclopropan-1-ol | [M + H]⁺= 449.1 ¹H NMR (400 MHz, DMSO- d₆) δ ppm 8.81 (s, 1H), 7.91 (s, 1H), 7.23-7.40 (m, 2H), 6.63 (s, 1H), 6.35 (s, 1H), 5.41 (s, 1H), 3.94 (s, 3H), 3.38 (d, J = 5.6 Hz, 2H), 1.33 (s, 9H), 0.49- 0.63 (m, 4H) |
| 209 | 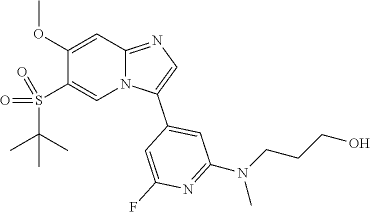 [SMILES: COc1cc2ncc(-c3cc(F)nc(N(C)CCCO)c3)n2cc1S(=O)(=O)C(C)(C)C] | 3-((4-(6-(tert- butylsulfonyl)-7- methoxyimidazo[1,2- a]pyridin-3-yl)-6- fluoropyridin-2- yl)(methyl)amino) propan-1-ol | [M + H]⁺= 451.2 ¹H NMR (400 MHz, DMSO- d₆) δ ppm 8.88 (s, 1H), 7.98 (s, 1H), 7.31 (s, 1H), 6.69 (s, 1H), 6.44 (s, 1H), 4.51 (t, J = 4.9 Hz, 1H), 3.94 (s, 3H), 3.55 (t, J = 7.1 Hz, 2H), 3.38-3.48 (m, 2H), 3.03 (s, 3H), 1.63-1.81 (m, 2H), 1.32 (s, 9H) |
| 210 | 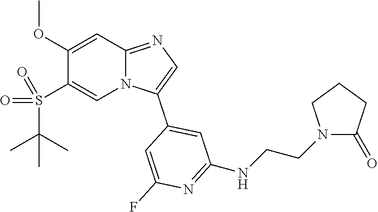 [SMILES: COc1cc2ncc(-c3cc(F)nc(NCCN4CCCC4=O)c3)n2cc1S(=O)(=O)C(C)(C)C] | 1-(2-((4-(6-(tert- butylsulfonyl)-7- methoxyimidazo[1,2- a]pyridin-3-yl)-6- fluoropyridin-2- yl)amino)ethyl) pyrrolidin-2-one | [M + H]⁺= 490.4 ¹H NMR (400 MHz, DMSO- d₆) δ ppm 8.81 (s, 1H), 7.93 (s, 1H), 7.27-7.36 (m, 2H), 6.53 (s, 1H), 6.40 (s, 1H), 3.94 (s, 3H), 3.34-3.45 (m, 6H), 2.11- 2.22 (m, 2H), 1.90 (t, J = 7.5 Hz, 2H), 1.32 (s, 9H) |
| 211 | 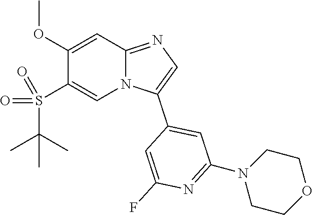 [SMILES: COc1cc2ncc(-c3cc(F)nc(N4CCOCC4)c3)n2cc1S(=O)(=O)C(C)(C)C] | 4-(4-(6-(tert- butylsulfonyl)-7- methoxyimidazo[1,2- a]pyridin-3-yl)-6- fluoropyridin-2- yl)morpholine | [M + H]⁺ = 449.4 ¹H NMR (400 MHz, DMSO- d₆) δ ppm 8.83 (s, 1H), 7.99 (s, 1H), 7.31 (s, 1H), 6.95 (d, J = 1.0 Hz, 1H), 6.59 (s, 1H), 3.94 (s, 3H), 3.67-3.74 (m, 4H), 3.48-3.54 (m, 4H), 1.32 (s, 9H) |
| 212 | 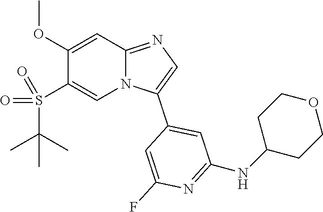 [SMILES: COc1cc2ncc(-c3cc(F)nc(NC4CCOCC4)c3)n2cc1S(=O)(=O)C(C)(C)C] | 4-(6-(tert- butylsulfonyl)-7- methoxyimidazo[1,2- a]pyridin-3-yl)-6- fluoro-N- (tetrahydro-2H- pyran-4-yl)pyridin- 2-amine | [M + H]⁺ = 463.2 ¹H NMR (400 MHz, DMSO- d₆) δ ppm 8.80 (s, 1H), 7.92 (s, 1H), 7.31 (s, 1H), 7.25 (br d, J = 7.6 Hz, 1H), 6.54 (s, 1H), 6.37 (s, 1H), 3.94 (s, 3H), 3.75- 3.90 (m, 3H), 3.37-3.46 (m, 2H), 1.83-1.94 (m, 2H), 1.39- 1.53 (m, 2H), 1.32 (s, 9H) |
| 213 | 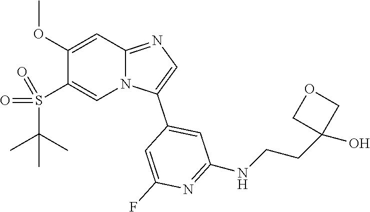 [SMILES: COc1cc2ncc(-c3cc(F)nc(NCCC4(O)COC4)c3)n2cc1S(=O)(=O)C(C)(C)C] | 3-(2-((4-(6-(tert- butylsulfonyl)-7- methoxyimidazo[1,2- a]pyridin-3-yl)-6- fluoropyridin-2- yl)amino)ethyl)oxetan- 3-ol | [M + H]⁺ = 479.2 ¹H NMR (400 MHz, DMSO- d₆) δ ppm 8.90 (s, 1H), 7.98 (s, 1H), 7.31 (s, 1H), 6.47-6.55 (m, 1H), 6.44 (s, 1H), 4.84- 4.95 (m, 2H), 3.94 (s, 3H), 3.41-3.54 (m, 4H), 2.51-2.53 (m, 2H), 2.04-2.16 (m, 1H), 1.74-1.84 (m, 1H), 1.33 (s, 9H) |
| 214 | 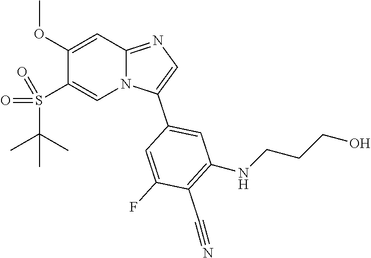 [SMILES: COc1cc2ncc(-c3cc(F)c(C#N)c(NCCCO)c3)n2cc1S(=O)(=O)C(C)(C)C] | 4-(6-(tert- butylsulfonyl)-7- methoxyimidazo[1,2- a]pyridin-3-yl)-2- fluoro-6-((3- hydroxypropyl)amino) benzonitrile | [M + H]⁺ = 461.1 ¹H NMR (400 MHz, DMSO- d₆) δ ppm 8.81 (s, 1H), 7.93 (s, 1H), 7.31 (s, 1H), 6.88-6.75 (m, 3H), 4.64-4.54 (m, 1H), 3.94 (s, 3H), 3.53-3.44 (m, 2H), 3.30-3.27 (m, 2H), 1.76- 1.71 (m, 2H), 1.32 (s, 9H) |
| 215 | 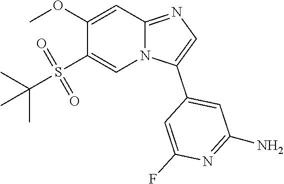 [SMILES: COc1cc2ncc(-c3cc(N)nc(F)c3)n2cc1S(=O)(=O)C(C)(C)C] | 4-(6-(tert- butylsulfonyl)-7- methoxyimidazo[1,2- a]pyridin-3-yl)-6- fluoropyridin-2- amine | [M + H]⁺ = 379.4 ¹H NMR (400 MHz, DMSO- d₆) δ ppm 8.80 (s, 1H), 7.91 (s, 1H), 7.31 (s, 1H), 6.65 (s, 2H), 6.46 (s, 1H), 6.38 (s, 1H), 3.94 (s, 3H), 1.32 (s, 9H) |
| 216 | 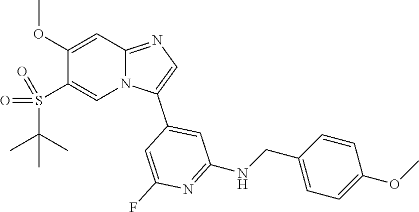 [SMILES: COc1ccc(CNc2cc(-c3cnc4cc(OC)c(S(=O)(=O)C(C)(C)C)cn34)cc(F)n2)cc1] | 6-(tert- butylsulfonyl)-7- methoxyimidazo[1,2- a]pyridin-3-yl)-6- fluoro-N-(4- methoxybenzyl) pyridin-2-amine | [M + H]⁺ = 499.2 ¹H NMR (400 MHz, DMSO- d₆) δ ppm 8.81 (s, 1H), 7.91 (s, 1H), 7.71-7.78 (m, 1H), 7.30- 7.32 (m, 1H), 7.25-7.29 (m, 2H), 6.86-6.93 (m, 2H), 6.56 (s, 1H), 6.40 (s, 1H), 4.39 (d, J = 5.8 Hz, 2H), 3.94 (s, 3H), 3.72 (s, 3H), 1.32 (s, 9H) |
| 217 | 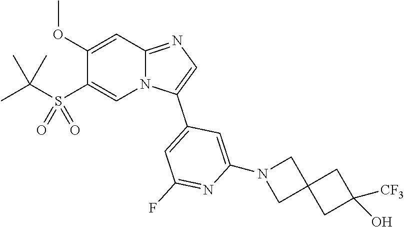 [SMILES: COc1cc2ncc(-c3cc(F)nc(N4CC5(C4)CC(O)(C(F)(F)F)C5)c3)n2cc1S(=O)(=O)C(C)(C)C] | 2-{6-fluoro-4-[7- methoxy-6-(2- methylpropane-2- sulfonyl)imidazo[1,2- a]pyridin-3- yl]pyridin-2-yl}-6- (trifluoromethyl)-2- azaspiro[3.3]heptan- 6-ol | [M + H]⁺ = 543.4 ¹H NMR (400 MHz, DMSO- d₆) δ ppm 8.84 (s, 1 H), 7.98 (s, 1 H), 7.31 (s, 1 H), 6.57 (s, 1 H), 6.54 (s, 1 H), 6.44 (s, 1 H), 4.02 (d, J = 18.25 Hz, 4 H), 3.94 (s, 3 H), 2.64-2.70 (m, 2 H), 2.40-2.46 (m, 2 H), 1.33 (s, 9 H) |
| 218 | 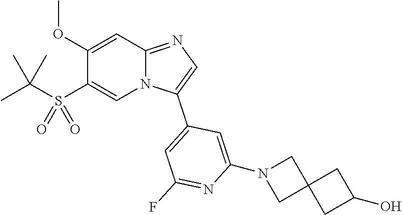 [SMILES: COc1cc2ncc(-c3cc(F)nc(N4CC5(CC(O)C5)C4)c3)n2cc1S(=O)(=O)C(C)(C)C] | 2-{6-fluoro-4-[7- methoxy-6-(2- methylpropane-2- sulfonyl)imidazo[1,2- a]pyridin-3- yl]pyridin-2-yl}-2- azaspiro[3.3]heptan- 6-o1 | [M + H]⁺ = 475.4 ¹H NMR (400 MHz, DMSO- d₆) δ ppm 8.84 (s, 1 H), 7.97 (s, 1 H), 7.31 (s, 1 H), 6.52 (s, 1 H), 6.39 (s, 1 H), 5.08 (d, J = 6.34 Hz, 1 H), 4.05-3.90 (m, 8 H), 2.44-2.49 (m, 2 H), 1.99- 2.06 (m, 2 H), 1.33 (s, 9 H) |
| 219 | 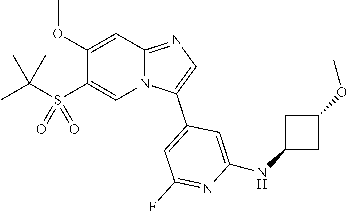 [SMILES: COc1cc2ncc(-c3cc(F)nc(N[C@H]4C[C@H](OC)C4)c3)n2cc1S(=O)(=O)C(C)(C)C] | 6-fluoro-4-[7- methoxy-6-(2- methylpropane-2- sulfonyl)imidazo[1,2- a]pyridin-3-yl]-N- [(trans)-3- methoxycyclobutyl] pyridin-2-amine | [M + H]⁺ = 463.4 ¹H NMR (400 MHz, DMSO- d₆) δ ppm 8.82 (s, 1 H), 7.94 (s, 1 H), 7.56 (br d, J = 5.83 Hz, 1 H), 7.31 (s, 1 H), 6.42 (s, 2 H), 4.15 (br d, J = 4.06 Hz, 1 H), 3.96-4.02 (m, 1 H), 3.91- 3.96 (m, 3 H), 3.15 (s, 3 H), 2.27-2.34 (m, 2 H), 2.12- 2.21 (m, 2 H), 1.33 (s, 9 H) |
| 220 | 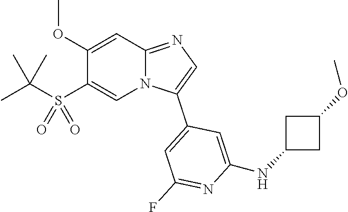 [SMILES: COc1cc2ncc(-c3cc(F)nc(N[C@H]4C[C@@H](OC)C4)c3)n2cc1S(=O)(=O)C(C)(C)C] | 6-fluoro-4-[7- methoxy-6-(2- methylpropane-2- sulfonyl)imidazo[1,2- a]pyridin-3-yl]-N- [cis-3- methoxycyclobutyl] pyridin-2-amine | [M + H]⁺ = 463.4 ¹H NMR (500 MHz, DMSO- d₆) δ ppm 8.81 (s, 1 H), 7.93 (s, 1 H), 7.51 (br d, J = 6.94 Hz, 1 H), 7.31 (s, 1 H), 6.45 (s, 1 H), 6.40 (s, 1 H), 3.94 (s, 3 H), 3.78 (br d, J = 12.61 Hz, 1 H), 3.61 (quin, J = 7.09 Hz, 1 H), 3.14 (s, 3 H), 2.64-2.74 (m, 2 H), 1.73-1.82 (m, 2 H), 1.33 (s, 9 H) |
| 221 | 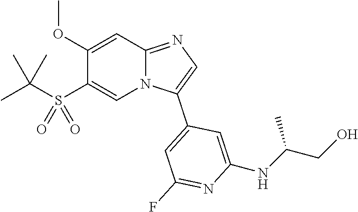 [SMILES: COc1cc2ncc(-c3cc(F)nc(N[C@H](C)CO)c3)n2cc1S(=O)(=O)C(C)(C)C] | (2R)-2-({6-fluoro-4- [7-methoxy-6-(2- methylpropane-2- sulfonyl)imidazo[1,2- a]pyridin-3- yl]pyridin-2- yl}amino)propan-1- ol | M + H]⁺ = 437.2 ¹H NMR (400 MHz, DMSO- d₆) δ ppm 8.80 (s, 1 H), 7.90 (s, 1 H), 7.31 (s, 1 H), 7.06 (br d, J = 7.60 Hz, 1 H), 6.54 (s, 1 H), 6.34 (s, 1 H), 4.75 (t, J = 5.58 Hz, 1 H), 3.94 (s, 3 H), 3.85-3.93 (m, 1 H), 3.48 (dt, J = 10.52, 5.13 Hz, 1 H), 3.34- 3.37 (m, 1 H), 1.32 (s, 9 H), 1.14 (d, J = 6.59 Hz, 3 H) |
| 222 | 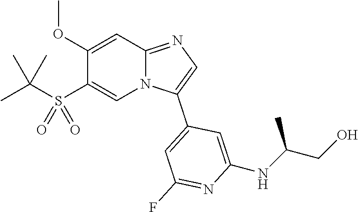 [SMILES: COc1cc2ncc(-c3cc(F)nc(N[C@@H](C)CO)c3)n2cc1S(=O)(=O)C(C)(C)C] | (2S)-2-({6-fluoro-4- [7-methoxy-6-(2- methylpropane-2- sulfonyl)imidazo[1,2- a]pyridin-3- yl]pyridin-2- yl}amino)propan-1- ol | [M + H]⁺ = 437.2 ¹H NMR (400 MHz, DMSO- d₆) δ ppm 8.80 (s, 1 H), 7.90 (s, 1 H), 7.31 (s, 1 H), 7.06 (br d, J = 7.60 Hz, 1 H), 6.54 (s, 1 H), 6.34 (s, 1 H), 4.75 (t, J = 5.58 Hz, 1 H), 3.94 (s, 3 H), 3.85-3.93 (m, 1 H), 3.48 (dt, J = 10.52, 5.13 Hz, 1 H), 3.33- 3.37 (m, 1 H), 1.32 (s, 9 H), 1.14 (d, J = 6.59 Hz, 3 H) |
| 223 | 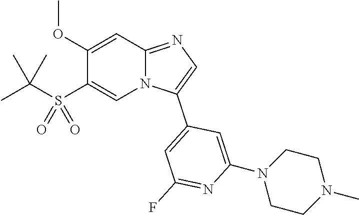 [SMILES: COc1cc2ncc(-c3cc(F)nc(N4CCN(C)CC4)c3)n2cc1S(=O)(=O)C(C)(C)C] | 1-{6-fluoro-4-[7- methoxy-6-(2- methylpropane-2- sulfonyl)imidazo[1,2- a]pyridin-3- yl]pyridin-2-yl}-4- methylpiperazine | [M + H]⁺ = 462.2 ¹H NMR (400 MHz, DMSO- d₆) δ ppm 8.83 (s, 1 H), 7.98 (s, 1 H), 7.30 (s, 1 H), 6.94 (s, 1 H), 6.54 (s, 1 H), 3.94 (s, 3 H), 3.50-3.56 (m, 4 H), 2.35- 2.44 (m, 4 H), 2.22 (s, 3 H), 1.32 (s, 9 H) |
| 224 | 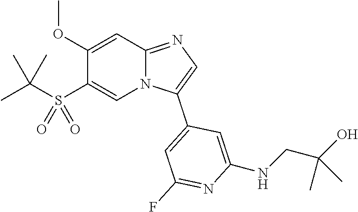 [SMILES: COc1cc2ncc(-c3cc(F)nc(NCC(C)(C)O)c3)n2cc1S(=O)(=O)C(C)(C)C] | 1-({6-fluoro-4-[7- methoxy-6-(2- methylpropane-2- sulfonyl)imidazo[1,2- a]pyridin-3- yl]pyridin-2- yl}amino)-2- methylpropan-2-ol | [M + H]⁺ = 451.2 ¹H NMR (400 MHz, DMSO- d₆) δ ppm 8.81 (s, 1 H), 7.90 (s, 1 H), 7.31 (s, 1 H), 7.08 (br s, 1 H), 6.69 (s, 1 H), 6.34 (s, 1 H), 4.52 (s, 1 H), 3.94 (s, 3 H), 3.16-3.29 (m, 2 H), 1.32 (s, 9 H), 1.14 (s, 6 H) |
| 225 | 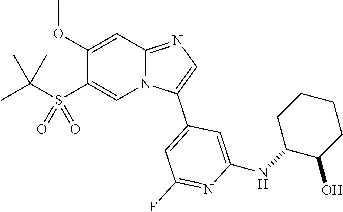 | Mixture of 2 Enantiomers, trans: (1R,2R & 1S,2S)-2- ((4-(6-(tert- butylsulfonyl)-7- methoxyimidazo[1,2- a]pyridin-3-yl)-6- fluoropyridin-2- yl)amino)cyclohexan- 1-ol | [M + H]⁺ = 477.5 ¹H NMR (400 MHz, DMSO- d₆) δ ppm 8.81 (s, 1 H), 7.90 (s, 1 H), 7.31 (s, 1 H), 7.00- 7.10 (m, 1 H), 6.55 (s, 1 H), 6.32 (s, 1 H), 4.61 (d, J = 5.07 Hz, 1 H), 3.94 (s, 3 H), 3.34-3.38 (m, 1 H), 2.00- 1.84 (m, 2H), 1.68-1.56 (m, 2H), 1.33 (s, 9H), 1.30-1.10 (m, 5H). |
| 226 | 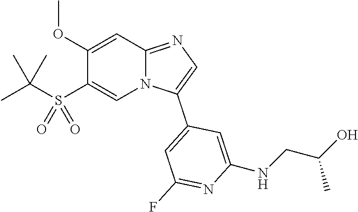 [SMILES: COc1cc2ncc(-c3cc(F)nc(NC[C@@H](C)O)c3)n2cc1S(=O)(=O)C(C)(C)C] | (2R)-1-({6-fluoro-4- [7-methoxy-6-(2- methylpropane-2- sulfonyl)imidazo[1,2- a]pyridin-3- yl]pyridin-2- yl}amino)propan-2- ol | [M + H]⁺ = 437.1 ¹H NMR (400 MHz, DMSO- d₆) δ ppm 8.81 (s, 1 H), 7.91 (s, 1 H), 7.31 (s, 1 H), 7.24 (br s, 1 H), 6.59 (s, 1 H), 6.36 (s, 1 H), 4.74 (d, J = 4.82 Hz, 1 H), 3.94 (s, 3 H), 3.75-3.85 (m, 1 H), 3.08-3.26 (m, 2 H), 1.33 (s, 9 H), 1.09 (d, J = 6.08 Hz, 3 H) |
| 227 | 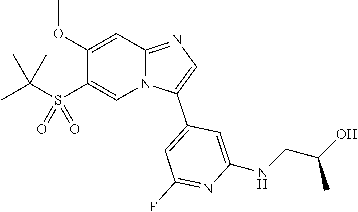 [SMILES: COc1cc2ncc(-c3cc(F)nc(NC[C@H](C)O)c3)n2cc1S(=O)(=O)C(C)(C)C] | (2S)-1-({6-fluoro-4- [7-methoxy-6-(2- methylpropane-2- sulfonyl)imidazo[1,2- a]pyridin-3- yl]pyridin-2- yl}amino)propan-2- ol | [M + H]⁺ = 437.3 ¹H NMR (400 MHz, DMSO- d₆) δ ppm 8.81 (s, 1 H), 7.91 (s, 1 H), 7.31 (s, 1 H), 7.24 (br s, 1 H), 6.59 (s, 1 H), 6.35 (s, 1 H), 4.74 (d, J = 4.82 Hz, 1 H), 3.94 (s, 3 H), 3.75-3.85 (m, 1 H), 3.10-3.24 (m, 2 H), 1.33 (s, 9 H), 1.09 (d, J = 6.08 Hz, 3 H) |
| 228 | 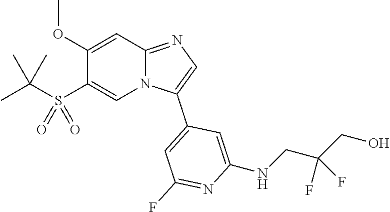 [SMILES: COc1cc2ncc(-c3cc(F)nc(NCC(F)(F)CO)c3)n2cc1S(=O)(=O)C(C)(C)C] | 2,2-difluoro-3-({6- fluoro-4-[7- methoxy-6-(2- methylpropane-2- sulfonyl)imidazo[1,2- a]pyridin-3- yl]pyridin-2- yl}amino)propan-1- ol | [M + H]⁺ = 473.4 ¹H NMR (400 MHz, DMSO- d₆) δ ppm 8.80 (s, 1 H), 7.94 (s, 1 H), 7.59 (t, J = 6.10 Hz, 1 H), 7.32 (s, 1 H), 6.69 (s, 1 H), 6.48 (s, 1 H), 5.56 (t, J = 6.34 Hz, 1 H), 3.94 (s, 3 H), 3.75-3.88 (m, 2 H), 3.66 (td, J = 13.56, 6.34 Hz, 2 H), 1.33 (s, 9 H) |
| 229 | 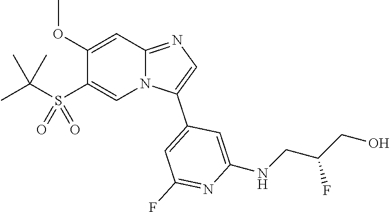 [SMILES: COc1cc2ncc(-c3cc(F)nc(NC[C@@H](F)CO)c3)n2cc1S(=O)(=O)C(C)(C)C] | (2R)-2-fluoro-3-({6- fluoro-4-[7- methoxy-6-(2- methylpropane-2- sulfonyl)imidazo[1,2- a]pyridin-3- yl]pyridin-2- yl}amino)propan-1- ol | [M + H]⁺ = 455.2 ¹H NMR (400 MHz, DMSO- d₆) δ ppm 8.81 (s, 1 H), 7.93 (s, 1 H), 7.51 (br s, 1 H), 7.31 (s, 1 H), 6.60 (s, 1 H), 6.42 (s, 1 H), 5.01 (t, J = 5.70 Hz, 1 H), 4.49-4.79 (m, 1 H), 3.94 (s, 3 H), 3.40-3.71 (m, 4 H), 1.33 (s, 9 H) |
| 230 | 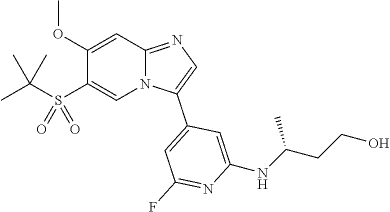 [SMILES: COc1cc2ncc(-c3cc(F)nc(N[C@H](C)CCO)c3)n2cc1S(=O)(=O)C(C)(C)C] | (3R)-3-({6-fluoro-4- [7-methoxy-6-(2- methylpropane-2- sulfonyl)imidazo[1,2- a]pyridin-3- yl]pyridin-2- yl}amino)butan-1-ol | [M + H]⁺ = 451.2 ¹H NMR (400 MHz, DMSO- d₆) δ ppm 8.80 (s, 1 H), 7.90 (s, 1 H), 7.31 (s, 1 H), 7.10 (br d, J = 7.60 Hz, 1 H), 6.49 (s, 1 H), 6.33 (s, 1 H), 4.44 (t, J = 5.07 Hz, 1 H), 3.94 (s, 4 H), 3.44-3.50 (m, 2 H), 1.55- 1.75 (m, 2 H), 1.32 (s, 9 H), 1.14 (d, J = 6.34 Hz, 3 H) |
| 231 | 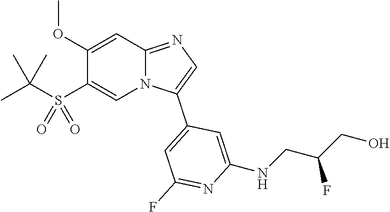 [SMILES: COc1cc2ncc(-c3cc(F)nc(NC[C@H](F)CO)c3)n2cc1S(=O)(=O)C(C)(C)C] | (2S)-2-fluoro-3-({6- fluoro-4-[7- methoxy-6-(2- methylpropane-2- sulfonyl)imidazo[1,2- a]pyridin-3- yl]pyridin-2- yl}amino)propan-1- ol | [M + H]⁺ = 455.2 ¹H NMR (400 MHz, DMSO- d₆) δ ppm 8.80 (s, 1 H), 7.85- 7.97 (m, 1 H), 7.43-7.62 (m, 1 H), 7.31 (s, 1 H), 6.60 (s, 1 H), 6.42 (s, 1 H), 4.96-5.06 (m, 1 H), 4.53-4.75 (m, 1 H), 3.94 (s, 3 H), 3.39-3.72 (m, 4 H), 1.33 (s, 9 H) |
| 232 | 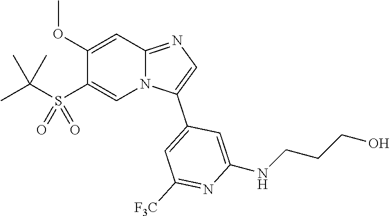 [SMILES: COc1cc2ncc(-c3cc(NCCCO)nc(C(F)(F)F)c3)n2cc1S(=O)(=O)C(C)(C)C] | 3-({4-[7-methoxy-6- (2-methylpropane-2- sulfonyl)imidazo[1,2- a]pyridin-3-yl]-6- (trifluoromethyl)pyridin- 2- yl}amino)propan-1- ol | [M + H]⁺ = 487.5 ¹H NMR (400 MHz, DMSO- d₆) δ ppm 8.80 (s, 1 H), 7.99 (s, 1 H), 7.38 (t, J = 5.20 Hz, 1 H), 7.31 (s, 1 H), 7.12 (s, 1 H), 6.87 (s, 1 H), 4.49 (br s, 1 H), 3.94 (s, 3 H), 3.45-3.53 (m, 2 H), 2.52-2.56 (m, 2 H), 1.72 (quin, J = 6.65 Hz, 2 H), 1.33 (s, 9 H) |
| 233 | 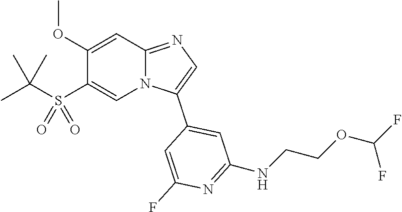 [SMILES: COc1cc2ncc(-c3cc(F)nc(NCCOC(F)F)c3)n2cc1S(=O)(=O)C(C)(C)C] | 4-(6-(tert- butylsulfonyl)-7- methoxyimidazo[1,2- a]pyridin-3-yl)-N- (2- (difluoromethoxy) ethyl)-6-fluoropyridin- 2-amine | [M + H]⁺ = 473.0 ¹H NMR (400 MHz, DMSO- d₆) δ ppm 8.81 (s, 1H), 7.93 (s, 1H), 7.47 (t, J = 4.8 Hz, 1H), 7.31 (s, 1H), 6.90-6.51 (m, 2H), 6.42 (s, 1H), 3.99-3.95 (m, 2H), 3.94 (s, 3H), 3.56- 3.47 (m, 2H), 1.33 (s, 9H). |
| 234 | 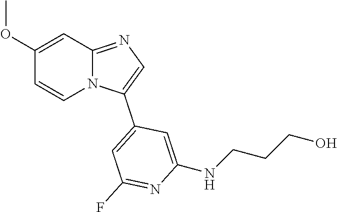 [SMILES: COc1ccn2c(-c3cc(F)nc(NCCCO)c3)cnc2c1] | 3-[(6-fluoro-4-{7- methoxyimidazo[1,2- a]pyridin-3- yl}pyridin-2- yl)amino]propan-1- ol | [M + H]⁺ = 317.1 ¹H NMR (400 MHz, DMSO- d6) δ ppm 8.52 (d, J = 7.4 Hz, 1H), 7.79 (s, 1H), 7.07 (d, J = 2.3 Hz, 1H), 7.02 (br t, J = 5.2 Hz, 1H), 6.74 (dd, J = 7.6, 2.8 Hz, 1H), 6.60-6.57 (m, 1H), 6.37 (s, 1H), 4.50 (t, J = 5.1 Hz, 1H), 3.87 (s, 3H), 3.53- 3.46 (m, 2H), 3.31-3.23 (m, 2H), 1.69 (quin, J = 6.6 Hz, 2H) |
| 235 | 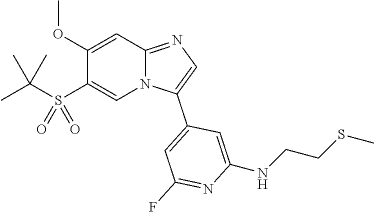 [SMILES: COc1cc2ncc(-c3cc(F)nc(NCCSC)c3)n2cc1S(=O)(=O)C(C)(C)C] | 4-(6-(tert- butylsulfonyl)-7- methoxyimidazo[1,2- a]pyridin-3-yl)-6- fluoro-N-(2- (methylthio)ethyl) pyridin-2-amine | [M + H]⁺ = 453.2 ¹H NMR (400 MHz, DMSO- d₆) δ ppm 8.81 (s, 1H), 7.93 (s, 1H), 7.46-7.38 (m, 1H), 7.31 (s, 1H), 6.57-6.53 (m, 1H), 6.40 (s, 1H), 3.94 (s, 3H), 3.49- 3.39 (m, 2H), 2.67 (t, J = 7.1 Hz, 2H), 2.11 (s, 3H), 1.33 (s, 9H) |
| 236 | 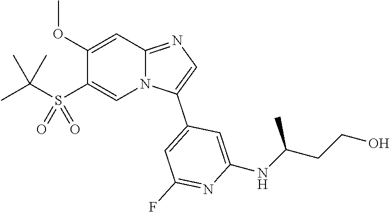 [SMILES: COc1cc2ncc(-c3cc(F)nc(N[C@@H](C)CCO)c3)n2cc1S(=O)(=O)C(C)(C)C] | (3S)-3-({6-fluoro-4- [7-methoxy-6-(2- methylpropane-2- sulfonyl)imidazo[1,2- a]pyridin-3- yl]pyridin-2- yl}amino)butan-1-ol | [M + H]⁺ = 451.5 ¹H NMR (400 MHz, DMSO- d₆) δ ppm 8.80 (s, 1 H), 7.90 (s, 1 H), 7.31 (s, 1 H), 7.10 (br d, J = 7.86 Hz, 1 H), 6.49 (s, 1 H), 6.33 (s, 1 H), 4.44 (t, J = 5.07 Hz, 1 H), 3.90-3.98 (m, 4 H), 3.42-3.52 (m, 2 H), 1.54- 1.75 (m, 2 H), 1.32 (s, 9 H), 1.14 (d, J = 6.34 Hz, 3 H) |
| 237 | 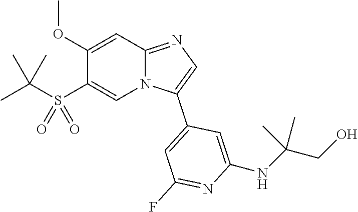 [SMILES: COc1cc2ncc(-c3cc(F)nc(NC(C)(C)CO)c3)n2cc1S(=O)(=O)C(C)(C)C] | 2-({6-fluoro-4-[7- methoxy-6-(2- methylpropane-2- sulfonyl)imidazo[1,2- a]pyridin-3- yl]pyridin-2- yl}amino)-2- methylpropan-1-ol | [M + H]⁺ = 451.6 ¹H NMR (400 MHz, DMSO- d₆) δ ppm 8.77 (s, 1 H), 7.85 (s, 1 H), 7.30 (s, 1 H), 6.76 (s, 1 H), 6.63 (s, 1 H), 6.33 (s, 1 H), 4.82-4.87 (m, 1 H), 3.94 (s, 3 H), 3.55 (d, J = 5.83 Hz, 2 H), 1.32 (m, 15 H). |
| 238 | 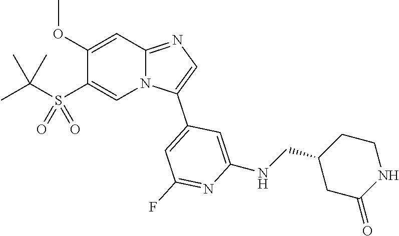 [SMILES: COc1cc2ncc(-c3cc(F)nc(NC[C@@H]4CCNC(=O)C4)c3)n2cc1S(=O)(=O)C(C)(C)C] | (4R or S)-4-[({6- fluoro-4-[7- methoxy-6-(2- methylpropane-2- sulfonyl)imidazo[1,2- a]pyridin-3- yl]pyridin-2- yl}amino)methyl]pi peridin-2-one (Enantiomer 1) | [M + H]⁺ = 490.5 ¹H NMR (400 MHz, DMSO- d₆) δ ppm 8.81 (s, 1 H), 7.93 (s, 1 H), 7.46 (br s, 1 H), 7.38 (br s, 1 H), 7.31 (s, 1 H), 6.56 (s, 1 H), 6.38 (s, 1 H), 3.94 (s, 3 H), 3.07-3.22 (m, 4 H), 2.22- 2.30 (m, 1 H), 2.02-2.14 (m, 1 H), 1.83-1.94 (m, 2 H), 1.32- 1.42 (m, 10 H) |
| 239 | 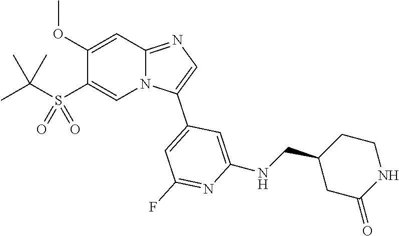 [SMILES: COc1cc2ncc(-c3cc(F)nc(NC[C@H]4CCNC(=O)C4)c3)n2cc1S(=O)(=O)C(C)(C)C] | (4R or S)-4-[({6- fluoro-4-[7- methoxy-6-(2- methylpropane-2- sulfonyl)imidazo[1,2- a]pyridin-3- yl]pyridin-2- yl}amino)methyl]pi peridin-2-one (Enantiomer 2) | [M + H]⁺ = 490.5 ¹H NMR (400 MHz, DMSO- d₆) δ ppm 8.81 (s, 1 H), 7.93 (s, 1 H), 7.46 (br s, 1 H), 7.38 (br s, 1 H), 7.31 (s, 1 H), 6.56 (s, 1 H), 6.38 (s, 1 H), 3.94 (s, 3 H), 3.07-3.22 (m, 4 H), 2.22- 2.30 (m, 1 H), 2.02-2.14 (m, 1 H), 1.83-1.94 (m, 2 H), 1.32- 1.42 (m, 10 H) |
| 240 | 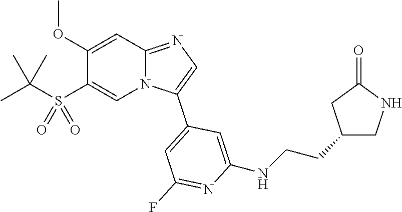 [SMILES: COc1cc2ncc(-c3cc(F)nc(NCC[C@H]4CNC(=O)C4)c3)n2cc1S(=O)(=O)C(C)(C)C] | (4R or S)-4-[2-({6- fluoro-4-[7- methoxy-6-(2- methylpropane-2- sulfonyl)imidazo[1,2- a]pyridin-3- yl]pyridin-2- yl}amino)ethyl] pyrrolidin-2-one (Enantiomer 1) | [M + H]⁺ = 490.5 ¹H NMR (600 MHz, DMSO- d6) δ ppm 8.81 (s, 1 H), 7.93 (s, 1 H), 7.50 (s, 1 H), 7.34- 7.25 (m, 2 H), 6.51 (s, 1 H), 6.37 (s, 1 H), 3.94 (s, 3 H), 3.40-3.34 (m, 1 H), 3.26- 3.18 (m, 2 H), 2.91 (dd, J = 9.4, 7.1 Hz, 1 H), 2.41 (dt, J = 15.5, 7.8 Hz, 1 H), 2.26 (dd, J = 16.2, 8.6 Hz, 1 H), 1.88 (dd, J = 16.2, 8.4 Hz, 1 H), 1.74- 1.61 (m, 2 H), 1.32 (s, 9 H) |
| 241 | 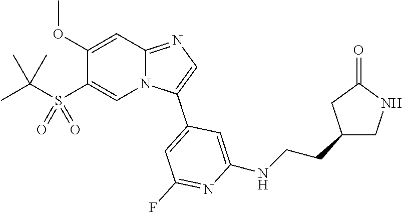 [SMILES: COc1cc2ncc(-c3cc(F)nc(NCC[C@@H]4CNC(=O)C4)c3)n2cc1S(=O)(=O)C(C)(C)C] | (4R or S)-4-[2-({6- fluoro-4-[7- methoxy-6-(2- methylpropane-2- sulfonyl)imidazo[1,2- a]pyridin-3- yl]pyridin-2- yl}amino)ethyl] pyrrolidin-2-one (Enantiomer 2) | [M + H]⁺ = 490.5 ¹H NMR (400 MHz, DMSO- d₆) δ ppm 8.81 (s, 1 H), 7.93 (s, 1 H), 7.50 (s, 1 H), 7.34- 7.24 (m, 2 H), 6.51 (s, 1 H), 6.37 (s, 1 H), 3.94 (s, 3 H), 3.40-3.34 (m, 1 H), 3.26- 3.18 (m, 2 H), 2.91 (dd, J = 9.4, 7.1 Hz, 1 H), 2.41 (dt, J = 15.5, 7.8 Hz, 1 H), 2.26 (dd, J = 16.2, 8.6 Hz, 1 H), 1.88 (dd, J = 16.2, 8.4 Hz, 1 H), 1.74- 1.61 (m, 2 H), 1.32 (s, 9 H) |
| 242 | 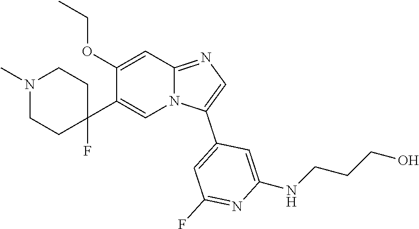 [SMILES: CCOc1cc2ncc(-c3cc(F)nc(NCCCO)c3)n2cc1C1(F)CCN(C)CC1] | 3-({4-[7-ethoxy-6- (4-fluoro-1- methylpiperidin-4- yl)imidazo[1,2- a]pyridin-3-yl]-6- fluoropyridin-2- yl}amino)propan-1- ol | [M + H]⁺ = 446.6 ¹H NMR (400 MHz, DMSO- d₆) δ ppm 8.42 (s, 1 H), 7.79 (s, 1 H), 7.18 (br s, 1 H), 7.15 (s, 1 H), 6.56 (s, 1 H), 6.36 (s, 1 H), 4.40-4.51 (m, 1 H), 4.19 (q, J = 7.01 Hz, 2 H), 3.49 (q, J = 6.08 Hz, 2 H), 3.24-3.31 (m, 2 H), 2.59-2.77 (m, 4 H), 2.18-2.27 (m, 5 H), 1.65- 1.85 (m, 4 H), 1.40 (t, J = 6.97 Hz, 3 H) |
| 243 | 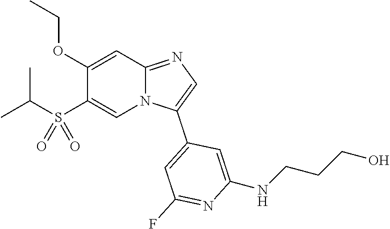 [SMILES: CCOc1cc2ncc(-c3cc(F)nc(NCCCO)c3)n2cc1S(=O)(=O)C(C)C] | 3-({4-[7-ethoxy-6- (propane-2- sulfonyl)imidazo[1,2- a]pyridin-3-yl]-6- fluoropyridin-2- yl}amino)propan-1- ol | [M + H]⁺ = 437.5 ¹H NMR (400 MHz, DMSO- d₆) δ ppm 8.83 (s, 1 H), 7.91 (s, 1 H), 7.32 (s, 1 H), 7.25 (s, 1 H), 6.55 (s, 1 H), 6.41 (s, 1 H), 4.49 (t, J = 5.07 Hz, 1 H), 4.24-4.32 (m, 2 H), 3.73 (quin, J = 6.84 Hz, 1 H), 3.45- 3.53 (m, 2 H), 3.24-3.31 (m, 2 H), 1.70 (quin, J = 6.59 Hz, 2 H), 1.40 (t, J = 6.97 Hz, 3 H), 1.23 (d, J = 6.84 Hz, 6 H). |
| 244 | 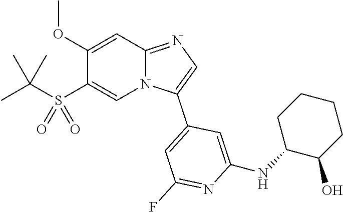 [SMILES: COc1cc2ncc(-c3cc(F)nc(N[C@@H]4CCCC[C@H]4O)c3)n2cc1S(=O)(=O)C(C)(C)C] | trans-(15,2S or 1R,2R)-2-({6-fluoro- 4-[7-methoxy-6-(2- methylpropane-2- sulfonyl)imidazo[1,2- a]pyridin-3- yl]pyridin-2- yl}amino)cyclohexan- 1-ol (Enantiomer 2) | [M + H]⁺ = 477.5 ¹H NMR (400 MHz, DMSO- d₆) δ ppm 8.81 (s, 1 H), 7.90 (s, 1 H), 7.31 (s, 1 H), 7.05 (br d, J = 7.35 Hz, 1 H), 6.55 (s, 1 H), 6.32 (s, 1 H), 4.62 (d, J = 5.07 Hz, 1 H), 3.94 (s, 3 H), 3.54-3.40 (m, 1 H), 2.55- 2.50 (m, 1 H), 2.00-1.84 (m, 2 H), 1.70-1.55 (m, 2 H), 1.33 (s, 9 H), 1.32-1.11 (m, 4 H) |
| 245 | 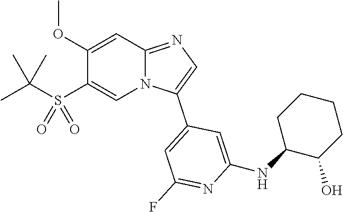 [SMILES: COc1cc2ncc(-c3cc(F)nc(N[C@H]4CCCC[C@@H]4O)c3)n2cc1S(=O)(=O)C(C)(C)C] | trans-(15,2S or 1R,2R)-2-({6-fluoro- 4-[7-methoxy-6-(2- methylpropane-2- sulfonyl)imidazo[1,2- a]pyridin-3- yl]pyridin-2- yl}amino)cyclohexan- 1-ol (Enantiomer 1) | [M + H]⁺ = 477.5 ¹H NMR (400 MHz, DMSO- d₆) δ ppm 8.81 (s, 1 H), 7.90 (s, 1 H), 7.31 (s, 1 H), 7.05 (br d, J = 7.35 Hz, 1 H), 6.55 (s, 1 H), 6.32 (s, 1 H), 4.62 (d, J = 5.07 Hz, 1 H), 3.94 (s, 3 H), 3.54-3.40 (m, 1 H), 2.55- 2.50 (m, 1 H), 2.00-1.84 (m, 2 H), 1.70-1.55 (m, 2 H), 1.33 (s, 9H), 1.32-1.11 (m, 4 H) |
| 246 | 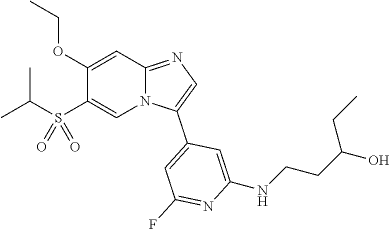 [SMILES: CCOc1cc2ncc(-c3cc(F)nc(NCCC(O)CC)c3)n2cc1S(=O)(=O)C(C)C] | Rac-1-({4-[7- ethoxy-6-(propane- 2- sulfonyl)imidazo[1,2- a]pyridin-3-yl]-6- fluoropyridin-2- yl}amino)pentan-3- ol | [M + H]⁺ = 465.5 ¹H NMR (400 MHz, DMSO- d₆) δ ppm 8.83 (s, 1 H), 7.91 (s, 1 H), 7.32 (s, 1 H), 7.21 (br s, 1 H), 6.54 (s, 1 H), 6.41 (s, 1 H), 4.44 (d, J = 5.32 Hz, 1 H), 4.28 (q, J = 7.10 Hz, 2 H), 3.73 (sept, J = 6.78 Hz, 1 H), 3.35- 3.48 (m, 1 H), 3.21-3.32 (m, 2 H), 1.61-1.72 (m, 1 H), 1.30- 1.55 (m, 3 H), 1.40 (t, J = 6.97 Hz, 3 H), 1.23 (d, J = 6.84 Hz, 6 H), 0.87 (t, J = 7.48 Hz, 3 H) |
| 247 | 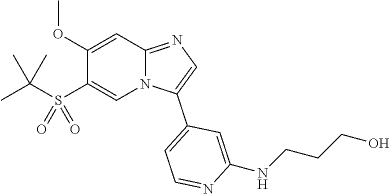 [SMILES: COc1cc2ncc(-c3ccnc(NCCCO)c3)n2cc1S(=O)(=O)C(C)(C)C] | 3-({4-[7-methoxy-6- (2-methylpropane-2- sulfonyl)imidazo[1,2- a]pyridin-3- yl]pyridin-2- yl}amino)propan-1- ol | [M + H]⁺ = 419.5 ¹H NMR (400 MHz, DMSO- d₆) δ ppm 8.80 (s, 1 H), 8.10 (d, J = 5.07 Hz, 1 H), 7.85 (s, 1 H), 7.29 (s, 1 H), 6.79 (t, J = 5.58 Hz, 1 H), 6.67 (dd, J = 5.32, 1.27 Hz, 1 H), 6.61 (s, 1 H), 4.52 (br s, 1 H), 3.94 (s, 3 H), 3.49 (t, J = 6.21 Hz, 2 H), 3.28-3.31 (m, 2 H), 1.64- 1.74 (m, 2 H), 1.32 (s, 9 H) |
| 248 | 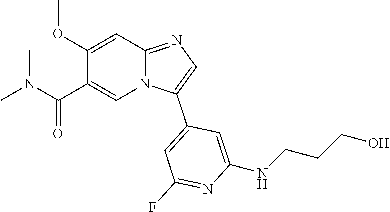 [SMILES: COc1cc2ncc(-c3cc(F)nc(NCCCO)c3)n2cc1C(=O)N(C)C] | 3-{2-fluoro-6-[(3- hydroxypropyl)amino ]pyridin-4-yl}-7- methoxy-N,N- dimethylimidazo[1,2- a]pyridine-6- carboxamide | [M + H]⁺ = 388.5 ¹H NMR (400 MHz, DMSO- d₆) δ ppm 8.46 (s, 1 H), 7.85 (s, 1 H), 7.19 (s, 1 H), 7.08 (br t, J = 5.20 Hz, 1 H), 6.58 (s, 1 H), 6.38 (s, 1 H), 4.49 (t, J = 5.20 Hz, 1 H), 3.90 (s, 3 H), 3.40-3.52 (m, 2 H), 3.24- 3.30 (m, 2 H), 2.99 (s, 3 H), 2.82 (s, 3 H), 1.68 (quin, J = 6.59 Hz, 2 H) |
| 249 | 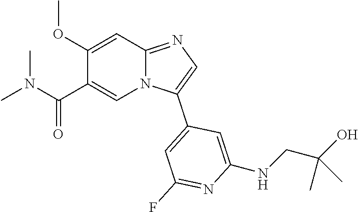 [SMILES: COc1cc2ncc(-c3cc(F)nc(NCC(C)(C)O)c3)n2cc1C(=O)N(C)C] | 3-{2-fluoro-6-[(2- hydroxy-2- methylpropyl)amino] pyridin-4-yl}-7- methoxy-N,N- dimethylimidazo[1,2- a]pyridine-6- carboxamide | [M + H]⁺ = 402.5 ¹H NMR (400 MHz, DMSO- d₆) δ ppm 8.48 (s, 1 H), 7.87 (s, 1 H), 7.19 (s, 1 H), 6.95 (br s, 1 H), 6.80 (s, 1 H), 6.37 (s, 1 H), 4.53 (s, 1 H), 3.91 (s, 3 H), 3.23 (d, J = 5.83 Hz, 2 H), 3.00 (s, 3 H), 2.83 (s, 3 H), 1.13 (s, 6 H) |
| 250 | 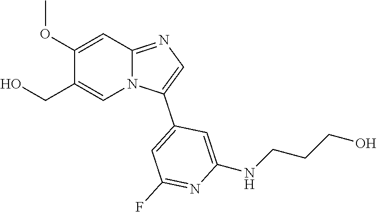 [SMILES: COc1cc2ncc(-c3cc(F)nc(NCCCO)c3)n2cc1CO] | 3-({6-fluoro-4-[6- (hydroxymethyl)-7- methoxyimidazo[1,2- a]pyridin-3- yl]pyridin-2- yl}amino)propan-1- ol | [M + H]⁺ = 347.4 ¹H NMR (400 MHz, DMSO- d₆) δ ppm 8.41 (s, 1 H), 7.75 (s, 1 H), 7.10 (br t, J = 5.07 Hz, 1 H), 7.06 (s, 1 H), 6.56 (s, 1 H), 6.36 (s, 1 H), 5.30 (t, J = 5.58 Hz, 1 H), 4.48-4.55 (m, 3 H), 3.89 (s, 3 H), 3.47-3.52 (m, 2 H), 3.24-3.30 (m, 2 H), 1.69 (quin, J = 6.65 Hz, 2 H) |
| 251 | 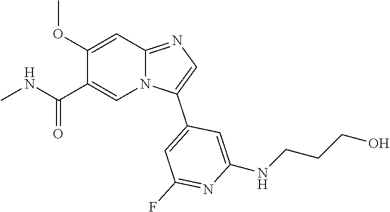 [SMILES: CNC(=O)c1cn2c(-c3cc(F)nc(NCCCO)c3)cnc2cc1OC] | 3-{2-fluoro-6-[(3- hydroxypropyl)amin o]pyridin-4-yl}-7- methoxy-N- methylimidazo[1,2- a]pyridine-6- carboxamide | [M + H]⁺ = 374.4 ¹H NMR (400 MHz, DMSO- d₆) δ ppm 8.86 (s, 1 H), 8.24- 8.35 (m, 1 H), 7.86 (s, 1 H), 7.19 (s, 1 H), 7.11-7.17 (m, 1 H), 6.55-6.62 (m, 1 H), 6.40 (s, 1 H), 4.46-4.53 (m, 1 H), 3.96 (s, 3 H), 3.46-3.53 (m, 2 H), 3.24-3.31 (m, 2 H), 2.82 (d, J = 4.56 Hz, 3 H), 1.64- 1.75 (m, 2 H) |
| Int. 85 | 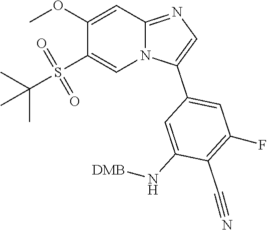 | 4-(6-(tert- butylsulfonyl)-7- methoxyimidazo[1,2- a]pyridin-3-yl)-2- ((2,4- dimethoxybenzyl) amino)-6- fluorobenzonitrile | [M + H]⁺ = 553.0 |
| 252 | 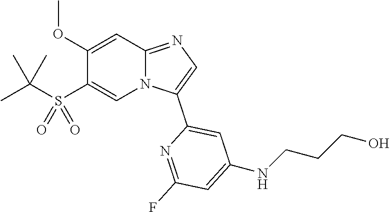 [SMILES: COc1cc2ncc(-c3cc(NCCCO)cc(F)n3)n2cc1S(=O)(=O)C(C)(C)C] | 3-((2-(6-(tert- butylsulfonyl)-7- methoxyimidazo[1,2- a]pyridin-3-yl)-6- fluoropyridin-4- yl)amino)propan-1- ol | [M + H]⁺ = 437.0 ¹H NMR (400 MHz, DMSO- d₆) δ ppm: 10.34 (s, 1H), 8.70 (br s, 1H), 7.46 (s, 1H), 7.31 (br s, 1H), 7.07 (s, 1H), 6.24- 6.20 (m, 1H), 4.05 (s, 3H), 3.54-3.49 (m, 2H), 3.28-3.22 (m, 2H), 1.76-1.67 (m, 2H), 1.34 (s, 9H). |

### Example 253

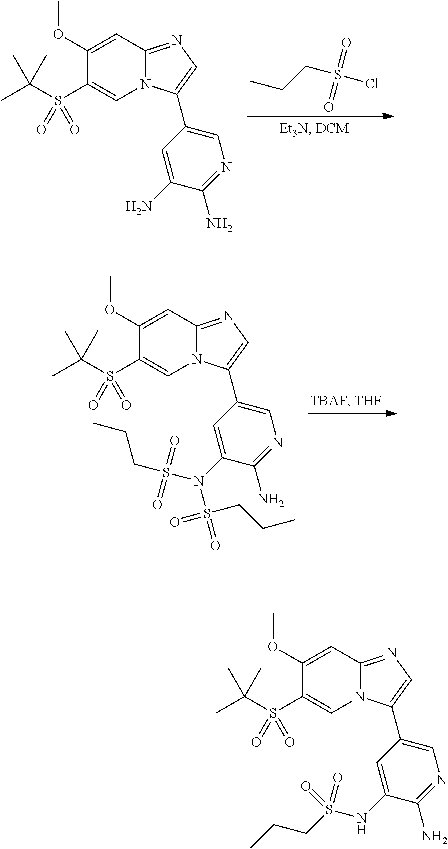

N-(2-amino-5-(6-(tert-butylsulfonyl)-7-methoxyimidazo[1,2-a]pyridin-3-yl)pyridin-3-yl)-N-(propylsulfonyl)propane-1-sulfonamide: To a solution of 5-(6-(tert-butylsulfonyl)-7-methoxyimidazo[1,2-a]pyridin-3-yl)pyridine-2,3-diamine (50 mg, 106.54 mol, 1 eq) and propane-1-sulfonyl chloride (75.97 mg, 532.70 mol, 59.82 μL, 5 eq) in DCM (3 mL) was added Et₃N (53.90 mg, 532.70 mol, 74.15 μL, 5 eq). The mixture was stirred at 25° C. for 4 h before it was filtered and concentrated under reduced pressure to give the title compound, which was used in the next step without further purification.

N-(2-amino-5-(6-(tert-butylsulfonyl)-7-methoxyimidazo[1,2-a]pyridin-3-yl)pyridin-3-yl)propane-1-sulfonamide: To a solution of N-(2-amino-5-(6-(tert-butylsulfonyl)-7-methoxyimidazo[1,2-a]pyridin-3-yl)pyridin-3-yl)-N-(propylsulfonyl)propane-1-sulfonamide (70 mg, 119.10, crude purity, 1 eq) in THF (2 mL) was added TBAF (1 M, 476.41 μL, 4 eq). The mixture was stirred at 25° C. for 4 h before it was filtered and concentrated under reduced pressure to give a residue that was purified by RP-HPLC to give the title compound. [M+H]⁺=482.1. ¹H NMR (400 MHz, DMSO-d₆) δ ppm 8.55 (s, 1H), 8.04-7.86 (m, 1H), 7.62 (s, 1H), 7.56 (d, J=2.0 Hz, 1H), 7.25-7.20 (m, 1H), 6.34-6.13 (m, 2H), 3.92 (s, 3H), 3.08-3.03 (m, 2H), 1.76-1.65 (m, 2H), 1.31 (s, 9H), 0.96 (t, J=5.2 Hz, 3H).

The following compounds were prepared following procedures analogous to that described for Example 253.

| Ex. | | | |
|---|---|---|---|
| No. | Structure | Name | LCMS, ¹H NMR |
| 254 | 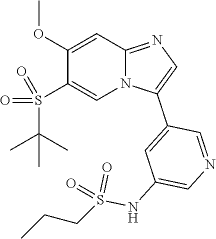 [SMILES: CCCS(=O)(=O)Nc1cncc(-c2cnc3cc(OC)c(S(=O)(=O)C(C)(C)C)cn23)c1] | 5-(6-(tert-butylsulfonyl)- 7-methoxyimidazo[1,2- a]pridin-3-yl)pyridin-3- yl)propane-1-sulfonamide | [M + H]+ = 467.1 ¹H NMR (400 MHz, DMSO- d₆) δ ppm 10.35 (s, 1H), 8.71 (s, 1H), 8.55 (d, J = 1.6 Hz, 1H), 8.45 (d, J = 2.8 Hz, 1H), 7.89 (s, 1H), 7.86-7.80 (m, 1H), 7.31 (s, 1H), 3.94 (s, 3H), 3.25-3.18 (m, 2H), 1.79-1.68 (m, 2H), 1.33 (s, 9H), 0.97 (t, J = 7.6 Hz, 3H). |
| 255 | 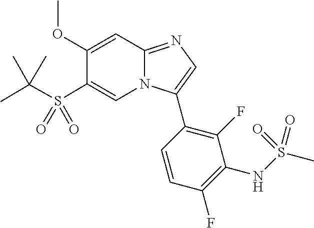 | N-(3-(6-(tert- butylsulfonyl)-7- methoxyimidazo[1,2- a]pyridin-3-yl)-2,6- difluorophenyl)propane-1- sulfonamide | [M + H]⁺ = 502.1 ¹H NMR (400 MHz, DMSO- d₆) δ ppm 8.38 (d, J = 2.8 Hz, 1H), 7.79 (s, 1H), 7.66-7.50 (m, 1H), 7.45-7.36 (m, 1H), 7.31 (s, 1H), 3.95 (s, 3H), 3.15-3.06 (m, 2H), 1.85-1.73 (m, 2H), 1.31 (s, 9H), 1.00 (t, J = 7.6 Hz, 3H) |
| 256 | 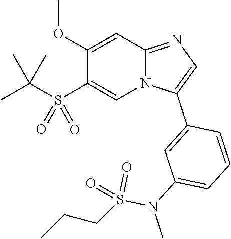 [SMILES: CCCS(=O)(=O)N(C)c1cccc(-c2cnc3cc(OC)c(S(=O)(=O)C(C)(C)C)cn23)c1] | N-(3-(6-(tert- butylsulfonyl)-7- methoxyimidazo[1,2- a]pyridin-3-yl)phenyl)-N- methylpropane-1- sulfonamide | [M + H]⁺ = 480.0 ¹H NMR (400 MHz, DMSO- d₆) δ ppm 8.73 (s, 1H), 7.81 (s, 1H), 7.70 (s, 1H), 7.63-7.58 (m, 1H), 7.53-7.48 (m, 2H), 7.28 (s, 1H), 3.94 (s, 3H), 3.32-3.31 (m, 3H), 3.19-3.14 (m, 2H), 1.74-1.67 (m, 2H), 1.32 (s, 9H), 0.97 (t, J = 7.2 Hz, 3H). |
| 257 | 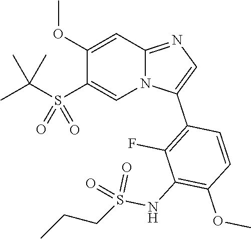 [SMILES: CCCS(=O)(=O)Nc1c(OC)ccc(-c2cnc3cc(OC)c(S(=O)(=O)C(C)(C)C)cn23)c1F] | N-(3-(6-(tert- butylsulfonyl)-7- methoxyimidazo[1,2- a]pyridin-3-yl)-2-fluoro- 6- methoxyphenyl)propane- 1-sulfonamide | [M + H]⁺ = 514.2 ¹H NMR (400 MHz, DMSO- d₆) δ ppm 9.13-8.92 (m, 1H), 8.33 (d, J = 2.4 Hz 1H), 7.72 (s, 1H), 7.52 (t, J = 8.4 Hz, 1H), 7.29 (s, 1H), 7.15 (d, J = 8.8 Hz, 1H), 3.95-3.90 (m, 6H), 3.14-3.03 (m, 2H), 1.89- 1.76 (m, 2H), 1.30 (s, 9H), 1.00 (t, J = 7.6 Hz, 3H). |
| 258 | 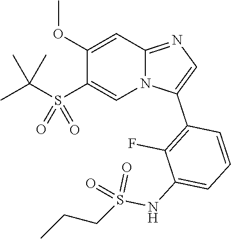 [SMILES: CCCS(=O)(=O)Nc1cccc(-c2cnc3cc(OC)c(S(=O)(=O)C(C)(C)C)cn23)c1F] | N-(3-(6-(tert- butylsulfonyl)-7- methoxyimidazo[1,2- a]pyridin-3-yl)-2- fluorophenyl)propane-1- sulfonamide | [M + H]⁺ = 484.0 ¹H NMR (400 MHz, DMSO- d₆) δ ppm 9.88 (s, 1H), 8.40 (d, J = 2.8 Hz, 1H), 7.80 (s, 1H), 7.59-7.50 (m, 1H), 7.49-7.41 (m, 1H), 7.40-7.34 (m, 1H), 7.32 (s, 1H), 3.95 (s, 3H), 3.17-3.09 (m, 2H), 1.84-1.68 (m, 2H), 1.31 (s, 9H), 0.99 (t, J = 7.2 Hz, 3H). |
| 259 | 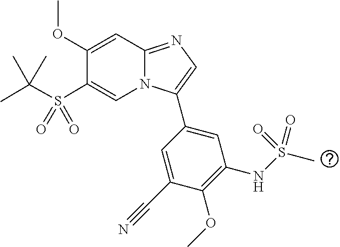 | N-(5-(6-(tert- butylsulfonyl)-7- methoxyimidazo[1,2- a]pyridin-3-yl)-3-cyano-2- methoxyphenyl)propane- 1-sulfonamide | [M + H]⁺ = 521.0 ¹H NMR (400 MHz, DMSO- d₆) δ ppm 8.68 (s, 1H), 7.86 (s, 1H), 7.79 (s, 1H), 7.69 (s, 1H), 7.28 (s, 1H), 4.03 (s, 3H), 3.93 (s, 3H), 3.19-3.11 (m, 2H), 1.82-1.66 (m, 2H), 1.33 (s, 9H), 0.97 (t, J = 7.2 Hz, 3H). |
| 260 | 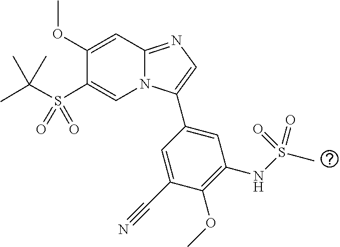 [SMILES: COc1cc2ncc(-c3cc(C#N)c(OC)c(NS(C)(=O)=O)c3)n2cc1S(=O)(=O)C(C)(C)C] | N-(5-(6-(tert- butylsulfonyl)-7- methoxyimidazo[1,2- a]pyridin-3-yl)-3-cyano-2- methoxyphenyl)methane- sulfonamide | [M + H]⁺ = 493.0 ¹H NMR (400 MHz, DMSO- d₆) δ ppm 9.78 (s, 1H), 8.68 (s, 1H), 7.89 (d, J = 2.0 Hz, 1H), 7.84 (d, J = 1.6 Hz, 1H), 7.82 (s, 1H), 7.29 (s, 1H), 4.05 (s, 3H), 3.94 (s, 3H), 3.15 (s, 3H), 1.33 (s, 9H). |
| 261 | 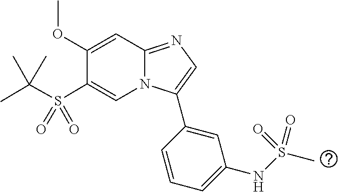 [SMILES: COc1cc2ncc(-c3cccc(NS(C)(=O)=O)c3)n2cc1S(=O)(=O)C(C)(C)C] | N-(3-(6-(tert- butylsulfonyl)-7- methoxyimidazo[1,2- a]pyridin-3- yl)phenyl)methanesulfon- amide | [M + H]⁺ = 438.0 ¹H NMR (400 MHz, DMSO- d₆) δ ppm 10.25-9.90 (m, 1H), 8.73 (s, 1H), 7.76 (s, 1H), 7.57-7.50 (m, 1H), 7.42 (s, 1H), 7.35-7.30 (m, 1H), 7.29- 7.22 (m, 2H), 3.94 (s, 3H), 3.07 (s, 3H), 1.32 (s, 9H). |
| 262 | 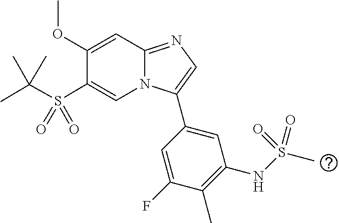 | N-(5-(6-(tert- butylsulfonyl)-7- methoxyimidazo[1,2- a]pyridin-3-yl)-3-fluoro- 2-methylphenyl)propane- 1-sulfonamide | [M + H]⁺ = 498.2 ¹H NMR (400 MHz, DMSO- d₆) δ ppm 9.61-9.27 (m, 1H), 8.73 (s, 1H), 7.81 (s, 1H), 7.38 (s, 1H), 7.37 (br s, 1H), 7.29 (s, 1H), 3.94 (s, 3H), 3.20-3.12 (m, 2H), 2.27 (d, J = 2.0 Hz, 3H), 1.79-1.70 (m, 2H), 1.32 (s, 9H), 0.98 (t, J = 7.6 Hz, 3H). |
| 263 | 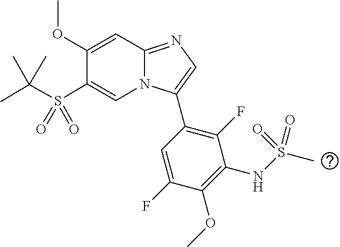 | N-(3-(6-(tert- butylsulfonyl)-7- methoxyimidazo[1,2- a]pyridin-3-yl)-2,5- difluoro-6- methoxyphenyl)propane- 1-sulfonamide | [M + H]⁺ = 532.0 ¹H NMR (400 MHz, DMSO- d₆) δ ppm 9.49 (s, 1H), 8.38 (d, J = 3.2 Hz, 1H), 8.13 (s, 0.3H), 7.80 (s, 1H), 7.66-7.55 (m, 1H), 7.32 (s, 1H), 4.02 (d, J = 1.6 Hz, 3H), 3.94 (s, 3H), 3.20- 3.10 (m, 2H), 1.88-1.76 (m, 2H), 1.31 (s, 9H), 1.01 (t, J = 7.2 Hz, 3H) |
| 264 | 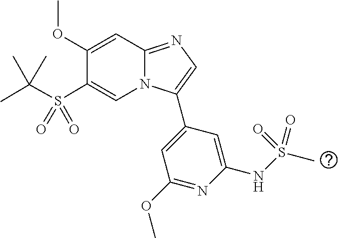 [SMILES: COc1cc(-c2cnc3cc(OC)c(S(=O)(=O)C(C)(C)C)cn23)cc(NS(C)(=O)=O)n1] | N-(4-(6-(tert- butylsulfonyl)-7- methoxyimidazo[1,2- a]pyridin-3-yl)-6- methoxypyridin-2- yl)methanesulfonamide | [M + H]⁺ = 469.0 ¹H NMR (400 MHz, DMSO- d₆) δ ppm 8.82 (s, 1H), 8.24 (s, 0.1H), 7.92 (s, 1H), 7.31 (s, 1H), 6.67-6.58 (m, 2H), 3.94 (s, 3H), 3.89 (s, 3H), 3.34 (s, 3H), 1.33 (s, 9H). |
| 265 | 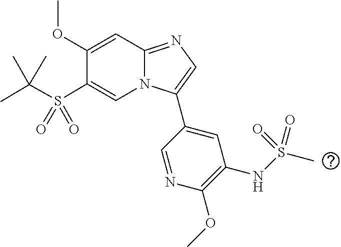 | N-(5-(6-(tert- butylsulfonyl)-7- methoxyimidazo[1,2- a]pyridin-3-yl)-2- methoxypyridin-3- yl)propane-1-sulfonamide | [M + H]⁺ = 497.2 ¹H NMR (400 MHz, DMSO- d₆) δ ppm 9.50 (s, 1H), 8.59 (s, 1H), 8.20 (d, J = 2.0 Hz, 1H), 7.87 (d, J = 1.6 Hz, 1H), 7.82 (s, 1H), 7.28 (s, 1H), 3.99 (s, 3H), 3.95 (s, 3H), 3.18-3.13 (m, 2H), 1.82-1.70 (m, 2H), 1.31 (s, 9H), 0.97 (t, J = 7.2 Hz, 3H) |
| 266 | 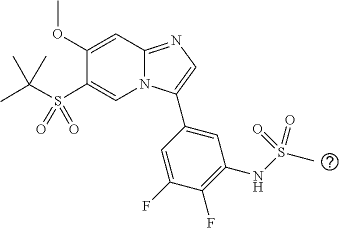 | N-(5-(6-(tert- butylsulfonyl)-7- methoxyimidazo[1,2- a]pyridin-3-yl)-2,3- difluorophenyl)propane-1- sulfonamide | [M + H]⁺ = 502.1 ¹H NMR (400 MHz, DMSO- d₆) δ ppm 10.19 (br s, 1H), 8.70 (s, 1H), 7.80 (s, 1H), 7.55-7.44 (m, 2H), 7.28 (s, 1H), 3.93 (s, 3H), 3.21-3.17 (m, 2H), 1.79-1.72 (m, 2H), 1.32 (s, 9H), 0.98 (t, J = 7.2 Hz, 3H) |
| 267 | 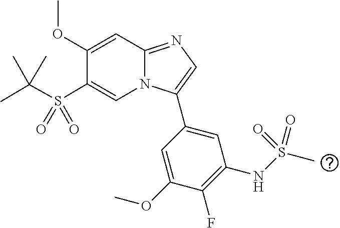 | N-(5-(6-(tert- butylsulfonyl)-7- methoxyimidazo[1,2- a]pyridin-3-yl)-2-fluoro- 3- methoxyphenyl)propane- 1-sulfonamide | [M + H]⁺ = 514.0 ¹H NMR (400 MHz, DMSO- d₆) δ ppm 10.54-9.22 (m, 1H), 8.73 (s, 1H), 7.78 (s, 1H), 7.28-7.22 (m, 2H), 7.21-7.17 (m, 1H), 3.93 (s, 3H), 3.89 (s, 3H), 3.18-3.09 (m, 2H), 1.79- 1.69 (m, 2H), 1.33 (s, 9H), 0.97 (t, J = 7.2 Hz, 3H). |
| 268 | 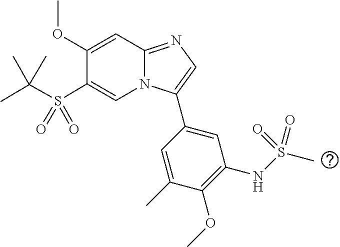 | N-(5-(6-(tert- butylsulfonyl)-7- methoxyimidazo[1,2- a]pyridin-3-yl)-2- methoxy-3- methylphenyl)propane-1- sulfonamide | [M + H]⁺ = 510.1 ¹H NMR (400 MHz, DMSO- d₆) δ ppm 9.26 (s, 1H), 8.70 (s, 1H), 7.70 (s, 1H), 7.47-7.42 (m, 1H), 7.26 (s, 1H), 7.21 (s, 1H), 3.93 (s, 3H), 3.77 (s, 3H), 3.18 (t, J = 7.2 Hz, 2H), 2.32 (s, 3H), 1.82-1.70 (m, 2H), 1.33 (s, 9H), 0.97 (t, J = 7.6 Hz, 3H). |
| 269 | 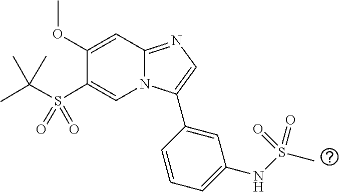 | N-(3-(6-(tert- butylsulfonyl)-7- methoxyimidazo[1,2- a]pyridin-3- yl)phenyl)propane-1- sulfonamide | [M + H]⁺ = 466.0 ¹H NMR (400 MHz, DMSO- d₆) δ ppm 10.07 (br s, 1H), 8.73 (s, 1H), 7.76 (s, 1H), 7.56-7.49 (m, 1H), 7.43 (s, 1H), 7.33-7.25 (m, 3H), 3.94 (s, 3H), 3.20-3.11 (m, 2H), 1.77-1.67 (m, 2H), 1.32 (s, 9H), 0.96 (t, J = 7.6 Hz, 3H) |
| 270 | 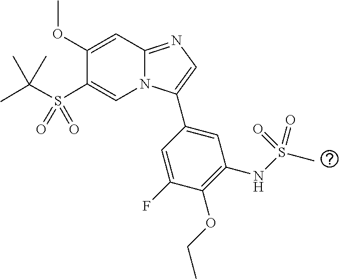 | N-(5-(6-(tert- butylsulfonyl)-7- methoxyimidazo[1,2- a]pyridin-3-yl)-2-ethoxy- 3-fluorophenyl)propane-1- sulfonamide | [M + H]⁺ = 528.1 ¹H NMR (400 MHz, DMSO- d₆) δ ppm 8.71 (s, 1H), 7.76 (s, 1H), 7.42 (s, 1H), 7.35-7.28 (m, 1H), 7.28 (s, 1H), 4.21- 4.14 (m, 2H), 3.93 (s, 3H), 3.16 (t, J = 7.6 Hz, 2H), 1.82- 1.70 (m, 2H), 1.39-1.34 (m, 2H), 1.33 (s, 9H), 0.97 (t, J = 7.2 Hz, 3H). |
| 271 | 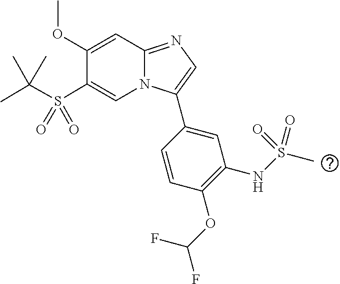 | N-(5-(6-(tert- butylsulfonyl)-7- methoxyimidazo[1,2- a]pyridin-3-yl)-2- (difluoromethoxy)phenyl) propane-1-sulfonamide | [M + H]⁺ = 532.1 ¹H NMR (400 MHz, DMSO- d₆) δ ppm 8.70 (s, 1H), 7.75 (s, 1H), 7.65 (s, 1H), 7.46-7.09 (m, 4H), 3.93 (s, 3H), 3.16 (t, J = 7.6, 2H), 1.82-1.69 (m, 2H), 1.33-1.30 (m, 9H), 1.03- 0.91 (m, 3H) |
| 272 | 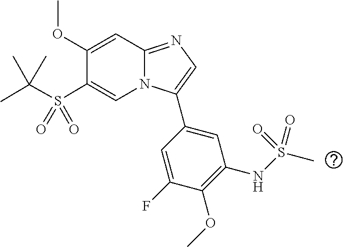 [SMILES: COc1cc2ncc(-c3cc(F)c(OC)c(NS(C)(=O)=O)c3)n2cc1S(=O)(=O)C(C)(C)C] | N-(5-(6-(tert- butylsulfonyl)-7- methoxyimidazo[1,2- a]pyridin-3-yl)-3-fluoro- 2- methoxyphenyl)methane- sulfonamide | [M + H]⁺ = 486.1 ¹H NMR (400 MHz, DMSO- d₆) δ ppm 9.56 (s, 1H), 8.71 (s, 1H), 7.77 (s, 1H), 7.41 (s, 1H), 7.38 (s, 1H), 7.27 (s, 1H), 3.93 (s, 6H), 3.12 (s, 3H), 1.32 (s, 9H) |
| 273 | 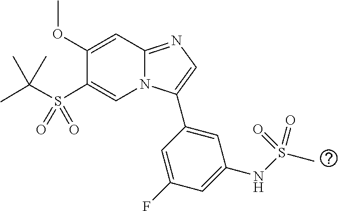 | N-(3-(6-(tert- butylsulfonyl)-7- methoxyimidazo[1,2- a]pyridin-3-yl)-5- fluorophenyl)propane-1- sulfonamide | [M + H]⁺ = 508.1 ¹H NMR (400 MHz, DMSO- d₆) δ ppm 8.74 (s, 1H), 7.82 (d, J = 6.0 Hz, 1H), 7.28 (s, 1H), 7.20 (s, 1H), 7.12 (d, J = 4.8 Hz, 1H), 7.03-6.95 (m, 1H), 3.20-3.11 (m, 3H), 1.76-1.65 (m, 2H), 1.32 (s, 9H), 0.96 (t, J = 7.6 Hz, 3H) |
| 274 | 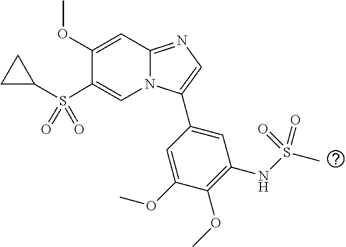 | N-(5-(6- (cyclopropylsulfonyl)-7- methoxyimidazo[1,2- a]pyridin-3-yl)-2,3- dimethoxyphenyl)propane- 1-sulfonamide | [M + H]⁺ = 510.1 ¹H NMR (400 MHz, DMSO- d₆) δ ppm 9.26 (s, 1H), 8.73 (s, 1H), 7.73 (s, 1H), 7.30 (s, 1H), 7.17 (dd, J = 1.6, 14.8 Hz, 2H), 4.02 (s, 3H), 3.88 (s, 3H), 3.82 (s, 3H), 3.19-3.10 (m, 2H), 3.10-3.02 (m, 1H), 1.83-1.67 (m, 2H), 1.18-1.11 (m, 2H), 1.10-1.03 (m, 2H), 0.97 (t, J = 7.6 Hz, 3H). |
| 275 | 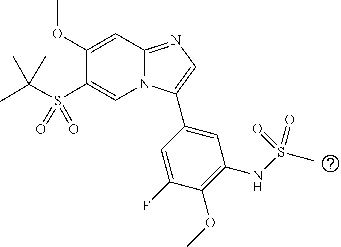 | N-(5-(6-(tert- butylsulfonyl)-7- methoxyimidazo[1,2- a]pyridin-3-yl)-3-fluoro- 2- methoxyphenyl)propane- 1-sulfonamide | [M + H]⁺ = 514.1 ¹H NMR (400 MHz, DMSO- d₆) δ ppm 9.56 (s, 1H), 8.71 (s, 1H), 7.77 (s, 1H), 7.42 (s, 1H), 7.39-7.33 (m, 1H), 7.27 (s, 1H), 3.93 (s, 6H), 3.21-3.14 (m, 2H), 1.83-1.68 (m, 2H), 1.33 (s, 9H), 0.98 (t, J = 7.2 Hz, 3H) |
| 276 | 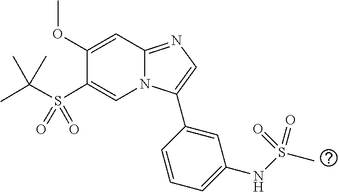 | N-(3-(6-(tert- butylsulfonyl)-7- methoxyimidazo[1,2- a]pyridin-3- yl)phenyl)ethanesulfon- amide | [M + H]⁺ = 452.1 ¹H NMR (400 MHz, DMSO- d₆) δ ppm 10.05 (s, 1H), 8.72 (s, 1H), 7.76 (s, 1H), 7.56- 7.50 (m, 1H), 7.43 (s, 1H), 7.33-7.25 (m, 3H), 3.93 (s, 3H), 3.18 (q, J = 7.3 Hz, 2H), 1.32 (s, 9H), 1.23 (t, J = 7.3 Hz, 3H). |
| 277 | 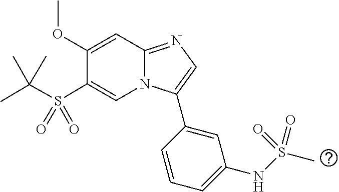 | N-(3-(6-(tert- butylsulfonyl)-7- methoxyimidazo[1,2- a]pyridin-3-yl)phenyl)-2- methylpropane-2- sulfonamide | [M + H]⁺ = 480.2 ¹H NMR (400 MHz, DMSO- d₆) δ ppm 8.75 (s, 1H), 8.03 (s, 1H), 7.73 (s, 1H), 7.43 (t, J = 7.9 Hz, 1H), 7.28 (s, 1H), 7.21 (d, J = 1.7 Hz, 1H), 7.13-7.04 (m, 2H), 3.94 (s, 3H), 1.36 (s, 9H), 1.32 (s, 9H). |
| 278 | 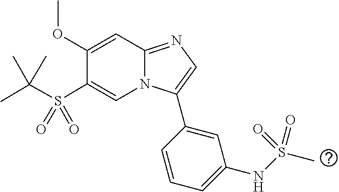 | N-(3-(6-(tert- butylsulfonyl)-7- methoxyimidazo[1,2- a]pyridin-3- yl)phenyl)propane-2- sulfonamide | [M + H]⁺= 466.2 ¹H NMR (400 MHz, DMSO- d₆) δ ppm 10.05 (s, 1H), 8.72 (s, 1H), 7.75 (s, 1H), 7.55- 7.48 (m, 1H), 7.46 (s, 1H), 7.32-7.26 (m, 3H), 3.93 (s, 3H), 1.32 (s, 9H), 1.27 (d, J = 6.8 Hz, 6H). |
| 279 | 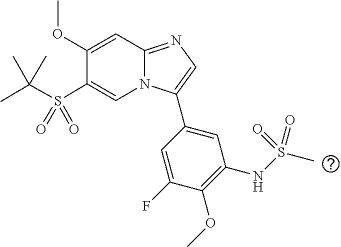 | N-(5-(6-(tert- butylsulfonyl)-7- methoxyimidazo[1,2- a]pyridin-3-yl)-3-fluoro- 2-methoxyphenyl)-2- methylpropane-1- sulfonamide | [M + H]⁺= 528.1 ¹H NMR (400 MHz, DMSO- d₆) δ ppm 8.71 (s, 1H), 7.76 (s, 1H), 7.41 (s, 1H), 7.38-7.30 (m, 1H), 7.28 (s, 1H), 3.95- 3.90 (m, 6H), 3.13-3.05 (m, 2H), 2.26-2.13 (m, 1H), 1.33 (s, 9H), 1.02 (d, J = 6.8 Hz, 6H). |
| 280 | 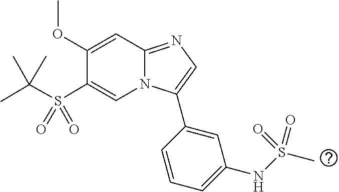 | N-(3-(6-(tert- butylsulfonyl)-7- methoxyimidazo[1,2- a]pyridin-3-yl)phenyl)-2- methylpropane-1- sulfonamide | [M + H]⁺ = 480.1 ¹H NMR (500 MHz, DMSO- d₆) δ ppm 10.09 (s, 1H), 8.72 (s, 1H), 7.75 (s, 1H), 7.53 (t, J = 7.9 Hz, 1H), 7.42 (s, 1H), 7.33-7.24 (m, 3H), 3.94 (s, 3H), 3.07 (d, J = 6.4 Hz, 2H), 2.16 (q, J = 6.6, 13.2 Hz, 1H), 1.32 (s, 9H), 1.01 (d, J = 6.7 Hz, 6H). |
| 281 | 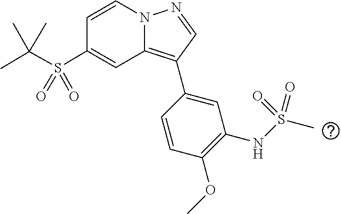 | N-(5-(5-(tert- butylsulfonyl)pyrazolo[1, 5-a]pyridin-3-yl)-2- methoxyphenyl)propane- 1-sulfonamide | [M + H]⁺ = 466.30 ¹H NMR (400 MHz, DMSO- d₆) δ ppm 9.06 (s, 1H), 8.94 (d, J = 7.2 Hz, 1H), 8.52 (s, 1H), 8.21 (d, J = 1.2 Hz, 1H), 7.58 (d, J = 2.4 Hz, 1H), 7.42 (d, J = 7.2 Hz, 1H), 7.21-7.16 (m, 2H), 3.87 (s, 3H), 3.01 (t, J = 7.6 Hz, 2H), 1.80-1.71 (m, 2H), 1.33 (s, 9H), 0.94 (t, J = 7.4 Hz, 3H). |
| 282 | 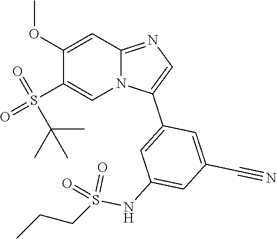 [SMILES: CCCS(=O)(=O)Nc1cc(C#N)cc(-c2cnc3cc(OC)c(S(=O)(=O)C(C)(C)C)cn23)c1] | N-(3-(6-(tert- butylsulfonyl)-7- methoxyimidazo[1,2- a]pyridin-3-yl)-5- cyanophenyl)propane-1- sulfonamide | [M + H]⁺ = 491.0 ¹H NMR (400 MHz, DMSO- d₆) δ ppm 8.72 (s, 1H), 8.30 (s, 0.3H), 7.85 (s, 1H), 7.66-7.58 (m, 2H), 7.46 (s, 1H), 7.29 (s, 1H), 3.94 (s, 3H), 3.18-3.11 (m, 2H), 1.74-1.65 (m, 2H), 1.32 (s, 9H), 0.95 (t, J = 7.6 Hz, 3H). |
| 283 | 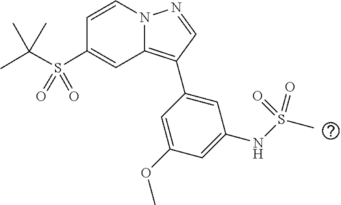 | N-(3-(5-(tert- butylsulfonyl)pyrazolo[1, 5-a]pyridin-3-yl)-5- methoxyphenyl)propane- 1-sulfonamide | [M + H]⁺ = 466.33 ¹H NMR (400 MHz, DMSO- d₆) δ ppm 9.92 (s, 1H), 8.97 (d, J = 7.2 Hz, 1H), 8.62 (s, 1H), 8.24 (d, J = 1.2 Hz, 1H), 7.21 (dd, J = 7.4, 1.8 Hz, 1H), 7.13 (s, 1H), 6.95 (s, 1H), 6.75 (s, 1H), 3.81 (s, 3H), 3.10 (t, J = 7.6 Hz, 2H), 1.76-1.67 (m, 2H), 1.34 (s, 9H), 0.93 (t, J = 7.4 Hz, 3H). |
| 284 | 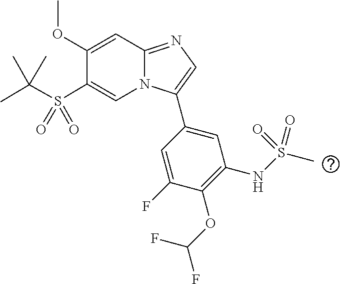 | N-(5-(6-(tert- butylsulfonyl)-7- methoxyimidazo[1,2- a]pyridin-3-yl)-2- (difluoromethoxy)-3- fluorophenyl)propane-1- sulfonamide | [M + H]⁺ = 550.0 ¹H NMR (400 MHz, DMSO- d₆) δ ppm 8.76 (s, 1H), 8.33- 8.14 (m, 1H), 7.68 (s, 1H), 7.44-7.04 (m, 3H), 6.79-6.66 (m, 1H), 3.93 (s, 3H), 2.87- 2.79 (m, 2H), 1.73-1.59 (m, 2H), 1.32 (s, 9H), 0.93 (t, J = 7.6 Hz, 3H). |
| 285 | 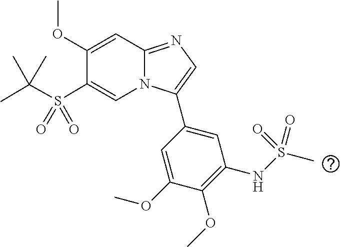 [SMILES: COc1cc2ncc(-c3cc(NS(C)(=O)=O)c(OC)c(OC)c3)n2cc1S(=O)(=O)C(C)(C)C] | N-(5-(6-(tert- butylsulfonyl)-7- methoxyimidazo[1,2- a]pyridin-3-yl)-2,3- dimethoxyphenyl)methane sulfonamide | [M + H]⁺ = 498.0 ¹H NMR (400 MHz, DMSO- d₆) δ ppm 9.26 (s, 1H), 8.76 (s, 1H), 7.75 (s, 1H), 7.26 (s, 1H), 7.19 (d, J = 2.0 Hz, 1H), 7.13 (d, J = 2.0 Hz, 1H), 3.93 (s, 3H), 3.87 (s, 3H), 3.83 (s, 3H), 3.08 (s, 3H), 1.33 (s, 9H). |
| 286 | 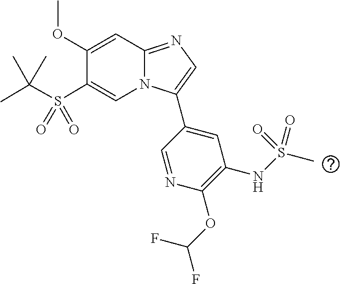 | N-(5-(6-(tert- butylsulfonyl)-7- methoxyimidazo[1,2- a]pyridin-3-yl)-2- (difluoromethoxy)pyridin- 3-yl)propane-1- sulfonamide | [M + H]⁺ = 533.0 ¹H NMR (400 MHz, DMSO- d₆) δ ppm 9.90 (br s, 1H), 8.63 (s, 1H), 8.29 (s, 1H), 8.05 (s, 1H), 7.97-7.60 (m, 2H), 7.30 (s, 1H), 3.94 (s, 3H), 3.24-3.17 (m, 2H), 1.85-1.71 (m, 2H), 1.32 (s, 9H), 0.98 (t, J = 7.2 Hz, 3H). |
| 287 | 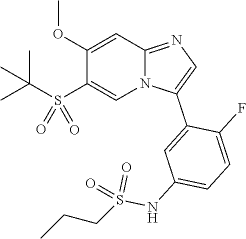 [SMILES: CCCS(=O)(=O)Nc1ccc(F)c(-c2cnc3cc(OC)c(S(=O)(=O)C(C)(C)C)cn23)c1] | N-(3-(6-(tert- butylsulfonyl)-7- methoxyimidazo[1,2- a]pyridin-3-yl)-4- fluorophenyl)propane-1- sulfonamide | [M + H]⁺ = 484.0 ¹H NMR (400 MHz, DMSO- d₆) δ ppm 10.00 (br s, 1H), 8.43 (d, J = 2.8 Hz, 1H), 7.77 (s, 1H), 7.49-7.39 (m, 2H), 7.36-7.33 (m, 1H), 7.32 (s, 1H), 3.95 (s, 3H), 3.17-3.11 (m, 2H), 1.79-1.62 (m, 2H), 1.31 (s, 9H), 0.96 (t, J = 7.2 Hz, 3H). |
| 288 | 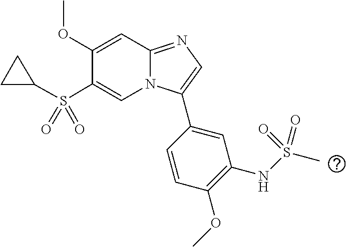 | N-(5-(6- (cyclopropylsulfonyl)-7- methoxyimidazo[1,2- a]pyridin-3-yl)-2- methoxyphenyl)propane- 1-sulfonamide | [M + H]⁺ = 480.29 ¹H NMR (400 MHz, DMSO- d₆) δ ppm 9.05 (s, 1H), 8.63 (s, 1H), 7.67 (s, 1H), 7.49-7.45 (m, 2H), 7.25 (t, J = 7.4 Hz, 2H), 4.01 (s, 3H), 3.90 (s, 3H), 3.06 (t, J = 4.6 Hz, 3H), 1.79- 1.71 (m, 2H), 1.24 (d, J = 5.6 Hz, 2H), 1.14-1.12 (m, 2H), 1.07 (dd, J = 7.0, 4.6 Hz, 2H), 0.95 (t, J = 7.4 Hz, 3H). |
| 289 | 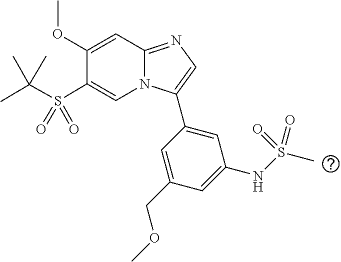 | N-(3-(6-(tert- butylsulfonyl)-7- methoxyimidazo[1,2- a]pyridin-3-yl)-5- (methoxymethyl)phenyl) propane-1-sulfonamide | [M + H]⁺ = 510.0 ¹H NMR (400 MHz, DMSO- d₆) δ ppm 10.08 (br s, 1H), 8.73 (s, 1H), 7.76 (s, 1H), 7.36 (s, 1H), 7.27 (s, 1H), 7.25-7.20 (m, 2H), 4.46 (s, 2H), 3.94 (s, 3H), 3.34-3.34 (m, 3H), 3.19- 3.11 (m, 2H), 1.77-1.66 (m, 2H), 1.32 (s, 9H), 0.96 (t, J = 7.2 Hz, 3H) |
| 290 | 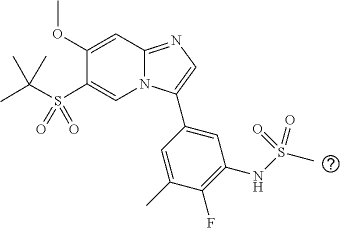 | N-(5-(6-(tert- butylsulfonyl)-7- methoxyimidazo[1,2- a]pyridin-3-yl)-2-fluoro- 3-methylphenyl)propane- 1-sulfonamide | [M + H]⁺ = 498.1 ¹H NMR (400 MHz, DMSO- d₆) δ ppm 10.08-9.52 (m, 1H), 8.67 (s, 1H), 7.73 (s, 1H), 7.46 (d, J = 6.8 Hz, 1H), 7.33 (d, J = 4.8 Hz, 1H), 7.27 (s, 1H), 3.93 (s, 3H), 3.18-3.12 (m, 2H), 2.32 (s, 3H), 1.81-1.71 (m, 2H), 1.32 (s, 9H), 0.98 (d, J = 7.6 Hz, 3H). |
| 291 | 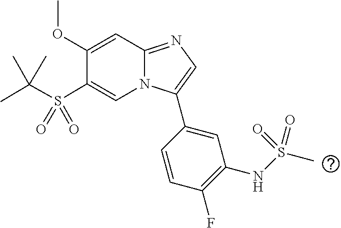 | N-(5-(6-(tert- butylsulfonyl)-7- methoxyimidazo[1,2- a]pyridin-3-yl)-2- fluorophenyl)propane-1- sulfonamide | [M + H]⁺ = 484.2 ¹H NMR (400 MHz, DMSO- d₆) δ ppm 9.87 (s, 1H), 8.66 (s, 1H), 7.76 (s, 1H), 7.63 (dd, J = 2.0, 7.6 Hz, 1H), 7.53-7.43 (m, 2H), 7.28 (s, 1H), 3.93 (s, 3H), 3.20-3.12 (m, 2H), 1.80-1.71 (m, 2H), 1.32 (s, 9H), 0.98 (t, J = 7.2 Hz, 3H) |
| 292 | 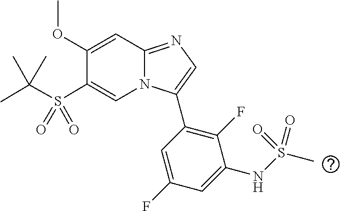 | N-(3-(6-(tert- butylsulfonyl)-7- methoxyimidazo[1,2- a]pyridin-3-yl)-2,5- difluorophenyl)propane-1- sulfonamide | [M + H]⁺ = 502.0 ¹H NMR (400 MHz, DMSO- d₆) δ ppm 10.34-10.03 (m, 1H), 8.44 (d, J = 3.2 Hz, 1H), 7.84 (s, 1H), 7.43-7.30 (m, 3H), 3.95 (s, 3H), 3.25-3.16 (m, 2H), 1.81-1.70 (m, 2H), 1.32 (s, 9H), 0.99 (t, J = 7.2 Hz, 3H) |
| 293 | 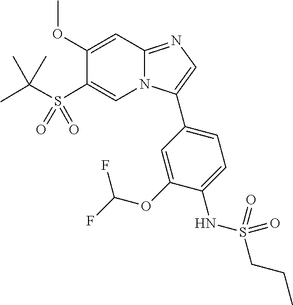 [SMILES: CCCS(=O)(=O)Nc1ccc(-c2cnc3cc(OC)c(S(=O)(=O)C(C)(C)C)cn23)cc1OC(F)F] | N-(4-(6-(tert- butylsulfonyl)-7- methoxyimidazo[1,2- a]pyridin-3-yl)-2- (difluoromethoxy)phenyl) propane-1-sulfonamide | [M + H]⁺ = 532.1 ¹H NMR (400 MHz, DMSO- d₆) δ ppm 9.64 (s, 1H), 8.71 (s, 1H), 8.14 (s, 0.05H), 7.79 (s, 1H), 7.62 (d, J = 8.8 Hz, 1H), 7.50-7.45 (m, 2H), 7.43-7.02 (m, 2H), 3.94 (s, 3H), 3.19- 3.10 (m, 2H), 1.83-1.72 (m, 2H), 1.32 (s, 9H), 0.99 (t, J = 7.6 Hz, 3H) |
| 294 | 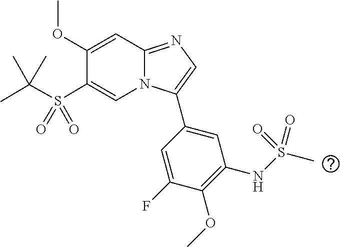 | N-(5-(6-(tert- butylsulfonyl)-7- methoxyimidazo[1,2- a]pyridin-3-yl)-3-fluoro- 2- methoxyphenyl)ethane- sulfonamide | [M + H]⁺ = 500.0 ¹H NMR (400 MHz, DMSO- d₆) δ ppm 9.57 (s, 1H), 8.70 (s, 1H), 7.77 (s, 1H), 7.42 (s, 1H), 7.40-7.34 (m, 1H), 7.27 (s, 1H), 3.96-3.91 (m, 6H), 3.24- 3.15 (m, 2H), 1.33 (s, 9H), 1.27 (t, J = 7.2 Hz, 3H). |
| 295 | 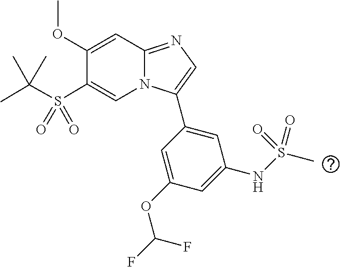 | N-(3-(6-(tert- butylsulfonyl)-7- methoxyimidazo[1,2- a]pyridin-3-yl)-5- (difluoromethoxy)phenyl) propane-1-sulfonamide | [[M + H]⁺ = 532.1 ¹H NMR (400 MHz, DMSO- d₆) δ ppm 10.41-10.18 (m, 1H), 8.74 (s, 1H), 7.82 (s, 1H), 7.50-7.08 (m, 4H), 7.03 (s, 1H), 3.94 (s, 3H), 3.26-3.15 (m, 2H), 1.80-1.64 (m, 2H), 1.33 (s, 9H), 0.97 (t, J = 7.6 Hz, 3H) |

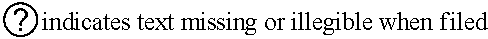

### Examples 296 and 297

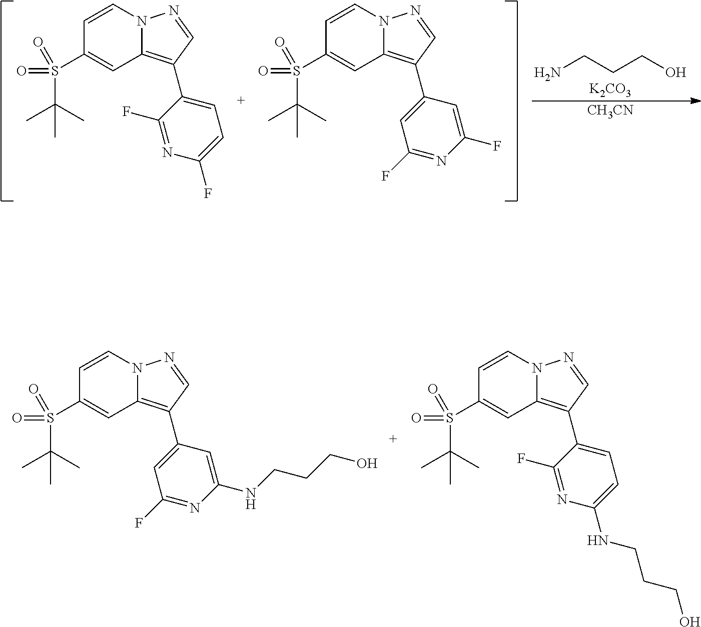

3-((4-(5-(tert-butylsulfonyl)pyrazolo [1,5-a]pyridin-3-yl)-6-fluoropyridin-2-yl)amino)propan-1-ol (Example 296) and 3-((5-(5-(tert-butylsulfonyl)pyrazolo[1,5-a]pyridin-3-yl)-6-fluoropyridin-2-yl)amino)propan-1-ol (Example 297): To a stirred solution of a 3:1 mixture of 5-(tert-butylsulfonyl)-3-(2,6-difluoropyridin-4-yl)pyrazolo[1,5-a]pyridine and 5-(tert-butylsulfonyl)-3-(2,6-difluoropyridin-3-yl)pyrazolo[1,5-a]pyridine (0.160 g, 0.455 mmol, 1 eq) in acetonitrile (1.6 mL) was added anhydrous potassium carbonate (0.157 g, 1.14 mmol, 2.5 eq) followed by 3-aminopropan-1-ol (0.376 g, 0.501 mmol, 1.1 eq) at rt. The reaction mixture was stirred at 90° C. for 2 h, after which it was diluted with water (10 mL) and extracted with EtOAc (2×20 mL). The combined organic layer was dried over anhydrous Mg₂SO₄ and concentrated under reduced pressure. The crude residue was purified by flash column chromatography over silica gel (100-200 mesh) eluting with 60-80% ethyl acetate/petroleum ether to get a mixture of regioisomers which was then subjected for achiral SFC purification to obtain Example 296 and Example 297.

Example 296: [M+H]=407.41. ¹H NMR (400 MHz, DMSO-d₆) δ 9.02 (d, J=7.2 Hz, 1H), 8.70 (s, 1H), 8.26 (d, J=0.8 Hz, 1H), 7.27 (dd, J=7.2, 5.6 Hz, 1H), 7.17 (br s, 1H), 6.63 (s, 1H), 6.46 (s, 1H), 4.49 (t, J=5.2 Hz, 1H), 3.50 (q, J=5.2 Hz, 2H), 3.28 (m, 2H), 1.70 (t, J=2 Hz, 2H), 1.34 (s, 9H).

Example 297: [M+H]=407.41. ¹H NMR (400 MHz, DMSO-d₆) δ 8.98 (d, J=8.0 Hz, 1H), 8.30 (s, 1H), 7.77 (s, 1H), 7.56 (t, J=8.4 Hz, 1H), 7.20 (dd, J=7.2, 2.0 Hz 1H), 6.33 (s, 1H), 6.27 (dd, J=8.0, 2.8 Hz 1H), 4.38 (t, J=5.2 Hz, 1H), 3.41 (q, J=6 Hz, 2H), 3.25 (m, 2H), 1.65 (m, 2H), 1.30 (s, 9H), 1.17 (t, J=7.2 Hz, 1H)

### Examples 298, 299, and 300

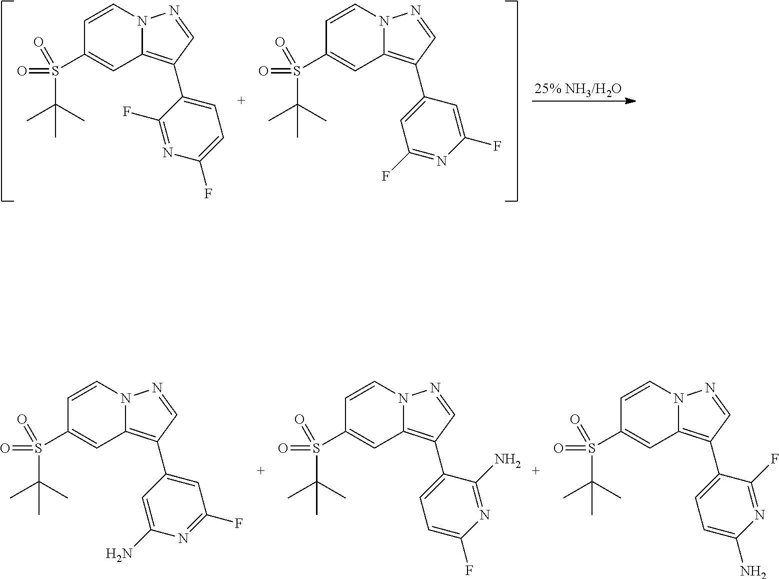

4-(5-(tert-Butylsulfonyl)pyrazolo[1,5-a]pyridin-3-yl)-6-fluoropyridin-2-amine (Example 298), 3-(5-(tert-butylsulfonyl)pyrazolo[1,5-a]pyridin-3-yl)-6-fluoropyridin-2-amine (Example 299), 5-(5-(tert-butylsulfonyl)pyrazolo[1,5-a]pyridin-3-yl)-6-fluoropyridin-2-amine (Example 300): A 3:1 mixture of 5-(tert-butylsulfonyl)-3-(2,6-difluoropyridin-4-yl)pyrazolo[1,5-a]pyridine and 5-(tert-butylsulfonyl)-3-(2,6-difluoropyridin-3-yl)pyrazolo[1,5-a]pyridine (60 mg, 0.171 mmol, 1 eq) and 25% NH₃ in H₂O were combined in a steel bomb. The reaction mixture was stirred at 110° C. for 15 h, after which it was concentrated under reduced pressure. The crude residue was purified by flash chromatography on neutral alumina, eluting with 50-60% EtOAc/petroleum ether. The material was then purified further by achiral SFC to separate the three regioisomers namely:

Example 298 [M+H]=349.32. ¹H NMR (400 MHz, DMSO-d₆) δ 9.01 (d, J=7.2 Hz, 1H), 8.70 (s, 1H), 8.26 (d, J=0.8 Hz, 1H), 7.27 (dd, J=7.2, 2.0 Hz, 1H), 6.61 (s, 2H), 6.51 (d, J=17.2 Hz, 2H), 1.34 (s, 9H),

Example 299 [M+H]=349.40. ¹H NMR (400 MHz, DMSO-d₆) δ 8.95 (d, J=7.2 Hz, 1H), 8.31 (s, 1H), 7.84 (d, J=0.8 Hz, 1H), 7.57 (t, J=8.2 Hz, 1H), 7.17 (dd, J=7.2, 2.0 Hz, 1H), 6.31 (dd, J=7.6, 2.8 Hz, 1H), 6.19 (s, 2H), 1.30 (s, 9H),

### Example 300

### Example 301

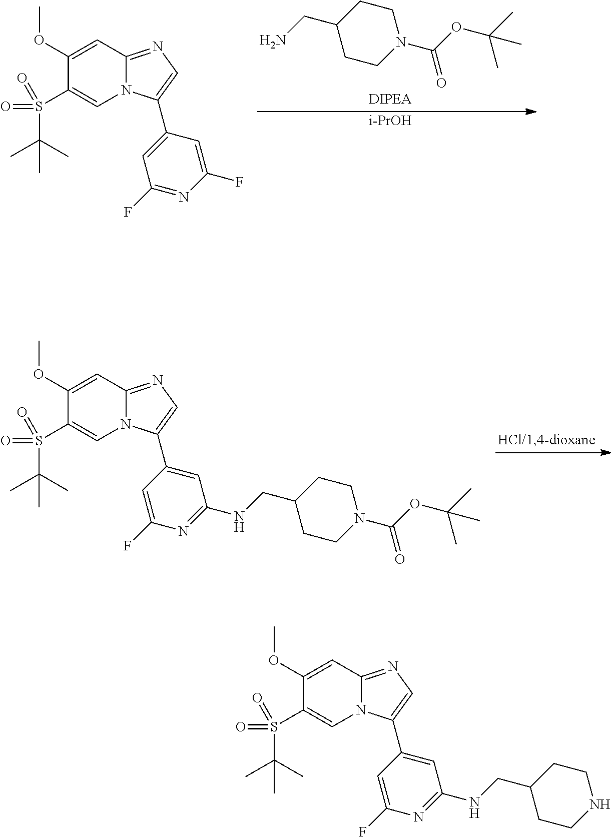 [SMILES: "i-PrOH" ; COc1cc2ncc(-c3cc(F)nc(F)c3)n2cc1S(=O)(=O)C(C)(C)C ; CC(C)(C)OC(=O)N1CCC(CN)CC1 ; COc1cc2ncc(-c3cc(F)nc(NCC4CCN(C(=O)OC(C)(C)C)CC4)c3)n2cc1S(=O)(=O)C(C)(C)C ; COc1cc2ncc(-c3cc(F)nc(NCC4CCNCC4)c3)n2cc1S(=O)(=O)C(C)(C)C]

tert-butyl 4-(((4-(6-(tert-butylsulfonyl)-7-methoxyimidazo[1,2-a]pyridin-3-yl)-6-fluoropyridin-2-yl)amino)methyl)piperidine-1-carboxylate: 6-(tert-butylsulfonyl)-3-(2,6-difluoropyridin-4-yl)-7-methoxyimidazo[1,2-a]pyridine (200 mg, 97% wt, 1 equiv, 509 mol) and tert-butyl 4-(aminomethyl)piperidine-1-carboxylate (236 mg, 233 μL, 97% wt, 2.1 equiv, 1.07 mmol) were dissolved in 2-propanol (10 mL). DIPEA (98.6 mg, 133 μL, 1.5 equiv, 763 μmol) was added and the resulting mixture was heated to 120° C. for 4 h. The solvent was evaporated, and the residue was used without any further purification.

4-(6-(tert-butylsulfonyl)-7-methoxyimidazo[1,2-a]pyridin-3-yl)-6-fluoro-N-(piperidin-4-ylmethyl)pyridin-2-amine: tert-butyl 4-(((4-(6-(tert-butylsulfonyl)-7-methoxyimidazo[1,2-a]pyridin-3-yl)-6-fluoropyridin-2-yl)amino)methyl)piperidine-1-carboxylate (509 mg, 82% wt, 1 equiv, 725 mol) was dissolved in dioxane (10 mL), and hydrogen chloride (264 mg, 1.81 mL, 4 molar in dioxane, 10 equiv, 7.25 mmol) was added dropwise. The mixture was stirred at rt for 16 h. The solvent and all volatiles were evaporated. A part of the crude material was dissolved in DMSO (2 mL) and submitted to HPLC purification (basic method) to give the title compound. [M+H]⁺=476.4. ¹H NMR (400 MHz, DMSO-d₆) δ ppm 8.81 (s, 1H), 8.38 (s, 1H), 7.92 (s, 1H), 7.35-7.46 (m, 1H), 7.31 (s, 1H), 6.54 (s, 1H), 6.37 (s, 1H), 3.94 (s, 3H), 3.12-3.20 (m, 4H), 2.67-2.75 (m, 2H), 2.54 (s, 1H), 1.79 (br d, J=11.7 Hz, 2H), 1.32 (s, 9H), 1.16-1.29 (m, 2H).

### Example 302

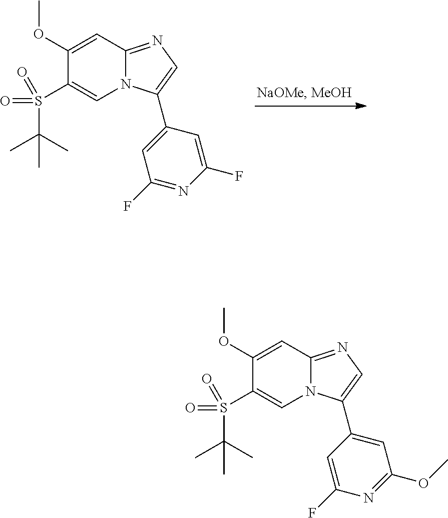 [SMILES: COc1cc2ncc(-c3cc(F)nc(F)c3)n2cc1S(=O)(=O)C(C)(C)C ; COc1cc(-c2cnc3cc(OC)c(S(=O)(=O)C(C)(C)C)cn23)cc(F)n1]

6-(tert-butylsulfonyl)-3-(2-fluoro-6-methoxypyridin-4-yl)-7-methoxyimidazo[1,2-a]pyridine: 6-(tert-butylsulfonyl)-3-(2,6-difluoropyridin-4-yl)-7-methoxyimidazo[1,2-a]pyridine (45 mg, 75% wt, 1 equiv, 88.5 μmol) was dissolved in methanol (2 mL). Sodium methoxide (10.0 mg, 11.0 μL, 2.1 equiv, 186 μmol) was added, and the resulting mixture was heated to 70° C. for 16 h. The solvent was evaporated, and the residue was dissolved in DMSO (2 mL) and submitted to HPLC purification to give the title compound. [M+H]⁺=394.4. ¹H NMR (400 MHz, DMSO-d₆) δ ppm 8.85 (s, 1H), 8.08 (s, 1H), 7.34 (s, 1H), 7.03 (dd, J=1.9, 1.1 Hz, 2H), 3.95 (s, 3H), 3.90 (s, 3H), 1.32 (s, 9H).

### Example 303

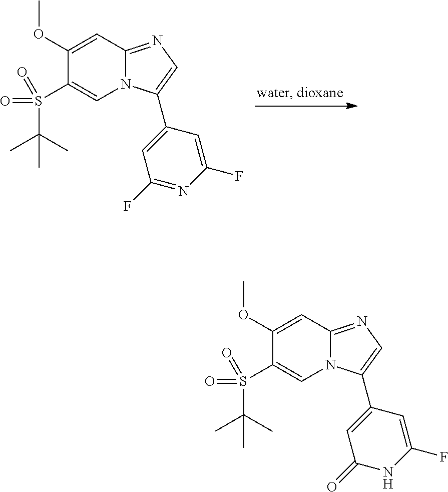 [SMILES: COc1cc2ncc(-c3cc(F)nc(F)c3)n2cc1S(=O)(=O)C(C)(C)C ; COc1cc2ncc(-c3cc(F)[nH]c(=O)c3)n2cc1S(=O)(=O)C(C)(C)C]

4-(6-(tert-butylsulfonyl)-7-methoxyimidazo[1,2-a]pyridin-3-yl)-6-fluoropyridin-2(11H)-one: 6-(tert-butylsulfonyl)-3-(2,6-difluoropyridin-4-yl)-7-methoxyimidazo[1,2-a]pyridine (45 mg, 97% wt, 1 equiv, 114 μmol) and sodium hydroxide (18 mg, 17 μL, 4 equiv, 458 μmol) were dissolved in dioxane (1.2 mL) and water (0.6 mL) and heated to 120° C. for 16 h. The mixture was cooled to rt and the solvent was evaporated. The residue was dissolved in DMSO (2 mL) and submitted to HPLC purification (basic method) to give the title compound. [M+H]⁺=380.4. ¹H NMR (400 MHz, DMSO-d₆) δ ppm 9.02 (s, 1H), 8.28-8.40 (m, 1H), 7.20 (s, 1H), 6.44 (br s, 1H), 6.12 (br s, 1H), 3.89 (s, 3H), 1.31 (s, 9H).

### Example 304

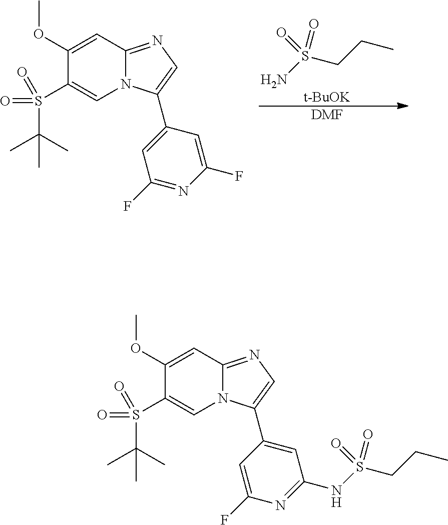

N-(4-(6-(tert-butylsulfonyl)-7-methoxyimidazo[1,2-a]pyridin-3-yl)-6-fluoropyridin-2-yl)propane-1-sulfonamide: Propane-1-sulfonamide (19.0 mg, 1 equiv, 154 μmol) and potassium tert-butoxide (17.3 mg, 1 equiv, 154 μmol) were dissolved in DMF (2 mL) and stirred at rt for 20 min. 6-(tert-butylsulfonyl)-3-(2,6-difluoropyridin-4-yl)-7-methoxyimidazo[1,2-a]pyridine (60 mg, 98% wt, 1 equiv, 154 μmol) was added and the mixture was stirred at 80° C. for 4 h. The solvent was evaporated under reduced pressure and elevated temperature. The residue was redissolved in DMSO (2 mL) and submitted to HPLC purification (basic method) to give the title compound. [M+H]⁺=485.3. ¹H NMR (400 MHz, DMSO-d₆) δ ppm 10.98 (br s, 1H), 9.12 (s, 1H), 8.60 (s, 1H), 7.44 (s, 1H), 7.26 (s, 1H), 7.21 (s, 1H), 3.92 (s, 3H), 3.40-3.52 (m, 2H), 1.74 (sxt, J=7.5 Hz, 2H), 1.32 (s, 9H), 0.98 (t, J=7.4 Hz, 3H).

### Example 305

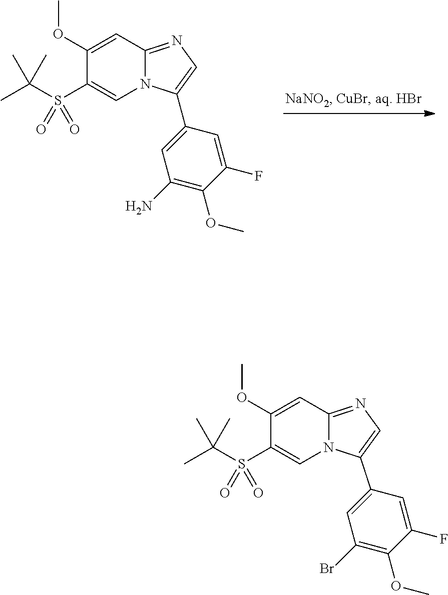 [SMILES: COc1cc2ncc(-c3cc(N)c(OC)c(F)c3)n2cc1S(=O)(=O)C(C)(C)C ; COc1cc2ncc(-c3cc(F)c(OC)c(Br)c3)n2cc1S(=O)(=O)C(C)(C)C]

3-(3-bromo-5-fluoro-4-methoxyphenyl)-6-(tert-butylsulfonyl)-7-methoxyimidazo[1,2-a]pyridine: To a solution of 5-(6-(tert-butylsulfonyl)-7-methoxyimidazo[1,2-a]pyridin-3-yl)-3-fluoro-2-methoxyaniline (20 mg, 39.3 μmol, 1 eq) in HBr (0.1 mL) was added H₂O (1 mL). The mixture was cooled to 0° C., and an ice-cooled solution of NaNO₂ (2.71 mg, 39.3 μmol, 1 eq) in H₂O (1 mL) was dropwise. CuBr (11.3 mg, 79 mol, 2.39 μL, 2 eq) was added to the mixture, and the resulting mixture was stirred at 70° C. for 1 h. The mixture was diluted with water (50 mL), extracted with DCM (50 mL×3), dried over Na₂SO₄, filtered, and concentrated under reduced pressure to give a residue, which was purified by prep-HPLC to give the title compound. [M+H]=472.9. ¹H NMR (400 MHz, DMSO-d₆) δ ppm: 8.67 (s, 1H), 7.81 (s, 1H), 7.74 (s, 1H), 7.67 (d, J=11.6 Hz, 1H), 7.27 (s, 1H), 3.97-3.90 (m, 6H), 1.32 (s, 9H).

### Example 306

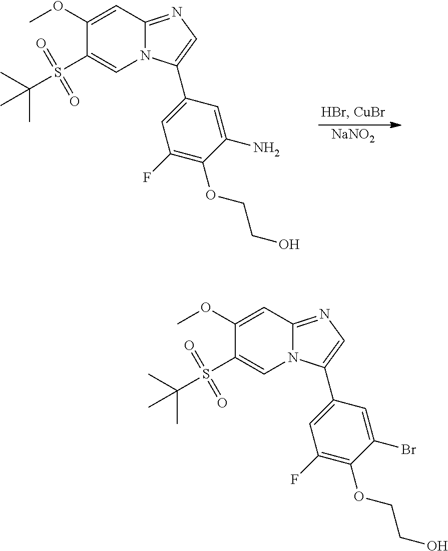

2-(2-bromo-4-(6-(tert-butylsulfonyl)-7-methoxyimidazo[1,2-a]pyridin-3-yl)-6-fluorophenoxy)ethan-1-ol: A solution of 2-(2-amino-4-(6-(tert-butylsulfonyl)-7-methoxyimidazo[1,2-a]pyridin-3-yl)-6-fluorophenoxy)ethan-1-ol (20 mg, 41.1 mol, 1 eq) in HBr (0.2 mL) was cooled to 0° C. A cooled solution of NaNO₂ (2.84 mg, 41.14 μmol, 1 eq) in H₂O (0.5 mL) was added dropwise during 0.2 h, then CuBr (2.51 μL, 82.3 mol, 2 eq) was added to the mixture. The resulting mixture was stirred at 0° C. for 1 h. The reaction mixture was quenched by the addition of sat. NaHCO₃ (10 mL), then diluted with water (20 mL) and extracted with DCM/MeOH (60 mL). The combined organic layers were dried over Na₂SO₄, filtered, and concentrated in vacuo. The resulting crude material was purified via prep RP-HPLC (neutral conditions) to yield the title compound. LCMS [M+H]⁺=503.0. ¹H NMR (400 MHz, DMSO-d₆) δ ppm 8.67 (s, 1H), 7.81 (s, 1H), 7.74 (s, 1H), 7.66 (d, J=12.0 Hz, 1H), 7.27 (s, 1H), 4.89 (t, J=5.2 Hz, 1H), 4.16 (t, J=4.8 Hz, 2H), 3.94 (s, 3H), 3.78-3.73 (m, 2H), 1.32 (s, 9H).

### Example 307

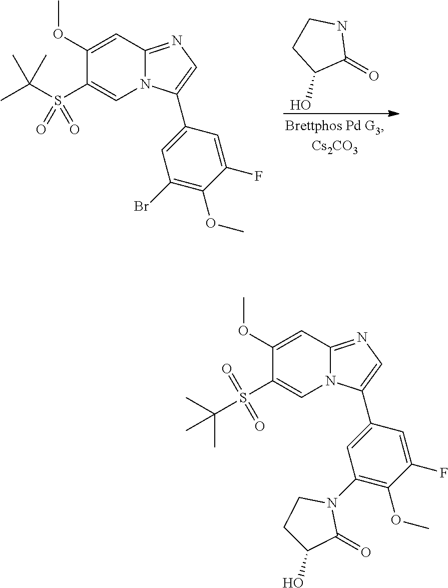 [SMILES: COc1cc2ncc(-c3cc(F)c(OC)c(Br)c3)n2cc1S(=O)(=O)C(C)(C)C ; O=C1NCC[C@H]1O ; COc1cc2ncc(-c3cc(F)c(OC)c(N4CC[C@@H](O)C4=O)c3)n2cc1S(=O)(=O)C(C)(C)C]

(R)-1-(5-(6-(tert-butylsulfonyl)-7-methoxyimidazo[1,2-a]pyridin-3-yl)-3-fluoro-2-methoxyphenyl)-3-hydroxypyrrolidin-2-one: A mixture of 3-(3-bromo-5-fluoro-4-methoxyphenyl)-6-(tert-butylsulfonyl)-7-methoxyimidazo[1,2-a]pyridine (50 mg, 95 mol, 1 eq), (R)-3-hydroxy-1λ2-pyrrolidin-2-one (48 mg, 477 μmol, 5 eq), BrettPhos Pd G3 (8.65 mg, 9.55 mol, 0.1 eq), Cs₂CO₃ (93.3 mg, 286 mol, 3 eq) in 1,4-dioxane (2 mL) was degassed and purged with N₂ three times, and then the mixture was stirred at 100° C. for 12 h under N₂. The reaction mixture was diluted with H₂O (10 mL) and extracted with EtOAc (10 mL×3). The combined organic layers were washed with brine (10 mL), dried over Na₂SO₄, filtered, and concentrated under reduced pressure to give a residue, which was purified by prep-HPLC to give the title compound. [M+H]⁺=492.1. ¹H NMR (400 MHz, DMSO-d₆) δ ppm: 8.69 (s, 1H), 8.46 (br s, 0.6H), 7.76 (s, 1H), 7.63-7.54 (m, 1H), 7.42 (s, 1H), 7.27 (s, 1H), 5.85-5.73 (m, 1H), 4.37-4.24 (m, 1H), 3.93 (s, 3H), 3.90 (s, 3H), 3.72-3.66 (m, 2H), 2.45-2.40 (m, 1H), 1.99-1.89 (m, 1H), 1.32 (s, 9H).

### Example 308

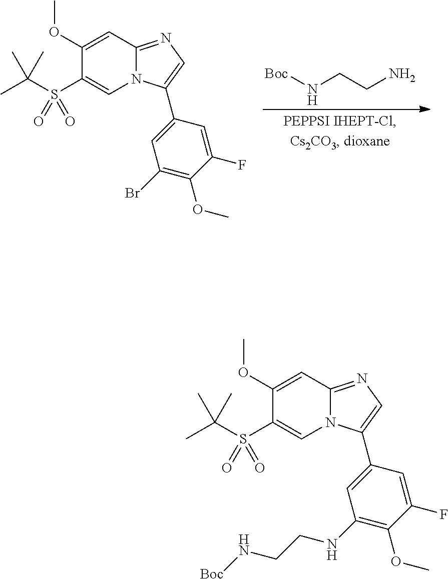 [SMILES: COc1cc2ncc(-c3cc(F)c(OC)c(Br)c3)n2cc1S(=O)(=O)C(C)(C)C ; CC(C)(C)OC(=O)NCCN ; COc1cc2ncc(-c3cc(F)c(OC)c(NCCNC(=O)OC(C)(C)C)c3)n2cc1S(=O)(=O)C(C)(C)C]

tert-butyl (2-((5-(6-(tert-butylsulfonyl)-7-methoxyimidazo[1,2-a]pyridin-3-yl)-3-fluoro-2-methoxyphenyl)amino)ethyl)carbamate: To a solution of tert-butyl (2-aminoethyl)carbamate (76.5 mg, 477 μmol, 75.3 μL, 5 eq), Cs₂CO₃ (93.3 mg, 286 μmol, 3 eq), PEPPSI IHPET-Cl (9.29 mg, 9.6 mol, 0.1 eq) in 1,4-dioxane (1 mL) was added 3-(3-bromo-5-fluoro-4-methoxyphenyl)-6-(tert-butylsulfonyl)-7-methoxyimidazo[1,2-a]pyridine. The mixture was stirred at 100° C. for 12 h under N₂. The reaction mixture was filtered and concentrated under reduced pressure to give a residue, which was purified by prep-HPLC to give the title compound. [M+H]⁺=551.0. ¹H NMR (400 MHz, DMSO-d₆) δ ppm: 8.75 (s, 1H), 7.71 (s, 1H), 7.25 (s, 1H), 7.01-6.92 (m, 1H), 6.72-6.64 (m, 2H), 5.83-5.76 (m, 1H), 3.93 (s, 3H), 3.82 (s, 3H), 3.14 (s, 4H), 1.32 (s, 18H).

### Example 309

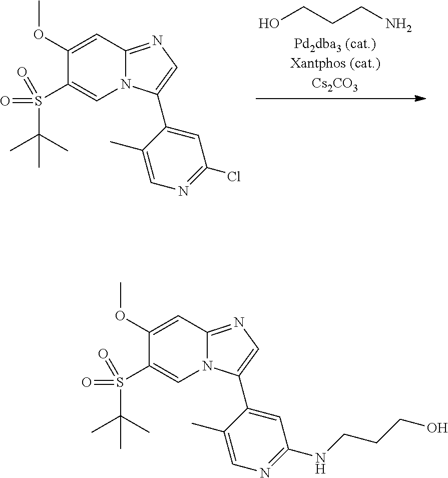 [SMILES: COc1cc2ncc(-c3cc(Cl)ncc3C)n2cc1S(=O)(=O)C(C)(C)C ; NCCCO ; COc1cc2ncc(-c3cc(NCCCO)ncc3C)n2cc1S(=O)(=O)C(C)(C)C]

3-({4-[7-Methoxy-6-(2-methylpropane-2-sulfonyl)imidazo[1,2-a]pyridin-3-yl]-5-methylpyridin-2-yl}amino)propan-1-ol: 6-(tert-Butylsulfonyl)-3-(2-chloro-5-methylpyridin-4-yl)-7-methoxyimidazo[1,2-a]pyridine (70.0 mg, 178 μmol), cesium carbonate (116 mg, 355 mol), 4,5-bis(diphenylphosphino)-9,9-dimethyl xanthene (20.6 mg, 35.5 μmol) and tris(dibezylideneacetone)dipalladium (16.3 mg, 17.8 μmol) were combined in toluene (3.0 mL). 3-Aminopropan-1-ol (40.0 mg, 533 μmol) was added and the mixture was stirred for 6 h at 100° C. under argon. To the mixture was added brine and the water layer was extracted with EtOAc. The combined organics were dried and concentrated under reduced pressure. The residue was purified by HPLC to give the title compound. [M+H]⁺=433.5. ¹H NMR (400 MHz, DMSO-d₆) δ ppm 8.30 (s, 1H), 8.02 (s, 1H), 7.72 (s, 1H), 7.28-7.33 (m, 1H), 6.53 (t, J=5.70 Hz, 1H), 6.48 (s, 1H), 4.49 (t, J=5.20 Hz, 1H), 3.91-3.96 (m, 3H), 3.44-3.50 (m, 2H), 3.21-3.30 (m, 2H), 1.96 (s, 3H), 1.62-1.75 (m, 2H), 1.30 (s, 9H).

The following compounds were prepared following procedures analogous to that described for Example 309.

| Ex. | | | |
|---|---|---|---|
| No. | Structure | Name | LCMS, ¹H NMR |
| 310 | 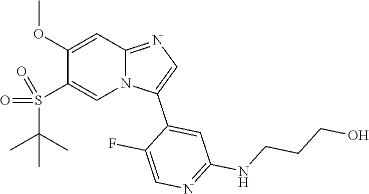 [SMILES: COc1cc2ncc(-c3cc(NCCCO)ncc3F)n2cc1S(=O)(=O)C(C)(C)C] | 3-((4-(6-(tert- butylsulfonyl)-7- methoxyimidazo[1, 2-a]pyridin-3-yl)- 5-fluoropyridin-2- yl)amino)propan- 1-ol | [M + H]⁺ = 437.5 ¹H NMR (400 MHz, DMSO-d₆) δ ppm 8.50 (d, J = 3.30 Hz, 1 H), 8.16 (d, J = 2.28 Hz, 1 H), 7.88 (s, 1 H), 7.33 (s, 1 H), 6.76 (t, J = 5.58 Hz, 1 H), 6.63 (d, J = 4.82 Hz, 1 H), 4.48 (t, J = 5.20 Hz, 1 H), 3.95 (s, 3 H), 3.49 (q, J = 6.08 Hz, 2 H), 3.23-3.30 (m, 2 H), 1.69 (quin, J = 6.59 Hz, 2 H), 1.31 (s, 9 H). |
| 311 | 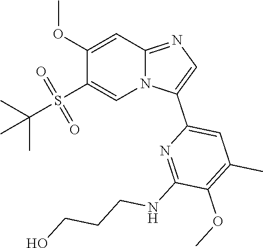 [SMILES: COc1cc2ncc(-c3cc(C)c(OC)c(NCCCO)n3)n2cc1S(=O)(=O)C(C)(C)C] | 3-((6-(6-(tert- butylsulfonyl)-7- methoxyimidazo[1, 2-a]pyridin-3-yl)- 3-methoxy-4- methylpyridin-2- yl)amino)propan- 1-ol | [M + H]⁺ = 462.9 ¹H NMR (400 MHz, DMSO-d₆) δ ppm 10.51 (s, 1H), 8.15 (s, 1H), 7.24 (s, 1H), 7.01 (s, 1H), 6.29-6.23 (m, 1H), 4.48-4.43 (m, 1H), 3.93 (s, 3H), 3.66 (s, 3H), 3.62-3.48 (m, 4H), 2.21 (s, 3H), 1.85-1.72 (m, 2H), 1.32 (s, 9H). |
| 312 | 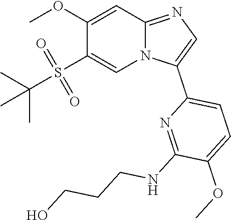 [SMILES: COc1cc2ncc(-c3ccc(OC)c(NCCCO)n3)n2cc1S(=O)(=O)C(C)(C)C] | 3-((6-(6-(tert- butylsulfonyl)-7- methoxyimidazo[1, 2-a]pyridin-3-yl)- 3-methoxypyridin- 2- yl)amino)propan- 1-ol | [M + H]⁺ = 449.0 ¹H NMR (400 MHz, DMSO-d₆) δ ppm 10.46 (s, 1H), 8.11 (s, 1H), 7.23 (s, 1H), 7.11 (d, J = 2.0 Hz, 2H), 6.16-6.13 (m, 1H), 4.48 (t, J = 5.2 Hz, 1H), 3.92 (s, 3H), 3.84 (s, 3H), 3.56-3.50 (m, 4H), 1.83-1.72 (m, 2H), 1.32 (s, 9H). |

### Example 313

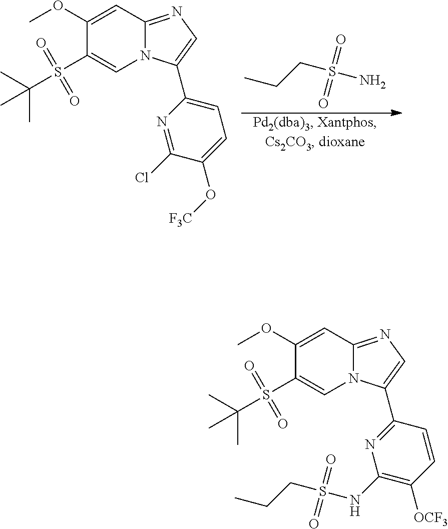

N-(6-(6-(tert-butylsulfonyl)-7-methoxyimidazo[1,2-a]pyridin-3-yl)-3-(trifluoromethoxy)pyridin-2-yl)propane-1-sulfonamide: A mixture of 6-(tert-butylsulfonyl)-3-(6-chloro-5-(trifluoromethoxy)pyridin-2-yl)-7-methoxyimidazo[1,2-a]pyridine (50 mg, 97.0 mol, 1 eq), propane-1-sulfonamide (36 mg, 291 mol, 3 eq), Pd(OAc)₂ (2.18 mg, 9.70 μmol, 0.1 eq), Xantphos (11.2 mg, 19.4 μmol, 0.2 eq) and Cs₂CO₃ (94.8 mg, 291 μmol, 3 eq) in dioxane (3 mL) was degassed and purged with N₂ three times, and then the mixture was stirred at 100° C. for 12 h under N₂. The reaction mixture was diluted with H₂O (20 mL) and extracted with EtOAc (20 mL×3). The combined organic layers were washed with brine (20 mL), dried over Na₂SO₄, filtered, and concentrated under reduced pressure to give a residue. The crude product was purified by prep-HPLC to give the title compound. [M+H]=551.0. ¹H NMR: (400 MHz, DMSO-d₆) δ ppm: 10.78 (s, 1H), 9.79 (s, 1H), 8.38 (s, 1H), 7.97-7.90 (m, 1H), 7.82-7.70 (m, 1H), 7.35 (s, 1H), 3.95 (s, 3H), 3.55-3.46 (m, 2H), 1.82-1.72 (m, 2H), 1.33 (s, 9H), 0.95 (t, J=7.6 Hz, 3H).

The following compound was prepared following procedures analogous to that described for Example 313.

| Ex. | | | |
|---|---|---|---|
| No. | Structure | Name | LCMS, ¹H NMR |
| 314 | 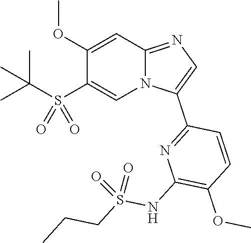 [SMILES: CCCS(=O)(=O)Nc1nc(-c2cnc3cc(OC)c(S(=O)(=O)C(C)(C)C)cn23)ccc1OC] | N-(6-(6-(tert- butylsulfonyl)-7- methoxyimidazo[1, 2-a]pyridin-3-yl)-3- methoxypyridin-2- yl)propane-1- sulfonamide | [M + H]⁺ = 497.0 ¹H NMR (400 MHz, DMSO-d₆) δ ppm 9.69 (s, 1H), 8.38 (s, 0.3H), 8.12 (s, 1H), 7.64-7.56 (m, 1H), 7.55-7.45 (m, 1H), 7.28 (s, 1H), 3.93 (s, 3H), 3.88 (s, 3H), 3.54- 3.26 (m, 2H), 1.84-1.67 (m, 2H), 1.32 (s, 9H), 1.01-0.87 (m, 3H). |

### Example 315

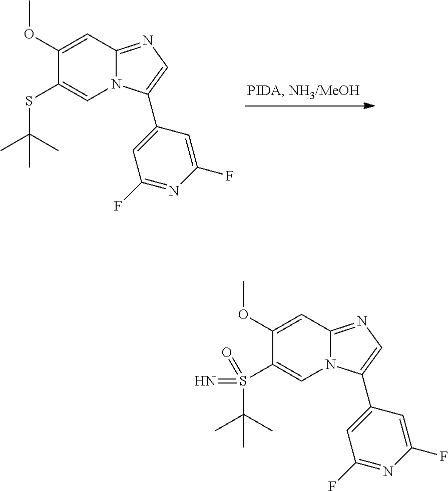 [SMILES: COc1cc2ncc(-c3cc(F)nc(F)c3)n2cc1SC(C)(C)C ; COc1cc2ncc(-c3cc(F)nc(F)c3)n2cc1S(=N)(=O)C(C)(C)C]

tert-butyl(3-(2,6-difluoropyridin-4-yl)-7-methoxyimidazo[1,2-a]pyridin-6-yl)(imino)-λ⁶-sulfanone: 6-(tert-butylthio)-3-(2,6-difluoropyridin-4-yl)-7-methoxyimidazo[1,2-a]pyridine (100 mg, 1 equiv, 286 μmol) was dissolved in methanol (5 mL). Ammonia in methanol (12 equiv.) and phenyl iodonium diacetate (PIDA) (461 mg, 5 equiv, 1.43 mmol) were added, and the mixture was stirred at 40° C. for 16 h. Water was added, and the aq. phase was extracted with EtOAc and CH₂Cl₂ twice. The combined organic phases were dried, and the solvent was evaporated. The residue was dissolved in DMSO (2 mL) and submitted to HPLC purification to give of the title compound. [M+H]⁺=381.4. ¹H NMR (400 MHz, DMSO-d₆) δ ppm 9.00 (s, 1H), 8.20 (s, 1H), 7.52 (s, 2H), 7.30 (s, 1H), 4.53 (s, 1H), 3.94 (s, 3H), 1.31 (s, 9H).

The following compounds were prepared following procedures analogous to that described for Example 315.

| Ex. | | | |
|---|---|---|---|
| No. | Structure | Name | LCMS, ¹H NMR |
| 316 | 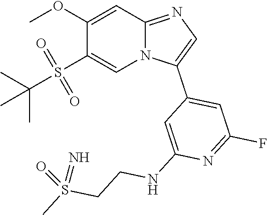 [SMILES: COc1cc2ncc(-c3cc(F)nc(NCCS(C)(=N)=O)c3)n2cc1S(=O)(=O)C(C)(C)C] | Rac-(2-((4-(6-(tert- butylsulfonyl)-7- methoxyimidazo[1,2- a]pyridin-3-yl)-6- fluoropyridin-2- yl)amino)ethyl)(imino) (methyl)-26-sulfanone | [M + H]⁺ = 484.2 ¹H NMR (400 MHz, DMSO-d₆) δ ppm 8.81 (s, 1 H), 7.94 (s, 1 H), 7.42-7.53 (m, 1 H), 7.31 (s, 1 H), 6.58 (s, 1 H), 6.46 (s, 1 H), 3.94 (s, 3 H), 3.80 (br s, 1 H), 3.67 (q, J = 6.42 Hz, 2 H), 3.27-3.35 (m, 2 H), 2.95 (s, 3 H), 1.32 (s, 9 H). |

### Example 317

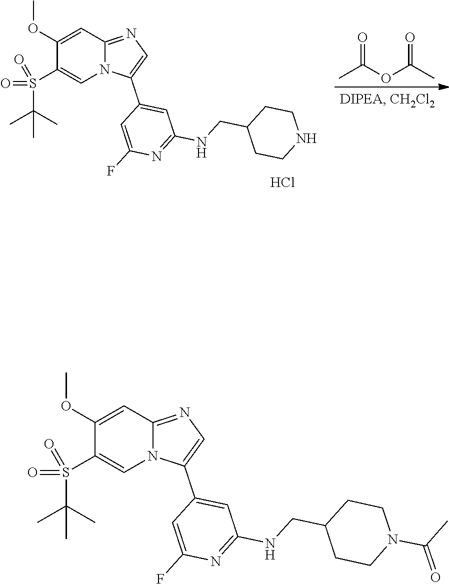

1-(4-(((4-(6-(tert-butylsulfonyl)-7-methoxyimidazo[1,2-a]pyridin-3-yl)-6-fluoropyridin-2-yl)amino)methyl)piperidin-1-yl)ethan-1-one: 4-(6-(tert-butylsulfonyl)-7-methoxyimidazo[1,2-a]pyridin-3-yl)-6-fluoro-N-(piperidin-4-ylmethyl)pyridin-2-amine hydrochloride (290 mg, 1 equiv, 566 μmol) was dissolved in CH₂Cl₂ (10 mL). Acetic anhydride (72 mg, 66.8 μL, 1.25 equiv, 708 μmol) and DIPEA (220 mg, 292 μL, 3 equiv, 1.70 mmol) were added, and the resulting mixture was stirred at rt for 22 h. The solvent was evaporated. The residue was dissolved in DMSO (2 mL) and submitted to HPLC purification (basic method) to give the title compound. [M+H]⁺=518.5. ¹H NMR (400 MHz, DMSO-d₆) δ ppm 8.81 (s, 1H), 7.92 (s, 1H), 7.32-7.42 (m, 1H), 7.31 (s, 1H), 6.54 (s, 1H), 6.36 (s, 1H), 4.32-4.43 (m, 1H), 3.94 (s, 3H), 3.81 (br d, J=13.7 Hz, 1H), 3.13 (br s, 2H), 2.93-3.04 (m, 1H), 2.47 (br s, 1H), 1.98 (s, 3H), 1.66-1.88 (m, 3H), 1.32 (s, 9H), 0.91-1.19 (m, 2H).

The following compounds were prepared following procedures analogous to that described for Example 317.

| Ex. | | | |
|---|---|---|---|
| No. | Structure | Name | LCMS, ¹H NMR |
| 318 | 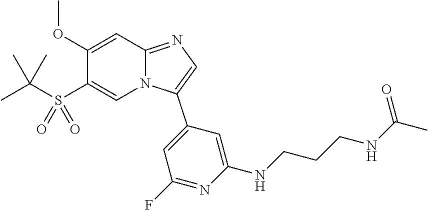 [SMILES: COc1cc2ncc(-c3cc(F)nc(NCCCNC(C)=O)c3)n2cc1S(=O)(=O)C(C)(C)C] | N-[3-({6-fluoro-4- [7-methoxy-6-(2- methylpropane-2- sulfonyl)imidazo [1,2-a]pyridin-3- yl]pyridin-2- yl}amino)propyl] acetamide | [M + H]+ = 478.4 ¹H NMR (400 MHz, DMSO- d₆) δ ppm 8.81 (s, 1 H), 7.92 (s, 1 H), 7.86 (br t, J = 5.32 Hz, 1 H), 7.31 (s, 1 H), 7.25 (br s, 1 H), 6.51 (s, 1 H), 6.37 (s, 1 H), 3.94 (s, 3 H), 3.19- 3.26 (m, 2 H), 3.10 (q, J = 6.76 Hz, 2 H), 1.79 (s, 3 H), 1.67 (quin, J = 6.91 Hz, 2 H), 1.32 (s, 9 H) |

### Example 319

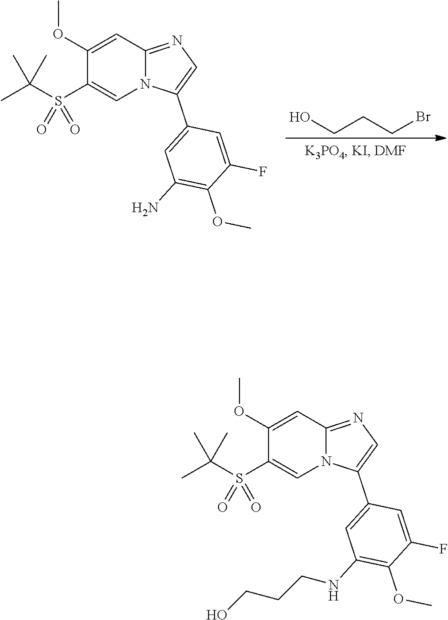 [SMILES: COc1cc2ncc(-c3cc(N)c(OC)c(F)c3)n2cc1S(=O)(=O)C(C)(C)C ; OCCCBr ; COc1cc2ncc(-c3cc(F)c(OC)c(NCCCO)c3)n2cc1S(=O)(=O)C(C)(C)C]

3-((5-(6-(tert-butylsulfonyl)-7-methoxyimidazo[1,2-a]pyridin-3-yl)-3-fluoro-2-methoxyphenyl)amino)propan-1-ol: To a solution of 5-(6-(tert-butylsulfonyl)-7-methoxyimidazo[1,2-a]pyridin-3-yl)-3-fluoro-2-methoxyaniline (50 mg, 98 μmol, 1 eq) in DMF (1 mL) was added 3-bromopropan-1-ol (68 mg, 491 μmol, 44.4 μL, 5 eq), K₃PO₄ (63 mg, 295 μmol, 3 eq) and KI (49 mg, 295 μmol, 3 eq). The mixture was stirred at 80° C. for 5 h. The mixture was filtered to give the residue, which was purified by prep-HPLC to give the title compound. [M+H]⁺=466.1. ¹H NMR (400 MHz, DMSO-d₆) δ ppm: 8.76 (s, 1H), 7.70 (s, 1H), 7.24 (s, 1H), 6.68-6.58 (m, 2H), 5.75 (t, J=5.6 Hz, 1H), 4.51 (t, J=4.8 Hz, 1H), 3.92 (s, 3H), 3.82 (s, 3H), 3.52-3.45 (m, 2H), 3.21-3.13 (m, 2H), 1.76-1.68 (m, 2H), 1.32 (s, 9H).

### Example 320

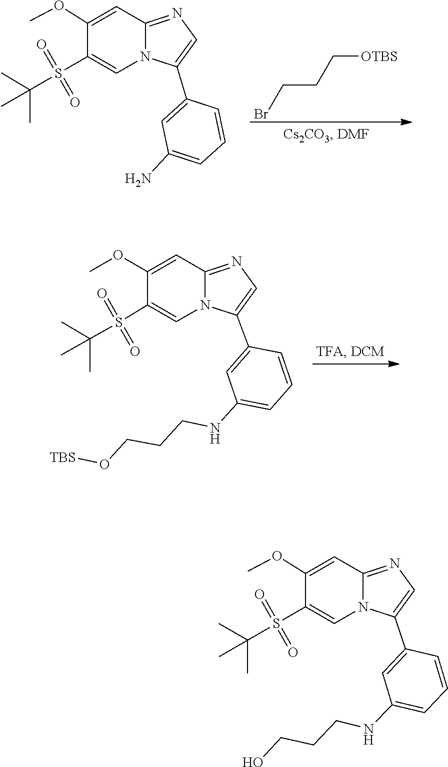

N-(3-((tert-butyldimethylsilyl)oxy)propyl)-3-(6-(tert-butylsulfonyl)-7-methoxyimidazo[1,2-a]pyridin-3-yl)aniline was prepared according to a procedure analogous to the one described for Example 319.

3-((3-(6-(tert-butylsulfonyl)-7-methoxyimidazo[1,2-a]pyridin-3-yl)phenyl)amino)propan-1-ol: To a solution of N-(3-((tert-butyldimethylsilyl)oxy)propyl)-3-(6-(tert-butylsulfonyl)-7-methoxyimidazo[1,2-a]pyridin-3-yl)aniline (60 mg, 113 μmol, 1 eq) in DCM (1 mL) was added TFA (0.2 mL). The mixture was stirred at 25° C. for 1 h under N₂. The reaction mixture was concentrated under reduced pressure to give a residue, which was purified by prep-HPLC to give the title compound. [M+H]⁺=418.2. ¹H NMR (400 MHz, DMSO-d₆) δ ppm: 8.77 (s, 1H), 7.67 (s, 1H), 7.29-7.19 (m, 2H), 6.75-6.61 (m, 3H), 5.92-5.81 (m, 1H), 4.47 (br s, 1H), 3.93 (s, 3H), 3.55-3.46 (m, 2H), 3.14-3.04 (m, 2H), 1.76-1.66 (m, 2H), 1.31 (s, 9H).

The following compound was prepared following procedures analogous to that described for Example 320.

| Ex. | | | |
|---|---|---|---|
| No. | Structure | Name | LCMS, ¹H NMR |
| 321 | 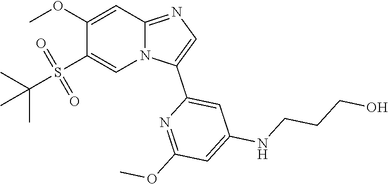 [SMILES: COc1cc(NCCCO)cc(-c2cnc3cc(OC)c(S(=O)(=O)C(C)(C)C)cn23)n1] | 3-((2-(6-(tert- butylsulfonyl)-7- methoxyimidazo[1, 2-a]pyridin-3-yl)-6- methoxypyridin-4- yl)amino)propan-1- ol | [M + H]⁺ = 449.1 ¹H NMR (400 MHz, DMSO-d₆) δ ppm 10.54 (s, 1H), 8.16 (br s, 1H), 7.26 (s, 1H), 6.79 (s, 1H), 6.61-6.54 (m, 1H), 5.81 (s, 1H), 4.52 (t, J = 4.8 Hz, 1H), 3.95- 3.89 (m, 6H), 3.55-3.45 (m, 2H), 3.19-3.13 (m, 2H), 1.74- 1.65 (m, 2H), 1.31 (s, 9H). |

### Example 322

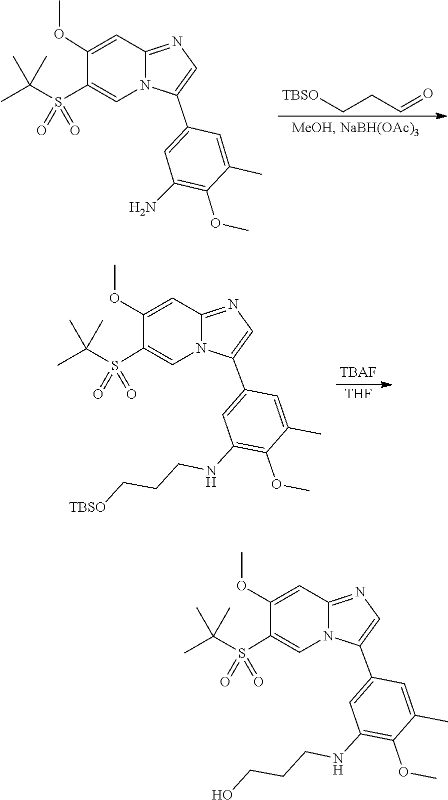

N-(3-((tert-butyldimethylsilyl)oxy)propyl)-5-(6-(tert-butylsulfonyl)-7-methoxyimidazo[1,2-a]pyridin-3-yl)-2-methoxy-3-methylaniline: A mixture of 5-(6-(tert-butylsulfonyl)-7-methoxyimidazo[1,2-a]pyridin-3-yl)-2-methoxy-3-methylaniline (50 mg, 111 μmol, 1 eq), 3-((tert-butyldimethylsilyl)oxy)propanal (42 mg, 223 μmol, 2 eq), and NaBH(OAc)₃ (14.0 mg, 223 μmol, 2 eq) in MeOH (8 mL) was degassed and purged with N₂ three times, and then the mixture was stirred at 25° C. for 12 h under N₂. The mixture was concentrated and then water (10 mL) was added. The mixture was extracted with EtOAc (30 mL×3). The combined organic layers were washed with brine (10 mL), dried over Na₂SO₄, filtered, and concentrated under reduced pressure to give a residue, which was purified by column chromatography (SiO₂, DCM/MeOH=100/0 to 9/1) to give the title compound.

3-((5-(6-(tert-butylsulfonyl)-7-methoxyimidazo[1,2-a]pyridin-3-yl)-2-methoxy-3-methylphenyl)amino)propan-1-ol: To a solution of N-(3-((tert-butyldimethylsilyl)oxy)propyl)-5-(6-(tert-butylsulfonyl)-7-methoxyimidazo[1,2-a]pyridin-3-yl)-2-methoxy-3-methylaniline (60 mg, 93.8 mol, 1 eq) in THE (12 mL) was added TBAF (1 M, 281 μL, 3 eq). The mixture was stirred at 25° C. for 2 h. The reaction mixture was filtered and concentrated under reduced pressure to give a residue, which was purified by prep-HPLC to give the title compound. [M+H]⁺=462.1. ¹H NMR (400 MHz, DMSO-d₆) δ ppm: 8.77 (s, 1H), 7.65 (s, 1H), 7.24 (s, 1H), 6.63 (s, 1H), 6.58 (s, 1H), 5.43-5.40 (m, 1H), 4.56-4.45 (s, 1H), 3.93 (s, 3H), 3.68 (s, 3H), 3.51-3.46 (m, 2H), 3.18-3.11 (m, 2H), 2.23 (s, 3H), 1.77-1.67 (m, 2H), 1.32 (s, 9H).

### Example 323

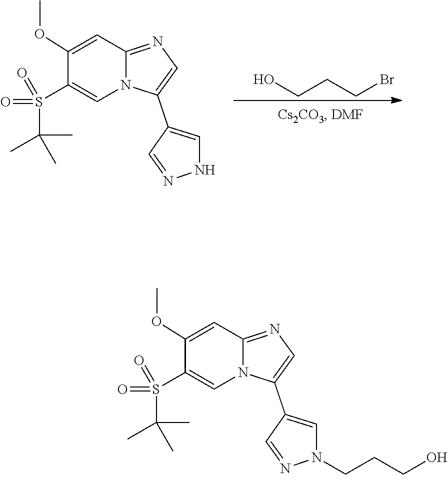

3-(4-(6-(tert-butylsulfonyl)-7-methoxyimidazo[1,2-a]pyridin-3-yl)-1H-pyrazol-1-yl)propan-1-ol: To a solution of 6-(tert-butylsulfonyl)-7-methoxy-3-(1H-pyrazol-4-yl)imidazo[1,2-a]pyridine (50 mg, 135 mol, 1 eq) in DMF (10 mL) was added Cs₂CO₃ (43.9 mg, 135 mol, 1 eq), and then added 3-bromopropan-1-ol (18.7 mg, 135 μmol, 12.2 μL, 1 eq). The mixture was stirred at 60° C. for 16 h under N₂. The reaction mixture was filtered and concentrated under reduced pressure. The resulting residue was purified by prep-HPLC to give the title compound. [M+H]⁺=393.1. ¹H NMR (400 MHz, DMSO-d₆) δ ppm: 8.53 (s, 1H), 8.15 (s, 1H), 7.79 (s, 1H), 7.63 (s, 1H), 7.23 (s, 1H), 4.66-4.61 (m, 1H), 4.32-4.23 (m, 2H), 3.92 (s, 3H), 3.47-3.39 (m, 2H), 2.02-1.92 (m, 2H), 1.31 (s, 9H).

### Example 324

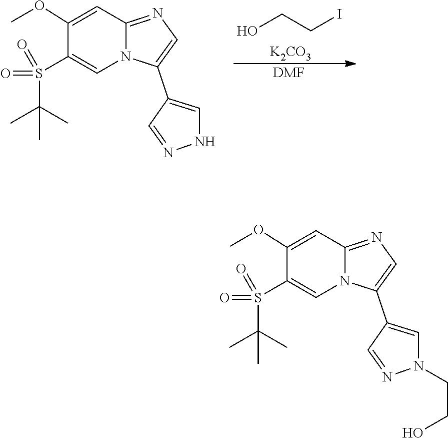

2-(4-(6-(tert-butylsulfonyl)-7-methoxyimidazo[1,2-a]pyridin-3-yl)-1H-pyrazol-1-yl)ethan-1-ol: A mixture of 6-(tert-butylsulfonyl)-7-methoxy-3-(1H-pyrazol-4-yl)imidazo[1,2-a]pyridine (70 mg, 188.40 μmol, 1 eq) and K₂CO₃ (78.11 mg, 565.20 μmol, 3 eq) in DMF (10 mL) was degassed and purged with N₂ (3×). 2-Iodoethanol (29.45 μL, 376.80 mol, 2 eq) was added, and the reaction mixture was stirred at 80° C. for 12 h under N₂ atmosphere. The reaction mixture was filtered and concentrated in vacuo. The resulting crude material was purified via prep RP-HPLC (neutral conditions) to yield the title compound. LCMS [M+H]⁺=379.1. ¹H NMR (400 MHz, DMSO-d₆) δ ppm 8.56 (s, 1H), 8.12 (s, 1H), 7.80 (s, 1H), 7.63 (s, 1H), 7.23 (s, 1H), 5.03-4.88 (m, 1H), 4.28-4.22 (m, 2H), 3.92 (s, 3H), 3.82-3.77 (m, 2H), 1.31 (s, 9H).

### Example 325

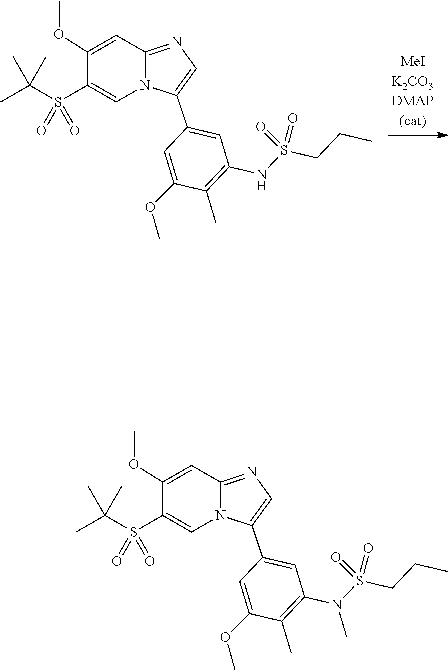

N-(5-(6-(tert-butylsulfonyl)-7-methoxyimidazo[1,2-a]pyridin-3-yl)-3-methoxy-2-methylphenyl)-N-methylpropane-1-sulfonamide: N-(5-(6-(tert-Butylsulfonyl)-7-methoxyimidazo[1,2-a]pyridin-3-yl)-3-methoxy-2-methylphenyl)propane-1-sulfonamide (20.0 mg, 39.2 μmol), methyl iodide (7.62 μL, 118 μmol) and potassium carbonate (10.9 mg, 78.5 mol) were combined in DMF (2.0 mL). DMAP (479 μg, 3.92 μmol) was added, and the mixture was stirred for 20 h at 50° C. The mixture was concentrated under reduced pressure. The crude was purified by high-throughput preparative HPLC using acidic method to give the title compound. [M+H]⁺=524.2. ¹H NMR (400 MHz, DMSO-d₆) δ ppm 8.76 (s, 1H), 7.83 (s, 1H), 7.29-7.34 (m, 1H), 7.28 (s, 1H), 7.25-7.23 (m, 1H), 3.94 (s, 3H), 3.86 (s, 3H), 3.16-3.20 (m, 5H), 2.19 (s, 3H), 1.70-1.82 (m, 2H), 1.29-1.33 (m, 9H), 1.01 (t, J=7.48 Hz, 3H).

### Example 326

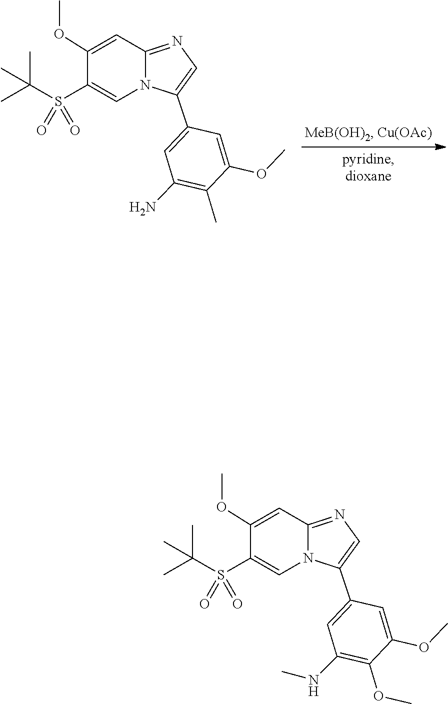

5-(6-(tert-butylsulfonyl)-7-methoxyimidazo[1,2-a]pyridin-3-yl)-2,3-dimethoxy-N-methylaniline: To a solution of 5-(6-(tert-butylsulfonyl)-7-methoxyimidazo[1,2-a]pyridin-3-yl)-2,3-dimethoxyaniline (40 mg, 86 μmol, 1 eq) and pyridine (23.8 mg, 300 mol, 24.2 μL, 3.5 eq) in 1,4-dioxane (2 mL) was added Cu(OAc)₂ (39 mg, 215 mol, 2.5 eq) and MeB(OH)₂ (12.8 mg, 215 μmol, 2.5 eq). The mixture was stirred at 100° C. for 3 h under air atmosphere. The reaction mixture was diluted with water (10 mL) and extracted with EtOAc (15 mL×3). The combined organic layers were washed with brine (15 mL), dried over Na₂SO₄, filtered, and concentrated under reduced pressure. The residue was purified by prep-HPLC to give the title compound. [M+H]⁺=434.1. ¹H NMR (400 MHz, DMSO-d₆) δ ppm: 8.91 (s, 1H), 8.30-8.10 (m, 1H), 7.37-6.95 (m, 1H), 6.53 (s, 1H), 6.39 (s, 1H), 5.57-5.50 (m, 1H), 3.93 (s, 3H), 3.79 (s, 3H), 3.70 (s, 3H), 2.71 (d, J=4.8 Hz, 3H), 1.32 (s, 9H).

### Example 327

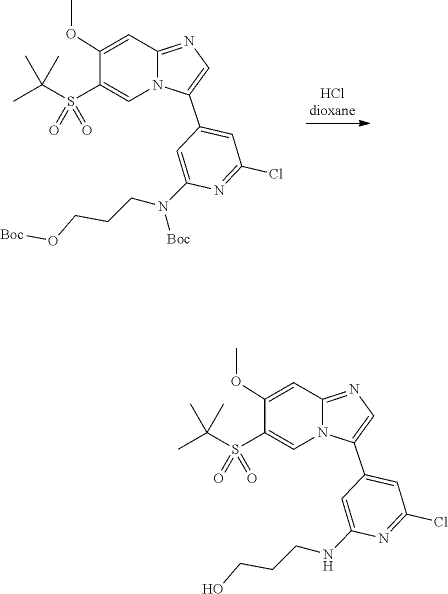 [SMILES: COc1cc2ncc(-c3cc(Cl)nc(N(CCCOC(=O)OC(C)(C)C)C(=O)OC(C)(C)C)c3)n2cc1S(=O)(=O)C(C)(C)C ; COc1cc2ncc(-c3cc(Cl)nc(NCCCO)c3)n2cc1S(=O)(=O)C(C)(C)C]

3-((4-(6-(tert-butylsulfonyl)-7-methoxyimidazo[1,2-a]pyridin-3-yl)-6-chloropyridin-2-yl)amino)propan-1-ol: To a mixture of tert-butyl (3-((tert-butoxycarbonyl)oxy)propyl)(4-(6-(tert-butylsulfonyl)-7-methoxyimidazo[1,2-a]pyridin-3-yl)-6-chloropyridin-2-yl)carbamate (76.0 mg, 116 μmol) in 1,4-dioxane (1160 μL), hydrochloric acid (4 M, 1.16 mL) was added. The mixture was stirred for 16 h at 25° C. The mixture was concentrated under reduced pressure. The residue was purified by high-throughput preparative HPLC using basic method, followed by a preparative HPLC using acidic method and a flash chromatography (silica gel, DCM/ethanol gradient) to give the title compound. [M+H]=453.5. ¹H NMR (400 MHz, DMSO-d₆) δ ppm 8.79 (s, 1H), 7.93 (s, 1H), 7.30 (s, 1H), 7.25 (br t, J=5.32 Hz, 1H), 6.75 (d, J=1.01 Hz, 1H), 6.58 (d, J=1.01 Hz, 1H), 4.49 (t, J=5.20 Hz, 1H), 3.94 (s, 3H), 3.45-3.52 (m, 2H), 3.25-3.32 (m, 2H), 1.70 (quin, J=6.65 Hz, 2H), 1.32 (s, 9H).

The following compounds were prepared following procedures analogous to that described for Example 327

| Ex. | | | |
|---|---|---|---|
| No. | Structure | Name | LCMS, ¹H NMR |
| 328 | 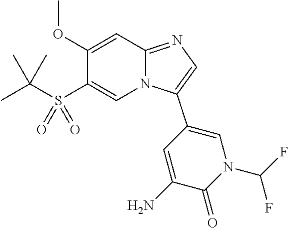 [SMILES: COc1cc2ncc(-c3cc(N)c(=O)n(C(F)F)c3)n2cc1S(=O)(=O)C(C)(C)C] | 3-amino-5-(6-(tert- butylsulfonyl)-7- methoxyimidazo[1,2- a]pyridin-3-yl)-1- (difluoromethyl) pyridin-2(1H)-one | [M + H]⁺ = 427.1 ¹H NMR (400 MHz, DMSO-d₆) δ ppm 8.56 (s, 1H), 8.38 (s, 0.24H), 8.16-7.83 (m, 1H), 7.66 (s, 1H), 7.28 (d, J = 2.0 Hz, 1H), 7.24 (s, 1H), 6.58 (d, J = 2.0 Hz, 1H), 5.83 (s, 2H), 3.92 (s, 3H), 1.31 (s, 9H). |
| 329 | 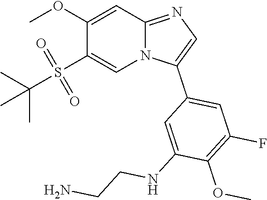 [SMILES: COc1cc2ncc(-c3cc(F)c(OC)c(NCCN)c3)n2cc1S(=O)(=O)C(C)(C)C] | N1-(5-(6-(tert- butylsulfonyl)-7- methoxyimidazo[1,2- a]pyridin-3-yl)-3- fluoro-2- methoxyphenyl) ethane-1,2-diamine | [M + H]⁺ = 450.9 ¹H NMR (400 MHz, DMSO-d₆) δ ppm 8.75 (s, 1H), 7.75-7.66 (m, 1H), 7.25 (s, 1H), 6.65 (s, 2H), 5.82-5.65 (m, 1H), 3.93 (s, 3H), 3.83 (s, 3H), 3.14-3.07 (m, 2H), 2.75 (t, J = 6.4 Hz, 2H), 1.32 (s, 9H). |

### Example 330

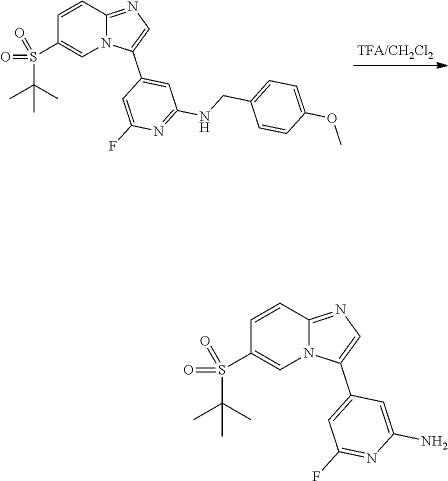 [SMILES: COc1ccc(CNc2cc(-c3cnc4ccc(S(=O)(=O)C(C)(C)C)cn34)cc(F)n2)cc1 ; CC(C)(C)S(=O)(=O)c1ccc2ncc(-c3cc(N)nc(F)c3)n2c1]

4-(6-(tert-butylsulfonyl)imidazo[1,2-a]pyridin-3-yl)-6-fluoropyridin-2-amine. 4-(6-(tert-butylsulfonyl)imidazo[1,2-a]pyridin-3-yl)-6-fluoro-N-(4-methoxybenzyl)pyridin-2-amine (280 mg, 87% wt, 1 equiv, 520 μmol) was dissolved in CH₂Cl₂ (8 mL). Trifluoroacetic acid (1.96 g, 1.31 mL, 33 equiv, 17.2 mmol) was added dropwise, and the mixture was stirred at rt for 3 h. The mixture was diluted with additional CH₂Cl₂ and then washed with a saturated aq. solution of NaHCO₃. The organic phase was dried and evaporated. The residue was dissolved in DMSO (2 mL) and submitted to HPLC purification to give the title compound. [M+H]⁺=349.4. ¹H NMR (400 MHz, DMSO-d₆) δ ppm 8.75-8.84 (m, 1H), 8.09 (s, 1H), 7.88 (dd, J=9.4, 0.8 Hz, 1H), 7.58 (dd, J=9.4, 1.8 Hz, 1H), 6.70 (s, 2H), 6.53 (s, 1H), 6.45 (s, 1H), 1.33 (s, 9H).

### Example 331

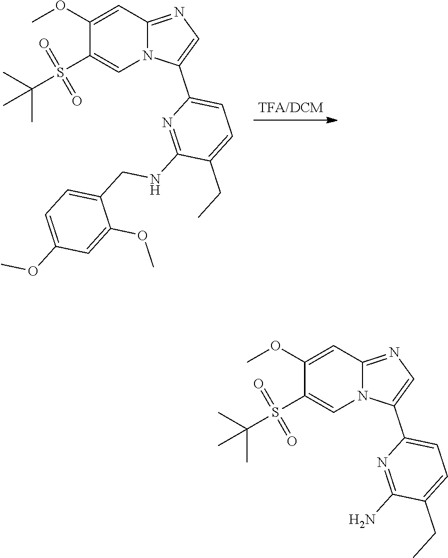 [SMILES: CCc1ccc(-c2cnc3cc(OC)c(S(=O)(=O)C(C)(C)C)cn23)nc1NCc1ccc(OC)cc1OC ; CCc1ccc(-c2cnc3cc(OC)c(S(=O)(=O)C(C)(C)C)cn23)nc1N]

6-(6-(tert-butylsulfonyl)-7-methoxyimidazo[1,2-a]pyridin-3-yl)-3-ethylpyridin-2-amine: To a solution of 6-(6-(tert-butylsulfonyl)-7-methoxyimidazo[1,2-a]pyridin-3-yl)-N-(2,4-dimethoxybenzyl)-3-ethylpyridin-2-amine (100 mg, 167 mol, 1 eq) in DCM (5 mL) was added TFA (1.78 g, 15.6 mmol, 1.15 mL). The mixture was stirred at 25° C. for 2 h under N₂. The reaction mixture was diluted with H₂O (20 mL) and extracted with DCM (20 mL×3), dried over Na₂SO₄, filtered, and concentrated under reduced pressure. The residue was purified by prep-HPLC to give the title compound. [M+H]=388.9. ¹H NMR (400 MHz, DMSO-d₆) δ ppm: 10.34 (s, 1H), 8.14 (s, 1H), 7.38 (d, J=7.6 Hz, 1H), 7.24 (s, 1H), 7.12 (d, J=7.6 Hz, 1H), 5.54 (s, 2H), 3.93 (s, 3H), 2.49-2.45 (m, 2H), 1.34 (s, 9H), 1.17 (t, J=7.2 Hz, 3H).

The following compounds were prepared following procedures analogous to that described for Example 331.

| Ex. | | | |
|---|---|---|---|
| No. | Structure | Name | LCMS, ¹H NMR |
| 332 | 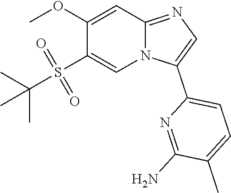 [SMILES: COc1cc2ncc(-c3ccc(C)c(N)n3)n2cc1S(=O)(=O)C(C)(C)C] | 6-(6-(tert- butylsulfonyl)-7- methoxyimidazo[1,2- a]pyridin-3-yl)-3- methylpyridin-2-amine | [M + H]⁺ = 375.0 ¹H NMR (400 MHz, DMSO-d₆) δ ppm 10.33 (s, 1H), 8.14 (s, 1H), 7.38 (d, J = 7.6 Hz, 1H), 7.24 (s, 1H), 7.08 (d, J = 7.2 Hz, 1H), 5.53 (s, 2H), 3.93 (s, 3H), 2.12 (s, 3H), 1.34 (s, 9H). |
| 333 | 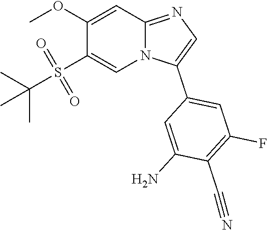 [SMILES: COc1cc2ncc(-c3cc(N)c(C#N)c(F)c3)n2cc1S(=O)(=O)C(C)(C)C] | 2-amino-4-(6-(tert- butylsulfonyl)-7- methoxyimidazo[1,2- a]pyridin-3-yl)-6- fluorobenzonitrile | [M + H]⁺ = 403.0 ¹H NMR (400 MHz, DMSO-d₆) δ ppm 8.74 (s, 1H), 7.85 (s, 1H), 7.30 (s, 1H), 6.87-6.68 (m, 4H), 3.94 (s, 3H), 1.32 (s, 9H). |

### Example 334

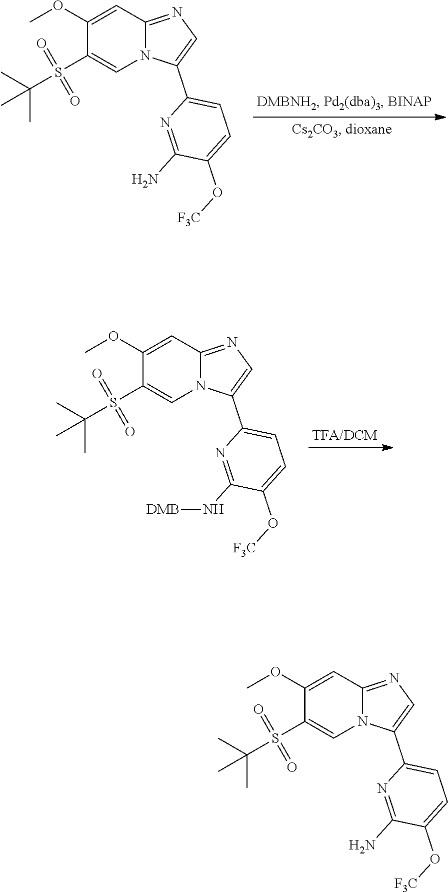

6-(6-(tert-butylsulfonyl)-7-methoxyimidazo[1,2-a]pyridin-3-yl)-N-(2,4-dimethoxybenzyl)-3-(trifluoromethoxy)pyridin-2-amine: A mixture of 6-(tert-butylsulfonyl)-3-(6-chloro-5-(trifluoromethoxy)pyridin-2-yl)-7-methoxyimidazo[1,2-a]pyridine (55 mg, 107 μmol, 1 eq), DMBNH₂ (178.43 mg, 1.07 mmol, 160.75 μL, 10 eq), Pd₂(dba)₃ (9.77 mg, 10.67 μmol, 0.1 eq), BINAP (13.3 mg, 21.3 mol, 0.2 eq) and Cs₂CO₃ (104 mg, 320 μmol, 3 eq) in dioxane (6 mL) was degassed and purged with N₂ three times, and then the mixture was stirred at 100° C. for 6 h under N₂. The reaction mixture was diluted with H₂O (20 mL) and extracted with EtOAc (20 mL×3). The combined organic layers were dried over Na₂SO₄, filtered, and concentrated under reduced pressure. The residue was purified by prep-TLC (DCM/MeOH=15/1) to give the title compound. ¹H NMR (400 MHz, DMSO-d₆) δ ppm: 10.40 (s, 1H), 8.32 (s, 1H), 7.62 (dd, J=1.6, 8.4 Hz, 1H), 7.29 (s, 1H), 7.17 (d, J=8.4 Hz, 1H), 7.10 (d, J=8.4 Hz, 1H), 6.83 (t, J=5.6 Hz, 1H), 6.50 (d, J=2.4 Hz, 1H), 6.42 (dd, J=2.4, 8.0 Hz, 1H), 4.64 (d, J=5.2 Hz, 2H), 3.93 (s, 3H), 3.73 (s, 3H), 3.71 (s, 3H), 1.21 (s, 9H).

6-(6-(tert-butylsulfonyl)-7-methoxyimidazo[1,2-a]pyridin-3-yl)-3-(trifluoromethoxy)pyridin-2-amine: To a solution of 6-(6-(tert-butylsulfonyl)-7-methoxyimidazo[1,2-a]pyridin-3-yl)-N-(2,4-dimethoxybenzyl)-3-(trifluoromethoxy)pyridin-2-amine (55 mg, 83 μmol, 1 eq) in DCM (3 mL) was added TFA (1.54 g, 13.5 mmol, 1 mL). The mixture was stirred at 20° C. for 2 h. The mixture was evaporated on a water bath under reduced pressure. The residue was dissolved in MeOH (1.5 mL), and the pH was adjusted to around 8 by progressively adding NH₃·H₂O at 0° C. The crude product was purified by prep-HPLC to give the title compound. [M+H]⁺=444.9. ¹H NMR (400 MHz, DMSO-d₆) δ ppm: 10.16 (s, 1H), 8.25 (s, 1H), 7.63 (dd, J=1.2, 8.4 Hz, 1H), 7.29 (s, 1H), 7.17 (d, J=8.4 Hz, 1H), 6.28 (s, 2H), 3.94 (s, 3H), 1.35 (s, 9H).

The following compounds were prepared following procedures analogous to that described for Example 334.

| Ex. | | | |
|---|---|---|---|
| No. | Structure | Name | LCMS, ¹H NMR |
| 335 | 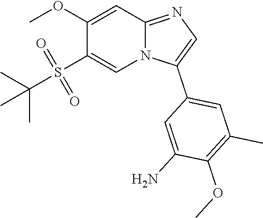 [SMILES: COc1cc2ncc(-c3cc(C)c(OC)c(N)c3)n2cc1S(=O)(=O)C(C)(C)C] | 6-(6-(tert- butylsulfonyl)-7- methoxyimidazo[1,2- a]pyridin-3-yl)-3- methoxy-4- methylpyridin-2-amine | [M + H]⁺ = 404.9 ¹H NMR (400 MHz, DMSO-d₆) δ ppm 10.22 (s, 1H), 8.08 (s, 1H), 7.23 (s, 1H), 7.02 (s, 1H), 5.64 (s, 2H), 3.93 (s, 3H), 3.68 (s, 3H), 2.22 (s, 3H), 1.34 (s, 9H). |
| 336 | 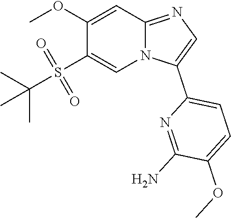 [SMILES: COc1cc2ncc(-c3ccc(OC)c(N)n3)n2cc1S(=O)(=O)C(C)(C)C] | 6-(6-(tert- butylsulfonyl)-7- methoxyimidazo[1,2- a]pyridin-3-yl)-3- methoxypyridin-2- amine | [M + H]⁺ = 391.0 ¹H NMR (400 MHz, DMSO-d₆) δ ppm 10.14 (s, 1H), 8.02 (s, 1H), 7.24-7.17 (m, 2H), 7.10 (d, J = 8.0 Hz, 1H), 5.62 (s, 2H), 3.92 (s, 3H), 3.84 (s, 3H), 1.33 (s, 9H). |

### Example 337

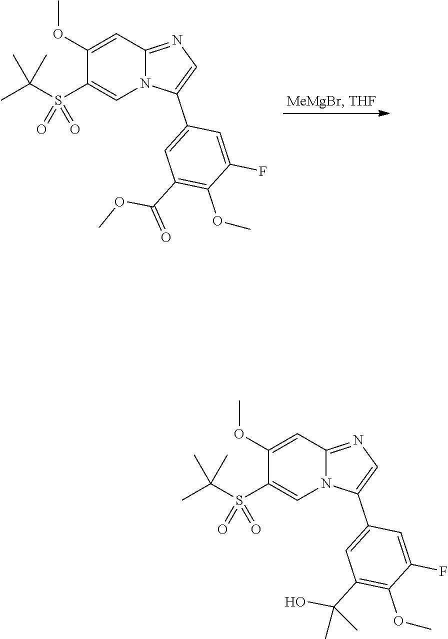 [SMILES: COC(=O)c1cc(-c2cnc3cc(OC)c(S(=O)(=O)C(C)(C)C)cn23)cc(F)c1OC ; COc1cc2ncc(-c3cc(F)c(OC)c(C(C)(C)O)c3)n2cc1S(=O)(=O)C(C)(C)C]

2-(5-(6-(tert-butylsulfonyl)-7-methoxyimidazo[1,2-a]pyridin-3-yl)-3-fluoro-2-methoxyphenyl)propan-2-ol: To a solution of methyl 5-(6-(tert-butylsulfonyl)-7-methoxyimidazo[1,2-a]pyridin-3-yl)-3-fluoro-2-methoxybenzoate (20 mg, 40 mol, 1 eq) in THF (5 mL) was added methylmagnesiumbromide (3 M, 133 μL, 10 eq) dropwise at 0° C. The mixture was stirred at 25° C. for 12 h. The reaction mixture was quenched by addition saturated aqueous NH₄Cl (10 mL) at 0° C., and then diluted with H₂O (20 mL) and extracted with EtOAc (20 mL×3). The combined organic layers were dried over Na₂SO₄, filtered, and concentrated under reduced pressure to give a residue, which was purified by prep-HPLC to give the title compound. [M+H]⁺=451.1. ¹H NMR (400 MHz, DMSO-d₆) δ ppm: 8.72 (s, 1H), 7.73 (s, 1H), 7.64 (s, 1H), 7.68 (dd, J=2.0, 12.4 Hz, 1H), 7.27 (s, 1H), 5.21 (s, 1H), 3.94 (s, 6H), 1.53 (s, 6H), 1.32 (s, 9H).

### Example 338

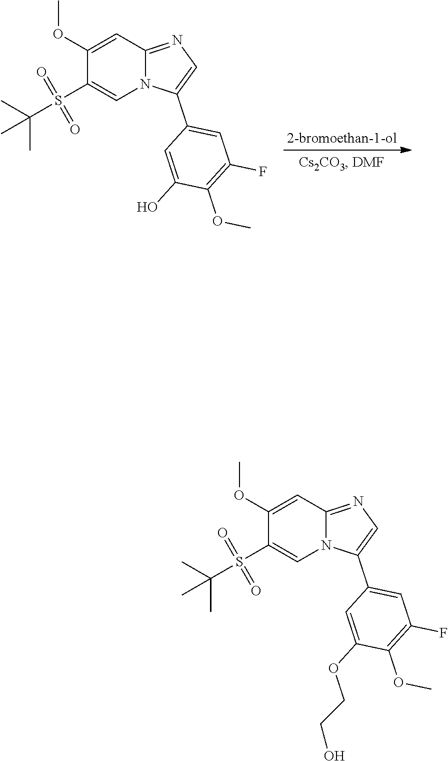 [SMILES: COc1cc2ncc(-c3cc(O)c(OC)c(F)c3)n2cc1S(=O)(=O)C(C)(C)C ; COc1cc2ncc(-c3cc(F)c(OC)c(OCCO)c3)n2cc1S(=O)(=O)C(C)(C)C]

2-(5-(6-(tert-butylsulfonyl)-7-methoxyimidazo[1,2-a]pyridin-3-yl)-3-fluoro-2-methoxyphenoxy)ethan-1-ol: A mixture of 5-(6-(tert-butylsulfonyl)-7-methoxyimidazo[1,2-a]pyridin-3-yl)-3-fluoro-2-methoxyphenol (20 mg, 44.1 μmol, 1 eq) and 2-bromoethanol (13.8 mg, 110 mol, 7.81 μL, 2.5 eq) in DMF (1 mL) was added Cs₂CO₃ (43.1 mg, 132 μmol, 3 eq). The mixture was stirred at 80° C. for 12 h. The reaction mixture was filtered and concentrated under reduced pressure to give a residue, which was purified by prep-HPLC to give the title compound. [M+H]⁺=452.9. ¹H NMR (400 MHz, DMSO-d₆) δ ppm: 8.72 (s, 1H), 7.77 (s, 1H), 7.27 (s, 1H), 7.19-7.12 (m, 2H), 4.94 (t, J=5.6 Hz, 1H), 4.09 (t, J=4.8 Hz, 2H), 3.93 (s, 3H), 3.89 (s, 3H), 3.78-3.74 (m, 2H), 1.32 (s, 9H).

The following compound was prepared following procedures analogous to that described for Example 338.

| Ex. | | | |
|---|---|---|---|
| No. | Structure | Name | LCMS, ¹H NMR |
| 339 | 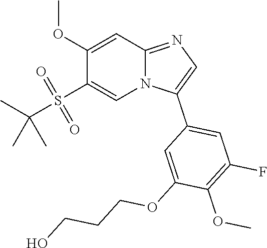 [SMILES: COc1cc2ncc(-c3cc(F)c(OC)c(OCCCO)c3)n2cc1S(=O)(=O)C(C)(C)C] | 3-(5-(6-(tert- butylsulfonyl)-7- methoxyimidazo[1,2- a]pyridin-3-yl)-3- fluoro-2- methoxyphenoxy) propan-1-ol | [M + H]⁻ = 467.1 ¹H NMR (400 MHz, DMSO-d₆) δ ppm: 8.71 (s, 1H), 7.77 (s, 1H), 7.26 (s, 1H), 7.21-7.09 (m, 2H), 4.57 (t, J = 5.2 Hz, 1H), 4.14 (t, J = 6.4 Hz, 2H), 3.93 (s, 3H), 3.87 (s, 3H), 3.61-3.24 (m, 2H), 1.96-1.86 (m, 2H), 1.32 (s, 9H). |

### Example 340

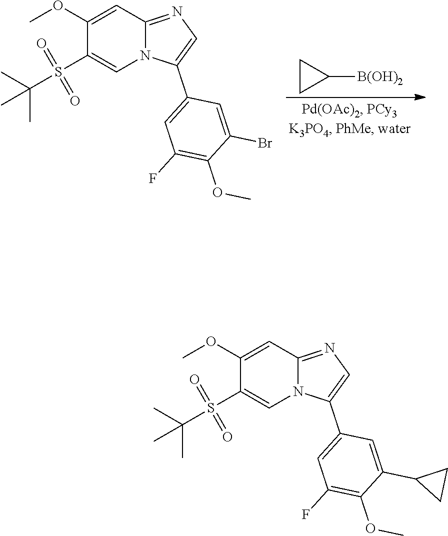 [SMILES: COc1cc2ncc(-c3cc(F)c(OC)c(Br)c3)n2cc1S(=O)(=O)C(C)(C)C ; OB(O)C1CC1 ; COc1cc2ncc(-c3cc(F)c(OC)c(C4CC4)c3)n2cc1S(=O)(=O)C(C)(C)C]

A mixture of 3-(3-bromo-5-fluoro-4-methoxyphenyl)-6-(tert-butylsulfonyl)-7-methoxyimidazo[1,2-a]pyridine (50.00 mg, 106.08 μmol, 1 eq), cyclopropylboronic acid (27.34 mg, 318.24 mol, 3 eq), PCy3 (5.95 mg, 21.22 μmol, 6.88 μL, 0.2 eq), Pd(OAc)₂ (4.76 mg, 21.22 mol, 0.2 eq) and K₃PO₄ (78.81 mg, 371.28 mol, 3.5 eq) in toluene (2 mL) and H₂O (0.5 mL) was degassed and purged with N₂ (3×). The mixture was stirred at 100° C. for 3 h under N₂ atmosphere. The reaction mixture was concentrated under reduced pressure to give a residue, which was purified by prep RP-HPLC (acidic conditions) to yield the title compound. [M+H]=433.1. ¹H NMR (400 MHz, DMSO-d₆) δ ppm: 8.64 (s, 1H), 7.74 (s, 1H), 7.34 (dd, J=1.6, 12.0 Hz, 1H), 7.25 (s, 1H), 6.91 (s, 1H), 3.93 (s, 6H), 2.29-2.18 (m, 1H), 1.32 (s, 9H), 1.08-0.95 (m, 2H), 0.85-0.70 (m, 2H).

Example 341 (N-(5-(6-(ethylsulfonyl)-7-methoxyimidazo[1,2-a]pyridin-3-yl)-3-fluoro-2-methoxyphenyl)propane-1-sulfonamide) and Example 342 (N-(3-fluoro-2-methoxy-5-(7-methoxy-6-(methylsulfonyl)imidazo[1,2-a]pyridin-3-yl)phenyl)propane-1-sulfonamide) can be prepared following procedures analogous to that described herein.

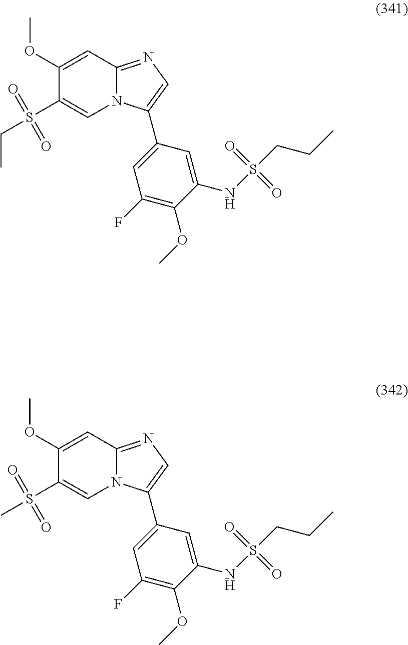

### Biological Data

### RIPK2 Binding Competition Assay

The ability of selected compounds of Formula (I) to inhibit the binding of an Alexa647-labelled ATP-competitive kinase inhibitor to a GST-RIPK2 fusion protein was quantified employing the TR-FRET-based RIPK2 binding competition assay as described in the following paragraphs.

Recombinant fusion protein of N-terminal Glutathione-S-Transferase (GST) and a fragment of human RTPK2 (amino acids 1-310 of accession number 043353), expressed in baculovirus infected Sf9 cells, purified via glutathione affinity chromatography, was used as GST-RIPK2 fusion protein. Tracer 199 from Life Technologies (catalogue no. PR9115B) was used as Alexa647-labelled ATP-competitive kinase inhibitor.

For the assay, 50 nL of a 100-fold concentrated solution of each test compound in DMSO was pipetted into either a black low volume 384-well microtiter plate or a black 1536-well microtiter plate (both Greiner Bio-One, Frickenhausen, Germany), 3 L solution of Tracer 199 (16.7 nM=>final concentration in 5 μL assay volume is 10 nM) in aqueous assay buffer (25 mM Tris/HCl pH 7.5, 10 mM magnesium chloride (MgCl₂), 5 mM β-glycerophosphate, 2.5 mM dithiothreitol (DTT), 0.5 mM ethylene glycol-bis(2-aminoethylether)-N,N,N′,N′-tetraacetic acid (EGTA), 0.5 mM sodium ortho-vanadate, 0.01% (w/v) bovine serum albumin (BSA), 0.005% (w/v) Pluronic F-127 (Sigma)) were added. Then the binding competition was started by the addition of 2 μL of a solution of the GST-RIPK2 fusion protein (2.5 nM=>final conc. in the 5 μL assay volume is 1 nM) and of Anti-GST-Tb (1.25 nM=>final conc. in the 5 μL assay volume is 0.5 nM), a Lumi4®-Tb cryptate-conjugated anti-GST antibody from PerkinElmer (catalogue no 61GSTTAH), in assay buffer.

The resulting mixture was incubated 45 min at 22° C. to allow the formation of a complex between the Tracer 199, the fusion protein and Anti-GST-Tb. Subsequently, the amount of this complex was evaluated by measurement of the resonance energy transfer from the Tb-cryptate to the Tracer 199. Therefore, the fluorescence emissions at 620 nm and 665 nm after excitation at 337 nm were measured in a TR-FRET reader, e.g. a Pherastar FS (BMG Labtechnologies, Offenburg, Germany) or a Viewlux (PerkinElmer). The ratio of the emissions at 665 nm and at 620 nm was taken as the measure of the amount of the complex. The data were normalized (assay reaction without inhibitor=0% activity, all other assay components but no GST-RIPK2 fusion protein=−100% activity). Typically the test compounds were tested on the same microtiter plate in 11 different concentrations in the range of 20 μM to 0.07 nM (20 M, 5.7 M, 1.6 M, 0.47 μM, 0.13 μM, 38 nM, 11 nM, 3.1 nM, 0.9 nM, 0.25 nM and 0.07 nM, is the dilution series prepared separately before the assay on the level of the 100-fold concentrated solutions in DMSO by serial dilutions; exact concentrations may vary depending on pipettors used) in duplicate values for each concentration and IC₅₀ values were calculated using Genedata Screener™ software.

### THP-1 Dual Cell SEAP and Viability Assays

THP1-Dual™ cells (InvivoGen Cat #thpd-nfis) allow simultaneous assessment of the NF-κB pathway, by monitoring the activity of secreted embryonic alkaline phosphatase (SEAP) as well as the TRF pathway, by assessing the activity of a secreted luciferase (Lucia). Cells were cultured according to manufacturers' recommendations. For the assay, cell concentration was adjusted to 7.14×10⁵ cells/mL in assay media (RPMI 1640, 2 mM L-Glutamine, 25 mM HEPES, 10% fetal bovine serum (heat-inactivated for 30 min at 56° C.), Pen-Strep (100 U/mL)) and 35 μL of cell suspension (˜25,000 cells) per well were added to a flat bottom 384-well plate (white opaque). Plate was centrifuged at 300 g for 2 min, compounds were added in serial dilution series according to the plate layout (volumes ˜500 nL-0.0152 nL) and plate was incubated at 37° C. in 5% C02 for 30 min. Using assay media, a L18-MDP stock solution, that results in a final concentration of 10 ng/mL L18-MDP in each well when adding 15 μL to each well, was prepared. 15 μL of L18-MDP stock solution was added to each well, while 15 μL of assay media was added to the negative control wells. Plates were subsequently incubated at 37° C. in 5% C02 overnight (20-24 h).

For the SEAP assay: without disturbing the cells at the bottom of the well, 15 μL supernatant was transferred to a clear 384-well flat bottom plate and 35 μL QUANTI-Blue Solution per well was added. Following a 2 h incubation at 37° C., the optical density (OD) was measured at 620-655 nm using a microplate reader (PheraStarFS microplate reader with protocol for OD 620-655 nm).

For the viability assay: the cells remaining in the plate were used to assess cell viability. For this, CellTiter-Glo buffer and lyophilized CellTiter-Glo substrate was allowed to equilibrate to room temperature and lyophilized CellTiter-Glo substrate was reconstituted according to the manufacturers' recommendations. The equal volume (here 35 μL) of CTG was added to each well, contents were mixed on an orbital shaker for 2 min to induce lysis followed by a 10 min incubation at room temperature. Luminescence signal was subsequently measured in a microplate reader (PheraStar).

### Analysis:

Values of media-only wells were subtracted and % inhibition for each compound concentration relative to the DMSO/L18-MDP-treated controls was calculated. Inhibition values+/−SD were fitted by non-linear regression using Prism software (GraphPad PRISM Software) to calculate IC₅₀ values.

Table A below lists IC₅₀ values of selected compounds of Formula (I) measured in the RIPK2 binding competition assay and the THIP-1 Dual cell SEAP and viability assays. For the RIPK2 competition assay IC₅₀ values, “A” denotes an IC₅₀ of <1 nM; “B” denotes an IC₅₀ of 1 nM≤B≤2.5; “C” denotes an IC₅₀ of 2.5 nM≤C≤20; and “D” denotes an IC₅₀ of 20 nM<D. For the THP assay IC₅₀ values, “A” denotes an IC₅₀ of <50 nM; “B” denotes an IC₅₀ of 50 nM≤B≤250; “C” denotes an IC₅₀ of 250 nM≤C≤1,000; and “D” denotes an IC₅₀ of 1,000 nM<D.

TABLE A

| | RIPK2 IC₅₀ | NFkB THP1 |
|---|---|---|
| Ex. No | (nM) | IC₅₀ (nM) |
| 1 | D | D |
| 2 | C | D |
| 3 | D | D |
| 4 | C | C |
| 5 | C | |
| 6 | C | D |
| 7 | C | C |
| 8 | D | D |
| 9 | D | D |
| 10 | A | A |
| 11 | C | C |
| 12 | C | |
| 13 | D | D |
| 14 | D | D |
| 15 | D | D |
| 16 | D | D |
| 17 | D | D |
| 18 | D | D |
| 19 | D | D |
| 20 | C | C |
| 21 | D | D |
| 22 | C | D |
| 23 | D | D |
| 24 | D | D |
| 25 | D | D |
| 26 | D | D |
| 27 | C | D |
| 28 | C | D |
| 29 | C | D |
| 30 | D | D |
| 31 | D | D |
| 32 | D | D |
| 33 | D | |
| 34 | D | D |
| 35 | D | D |
| 36 | D | D |
| 37 | D | D |
| 38 | B | B |
| 39 | C | B |
| 40 | C | C |
| 41 | C | B |
| 42 | C | C |
| 43 | C | B |
| 44 | C | B |
| 45 | D | D |
| 46 | C | B |
| 47 | C | D |
| 48 | D | D |
| 49 | D | C |
| 50 | D | D |
| 51 | D | D |
| 52 | C | C |
| 53 | C | B |
| 54 | D | D |
| 55 | D | D |
| 56 | D | D |
| 57 | D | D |
| 58 | C | D |
| 59 | C | C |
| 60 | C | D |
| 61 | D | C |
| 62 | D | D |
| 63 | C | |
| 64 | C | B |
| 65 | B | B |
| 66 | D | D |
| 67 | D | D |
| 68 | D | D |
| 69 | D | D |
| 70 | C | |
| 71 | D | D |
| 72 | D | D |
| 73 | D | D |
| 74 | C | D |
| 75 | C | B |
| 76 | D | D |
| 77 | D | D |
| 78 | D | D |
| 79 | D | D |
| 80 | C | C |
| 81 | C | C |
| 82 | C | C |
| 83 | D | D |
| 84 | D | D |
| 85 | C | C |
| 86 | C | D |
| 87 | C | D |
| 88 | D | D |
| 89 | C | D |
| 90 | C | C |
| 91 | C | D |
| 92 | D | D |
| 93 | D | D |
| 94 | D | D |
| 95 | C | C |
| 96 | C | C |
| 97 | C | C |
| 98 | D | D |
| 99 | D | D |
| 100 | C | C |
| 101 | D | D |
| 102 | C | C |
| 103 | D | D |
| 104 | C | D |
| 105 | C | B |
| 106 | C | C |
| 107 | C | D |
| 108 | D | D |
| 109 | D | D |
| 110 | C | D |
| 111 | B | B |
| 112 | C | D |
| 113 | D | D |
| 114 | B | B |
| 115 | C | C |
| 116 | C | D |
| 117 | D | D |
| 118 | C | C |
| 119 | C | D |
| 120 | D | D |
| 121 | D | D |
| 122 | D | D |
| 123 | D | D |
| 124 | C | D |
| 125 | C | B |
| 126 | D | D |
| 127 | D | D |
| 128 | D | D |
| 129 | D | D |
| 130 | B | C |
| 131 | A | A |
| 132 | A | B |
| 133 | C | C |
| 134 | C | C |
| 135 | C | D |
| 136 | C | D |
| 137 | C | C |
| 138 | D | D |
| 139 | B | B |
| 140 | A | A |
| 141 | B | B |
| 142 | C | C |
| 143 | C | D |
| 144 | D | D |
| 145 | B | C |
| 146 | B | B |
| 147 | D | D |
| 148 | D | D |
| 149 | C | C |
| 150 | D | D |
| 151 | D | D |
| 152 | D | D |
| 153 | D | D |
| 154 | C | B |
| 155 | D | D |
| 156 | D | D |
| 157 | C | B |
| 158 | D | D |
| 159 | D | |
| 160 | D | D |
| 161 | C | C |
| 162 | D | D |
| 163 | D | D |
| 164 | D | D |
| 165 | C | C |
| 166 | D | D |
| 167 | D | D |
| 168 | B | C |
| 169 | A | A |
| 170 | A | A |
| 171 | B | A |
| 172 | B | |
| 173 | C | |
| 174 | C | B |
| 175 | A | A |
| 176 | A | A |
| 177 | A | |
| 178 | A | A |
| 179 | A | A |
| 180 | A | B |
| 181 | A | A |
| 182 | A | A |
| 183 | A | A |
| 184 | A | B |
| 185 | A | A |
| 186 | A | A |
| 187 | A | |
| 188 | A | A |
| 189 | A | |
| 190 | A | |
| 191 | A | A |
| 192 | A | A |
| 193 | A | A |
| 194 | A | A |
| 195 | A | A |
| 196 | B | A |
| 197 | B | A |
| 198 | A | A |
| 199 | B | B |
| 200 | B | A |
| 201 | B | A |
| 202 | B | A |
| 203 | B | B |
| 204 | B | B |
| 205 | B | B |
| 206 | B | A |
| 207 | B | A |
| 208 | B | A |
| 209 | B | B |
| 210 | B | B |
| 211 | C | C |
| 212 | C | D |
| 213 | C | C |
| 214 | C | B |
| 215 | A | A |
| 216 | A | B |
| 217 | C | |
| 218 | C | |
| 219 | B | |
| 220 | B | |
| 221 | B | A |
| 222 | B | A |
| 223 | C | B |
| 224 | B | A |
| 225 | C | C |
| 226 | B | A |
| 227 | B | A |
| 228 | A | A |
| 229 | A | A |
| 230 | C | C |
| 231 | A | A |
| 232 | C | C |
| 233 | A | A |
| 234 | C | B |
| 235 | B | A |
| 236 | A | A |
| 237 | C | B |
| 238 | B | B |
| 239 | A | A |
| 240 | A | A |
| 241 | A | A |
| 242 | A | A |
| 243 | A | A |
| 244 | C | C |
| 245 | C | C |
| 246 | A | A |
| 247 | C | A |
| 248 | C | B |
| 249 | D | C |
| 250 | C | B |
| 251 | B | A |
| 252 | D | C |
| 253 | D | D |
| 254 | D | D |
| 255 | D | D |
| 256 | D | D |
| 257 | D | D |
| 258 | D | D |
| 259 | C | C |
| 260 | C | D |
| 261 | C | D |
| 262 | C | C |
| 263 | C | C |
| 264 | C | C |
| 265 | C | B |
| 266 | B | C |
| 267 | B | C |
| 268 | B | B |
| 269 | B | C |
| 270 | B | C |
| 271 | B | A |
| 272 | B | B |
| 273 | B | B |
| 274 | A | B |
| 275 | A | A |
| 276 | C | C |
| 277 | C | A |
| 278 | C | C |
| 279 | A | A |
| 280 | B | C |
| 281 | C | C |
| 282 | C | D |
| 283 | C | C |
| 284 | B | B |
| 285 | A | A |
| 286 | C | D |
| 287 | D | D |
| 288 | C | D |
| 289 | C | C |
| 290 | C | D |
| 291 | C | C |
| 292 | C | D |
| 293 | D | D |
| 294 | A | A |
| 295 | B | B |
| 296 | B | A |
| 297 | D | |
| 298 | C | B |
| 299 | D | D |
| 300 | D | D |
| 301 | B | B |
| 302 | C | C |
| 303 | D | |
| 304 | D | D |
| 305 | C | D |
| 306 | C | B |
| 307 | D | D |
| 308 | B | B |
| 309 | D | D |
| 310 | D | D |
| 311 | C | C |
| 312 | C | C |
| 313 | D | D |
| 314 | D | D |
| 315 | D | D |
| 316 | C | C |
| 317 | A | A |
| 318 | B | |
| 319 | B | B |
| 320 | D | D |
| 321 | D | D |
| 322 | C | D |
| 323 | D | D |
| 324 | D | D |
| 325 | D | D |
| 326 | B | B |
| 327 | B | A |
| 328 | D | D |
| 329 | C | C |
| 330 | C | C |
| 331 | D | D |
| 332 | D | D |
| 333 | C | C |
| 334 | D | D |
| 335 | D | D |
| 336 | D | D |
| 337 | B | C |
| 338 | C | D |
| 339 | C | D |
| 340 | B | B |

What is claimed is:

1. A compound of Formula (I): 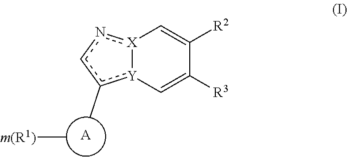
or a pharmaceutically acceptable salt thereof, wherein:
Ring A is phenyl, 5-10 membered heteroaryl, or 5-10 membered heterocyclyl;
one of X and Y is N and the other of X and Y is C;
each [symbol] is a single bond or a double bond, such that the bicyclic ring system of Formula (I) is imidazo[1,2-a]pyridine or pyrazolo[1,5-a]pyridine;
m is 0, 1, 2, 3, or 4;
each R¹ is independently selected from the group consisting of:
(i) halogen,
(ii) cyano,
(iii) C1-C6 alkyl optionally substituted with hydroxyl, C1-C6 alkoxy, or —S(O₂)C1-C6 alkyl,
(iv) C1-C6 haloalkyl,
(v) C1-C6 haloalkoxy,
(vi) C3-C6 cycloalkyl,
(vii) —NR^{A}R^{B},
(viii) C1-C6 alkoxy optionally substituted with hydroxyl or phenyl,
(ix) 4-8 membered heterocyclyl optionally substituted with 1-2 substituents independently selected from the group consisting of hydroxyl, C1-C6 alkyl, and C1-C6 haloalkyl,
(x) —S(O₂)C1-C6 alkyl,
(xi) hydroxyl,
(xii) nitro,
(xiii) —S(O₂)C3-C6 cycloalkyl, and
(xiv) when Ring A is phenyl, R¹ is further selected from —S(O₂)NR^{A}R^{A};
R^{A} is hydrogen or C1-C6 alkyl;
R^{B} is
(i) hydrogen,
(ii) —S(O₂)C1-C6 alkyl,
(iii) C3-C6 cycloalkyl optionally substituted with hydroxyl or C1-C6 alkoxy,
(iv) 4-8 membered heterocyclyl optionally substituted with 1-2 substituents independently selected from hydroxyl and C1-C6 haloalkyl, or
(v) C1-C6 alkyl optionally substituted with 1-4 substituents independently selected from:
(a) halogen,
(b) hydroxyl,
(c) —NR^{C}R^{D},
(d) C1-C6 alkoxy,
(e) C1-C6 haloalkoxy,
(f) C3-C6 cycloalkyl optionally substituted with hydroxyl,
(g) phenyl optionally substituted with C1-C6 alkoxy,
(h) 5-6 membered heteroaryl optionally substituted with C1-C6 alkyl,
(j) 4-8 membered heterocyclyl optionally substituted with hydroxyl, —C(═O)C1-C6 alkyl, or C1-C6 alkyl,
(k) C1-C6 thioalkyl, and
(l) —S(═NR)(═O)C1-C6 alkyl;
R² is hydrogen, halogen, C1-C6 alkoxy, or C1-C6 haloalkoxy;
R³ is
(i) C1-C6 thioalkyl, 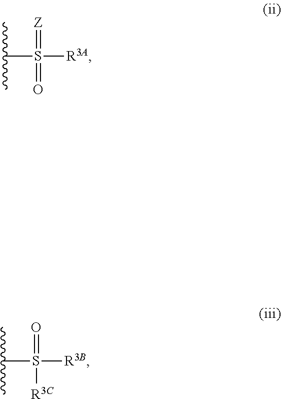
(iv) 4-8 membered heterocyclyl optionally substituted with 1-2 substituents independently selected from halogen and C1-C6 alkyl,
(v) C1-C6 alkyl optionally substituted with NR^{E}R^{F} or hydroxyl,
(vi) —CO₂H,
(vii) —C(═O)NR^{E}R^{F},
(viii) C1-C6 alkoxy optionally substituted with 4-10 membered heterocyclyl optionally substituted with C1-C6 alkoxy,
(ix) hydrogen;
Z is O or NR⁴;
R^{3A} is C1-C6 haloalkyl, C3-C6 cycloalkyl, or C1-C6 alkyl optionally substituted with C3-C6 cycloalkyl;
R^{3B} and R^{3C} are each independently C3-C6 cycloalkyl or C1-C6 alkyl optionally substituted with C3-C6 cycloalkyl;
R⁴ is hydrogen or C1-C6 alkyl; and
each R^{C} and R^{D} are each independently hydrogen, —C(═O)OC1-C6 alkyl, or C1-C6 alkyl optionally substituted with oxo;
each R^{E} and R^{F} are each independently hydrogen or C1-C6 alkyl; and
wherein
(i) when X is C, Y is N, Ring A is 4-pyridyl, R² is C1-C6 alkoxy, R³ is —S(O₂)—C1-C6 alkyl, and m is 1, then R¹ is not —NH₂ or —NH(C═O)CH₃;
(ii) when X is C, Y is N, Ring A is 4-pyridyl, R² is C1-C6 alkoxy, R³ is —S(O₂)—C1-C6 alkyl, m is 2, and one R¹ is fluoro, —NH-(p-methoxybenzyl), or —NH₂, then the other R¹ is not halogen;
(iii) when X is C, Y is N, Ring A is phenyl, R² is C1-C6 alkoxy, R³ is —S(O₂)—C1-C6 alkyl, and m is 1, R¹ is not fluoro, cyano, or unsubstituted C1-C6 alkoxy;
(iv) when X is C, Y is N, Ring A is phenyl, R² is C1-C6 alkoxy, R³ is —S(O₂)—C1-C6 alkyl, m is 2, and one R¹ is halogen, then the other R¹ is not unsubstituted C1-C6 alkoxy;
(v) when X is C, Y is N, Ring A is 4-pyridyl, R² is C1-C6 haloalkoxy, R³ is —S(O₂)—C1-C6 alkyl, m is 2, and one R¹ is —NH₂, then the other R¹ is not halogen;
(vi) when X is C, Y is N, R² is C1-C3 alkoxy, R³ is —S(O₂)—C1-C4 alkyl, Ring A is phenyl, and m is 1, R¹ is not halogen, —NH₂, cyano, or unsubstituted C1-C6 alkoxy;
(vii) when X is C, Y is N, R² is ethoxy, R³ is —S(O₂)—C1-C4 alkyl, and m is 2, and one R¹ is halogen, then the other R¹ is not —NH₂ or unsubstituted alkoxy;
(viii) when X is C, Y is N, R² is C1-C2 haloalkoxy, R³ is —S(O₂)—C1-C4 alkyl, and m is 2, and one R¹ is halogen, then the other R¹ is not —NH₂; and
(ix) when X is C, Y is N, R² is C1-C3 alkoxy, R³ is —S(O₂)—C1-C4 alkyl, and m is 0, then Ring A is not 1H-indazole or 1H-benzo[d]imidazole.

2. The compound of claim 1, wherein X is C, Y is N, and the bicyclic ring system of Formula (I) is imidazo[1,2-a]pyridine.

3. The compound of claim 1, wherein X is N, Y is C, and the bicyclic ring system of Formula (I) is pyrazolo[1,5-a]pyridine.

4. The compound of any one of claims 1-3, wherein Ring A is 5-6 membered heteroaryl.

5. The compound of any one of claims 1-3, wherein Ring A is 5-6 membered heterocyclyl.

6. The compound of any one of claims 1-3, wherein Ring A is phenyl.

7. The compound of any one of claims 1-7, wherein 1-3 R¹ are independently halogen.

8. The compound of any one of claims 1-7, wherein one R¹ is cyano.

9. The compound of any one of claims 1-8, wherein one R¹ is C1-C6 alkyl optionally substituted with hydroxyl, C1-C6 alkoxy, or —S(O₂)C1-C6 alkyl.

10. The compound of any one of claims 1-9, wherein one R¹ is C1-C6 haloalkyl.

11. The compound of any one of claims 1-10, wherein one R¹ is C1-C6 haloalkoxy.

12. The compound of any one of claims 1-11, wherein one R¹ is C3-C6 cycloalkyl.

13. The compound of any one of claims 1-12, wherein one R¹ is C1-C6 alkoxy optionally substituted with hydroxyl or phenyl.

14. The compound of any one of claims 1-13, wherein one R¹ is 4-8 membered heterocyclyl optionally substituted with 1-2 substituents independently selected from the group consisting of hydroxyl, C1-C6 alkyl, and C1-C6 haloalkyl.

15. The compound of any one of claims 1-14, wherein one R¹ is-S(O₂)C1-C6 alkyl.

16. The compound of any one of claims 1-15, wherein one R¹ is hydroxyl.

17. The compound of any one of claims 1-16, wherein one R¹ is —NR^{A}R^{B}.

18. The compound of any one of claims 1-17, wherein one R¹ is nitro,

19. The compound of any one of claims 1-18, wherein one R¹ is —S(O₂)C3-C6 cycloalkyl.

20. The compound of any one of claims 1-19, wherein R^{A} is hydrogen.

21. The compound of any one of claims 1-19, wherein R^{A} is C1-C6 alkyl.

22. The compound of any one of claims 1-21, wherein R^{B} is hydrogen.

23. The compound of any one of claims 1-22, wherein R^{B} is —S(O₂)C1-C6 alkyl.

24. The compound of any one of claims 1-22, wherein R^{B} is C3-C6 cycloalkyl optionally substituted with hydroxyl or C1-C6 alkoxy.

25. The compound of any one of claims 1-22, wherein R^{B} is 4-8 membered heterocyclyl optionally substituted with 1-2 substituents independently selected from hydroxyl and C1-C6 haloalkyl.

26. The compound of any one of claims 1-22, wherein R^{B} is C1-C6 alkyl optionally substituted with 1-4 substituents independently selected from:
(a) halogen,
(b) hydroxyl,
(c) —NR^{C}R^{D},
(d) C1-C6 alkoxy,
(e) C1-C6 haloalkoxy,
(f) C3-C6 cycloalkyl optionally substituted with hydroxyl,
(g) phenyl optionally substituted with C1-C6 alkoxy,
(h) 5-6 membered heteroaryl optionally substituted with C1-C6 alkyl,
(j) 4-8 membered heterocyclyl optionally substituted with hydroxyl, —C(═O)C1-C6 alkyl, or C1-C6 alkyl,
(k) C1-C6 thioalkyl, and
(l) —S(═NR^{E})(O)C1C6 alkyl.

27. The compound of any one of claims 1-22, wherein R^{B} is C1-C6 alkyl optionally substituted with 1-4 substituents independently selected from:
(h) 5-6 membered heteroaryl optionally substituted with C1-C6 alkyl, and
(j) 4-8 membered heterocyclyl optionally substituted with hydroxyl, —C(═O)C1-C6 alkyl, or C1-C6 alkyl.

28. The compound of any one of claims 1-27, wherein R^{C} is hydrogen.

29. The compound of any one of claims 1-27, wherein R^{C} is C1-C6 alkyl optionally substituted with oxo.

30. The compound of any one of claims 1-29, wherein R^{D} is hydrogen.

31. The compound of any one of claims 1-29, wherein R^{D} is C1-C6 alkyl optionally substituted with oxo.

32. The compound of any one of claims 1-31, wherein m is 1.

33. The compound of any one of claims 1-31, wherein m is 2.

34. The compound of any one of claims 1-31, wherein m is 3.

35. The compound of any one of claims 1-31, wherein m is 4.

36. The compound of any one of claims 1-6, wherein m is 0.

37. The compound of any one of claims 1-36, wherein R² is hydrogen.

38. The compound of any one of claims 1-36, wherein R² is halogen.

39. The compound of any one of claims 1-36, wherein R² is C1-C6 alkoxy.

40. The compound of any one of claims 1-36, wherein R² is C1-C6 haloalkoxy.

41. The compound of any one of claims 1-40, wherein R³ is hydrogen.

42. The compound of any one of claims 1-40, wherein R² is not hydrogen and R³ is not hydrogen.

43. The compound of any one of claims 1-40, wherein R³ is C1-C6 thioalkyl.

44. The compound of any one of claims 1-40, wherein R³ is —CO₂H.

45. The compound of any one of claims 1-40, wherein R³ is C1-C6 alkoxy optionally substituted with 4-10 membered heterocyclyl optionally substituted with C1-C6 alkoxy.

46. The compound of any one of claims 1-40, wherein R³ is 4-8 membered heterocyclyl optionally substituted with 1-2 substituents independently selected from halogen and C1-C6 alkyl.

47. The compound of any one of claims 1-40, wherein R³ is

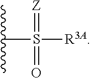

48. The compound of any one of claim 1-40 or 47, wherein R^{3A} is C1-C6 haloalkyl.

49. The compound of any one of claim 1-40 or 47, wherein R^{3A} is C3-C6 cycloalkyl.

50. The compound of any one of claim 1-40 or 47, wherein R^{3A} is C1-C6 alkyl optionally substituted with C3-C6 cycloalkyl.

51. The compound of any one of claim 1-40 or 47-50, wherein Z is NR⁴.

52. The compound of any one of claim 1-40 or 47-51, wherein R⁴ is hydrogen.

53. The compound of any one of claim 1-40 or 47-51, wherein R⁴ is C1-C6 alkyl.

54. The compound of any one of claim 1-40 or 47-50, wherein Z is O.

55. The compound of any one of claims 1-40, wherein R³ is

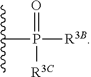

56. The compound of any one of claim 1-40 or 55, wherein R^{3B} is C3-C6 cycloalkyl.

57. The compound of any one of claim 1-40 or 55, wherein R^{3B} is or C1-C6 alkyl optionally substituted with C3-C6 cycloalkyl.

58. The compound of any one of claim 1-40 or 55-57, wherein R^{3C} is C3-C6 cycloalkyl.

59. The compound of any one of claim 1-40 or 55-57, wherein R^{3C} is or C1-C6 alkyl optionally substituted with C3-C6 cycloalkyl.

60. The compound of any one of claims 1-40, wherein R³ is C1-C6 alkyl optionally substituted with NR^{E}R^{F} or hydroxyl.

61. The compound of any one of claims 1-40, wherein R³ is —C(═O)NR^{E}R^{F}.

62. A compound selected from Examples 1-340, or a pharmaceutically acceptable salt of any of the foregoing.

63. A pharmaceutical composition comprising a compound of any one of claims 1-62, or a pharmaceutically acceptable salt thereof, and at least one pharmaceutically acceptable excipient.

64. A method of treating a RIPK2-associated disease or disorder in a subject in need thereof, comprising administering to the subject an effective amount of a compound of any one of claims 1-62, or a pharmaceutically acceptable salt thereof, or the pharmaceutical composition of claim 63.
